SEQUENCE LISTING (63 sequences)

SEQ ID NO: 1 - DNA, 27 bp, attB0
agcctgcttttttatactaacttgagc

SEQ ID NO: 2 - DNA, 27 bp, attP0
gttcagcttttttatactaagttggca

SEQ ID NO: 3 - DNA, 27 bp, attL0
agcctgcttttttatactaagttggca

SEQ ID NO: 4 - DNA, 27 bp, attR0
gttcagcttttttatactaacttgagc

SEQ ID NO: 5 - DNA, 25 bp, attB1
agcctgcttttttgtacaaacttgt

SEQ ID NO: 6 - DNA, 27 bp, attP1
gttcagcttttttgtacaaagttggca

SEQ ID NO: 7 - DNA, 27 bp, attL1
agcctgcttttttgtacaaagttggca

SEQ ID NO: 8 - DNA, 25 bp, attR1
gttcagcttttttgtacaaacttgt

SEQ ID NO: 9 - DNA, 25 bp, attB2
acccagctttcttgtacaaagtggt

SEQ ID NO: 10 - DNA, 27 bp, attP2
gttcagctttcttgtacaaagttggca

SEQ ID NO: 11 - DNA, 27 bp, attL2
acccagctttcttgtacaaagttggca

SEQ ID NO: 12 - DNA, 25 bp, attR2
gttcagctttcttgtacaaagtggt

SEQ ID NO: 13 - DNA, 22 bp, attB5
caactttattatacaaagttgt

SEQ ID NO: 14 - DNA, 27 bp, attP5
gttcaactttattatacaaagttggca

SEQ ID NO: 15 - DNA, 24 bp, attL5
caactttattatacaaagttggca

SEQ ID NO: 16 - DNA, 25 bp, attR5
gttcaactttattatacaaagttgt

SEQ ID NO: 17 - DNA, 22 bp, attB11
caacttttctatacaaagttgt

SEQ ID NO: 18 - DNA, 27 bp, attP11
gttcaacttttctatacaaagttggca

SEQ ID NO: 19 - DNA, 24 bp, attL11
caacttttctatacaaagttggca

SEQ ID NO: 20 - DNA, 25 bp, attR11
gttcaacttttctatacaaagttgt

SEQ ID NO: 21 - DNA, 22 bp, attB17
caacttttgtatacaaagttgt

SEQ ID NO: 22 - DNA, 27 bp, attP17
gttcaacttttgtatacaaagttggca

SEQ ID NO: 23 - DNA, 24 bp, attL17
caacttttgtatacaaagttggca

SEQ ID NO: 24 - DNA, 25 bp, attR17
gttcaacttttgtatacaaagttgt

SEQ ID NO: 25 - DNA, 22 bp, attB19
caactttttcgtacaaagttgt

SEQ ID NO: 26 - DNA, 27 bp, attP19
gttcaactttttcgtacaaagttggca

SEQ ID NO: 27 - DNA, 24 bp, attL19
caactttttcgtacaaagttggca

SEQ ID NO: 28 - DNA, 25 bp, attR19
gttcaactttttcgtacaaagttgt

SEQ ID NO: 29 - DNA, 22 bp, attB20
caactttttggtacaaagttgt

SEQ ID NO: 30 - DNA, 27 bp, attP20
gttcaactttttggtacaaagttggca

SEQ ID NO: 31 - DNA, 24 bp, attL20
caactttttggtacaaagttggca

SEQ ID NO: 32 - DNA, 25 bp, attR20
gttcaactttttggtacaaagttgt

SEQ ID NO: 33 - DNA, 22 bp, attB21
caactttttaatacaaagttgt

SEQ ID NO: 34 - DNA, 27 bp, attP21
gttcaactttttaatacaaagttggca

SEQ ID NO: 35 - DNA, 24 bp, attL21
caactttttaatacaaagttggca

SEQ ID NO: 36 - DNA, 25 bp, attR21
gttcaactttttaatacaaagttgt

SEQ ID NO: 37 - DNA, 2717 bp, Artificial sequence [sequence omitted]

SEQ ID NO: 38 - DNA, 6464 bp, Artificial sequence
  plasmid pDEST1
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggtccgtataatctgtggaattgtgagcgggataacaatttcatcgcgaggtaccaagctatcacaagtttgtacaaaaaagctgaacgagaaacgtaaaatgatataaatatcaatatattaaattagattttgcataaaaaacagactacataatactgtaaaacacaacatatccagtcactatggcggccgctaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgctatggtgcactctcagtacaatctgctctgatgccgcatagttaagccagtaccagtcacgtagcgatatcggagtgtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcacccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 39 - DNA, 5584 bp, Artificial sequence
  plasmid pDONR207
gcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctggctagcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagdtcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcttgcgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattagtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggcctaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccctgtattactgtttatgtaagcagacagttttattgttcatgatgatatatttttatcttgtgcaatgtaacatcagagattttgagacacgggccagagctgcagctggatggcaaataatgattttattttgactgatagtgacctgttcgttgcaacaaattgataagcaatgctttcttataatgccaactttgtacaagaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgactaagttggcagcatcacccgacgcactttgcgccgaataaatacctgtgacggaagatcacttcgcagaataaataaatcctggtgtccctgttgataccgggaagccctgggccaactttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatcgcgtggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgatacagtagaaattacagaaactttatcacgtttagtaagtatagaggctgaaaatccagatgaagccgaacgacttgtaagagaaaagtataagagttgtgaaattgttcttgatgcagatgattttcaggactatgacactagcgtatatgaataggtagatgtttttattttgtcacacaaaaaagaggctcgcacctctttttcttatttctttttatgatttaatacggcattgaggacaatagcgagtaggctggatacgacgattccgtttgagaagaacatttggaaggctgtcggtcgactaagttggcagcatcacccgaagaacatttggaaggctgtcggtcgactacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttggggcccgagatccatgctagcgttaac

SEQ ID NO: 40 - DNA, 4470 bp, Artificial sequence
  plasmid pDONR201
gttaacgctagcatggatctcgggccccaaataatgattttattttgactgatagtgacctgttcgttgcaacaaattgatgagcaatgcttttttataatgccaactttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgc

SEQ ID NO: 41 - DNA, 4428 bp, Artificial sequence
  plasmid pDONR212
cttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcaccagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatctcgggccccaaataatgattttattttgactgatagtgacctgttcgttgcaacaaattgatgagcaatgcttttttataatgccaactttgtacaaaaaagctgatatcgaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgttagaaaaactcatcgagcatcaaatgaaactgcaatttattcatatcaggattatcaataccatatttttgaaaaagccgtttctgtaatgaaggagaaaactcaccgaggcagttccataggatggcaagatcctgctatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcatagatcttttctccatcactgatagggagtggtaaaataactccatcaatgatagagtgtcaacaacatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactgg

SEQ ID NO: 42 - DNA, 4627 bp, Artificial sequence
  plasmid pDONR212(F)
atggatctcgggccccaaataatgattttattttgactgatagtgacctgttcgttgcaacaaattgatgagcaatgcttttttataatgccaactttgtacaaaaaagctgatatcgaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcatagatcttttctccatcactgatagggagtggtaaaataactccatcaatgatagagtgtcaacaacatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagc

SEQ ID NO: 43 - DNA, 4627 bp, Artificial sequence
  plasmid pDONR212(R)
atggatctcgggccccaaataatgattttattttgactgatagtgacctgttcgttgcaacaaattgatgagcaatgcttttttataatgccaactttgtacaaaaaagctgatatcgaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcatagatcttttctccatcactgatagggagtggtaaaataactccatcaatgatagagtgtcaacaacatgaccaaaatcccttaacgtgagttacgcgtattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagc

SEQ ID NO: 44 - DNA, 4396 bp, Artificial sequence
  attB cloning vector pCMVSPORT6.0
ccattcgccattcaggctgcgcaactgttgggaagggcgatcggtgcgggcctcttcgctattacgccagccaatacgcaaaccgcctctccccgcgcgttggccgattcattaatgcaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcactagctcctgtgtataatattttcataaatcatactcagtaagcaaaactctcaagcagcaagcatatgcagctagtttaacacattatacacttaaaaattttatatttaccttagagctttaaatctctgtaggtagtttgtccaattatgtcacaccacagaagtaaggttccttcacaaagatcccaagctagcagttttcccagtcacgacgttgtaaaacgacggccagtgcctagcttataatacgactcactatagggaccactttgtacaagaaagctgggtacgcgtaagcttgggcccctcgagggatcctctagagcggccgccgactagtgagctcgtcgacgatatcccgggaattccggaccggtaccagcctgcttttttgtacaaacttgttctatagtgtcacctaaataggcctaatggtcatagctgtttcctgtgtgaaattgttatccgctccgcggcctaggctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgacatgcatctaatgagtgaaagggcctcgtactacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaacgcgcgaattgcaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccataacttcgtatagcatacattatacgaagttatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgccaggggtgggcacacatatttgataccagcgatccctacacagcacataattcaatgcgacttccctctatcgcacatcttagacctttattctccctccagcacacatcgaagctgccgagcaagccgttctcaccagtccaagacctggcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 45 - DNA, 15 bp, Artificial sequence
  Fis binding site
gntyaawwwttranc

SEQ ID NO: 46 - DNA, 20 bp, Artificial sequence
  Fis binding site
agtctgttttttatgcaaaa

SEQ ID NO: 47 - DNA, 15 bp, Artificial sequence
  att core region
gcttttttatactaa

SEQ ID NO: 48 - DNA, 21 bp, Artificial sequence
  att functional core region
caacttttttatacaaagttg

SEQ ID NO: 49 - protein, 98 aa, Escherichia coli
  Fis protein
MFEQRVNSDVLTVSTVNSQDQVTQKPLRDSVKQALKNYFAQLNGQDVNDLYELVLAEVEQPLLDMVMAYTRGNQTRAALMMGINRGTLRKKLKKYGMN

SEQ ID NO: 50 - protein, 93 aa, Klebsiella pneumoniae
  Fis protein
MFEQRVNSDVLTVSTVNSQDQVTQKPLRDSVKQALKNYFAQLNGQDVNDLYELVLAEVEQPLLDMVMQYTRGNQTRAALMMGINRGTLRKKLK

SEQ ID NO: 51 - protein, 98 aa, Vibrio cholera
  Fis protein
MFEQNLTSEALTVTTVTSQDQITQKPLRDSVKASLKNYLAQLNGQEVTELYELVLAEVEQPLLDTIMQYTRGNQTRAATMMGINRGTLRKKLKKYGMN

SEQ ID NO: 52 - protein, 99 aa, Haemophilus influenzae
  Fis protein
MLEQQRNSADALTVSVLNAQSQVTSKPLRDSVKQALRNYLAQLDGQDVNDLYELVLAEVEHPMLDMIMQYTRGNQTRAANMLGINRGTLRKKLKKYGMG

SEQ ID NO: 53 - protein, 107 aa, Pseudomonas aeruginosa
  Fis protein
MTTMTTETLVSGTTPVSDNANLKQHLTTPTQEGQTLRDSVEKALHNYFAHLEGQPVTDVYNMVLCEVEAPLLETVMNHVKGNQTKASELLGLNRGTLRKKLKQYDLL

SEQ ID NO: 54 - protein, 98 aa, Salmonella typhimurium [sequence omitted]

SEQ ID NO: 55 - DNA, 37 bp, Artificial sequence
  DE09 oligonucleotide
gggggctgcaggcaagaagacaaaaatcaccttgcgc

SEQ ID NO: 56 - DNA, 37 bp, Artificial sequence
  DE10 oligonucleotide
gggggcccgggcagaggcagggagtgggacaaaattg

SEQ ID NO: 57 - DNA, 51 bp, Artificial sequence
  DE46 oligonucleotide
ggagggaattcaggaggtataaattaatgttcgaacaacgcgtaaattctg

SEQ ID NO: 58 - DNA, 29 bp, Artificial sequence
  DE49 oligonucleotide
ggaggggatccttattagttcatgccgta

SEQ ID NO: 59 - DNA, 48 bp, Artificial sequence
  DE162 oligonucleotide
ggaaggagatcttgctcaaaatttgagctacataatactgtaaaacac

SEQ ID NO: 60 - DNA, 113 bp, Artificial sequence
  plasmid pENTR1A
actttgtacaaaaaagcaggctttaaaggaaccaattcagtcgactggatccggtaccgaattcgaattcgcggccgcactcgagatatctagacccagctttcttgtacaaa

SEQ ID NO: 61 - protein, 21 aa, Unknown
  vector pENTR1A
TLYKKAGFKGTNSVDWIRYRI

SEQ ID NO: 62 - DNA, 120 bp, Artificial sequence
  plasmid pDEST1
atgagctgttgacaattaatcatccggctcgtataatgtgtggaattgtgagcggataacaatttcacacaggaaacagacaggtataggatcacaagtttgtacaaaaaagctgaacga

SEQ ID NO: 63 - DNA, 405 bp, Artificial sequence
  attB cloning vector pCMVSPORT6.0
agagctcgtttagtgaaccgtcagatcgcctggagacgccatccacgctgttttgacctccatagaagacaccgggaccgatccagcctccggactctagcctaggccgcggagcggataacaatttcacacaggaaacagctatgaccattaggcctatttaggtgacactatagaacaagtttgtacaaaaaagcaggctggtaccggtccggaattcccgggatatcgtcgacgacgtcactagtcggcggccgctctagagtatccctcgaggggcccaagcttacgcgtacccagctttcttgtacaaagtggtccctatagtgagtcgtattgtaagctaggcactggccgtcgttttacaacgtcgtgactgggaaaactgctagcttgggatctttg

(19) United States
(12) Patent Application Publication
(10) Pub. No.: US 2006/0035272 A1
(43) Pub. Date: Feb. 16, 2006

(54) Compositions and methods for use in isolation of nucleic acid molecules

(75) Inventors: Michael A. Brasch, Gaithersburg, MD (US); David Cheo, Kensington, MD (US); Xiao Li, Germantown, MD (US); Dominic Esposito, Columbia, MD (US); Devon R.N. Byrd, Fredericksburg, VA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation, Carlsbad, CA (US)

(21) Appl. No.: 11/251,821

(22) Filed: Oct. 18, 2005

Related U.S. Application Data
(63) Continuation of application No. 10/151,690, filed on May 21, 2002, now abandoned.
(63) Continuation-in-part of application No. 09/907,719, filed on Jul. 19, 2001.
Application No. 09/907,719 is a division of application No. 09/177,387, filed on Oct. 23, 1998, now abandoned.
(63) Continuation-in-part of application No. 10/640,422, filed on Aug. 14, 2003.
(63) Continuation-in-part of application No. 09/732,914, filed on Dec. 11, 2000.
(60) Provisional application No. 60/291,973, filed on May 21, 2001.
(60) Provisional application No. 60/065,930, filed on Oct. 24, 1997.
(60) Provisional application No. 60/402,920, filed on Aug. 14, 2002.
(60) Provisional application No. 60/169,983, filed on Dec. 10, 1999.
(60) Provisional application No. 60/188,020, filed on Mar. 9, 2000.

(51) Int. Cl. C12Q 1/68; C12N 15/63; C40B 40/08

(52) U.S. Cl. 435/6; 435/455

(57) ABSTRACT

The present invention relates generally to recombinant genetic technology. More particularly, the present invention relates to compositions and methods for use in selection and isolation of nucleic acid molecules. The invention further relates to methods for the preparation of individual nucleic acid molecules and populations of nucleic acid molecules, as well as nucleic acid molecules produced by these methods. The invention also relates to screening and/or selection methods for identifying and/or isolating nucleic acid molecules which have one or more common features (e.g., characteristics, activities, etc) and populations of nucleic acid molecules which share one or more features.

### CROSS-REFERENCE TO RELATED APPLICATIONS

The present application is a continuation of U.S. application Ser. No. 10/151,690, filed May 21, 2002, which claims the benefit of the filing date of U.S. Provisional Application No. 60/291,973, filed May 21, 2001. The present application is also a continuation-in-part of, and claims the benefit under 35 U.S.C. § 120 of, U.S. application Ser. No. 09/907,719, filed Jul. 19, 2001, which is a Divisional of U.S. application Ser. No. 09/177,387 (Abandoned), filed Oct. 23, 1998, which claims the benefit of the filing date of U.S. Provisional Application No. 60/065,930, filed Oct. 24, 1997. The present application is also a continuation-in-part of, and claims the benefit under 35 U.S.C. § 120 of, U.S. application Ser. No. 10/640,422, filed Aug. 14, 2003, which claims the benefit of the filing date of U.S. Provisional Application No. 60/402,920, filed Aug. 14, 2002. The present application is also a continuation-in-part of, and claims the benefit under 35 U.S.C. § 120 of, U.S. application Ser. No. 09/732,914, filed Dec. 11, 2000, which claims the benefit of the filing dates of U.S. Provisional Application Nos. 60/169,983, filed Dec. 10, 1999, and 60/188,020, filed Mar. 9, 2000. The disclosures of all of these referenced applications are incorporated herein by reference in their entireties.

### BACKGROUND OF THE INVENTION

1. Field of the Invention

The present invention relates generally to recombinant genetic technology. More particularly, the present invention relates to compositions and methods for use in selection and isolation of nucleic acid molecules. The invention further relates to methods for the preparation of individual nucleic acid molecules and populations of nucleic acid molecules, as well as nucleic acid molecules produced by these methods. The invention also relates to screening and/or selection methods for identifying and/or isolating nucleic acid molecules which have one or more common features (e.g., characteristics, activities, etc.) and populations of nucleic acid molecules which share one or more features.

2. Related Art

Site-specific recombinases. Site-specific recombinases are proteins that are present in many organisms (e.g., viruses and bacteria) and have been characterized to have both endonuclease and ligase properties. These recombinases (along with associated proteins in some cases) recognize specific sequences of bases in DNA and exchange the DNA segments flanking those segments. The recombinases and associated proteins are collectively referred to as “recombination proteins”. See, e.g., Landy, A., Current Opinion in Biotechnology 3:699-707 (1993).

Numerous recombination systems from various organisms have been described. See, e.g., Hoess et al., Nucleic Acids Research 14(6):2287 (1986); Abremski et al., J. Biol. Chem. 261:391 (1986); Campbell, J. Bacteriol. 174(23):7495 (1992); Qian et al., J. Biol. Chem. 267:7794 (1992); Araki et al., J. Mol. Biol. 225:25 (1992); Maeser and Kahnmann Mol. Gen. Genet. 230:170-176 (1991); Esposito et al., Nucl. Acids Res. 25:3605 (1997). Many of these belong to the integrase family of recombinases (Argos et al. EMBO J. 5:433-440 (1986); Voziyanov et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2μ circle plasmid (Broach et al. Cell 29:227-234 (1982)).

Backman (U.S. Pat. No. 4,673,640) discloses the in vivo use of λ recombinase to recombine a protein producing DNA segment by enzymatic site-specific recombination using wild-type recombination sites attB and attP.

Hasan and Szybalski (Gene 56:145-151 (1987)) disclose the use of λ Int recombinase in vivo for intramolecular recombination between wild-type attP and attB sites which flank a promoter. Because the orientations of these sites are inverted relative to each other, this causes an irreversible flipping of the promoter region relative to the gene of interest.

Palazzolo et al. (Gene 88:25-36 (1990)) disclose phage lambda vectors having bacteriophage λ arms that contain restriction sites positioned outside a cloned DNA sequence and between wild-type loxP sites. Infection of Escherchia coli cells that express the Cre recombinase with these phage vectors results in recombination between the loxP sites and the in vivo excision of the plasmid replicon, including the cloned cDNA.

Pósfai et al. (Nucl. Acids Res. 22:2392-2398 (1994)) disclose a method for inserting into genomic DNA partial expression vectors having a selectable marker, flanked by two wild-type FRT recognition sequences. FLP site-specific recombinase as present in the cells is used to integrate the vectors into the genome at predetermined sites. Under conditions where the replicon is functional, this cloned genomic DNA can be amplified.

Bebee et al. (U.S. Pat. No. 5,434,066) disclose the use of site-specific recombinases such as Cre for DNA containing two loxP sites for in vivo recombination between the sites.

Boyd (Nucl. Acids Res. 21:817-821 (1993)) discloses a method to facilitate the cloning of blunt-ended DNA using conditions that encourage intermolecular ligation to a dephosphorylated vector that contains a wild-type loxP site acted upon by a Cre site-specific recombinase present in Escherchia coli host cells.

Waterhouse et al. (WO 93/19172 and Nucleic Acids Res. 21:2265 (1993)) disclose an in vivo method where light and heavy chains of a particular antibody were cloned in different phage vectors between loxP and loxP511 sites and used to transfect new E. coli cells. Cre, acting in the host cells on the two parental molecules (one plasmid, one phage), produced four products in equilibrium: two different cointegrates (produced by recombination at either loxP or loxP511 sites), and two daughter molecules, one of which was the desired product.

Schlake & Bode (Biochemistry 33:12746-12751 (1994)) disclose an in vivo method to exchange expression cassettes at defined chromosomal locations, each flanked by a wild-type and a spacer-mutated FRT recombination site. A double-reciprocal crossover was mediated in cultured mammalian cells by using this FLP/FRT system for site-specific recombination.

Hartley et al. (U.S. Pat. No. 5,888,732) disclose compositions and methods for recombinational exchange of nucleic acid segments and molecules, including for use in recombinational cloning of a variety of nucleic acid molecules in vitro and in vivo, using a variety of wild-type and/or mutated recombination sites and recombination proteins.

Transposases. The family of enzymes, the transposases, has also been used to transfer genetic information between replicons. Transposons are structurally variable, being described as simple or compound, but typically encode a transposase gene flanked by DNA sequences organized in inverted orientations. Integration of transposons can be random or highly specific. Representative transposons such as Tn7, which are highly site-specific, have been applied to the in vivo movement of DNA segments between replicons (Lucklow et al., J. Virol. 67:4566-4579 (1993)).

Devine and Boeke (Nucl. Acids Res. 22:3765-3772 (1994)), disclose the construction of artificial transposons for the insertion of DNA segments, in vitro, into recipient DNA molecules. The system makes use of the integrase of yeast TY1 virus-like particles. The DNA segment of interest is cloned, using standard methods, between the ends of the transposon-like element TY1. In the presence of the TY1 integrase, the resulting element integrates randomly into a second target DNA molecule.

Recombination Sites. Also key to the integration/recombination reactions mediated by the above-noted recombination proteins and/or transposases are recognition sequences, often termed “recombination sites,” on the DNA molecules participating in the integration/recombination reactions. These recombination sites are discrete sections or segments of DNA on the participating nucleic acid molecules that are recognized and bound by the recombination proteins during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. See FIG. 1 of Sauer, B., Curr. Opin. Biotech. 5:521-527 (1994). Other examples of recognition sequences include the attB, attP, attL, and attR sequences which are recognized by the recombination protein λ Int. AttB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), Fis and excisionase (Xis). See Landy, Curr. Opin. Biotech. 3:699-707 (1993); see also U.S. Pat. No. 5,888,732, which is incorporated by reference herein.

Stop Codons and Suppressor tRNAs. Three codons are used by both eukaryotes and prokaryotes to signal the end of gene. When transcribed into mRNA, the codons have the following sequences: UAG (amber), UGA (opal) and UAA (ochre). Under most circumstances, the cell does not contain any tRNA molecules that recognize these codons. Thus, when a ribosome translating an mRNA reaches one of these codons, the ribosome stalls and falls of the RNA, terminating translation of the mRNA. The release of the ribosome from the mRNA is mediated by specific factors (see S. Mottagui-Tabar, NAR 26(11), 2789, 1998). A gene with an in-frame stop codon (TAA, TAG, or TGA) will ordinarily encode a protein with a native carboxy terminus. However, suppressor tRNAs, can result in the insertion of amino acids and continuation of translation past stop codons.

Mutant tRNA molecules that recognize what are ordinarily stop codons suppress the termination of translation of an mRNA molecule and are termed suppressor tRNAs. A number of such suppressor tRNAs have been found. Examples include, but are not limited to, the supE, supP, supD, supF and supZ suppressors which suppress the termination of translation of the amber stop codon, supB, g/T, supL, supN, supC and supM suppressors which suppress the function of the ochre stop codon and glyT, trpT and Su-9 which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. For a more detailed discussion of suppressor tRNAs, the reader may consult Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.

DNA cloning. The cloning of DNA segments occurs as a daily routine in many research labs and as a prerequisite step in many genetic analyses. While the purpose of these clonings varies, two general purposes can be considered: (1) the initial cloning of DNA from large DNA or RNA segments (chromosomes, YACs, PCR fragments, mRNA, etc.), done in a relative handful of known vectors such as pUC, pGem, pBlueScript, and (2) the subcloning of these DNA segments into specialized vectors for functional analysis. A great deal of time and effort is expended in the transfer of DNA segments from the initial cloning vectors to the more specialized vectors. This transfer is called subcloning.

The basic methods for cloning have been known for many years and have changed little during that time. A typical cloning protocol is as follows:
(1) digest the DNA of interest with one or two restriction enzymes;
(2) gel purify the DNA segment of interest when known;
(3) prepare the vector by cutting with appropriate restriction enzymes, treating with alkaline phosphatase, gel purify etc., as appropriate;
(4) ligate the DNA segment to the vector, with appropriate controls to eliminate background of uncut and self-ligated vector;
(5) introduce the resulting vector into an Escherchia coli host cell;
(6) pick selected colonies and grow small cultures overnight;
(7) make DNA minipreps; and
(8) analyze the isolated plasmid on agarose gels (often after diagnostic restriction enzyme digestions) or by PCR.

Specialized vectors used for subcloning DNA segments are generally functionally diverse. These include, but are not limited to, vectors for expressing nucleic acid molecules in various organisms, vectors for regulating nucleic acid molecule expression, vectors for providing tags to aid in protein purification or to allow tracking of proteins in cells, vectors for modifying the cloned DNA segment (e.g., generating deletions), vectors for the synthesis of probes (e.g., riboprobes), vectors for the preparation of templates for DNA sequencing, vectors for the identification of protein coding regions, vectors for the fusion of various protein-coding regions, vectors designed to provide large amounts of the DNA of interest, etc. It is common that a particular investigation will involve subcloning the DNA segment of interest into several different specialized vectors.

Subcloning is a particularly time consuming process when multiple selection criteria are used sequentially to select subpopulations of DNA molecules. Because vector backbones can impart a large variety of functions upon the nucleic acid molecules being analyzed, nucleic acid molecules of interest within a population or subpopulation can be identified based on these properties. These populations of nucleic acid molecules can then be isolated and transferred into one or more subsequent vectors which impose additional sets of conditions that can be used for selection of additional subpopulations. By this reiterative process of sequential selections and transfers, populations or subpopulations possessing one or more predefined sets of properties, features, or activities can be separated, selected, identified and/or isolated. One of the major problems confronted when using this approach is the need to constantly subclone the selected populations into new vectors for additional selections.

As known in the art, simple subclonings (e.g., subclonings in which the nucleic acid molecule is not large and the restriction sites are compatible with those of the subcloning vector) can be done in one day. However, complex subclonings can take several weeks, especially those involving unknown sequences, long fragments, toxic genes, unsuitable placement of restriction sites, high backgrounds, impure enzymes, etc. Subcloning of nucleic acid molecules is thus often viewed as a chore to be done as few times as possible.

Several methods for facilitating the cloning of nucleic acid molecules have been described, e.g., as in the following references.

Ferguson, J. et al. (Gene 16:191 (1981)), disclose a family of vectors for subcloning fragments of yeast DNA. The vectors encode kanamycin resistance. Clones of longer yeast DNA segments can be partially digested and ligated into the subcloning vectors. If the original cloning vector conveys resistance to ampicillin, no purification is necessary prior to transformation, since the selection will be for kanamycin.

Hashimoto-Gotoh, T. et al. (Gene 41:125 (1986)), disclose a subcloning vector with unique cloning sites within a streptomycin sensitivity gene; in a streptomycin-resistant host, only plasmids with insertions or deletions in the dominant sensitivity gene will survive streptomycin selection.

Accordingly, traditional subcloning methods using restriction enzymes and ligase are time consuming and relatively unreliable. Considerable labor is expended, and if two or more days later the desired subclone cannot be found among the candidate plasmids, the entire process must then be repeated using alternative conditions.

Although site specific recombinases have been used to recombine DNA in vivo, the successful use of such enzymes in vitro was expected to suffer from several problems. For example, the site specificities and efficiencies were expected to differ in vitro; topologically linked products were expected; and the topology of the DNA substrates and recombination proteins was expected to differ significantly in vitro (see, e.g., Adams et al, J. Mol. Biol. 226:661-73 (1992)). Reactions that could go on for many hours in vivo were expected to occur in significantly less time in vitro before the enzymes became inactive. In addition, the stabilities of the recombination enzymes after incubation for extended periods of time in in vitro reactions was unknown, as were the effects of the topologies (i.e., linear, coiled, supercoiled, etc.) of the nucleic acid molecules involved in the reaction. Multiple DNA recombination products were expected in the biological host used, resulting in unsatisfactory reliability, specificity or efficiency of subcloning. Thus, in vitro recombination reactions were not expected to be sufficiently efficient to yield the desired levels of product.

Recombinational Cloning. Cloning systems that utilize recombination at defined recombination sites have been previously described in U.S. Pat. Nos. 5,888,732 and 6,143,557 and the following related applications: U.S. application Ser. No. 09/177,387, filed Oct. 23, 1998; U.S. application Ser. No. 09/517,466, filed Mar. 2, 2000; and U.S. application Ser. No. 09/732,914, filed Dec. 11, 2000, all of which are specifically incorporated herein by reference. In brief, the GATEWAY™ Cloning System, described in this application and the patents and applications referred to immediately above, utilizes vectors that contain at least one recombination site to clone desired nucleic acid molecules in vivo or in vitro. More specifically, the system utilizes vectors that contain one or more site-specific recombination sites based on the bacteriophage lambda system (e.g., att1 and att2) which is/are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY™ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms, such as thymidine kinase (TK) in mammalian and insect cells.

Mutating specific residues in the core region of the att site can generate a large number of different att sites. As with the att1 and att2 sites utilized in GATEWAY™, each additional mutation potentially creates a novel att site with unique specificity that will recombine only with its cognate partner att site bearing the same mutation and will not cross-react with any other mutant or wild-type att site. Novel mutated att sites (e.g., attB1-10, attP1-10, attR1-10 and attL1-10) are described in previous patent application Ser. No. 09/517,466, filed Mar. 2, 2000, which is specifically incorporated herein by reference.

Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives. Such recombination sites may be used to join or link multiple nucleic acid molecules or segments and more specifically to clone such multiple segments (e.g., two, three, four, five, seven, ten, twelve, fifteen, twenty, thirty, fifty, seventy-five, one hundred, two hundred, etc.) into one or more vectors (e.g., two, three, four, five, seven, ten, twelve, etc.) containing one or more recombination sites (e.g., two, three, four, five, seven, ten, twelve, fifteen, twenty, thirty, fifty, seventy-five, one hundred, two hundred, etc.), such as any GATEWAY™ Vector including Destination Vectors.

Selection. Selection is one of the most common methods used to obtain nucleic acid molecules with desired or predefined properties, features, or activities. When a nucleic acid molecule of interest is cloned into a vector, the vector can provide the nucleic acid molecule of interest with particular structural and/or functional characteristics (e.g., altered expression levels, additional nucleotide sequences, etc.). Similarly, insertion of a nucleic acid molecule into a vector can alter the characteristics of the vector. These altered characteristics can be used to select or identify nucleic acid molecules in a more complex population or subpopulation of nucleic acid molecules. Once a subpopulation has been selected or identified it is often necessary to repeat the process in a different vector which provides a different property, feature, or activity to be used in selection, separation, or identification. The change from one vector to a different vector is generally accomplished using standard cloning techniques described above. However, when many rounds of selection are utilized, or a large population of nucleic acids is involved, traditional cloning techniques can be inefficient, tedious and expensive. Further, mistakes in the cloning process can lead to the complete loss of selected or isolated nucleic acid molecules, or populations or subpopulations thereof, thereby wasting the time and expense used to select or isolate them.

Accordingly, there is a long felt need to provide alternative methods for isolating and manipulating populations, subpopulations or libraries of nucleic acid molecules that provide advantages over the known use of restriction enzymes and ligases.

### SUMMARY OF THE INVENTION

The invention relates to methods for the preparation of individual nucleic acid molecules and populations of nucleic acid molecules, as well as nucleic acid molecules produced by these methods. The invention also relates to screening and/or selection methods for identifying and/or isolating nucleic acid molecules which have one or more common features (e.g., characteristics, activities, etc.) and populations of nucleic acid molecules which share one or more features.

The invention also relates to methods involving the insertion or transfer (in vivo or in vitro) of one or more populations of nucleic acid molecules into one or more target nucleic acid molecules by recombinational cloning to generate new populations of nucleic acid molecules. The nucleic acid molecules inserted or transferred into target nucleic acid molecules, as described above, may then be inserted or transferred to one or more new or different target nucleic acid molecules. Further, at each or any step in the process described above, one nucleic acid molecule or a population or subpopulation of nucleic acid molecules may be screened or selected to identify one or more characteristics or activities present or conferred by either the nucleic acid insert and/or by the target nucleic acid molecule.

In one aspect, the invention relates to the transfer of some or all of a population of nucleic acid molecules by recombinational cloning (in vivo or in vitro) into one or more desired target nucleic acid molecules. Preferably, the population or subpopulation of molecules to be transferred comprise one or more recombination sites and the target nucleic acid molecules comprise one or more recombination sites and the transfer is accomplished by recombination of at least one recombination site on each of such molecules. Such recombination preferably accomplished in the presence of at least one recombination protein. Moreover, such transfer of a population or subpopulation of molecules by recombination into new or different target molecules may be done any number of times in accordance with the invention.

In a more specific aspect, the invention relates, in part, to methods for inserting or transferring a population of nucleic acid molecules into one or more second target molecules (e.g., target molecules which are the same or different), these methods comprise:
(a) mixing at least a first population of nucleic acid molecules comprising one or more recombination sites with at least one first target nucleic acid molecule comprising one or more recombination sites;
(b) causing some or all of the nucleic acid molecules of the at least first population to recombine with some or all of the first target nucleic acid molecules, thereby forming a second population of nucleic acid molecules;
(c) mixing at least the second population of nucleic acid molecules with at least one second target nucleic acid molecule comprising one or more recombination sites; and
(d) causing some or all of the nucleic acid molecules of the at least second population to recombine with some or all of the second target nucleic acid molecules, thereby forming a third population of nucleic acid molecules.

In related aspects, the recombination in step (b) or (d) above is caused by mixing the first population of nucleic acid molecules and the first target nucleic acid molecule with one or more recombination proteins under conditions which favor the recombination.

In additional related aspects, the one or more recombination proteins comprise one or more proteins selected from the group consisting of:
(a) Cre;
(b) Int;
(c) IHF;
(d) Xis;
(e) Hin;
(f) Gin;
(g) Cin;
(h) Tn3 resolvase;
(i) TndX;
(j) XerC; and
(k) XerD.

In yet other related aspects, the one or more recombination proteins are in admixture with at least one second protein which (1) has a molecular weight below about 14,000 daltons, (2) contains at least 15% basic amino acid residues, and (3) enhances recombination.

In certain related aspects, the one or more second proteins comprises Fis, a ribosomomal protein, or a fragment of either Fis or a ribosomomal protein. Further, the ribosomal protein may be a prokaryotic ribosomal protein (e.g., a ribosomal protein selected from the group of Escherchia coli ribosomal proteins S10, S14, S15, S16, S17, S18, S19, S20, S21, L14, L21, L23, L24, L25, L27, L28, L29, L30, L31, L32, L33 and L34).

In additional related aspects, some or all members of the population of nucleic acid molecules (e.g., the first population of nucleic acid molecules) comprises a synthetic library, a cDNA library, a genomic library, a library which encodes peptides, or a combination of these libraries. The library may also be a normalized library.

In other related aspects, some or all of the target nucleic acid molecules (e.g., the first or second target nucleic acid molecules), some or all of the individual members of the population of nucleic acid molecules (e.g., the first or second population of nucleic acid molecules), or both the target nucleic acid molecules and the individual members of the population of nucleic acid molecules are linear nucleic acid molecules. In any event, such molecules may generally be in any form including linear, circular, supercoiled, etc.

In yet other related aspects, some or all of the target nucleic acid molecules and/or some or all of the individual members of the population of nucleic acid molecules comprise (1) at least two recombination sites or (2) at least one recombination site and at least one restriction endonuclease site, at least one topoisomerase cloning site, at least one site for homologous recombination, or at least one other site which can be ligated to another nucleic acid molecule. In another aspect, all or at least some portion of such target molecules and/or such populations are flanked by (1) at least two recombination sites or (2) at least one recombination site and at least one restriction endonuclease site, at least one topoisomerase cloning site, at least one site for homologous recombination, or at least one other site which can be ligated to another nucleic acid molecule.

In additional related aspects, the individual members of the first population of nucleic acid molecules are flanked by one recombination site and one restriction endonuclease site.

In specific embodiments, recombination sites of molecules used in methods of the invention may comprise one or more recombination sites selected from the group consisting of:
(a) lox sites;
(b) psi sites;
(c) dif sites;
(d) cer sites;
(e) frt sites;
(f) att sites; and
(g) mutants, variants, and derivatives of the recombination sites of (a), (b), (c), (d), (e), or (f) which retain the ability to undergo recombination.

In related embodiments, recombination sites of molecules used in methods of the invention may comprise att sites having identical seven base pair overlap regions. In more specific embodiments, the first three nucleotides of the seven base pair overlap regions of these recombination sites may comprise nucleotide sequences selected from the group consisting of:
(a) AAA;
(b) AAC;
(c) AAG;
(d) AAT;
(e) ACA;
(f) ACC;
(g) ACG;
(h) ACT;
(i) AGA;
(j) AGC;
(k) AGG;
(l) AGT;
(m) ATA;
(n) ATC;
(o) ATG; and
(p) ATT.

In additional specific embodiments, the first three nucleotides of the seven base pair overlap regions of these recombination sites may comprise nucleotide sequences selected from the group consisting of:
(a) CAA;
(b) CAC;
(c) CAG;
(d) CAT;
(e) CCA;
(f) CCC;
(g) CCG;
(h) CCT;
(i) CGA;
(j) CGC;
(k) CGG;
(l) CGT;
(m) CTA;
(n) CTC;
(o) CTG; and
(p) CTT.

In additional specific embodiments, the first three nucleotides of the seven base pair overlap regions of these recombination sites may comprise nucleotide sequences selected from the group consisting of:
(a) GAA;
(b) GAC;
(c) GAG;
(d) GAT;
(e) GCA;
(f) GCC;
(g) GCG;
(h) GCT;
(i) GGA;
(j) GGC;
(k) GGG;
(l) GGT;
(m) GTA;
(n) GTC;
(o) GTG; and
(p) GTT.

In additional specific embodiments, the first three nucleotides of the seven base pair overlap regions of these recombination sites may comprise nucleotide sequences selected from the group consisting of:
(a) TAA;
(b) TAC;
(c) TAG;
(d) TAT;
(e) TCA;
(f) TCC;
(g) TCG;
(h) TCT;
(i) TGA;
(j) TGC;
(k) TGG;
(l) TGT;
(m) TTA;
(n) TTC;
(o) TTG; and
(p) TTT.

In specific embodiments, some or all of the target nucleic acid molecules (e.g., the first or second target nucleic acid molecule) are vectors (e.g., a vector selected from the group consisting of pDONR201, pDONR212, pDONR212(F), pDONR212(R), pDONR205 and pDONR207). In another aspect, some or all of the members of the population of molecules are vectors.

In additional specific embodiments, populations of nucleic acid molecules (e.g., cDNA molecules) may be prepared so that the individual members of these populations have at least one recombination site (e.g., attL sites) at one or both termini. In one specific aspect, such recombination sites are attL sites or mutants, variants, or derivatives thereof. Further, these attL sites (or mutants, variants, or derivatives thereof) may be positioned so that, upon recombination with attR sites (or mutants, variants, or derivatives thereof), the individual members of the populations have attB sites (or mutants, variants, or derivatives thereof) at one or both termini. Thus, the invention includes the construction of populations of nucleic acid molecules (e.g., cDNA molecules) which contain attL sites (or mutants, variants, or derivatives thereof) at at least one terminus. Such populations of nucleic acid molecules may be inserted directly into vectors to generate expression clones.

The invention also provides populations of nucleic acid molecules prepared by the above methods, as well as compositions comprising these nucleic acid molecules, individual members of these populations of molecules, populations of host cells (e.g., prokaryotic or eukaryotic cells) which comprise these populations, and individual host cells (e.g., individual bacterial cells such as E. coli cells or individual eukaryotic cells such as yeast cells, plant cells, or animal cells) of these populations.

The invention further provides methods for identifying one or more nucleic acid molecules having at least one specific property, feature, or activity, these methods comprise:
(a) mixing at least a first population of nucleic acid molecules comprising one or more recombination sites with at least one first target nucleic acid molecule comprising one or more recombination sites;
(b) causing some or all of the nucleic acid molecules of the at least first population to recombine with some or all of the first target nucleic acid molecules, thereby forming a second population of nucleic acid molecules;
(c) separating, identifying or selecting one or more nucleic acid molecules or a subpopulation of the second population which have at least one specific property, activity, or feature different from other members of the second population, thereby generating a third population of nucleic acid molecules which share the at least one specific property, activity, or feature, and optionally;
(d) mixing at least the third population of nucleic acid molecules with at least one second target nucleic acid molecule comprising one or more recombination sites;
(e) causing some or all of the nucleic acid molecules of the at least third population to recombine with some or all of the second target nucleic acid molecules, thereby forming a fourth population of nucleic acid molecules; and
(f) separating, identifying or selecting one or more nucleic acid molecules or a subpopulation of the fourth population which have at least one specific property, activity, or feature different from other members of the fourth population, thereby generating a fifth population of nucleic acid molecules which share the at least one specific property, activity, or feature.

Further, steps (a)-(c) and/or (d)-(f) above may be repeated any number of times. Thus, according to the invention, single or multiple rounds of recombination and selection or identification may be accomplished to obtain one or a number of molecule having one or multiple desired properties, activities, or features. The invention therefore provides a powerful and efficient tool to isolate and identify selected members from a population.

In related aspects, the at least one specific property, feature, or activity identified according to the invention may be either the same or different properties, features, or activities. Further, the at least one specific property, feature, or activity may not be properties, features, or activities of expression products of individual members any of the selected, identified, or separated members or other molecules present in populations of nucleic acid molecules (e.g., the at least one specific property, feature, or activity may be a property, feature, or activity of a target nucleic acid molecule). In addition, the at least one specific property, feature, or activity may be, but is not limited to, a properties, features, or activities selected from the group consisting of:
(a) the ability to hybridize intramolecularly (e.g., to form intramolecular “secondary” structures) or to another nucleic acid molecule under stringent hybridization conditions;
(b) the ability to activate transcription;
(c) the ability to bind proteins;
(d) the ability to initiate replication of nucleic acid molecules;
(e) the ability to segregate nucleic acid molecules during cell division;
(f) the ability to direct the packaging of nucleic acid molecules into viral particles;
(g) the ability to be cleaved by one or more restriction endonucleases;
(i) the ability to be joined to another nucleic acid molecule by topoisomerase (e.g., by topoisomerase cloning);
(j) the ability to be ligated to another nucleic acid molecule;
(k) the ability to recombine with another nucleic acid molecule by homologous recombination;
(l) the ability to anneal to another nucleic acid molecule; and
(m) the ability to recombine with another nucleic acid molecule by site specific recombination.

In additional related aspects, the at least one specific property, feature, or activity may be properties, features, or activities of encoded expression products. For example, the at least one specific property, feature, or activity may be properties, features, or activities selected from the group consisting of:
(a) ribozyme activity;
(b) tRNA activity;
(c) antisense activity;
(d) being encoded by nucleic acid which is in-frame with nucleic acid that encodes another polypeptide;
(e) the ability to induce an immunological response;
(f) having binding affinity for a particular ligand;
(g) the ability to target a protein to a particular location in a cell;
(h) the ability to undergo proteolytic cleavage; and
(i) the ability to undergo post-translational modification.

The invention also provides methods for identifying one or more nucleic acid molecules having at least one specific property, feature, or activity, these methods comprise:
(a) providing a first population of nucleic acid molecules comprising one or more recombination sites;
(b) separating, identifying, or selecting two or more nucleic acid molecules of the first population which have at least one specific property, feature, or activity different from other nucleic acid molecules in the population, thereby generating at least one a second population of nucleic acid molecules which share the at least one specific property, feature, or activity;
(c) mixing at least the second population of nucleic acid molecules with at least one target nucleic acid molecule comprising one or more recombination sites;
(d) causing some or all of the nucleic acid molecules of the at least second population to recombine with some or all of the target nucleic acid molecules, thereby forming a third population of nucleic acid molecules; and
(e) separating, identifying or selecting one or more nucleic acid molecules of the third population which have at least one specific property, feature, or activity different from other nucleic acid molecules in the population.

The invention additionally provides methods for identifying one or more nucleic acid molecules having at least one specific property, feature, or activity which can be detected by in vitro screening, these methods comprise:
(a) mixing at least a first population of nucleic acid molecules comprising one or more recombination sites with at least one first target nucleic acid molecule comprising one or more recombination sites;
(b) causing some or all of the nucleic acid molecules of the at least first population to recombine with some or all of the first target nucleic acid molecules, thereby forming a second population of nucleic acid molecules; and
(c) separating, identifying or selecting one or more nucleic acid molecules of the second population which have at least one specific property, feature, or activity different from other members of the population, thereby generating a third population of nucleic acid molecules which share the at least one specific property, feature, or activity.

The invention thus provides methods described immediately above in which in vitro screening is performed to identify one or more nucleic acid molecules having at least one specific property, feature, or activity, as well as nucleic acid molecules identified by the above methods and expression products of these nucleic acid molecules.

Examples of properties, features, and/or activities which can be detected by in vitro screening include, but are not limited to, the ability to hybridize either intramolecularly or to another nucleic acid molecule under stringent hybridization conditions, the ability to activate transcription, the ability to bind proteins, the ability to initiate replication of nucleic acid molecules, the ability to be cleaved by one or more restriction endonucleases, the ability to be joined to another nucleic acid molecule by topoisomerase, the ability to be ligated to another nucleic acid molecule, the ability to anneal to another nucleic acid molecule, and the ability to recombine with another nucleic acid molecule by site specific recombination.

In addition, nucleic acid molecules may be screened using in vitro methods to detect properties, features, or activities associated with encoded expression products. Properties, features, or activities of such expression products include, but are not limited to, the following: ribozyme activity, tRNA activity, antisense activity, being encoded by nucleic acid which is in-frame with nucleic acid that encodes another polypeptide, the ability to induce an immunological response, having binding affinity for a particular ligand, the ability to undergo proteolytic cleavage, and the ability to undergo post-translational modification.

The invention further provides compositions comprising two or more genetic elements which confer a temperature sensitive phenotype upon host cells. In specific embodiments, at least one of the genetic elements is either an origin of replication (e.g., E. coli origin of replication) or an antibiotic resistance marker (e.g., kanamycin resistance marker, an ampicillin resistance marker, a gentamycin resistance marker, etc.).

In additional specific embodiments, the two or more genetic elements which confer the temperature sensitive phenotype are located on the same nucleic acid molecule. Further, when two genetic elements are located on the same nucleic acid molecule, these elements may be separated by less than 200 nucleotides of intervening nucleic acid.

The invention additionally provides kits for inserting a population of nucleic acid molecules into a second target molecule according to the methods described above, these kits may comprise one or more components selected from the group consisting of:
(a) one or more first population of nucleic acid molecules;
(b) one or more first target nucleic acid molecule;
(c) one or more second target nucleic acid molecule;
(d) one or more recombination proteins or compositions comprising one or more recombination proteins;
(e) one or more enzymes having ligase activity;
(f) one or more enzymes having polymerase activity;
(g) one or more enzymes having reverse transcriptase activity;
(h) one or more enzymes having restriction endonuclease activity;
(i) one or more primers;
(j) one or more buffers;
(k) one or more transfection reagents;
(l) one or more host cells;
(m) one or more enzymes having UDG glycosylase activity (e.g., Invitrogen Corp., Carlsbad, Calif., Catalog No. 18054-015);
(n) one or more enzymes having topoisomerase activity;
(o) one or more proteins which facilitate homologous recombination; and
(p) instructions for using the kit components.

In specific embodiments, the kits contain the one or more recombination proteins or composition comprising one or more recombination proteins capable of catalyzing recombination between att sites. In more specific embodiments, the composition comprising one or more recombination proteins capable of catalyzing a BP reaction, an LR reaction, or both BP and LR reactions.

In related embodiments, kits of the invention contain at least one first population of nucleic acid molecules comprising one or more library which encode either variable heavy or variable light domains of antibody molecules.

Other embodiments of the present invention will be apparent to one of ordinary skill in light of what is known in the art, in light of the following drawings and description of the invention, and in light of the claims.

### BRIEF DESCRIPTION OF THE DRAWINGS

FIG. 1 depicts one general method of the invention. In particular, a first population of nucleic acid molecules (e.g., cDNA molecules) is mixed with a target nucleic acid molecule (labeled “first target molecule”). The individual members of the first population of nucleic acid molecules and/or the first target molecule shown have one or more recombination sites. One such site is labeled “insertion site” on the first target molecule. The individual members of the first population of nucleic acid molecules are inserted into the target molecule by a recombination reaction (labeled “first recombination”) and, optionally, subjected to one or more selection, identification, or isolation steps, thereby forming the second population of nucleic acid molecules (labeled “second population”). The second population of nucleic acid molecules is then mixed with a second target nucleic acid molecule (labeled “second target molecule”). The nucleic acid inserts of the second population of nucleic acid molecules are then transferred to the second target nucleic acid molecule by a recombination reaction (labeled “second recombination”) and, optionally, subjected to one or more selection, identification, or isolation steps, thereby forming a third population of nucleic acid molecules (labeled “third population”).

FIG. 2 shows one example of a process of the invention for the generation of Expression Clones by the transfer of nucleic acid molecules of a cDNA library flanked by attB sites. The nucleic acid molecules of the cDNA library initially reside in supercoiled plasmids which contain an ampicillin resistance marker (labeled “amp”), an origin of replication (labeled “ORI”), and a site which can be used to linearize the vector (labeled “cut site”). The nucleic acid molecules of the cDNA library are then inserted into a linear pDONR plasmid (also abbreviated “pDONOR”) (which contains attP sites, an origin of replication and a kanamycin resistance marker (labeled “kan”)) by a BP reaction in the presence of Fis protein. The resulting products of this reaction are Entry Clones. The nucleic acid molecules of the cDNA library can then be transferred from the Entry Clones to a Destination Vector by an LR reaction to generate new Expression Clones. As one skilled in the art would recognize, populations of nucleic acid molecules other than cDNA libraries (e.g., genomic libraries, synthetic libraries, etc.) may be used in similar processes.

FIG. 3 shows another example of a process of the invention for the generation of Expression Clones by the transfer of nucleic acid molecules of a cDNA library flanked by attB sites. In this instance, the cDNA library and attP site donor molecules are linear. BP CLONASE™ catalyzed recombination results in the cDNA molecules of the library being flanked by attL sites. The cDNA molecules are then inserted into a Destination Vector by LR CLONASE™ catalyzed recombination to generate new Expression Clones. As one skilled in the art would recognize, populations of nucleic acid molecules other than cDNA libraries may be used in similar processes.

FIG. 4 shows a schematic representation of a Destination Vector which can be used for the insertion and subsequent transfer of nucleic acid molecules flanked by attL1 and attL2 sites. cDNA molecules flanked by attL1 and attL2 sites which can be inserted into the vector using LR CLONASE™ catalyzed recombination are also shown. Subsequent recombination with, for example, any attP Donor plasmid can be used to create new populations of Destination Vectors or Entry Clones. For example, linear pDONOR molecules which have been cut in the backbone of the vector (e.g., between kan and ori) may be used to generate/regenerate Destination Vectors (e.g., the first target molecule shown in this figure). As one skilled in the art would recognize, populations of nucleic acid molecules other than cDNA libraries may be used in similar processes. Further, any of the molecules which undergo recombination may be linear or closed, circular.

FIG. 5 shows one example of a process of the invention for the generation of Expression Clones by the transfer of nucleic acid molecules of a cDNA library flanked by an attB site and a site which can be used for nucleic acid cleavage (labeled “cut site 2”). In this instance, cut site 2 is a site which is cleaved by a restriction endonuclease, referred to as “restriction enzyme 2”. The population of cDNA is transferred by combining recombination and ligation. As one skilled in the art would recognize, populations of nucleic acid molecules other than cDNA libraries may be used in similar processes.

FIGS. 6A-6D represents nucleic acid segments, each of which contains an origin of replication (OR1) and a kanamycin resistance marker (Kan). Each of these genetic elements has particular directionalities of function, which are indicated by the arrows.

FIG. 7 shows a schematic of a selection process for the use of conjugative transfer to select for nucleic acid molecules having particular nucleic acid segments. In this case oriT, is an origin of conjugative DNA transfer (CDT). Thus, only nucleic acid molecules which contain oriT will be transferred from one cell to another during conjugation. As one skilled in the art would recognize, populations of nucleic acid molecules other than cDNA libraries may be used in similar processes.

FIG. 8 shows a two step selection and screening process of the invention for identifying cDNA molecules which have particular properties. As part of the first step in the process, Expression Clones are generated using cDNA molecules of a cDNA library. A Gal1 promoter is located at one end of the molecules of the cDNA library inserted into the vector. Nucleic acid which encodes the encodes Galactose 4 gene Activation Domain (Gal4 AD) is located between the Gal1 promoter and the cDNA inserts. The Expression Clone library is then inserted into yeast cells and selection occurs using a two-hybrid assay to identify cDNAs which encode proteins (i.e., “prey” proteins) that associate with a “bait” protein. Two-hybrid assay systems are described, for example, in Yavuzer and Goding, Gene 165:93-96 (1995); Vidal et al., U.S. Pat. No. 5,955,280; and Fields et al., U.S. Pat. No. 5,283,173, the entire disclosures of each of which are incorporated herein by reference.

The cDNAs of a cDNA library identified by the two-hybrid selection process described above are then transferred to another vector which contains nucleic acid encoding a HIS6 tag located between a T7 promoter and the cDNA inserts. These vectors are then inserted in cells, fusion proteins are expressed, and the resulting protein is precipitated by immune precipitation in the presence of extracts containing the putative interaction protein(s). As one skilled in the art would recognize, populations of nucleic acid molecules other than cDNA libraries may be used in similar processes.

FIG. 9 depicts one general description of recombinational cloning processes which can be used in the practice of the invention. The goal is to exchange the new subcloning vector D for the original cloning vector B. Thus, in certain embodiments, it is desirable to select for AD and against all the other molecules, including the Cointegrate. The square and circle are recombination sites (e.g., lox (such as loxP) sites, att sites, etc.). Further, Segment D can contain expression signals, protein fusion domains, drug markers, origins of replication, or specialized functions for mapping or sequencing DNA. It should be noted that the Cointegrate molecule contains Segment D adjacent to Segment A (Insert), thereby juxtaposing functional elements in Segment D with the Insert. Such molecules can be used directly in vitro (e.g., if a promoter is positioned adjacent to a gene—for in vitro transcription/translation) or in vivo (e.g., following isolation in a cell capable of propagating ccdB-containing vectors) by selecting for selection markers in Segments B+D. As one skilled in the art will recognize, this single step recombination cloning process has utility in certain envisioned applications of the invention.

FIG. 10 is a depiction of the recombinational cloning system referred to herein as the “GATEWAY™ Cloning System” (FIG. 10A). This figure depicts the production of Expression Clones via a “Destination Reaction,” also referred to herein as an “LR Reaction” or an “LR CLONASE™ Reaction.” A kan^{r} vector (labeled “Entry Clone”) containing a DNA molecule of interest (e.g., a gene) located between an attL1 site and an attL2 site is reacted with an amp^{r} vector (labeled “Destination Vector”) containing a toxic or “death” gene located between an attR1 site and an attR2 site, in the presence of GATEWAY™ LR CLONASE™ Enzyme Mix (a mixture of Int, IHF and Xis). After incubation at 25° C. for about 60 minutes, the reaction yields an amp^{r} Expression Clone containing the DNA molecule of interest located between an attB1 site and an attB2 site, and a kan^{r} By-product molecule, as well as intermediates. The reaction mixture may then be transformed into host cells (e.g., Escherchia coli) and clones containing the nucleic acid molecule of interest may be selected by plating the cells onto ampicillin-containing media and picking amp^{r} colonies.

FIG. 10B is a depiction of the production of Entry Clones via an “Entry Reaction,” also referred to herein as a “BP reaction” or a “BP CLONASE™ Reaction.” In the example shown in this figure, an amp^{r} expression vector containing a DNA molecule of interest (e.g., a gene) localized between an attB1 site and an attB2 site is reacted with a kan^{r} Donor vector containing a toxic or “death” gene localized between an attP1 site and an attP2 site, in the presence of GATEWAY™ BP CLONASE™ Enzyme Mix (a mixture of Int and IHF). After incubation at 25° C. for about 45 minutes, the reaction yields a kan^{r} Entry Clone containing the DNA molecule of interest localized between an attL1 site and an attL2 site, and an amp^{r} By-product molecule. The Entry Clone may then be transformed into host cells (e.g., E. coli) and clones containing the Entry Clone (and therefore the nucleic acid molecule of interest) may be selected by plating the cells onto kanamycin-containing media and picking kan^{r} colonies. Although this figure shows an example of use of a kan^{r} Donor vector, it is also possible to use Donor vectors containing other selection markers, such as the gentamycin resistance or tetracycline resistance markers, as discussed herein.

FIG. 11 is a schematic depiction of the cloning of a nucleic acid molecule from an Entry Clone into multiple types of Destination vectors, to produce a variety of Expression Clones. Recombination between a given Entry clone and different types of Destination Vectors (not shown), via the LR Reaction depicted in FIG. 10, produces multiple different Expression Clones for use in a variety of applications and host cell types.

FIG. 12 shows the sequences of the attB1 (SEQ ID NO:5) and attB2 (SEQ ID NO: 9) sites flanking a gene of interest after subcloning into a Destination Vector to create an Expression Clone. One reading frame of each recombination site is indicated. The seven base pair overlap regions of each site are also shown.

FIGS. 13A-13C show the sequences of a number of att sites (SEQ ID NOs:1-36) suitable for use in methods and compositions of the invention. Sequences are written conventionally, from 5′ to 3′. The seven base pair overlap regions of each site is indicated by underlining.

FIG. 14 is a schematic depiction of four ways to make Entry Clones using the compositions and methods of the invention: (1) using restriction enzymes and ligase; (2) starting with a cDNA library prepared in an attL Entry Vector; (3) using an Expression Clone from a library prepared in an attB Expression Vector via the BP reaction; and (4) recombinational cloning of PCR fragments with terminal attB sites, via the BP reaction. Approaches 3 and 4 rely on recombination with a Donor vector (shown here as an attP vector, such as pDONR201 (Invitrogen Corp., Carlsbad, Calif., Catalog No. 11798-014), or pDONR207 (see FIGS. 19A-19C), for example) that provides the Entry Clone with a selection marker such as kan^{r}, gen^{r}, tet^{r}, or the like. Numerous additional methods (e.g., topoisomerase cloning) may used to make Entry Clones.

FIG. 15 is a schematic depiction of a method for cloning of a PCR product using a BP reaction. A PCR product with 25 base pair terminal attB sites (plus four guanine residues) is shown as a substrate for the BP reaction. Recombination between the attB-PCR product of a gene and a Donor vector (which donates an Entry Vector that carries kan^{r}) results in the generation of an Entry Clone containing the PCR product.

FIG. 16 shows the plasmid backbone (FIG. 16A, SEQ ID NO:60, SEQ ID NO: 61) and nucleotide sequence (FIG. 16B, SEQ ID NO:37) of the Entry Vector pENTR1A). Plasmid specific maps, sequences and schematic depiction of structural and functional features for a variety of Entry Vectors are disclosed in U.S. application Ser. No. 09/177,387, filed Oct. 23, 1998; U.S. application Ser. No. 09/517,466, filed Mar. 2, 2000; and PCT Publication WO 00/52027 the disclosures of which are incorporated herein by reference in their entireties.

FIG. 17A-17D depictions the physical map (FIG. 17A, SEQ ID NO:62) and nucleotide sequence (FIGS. 17B-17D, SEQ ID NO:38) of the Destination plasmid pDEST1.

FIG. 18A-18C depictions the physical map (FIG. 18A) and nucleotide sequence (FIGS. 18B-18C, SEQ ID NO:39) of the Donor plasmid pDONR207, which donates a gentamycin-resistance marker in the BP reaction.

FIG. 19 is a schematic representation of the use of the present invention to clone two nucleic acid segments by performing an LR recombination reaction.

FIG. 20A is a plasmid map showing a construct for providing a C-terminal fusion to a polypeptide encoded by nucleic acid inserted into the plasmid. SupF encodes a suppressor function. Thus, when supF is expressed, a GUS-GST fusion protein is produced. Variations of this molecule can be used to express GUS (or any other nucleic acid segment) fused to essentially any polypeptide.

FIG. 20B is a schematic representation of method for controlling both gene suppression and expression. The T7 RNA polymerase gene contains one or more (two are shown) amber stop codons (labeled “am”) in place of tyrosine codons. Leaky (uninduced) transcription from the inducible promoter makes insufficient supF to result in the production of active T7 RNA polymerase. Upon induction, sufficient supF is produced to make active T7 RNA polymerase, which results in increased expression of supF, which results in further increased expression of T7 RNA polymerase. The T7 RNA polymerase further induces expression of Gene. Further, expression of supF results in the addition of a C-terminal tag to the Gene expression product by suppression of the intervening amber stop codon.

FIG. 21 is a plasmid map showing a construct for the production of N- and/or C-terminal fusions of a gene of interest. Circled numbers represent anber, ochre, or opal stop codons. Suppression of these stop codons result in expression of fusion tags on the N-terminus, the C-terminus, or both termini. In the absence of suppression, native protein is produced.

FIG. 22 shows experiments related to Fis stimulation of single-site LR recombination reactions. Reactions (20 μl) were performed using 100 fmol pATTL2 and 100 fmol pATTR2-BamHI substrates (see “Experimental Methods” in Example 9 below). The percentage of recombination product observed at given Fis concentrations is plotted for three different concentrations of Xis. Percent product was determined by dividing the amount of radioactivity in the product band by the sum of the amount of radioactivity in the substrate and product bands.

FIG. 23 shows experiments related to Fis stimulation of double-site BP recombination reactions. Reactions (20 μl) were performed using 100 fmol pDONR201 and 100 fmol pBGFP2-XhoI substrates (see “Experimental Methods” in Example 9 below). The percentage of recombination product observed at given Fis concentrations is plotted for two different concentrations of NaCl. Percent product was determined by dividing the amount of radioactivity in the product band by the sum of the amount of radioactivity in the substrate, cointegrate, and product bands.

FIG. 24 shows experiments related to the effect of salt concentration on Fis stimulation of double-site BP recombination reactions. Reactions (20 μl) were performed using 100 fmol pDONR201 and 100 fmol pBGFP2-XhoI substrates (see “Experimental Methods” in Example 9 below). The percentage of recombination product observed at given NaCl concentrations is plotted for four different concentrations of Fis. Data shown are averages of 3 experiments, with standard deviation shown by error bars.

FIG. 25 shows experiments which demonstrate that Fis stimulation of single-site BP recombination reactions is evident at lower Int concentrations. Reactions (20 μl) were performed using 100 fmol pATTP2 and 100 fmol pATTB2-Hind substrates (see “Experimental Methods” in Example 9 below). The percentage of recombination product observed at given Int concentrations is plotted for three different Fis concentrations.

FIG. 26A-26C depictions the physical map (FIG. 26A) and nucleotide sequence (FIGS. 26B-26C, SEQ ID NO:40) of the Destination plasmid pDONR201.

FIG. 27A-27C depictions the physical map (FIG. 27A) and nucleotide sequence (FIGS. 27B-27C, SEQ ID NO:41) of the Destination plasmid pDONR212.

FIG. 28A-28C depictions the physical map (FIG. 28A) and nucleotide sequence (FIGS. 28B-28C, SEQ ID NO:42) of the Destination plasmid pDONR212(F), which contains a full length pUC plasmid derived origin of replication.

FIG. 29A-29C depictions the physical map (FIG. 29A) and nucleotide sequence (FIGS. 29B-29C, SEQ ID NO:43) of the Destination plasmid pDONR212(R), which contains a full length pUC plasmid derived origin of replication in a reverse orientation as compared to pDONR212(F).

FIG. 30 shows an example of a process of the invention for the generation of circularized vectors which contain cDNA molecules flanked by recombination sites. In particular, single site recombination is used to attach cDNA molecules to linearized vectors. One end of the cDNA molecule, which does not contain a recombination site, is then attached to the free end of the vector to circularize the molecule. Circularization may be accomplished by any number of means, including homologous recombination, annealing, ligation, or the use of topoisomerases (e.g., a Vaccinia virus topoisomerase; see U.S. Pat. No. 5,766,891, the entire disclosure of which is incorporated herein by reference).

FIG. 31 shows an example of a process of the invention for the insertion of two nucleic acid segments into a target nucleic acid molecule, and the subsequent connection of these two nucleic acid segments, to generate a circular nucleic acid molecule. The abbreviation “RS” stands for recombination site. Further, RS1 and RS2 are recombination sites which differ in recombination specificity. Nucleic acid segments A and B may be connected to each other by any number of means (e.g., homologous recombination, annealing, site specific recombination, topoisomerase cloning, etc.). Either one or both of nucleic acid segments A and B, for example, can be individual members of one or more libraries (e.g., combinatorial libraries). Further, in many embodiments, the nucleic acid segments which are connected to each other will be flanked by recombination sites that allow for the transferred of the joined segments to other target nucleic acid molecules by recombinational cloning.

FIG. 32 shows an example of a process of the invention by which nucleic acid molecules can be attached and removed from a support using recombinational reactions. In many embodiments (e.g., when beads are used in a single tube reaction), the first population of nucleic acid molecules will be in excess (e.g., two, five, ten, fifteen, twenty, etc. fold excess) with respect to the second target molecule.

FIG. 33 shows another example of a process of the invention by which nucleic acid molecules can be attached and removed from a support using recombinational reactions. Again, in many embodiments (e.g., when beads are used in a single tube reaction), the first population of nucleic acid molecules will be in excess (e.g., two, five, ten, fifteen, twenty, etc. fold excess) with respect to the second target molecule.

FIG. 34A-34D depictions the physical map (FIG. 34A) and nucleotide sequence (FIG. 34B, SEQ ID NO:63; FIGS. 34C-34D, SEQ ID NO:44) of the attB cloning vector pCMVSPORT6.0.

FIG. 35 shows another example of a process of the invention by which nucleic acid molecules that are attached to supports are released using recombinational reactions. Restriction endonuclease is abbreviated “RE”. Streptavidin is abbreviated “SA”. Origin of replication is abbreviated “ori”. Kanamycin resistance marker is abbreviated “kan”. Ampicillin resistance marker is abbreviated “amp”. Terminal transferase is used to attach biotin to the vector, which has been linearized with the restriction endonuclease.

FIG. 36 shows yet another example of a process of the invention by which nucleic acid molecules that are attached to supports are released using recombinational reactions. Abbreviations are the same as above for FIG. 35.

FIG. 37 shows an additional example of a process of the invention by which nucleic acid molecules that are attached to supports are released using recombinational reactions. Abbreviations are the same as above for

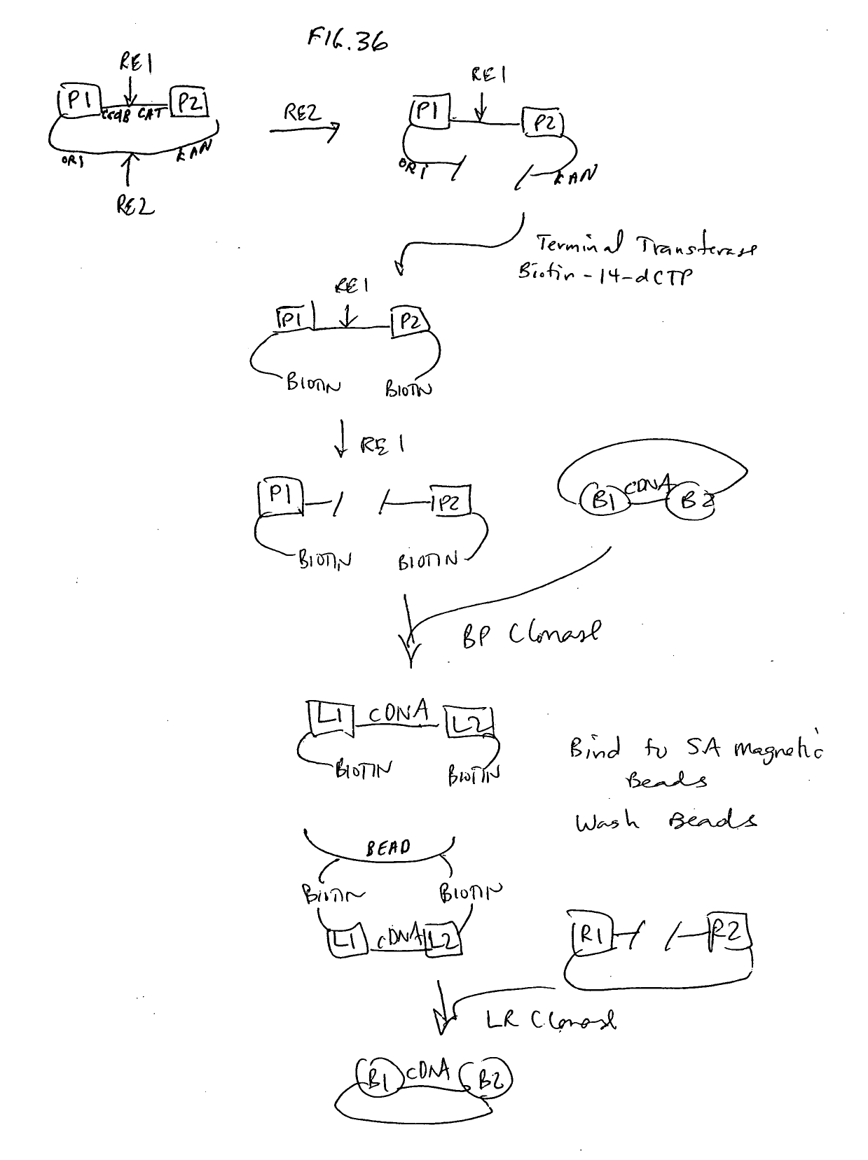

FIG. 35. Restriction endonucleases 3 and 4 are shown as restricting attP sites to generate attL and attR sites.

### DETAILED DESCRIPTION OF THE INVENTION

Definitions

In the description that follows, a number of terms used in recombinant DNA technology are utilized extensively. In order to provide a clear and consistent understanding of the specification and claims, including the scope to be given such terms, the following definitions are provided.

By-product: As used herein, the term “By-product” refers to a daughter molecule (a new clone produced after the second recombination event during the recombinational cloning process) lacking the segment which is desired to be cloned or subcloned.

Cointegrate: As used herein, the term “Cointegrate” refers to at least one recombination intermediate nucleic acid molecule of the present invention that contains both parental (starting) molecules. Cointegrates may be linear or circular. RNA and polypeptides may be expressed from Cointegrates using in vitro transcription and translation systems or an appropriate host cell strain, for example E. coli DB3.1 (particularly E. coli LIBRARY EFFICIENCY® DB3.1™ Competent Cells). Further, Cointegrates may be selected for using selection markers found on the Cointegrate molecule. Cointegrates may contain markers which allow for either in vitro or in vivo selection.

Host: As used herein, the term “host” refers to any prokaryotic or eukaryotic organism that is a recipient of a replicable expression vector, cloning vector or any nucleic acid molecule. The nucleic acid molecule may contain, but is not limited to, a structural gene, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Maniatis et al., Molecular Cloning: A Laboratory Manual, Cold Spring Harbor Laboratory, Cold Spring Harbor, N.Y. (1982).

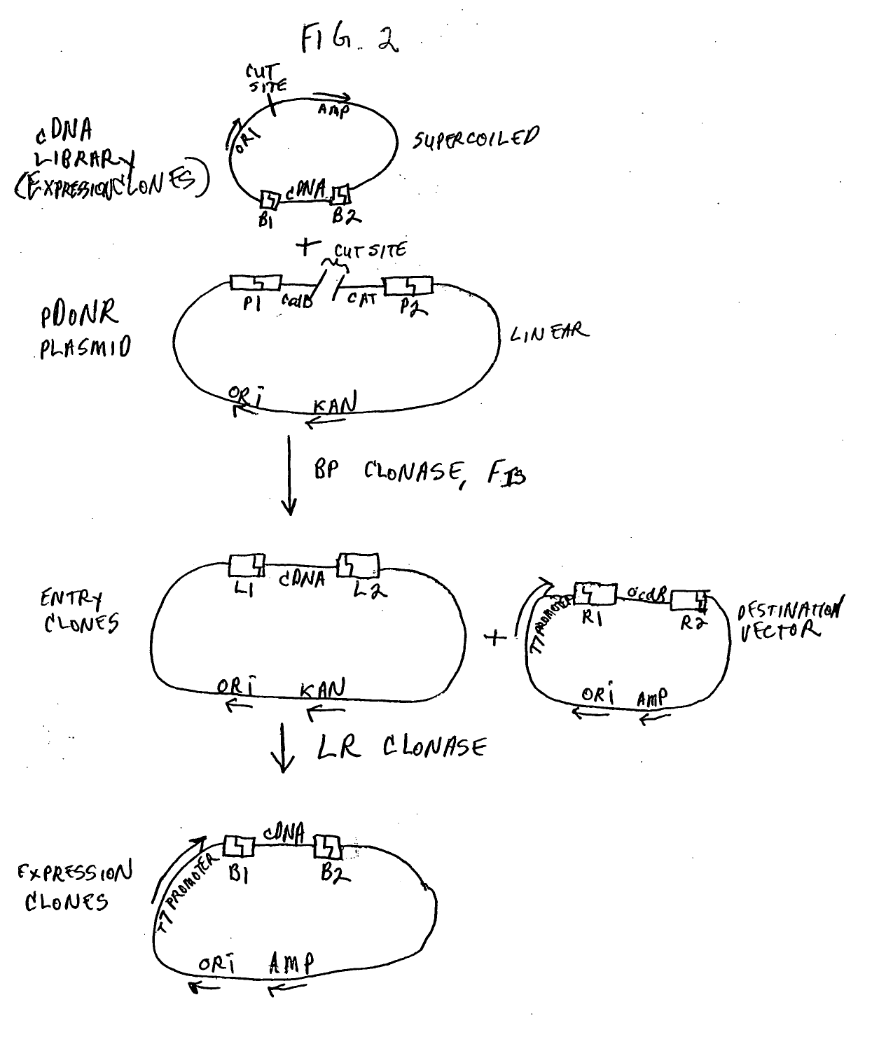

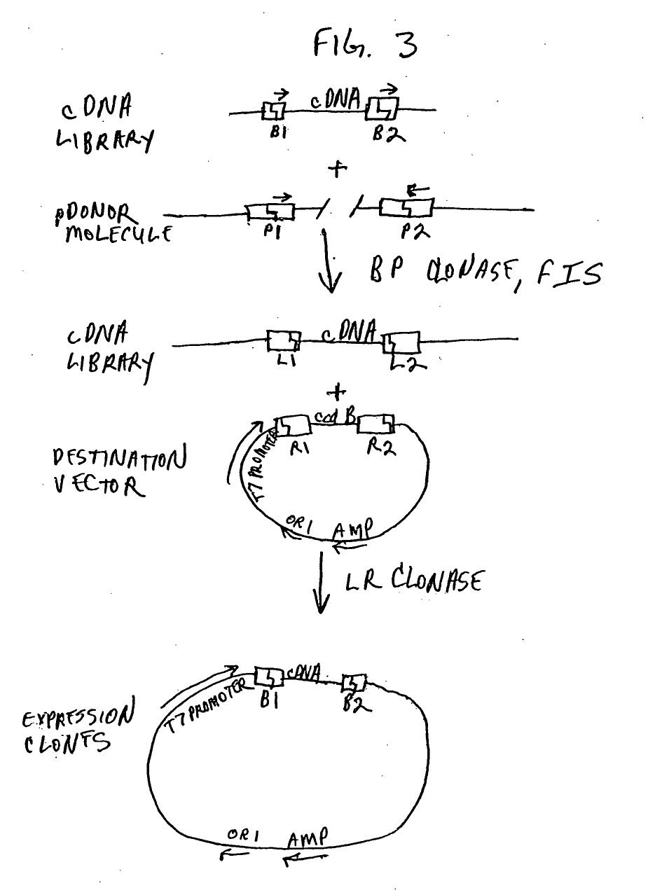

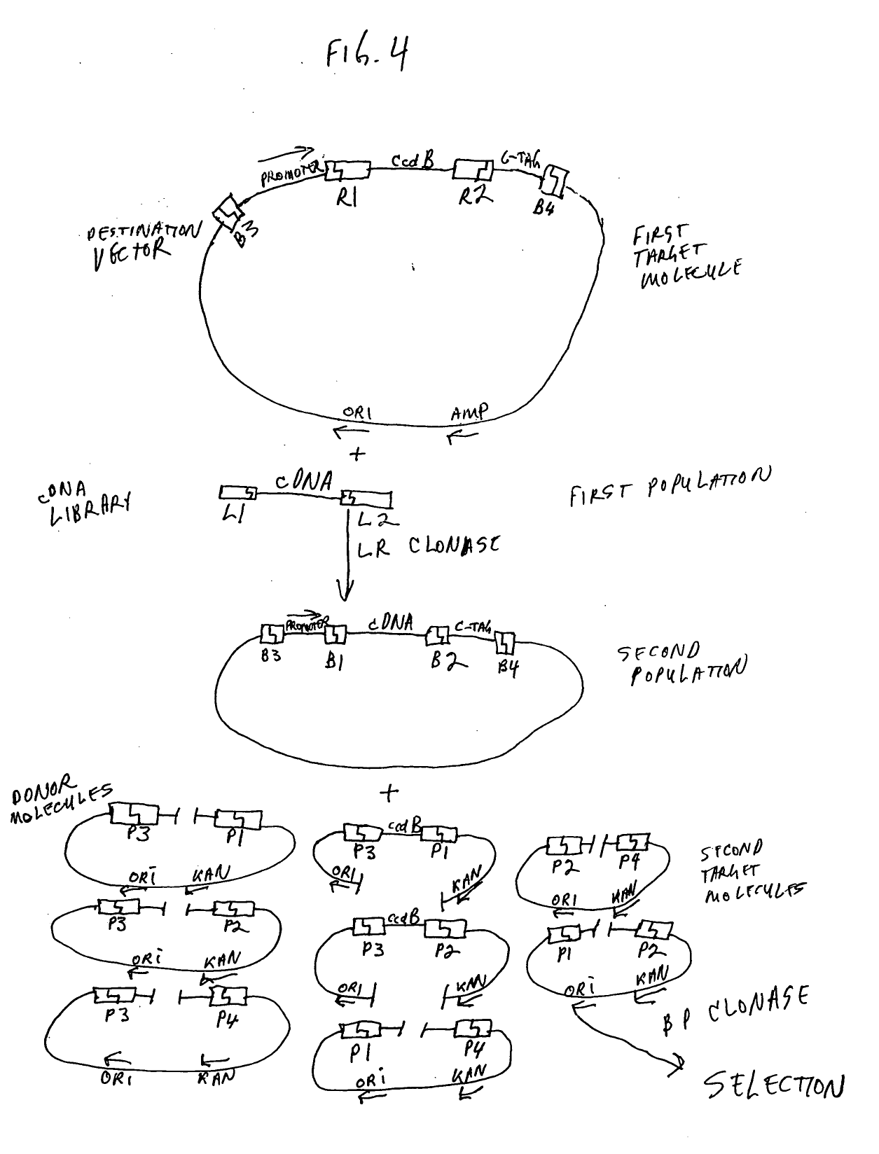

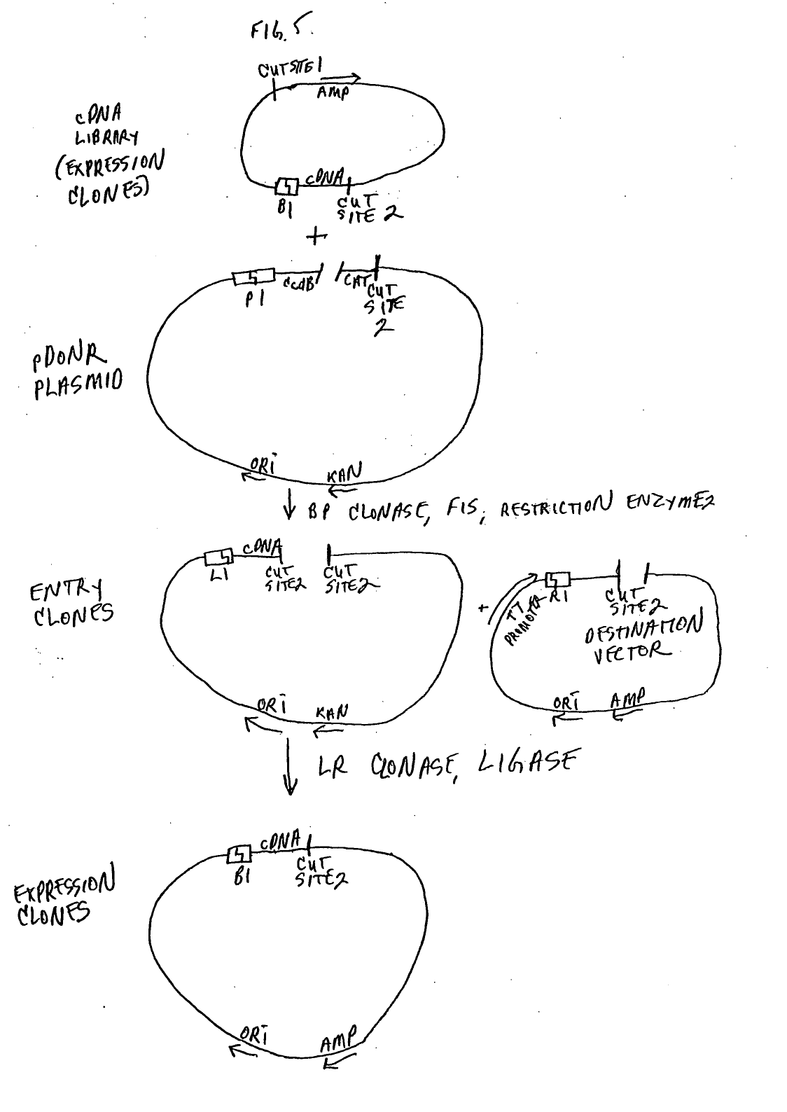

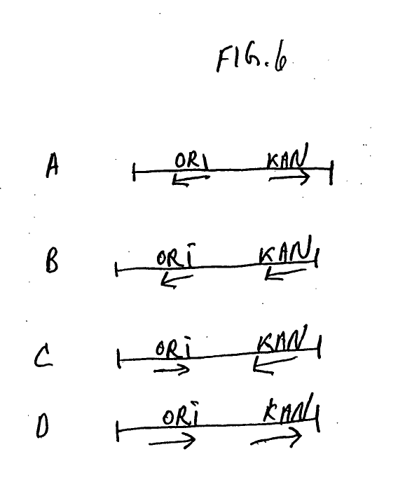

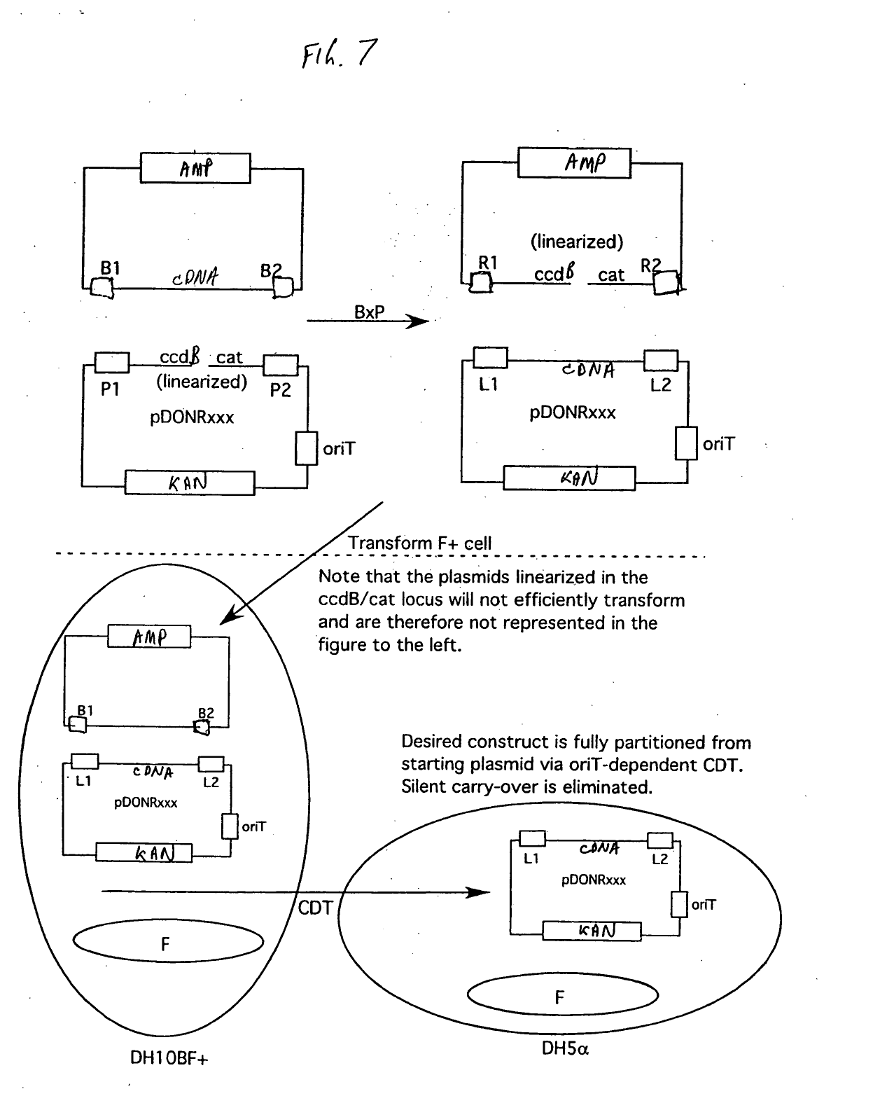

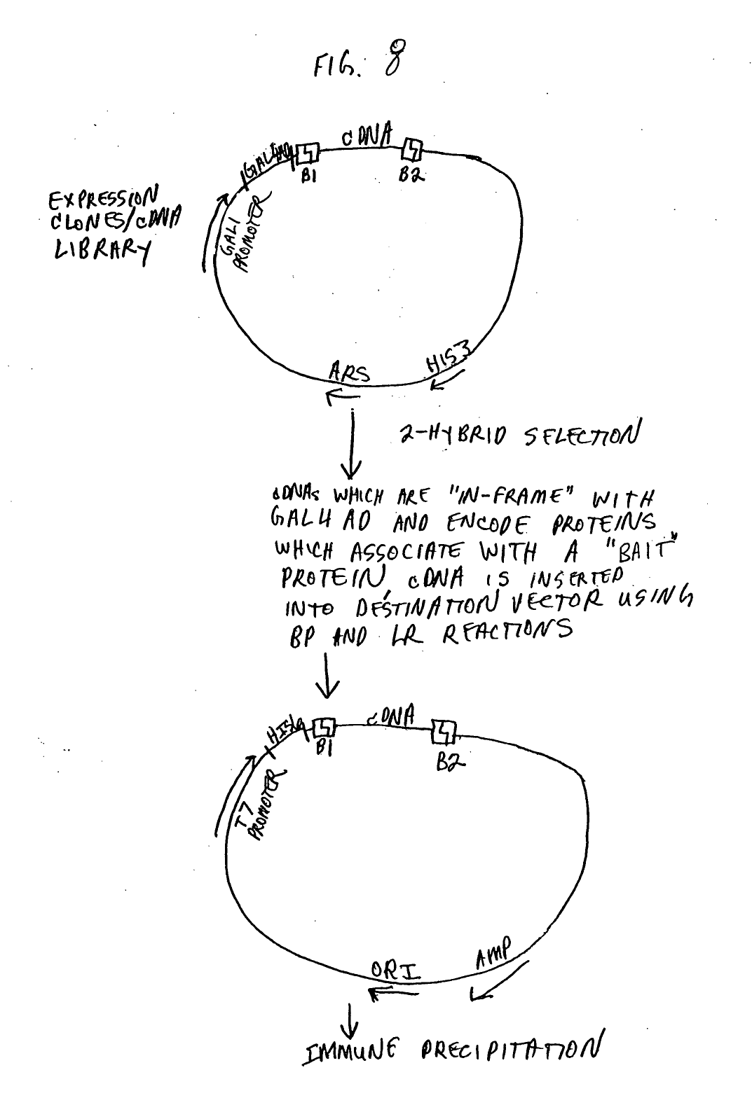

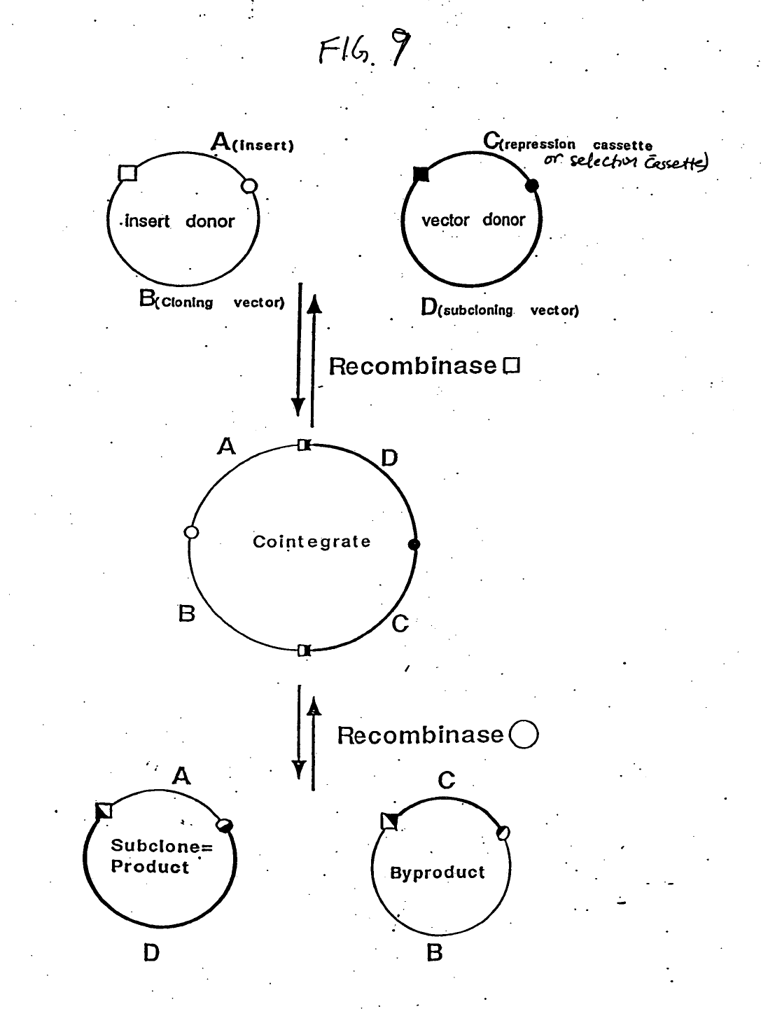

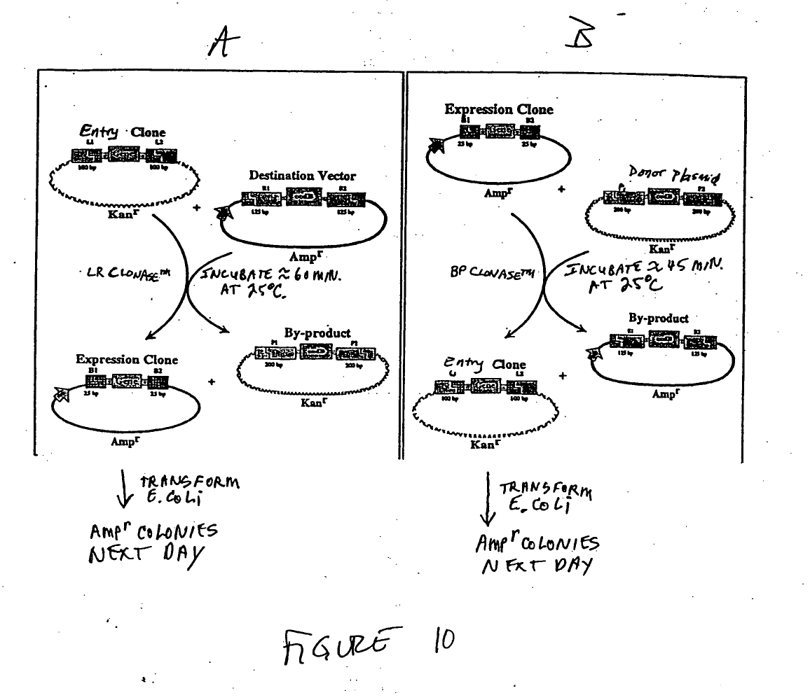

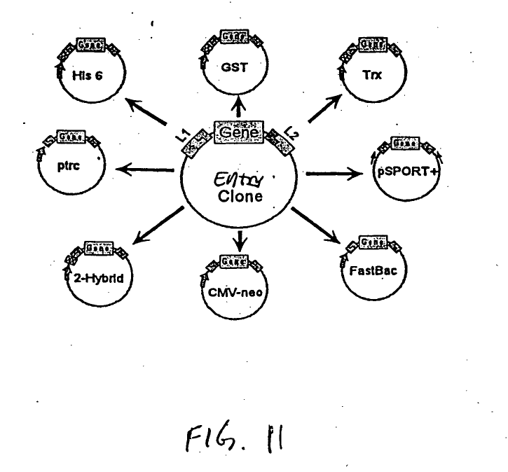

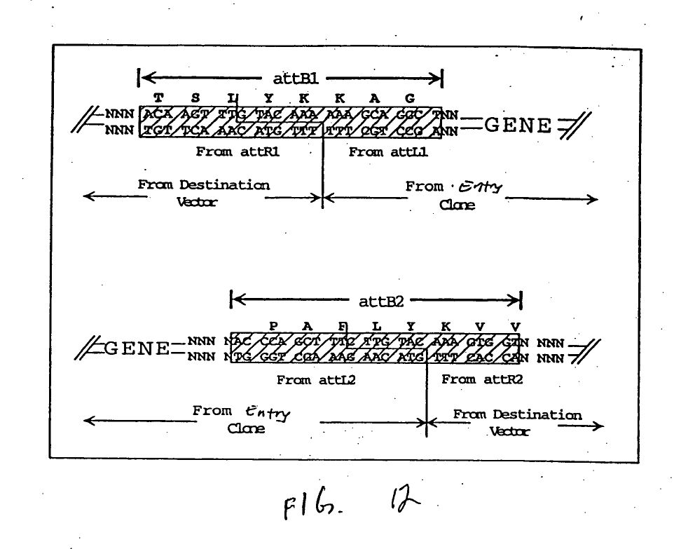

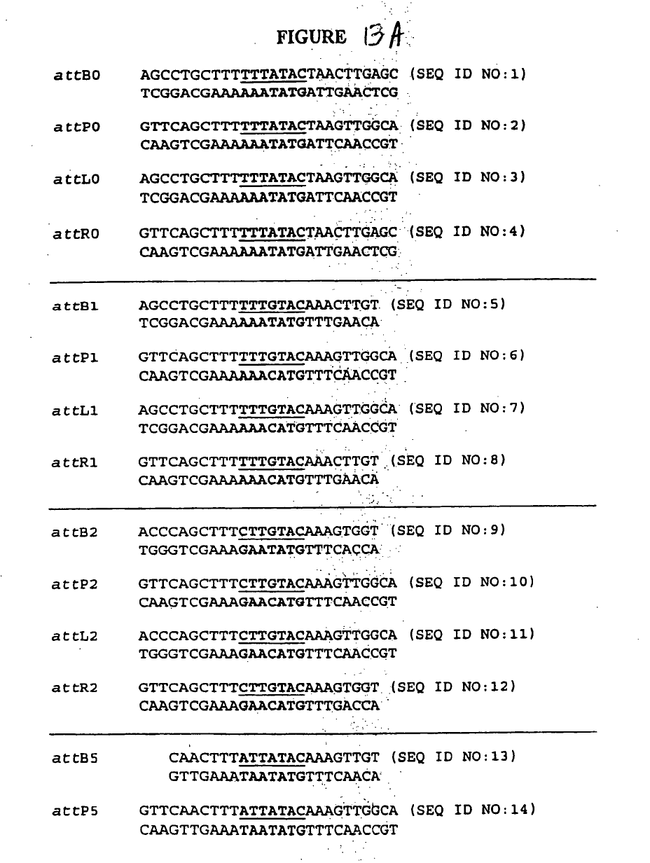

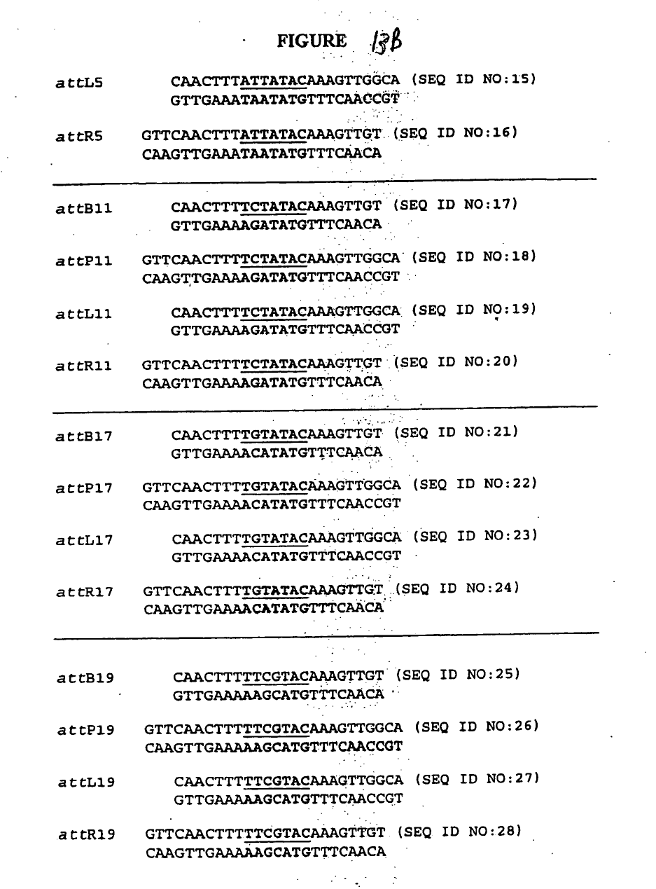

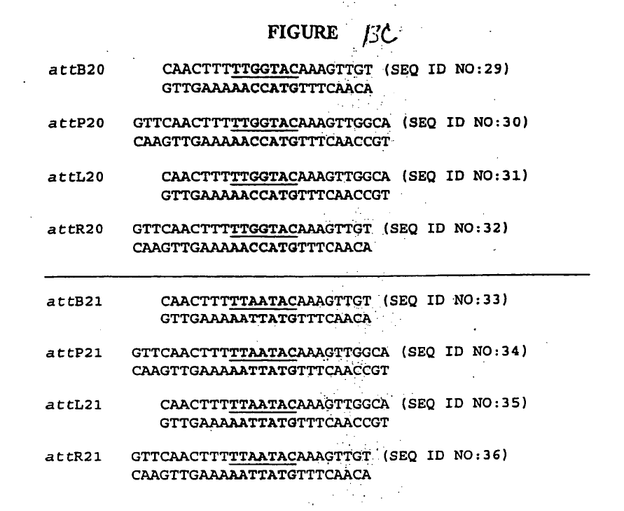

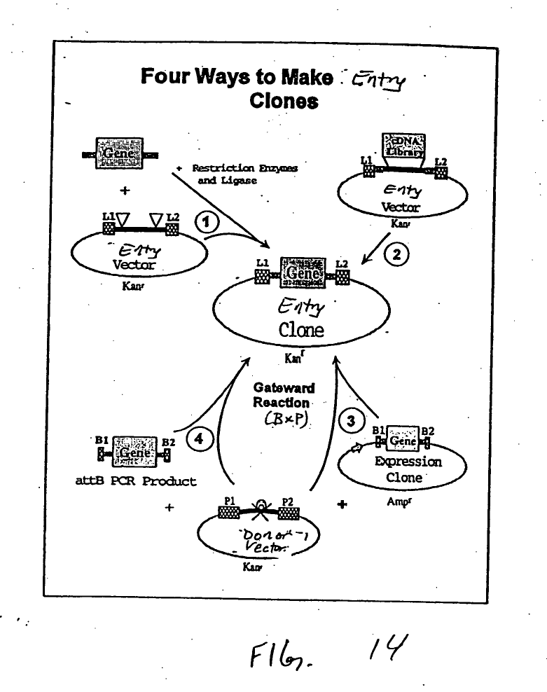

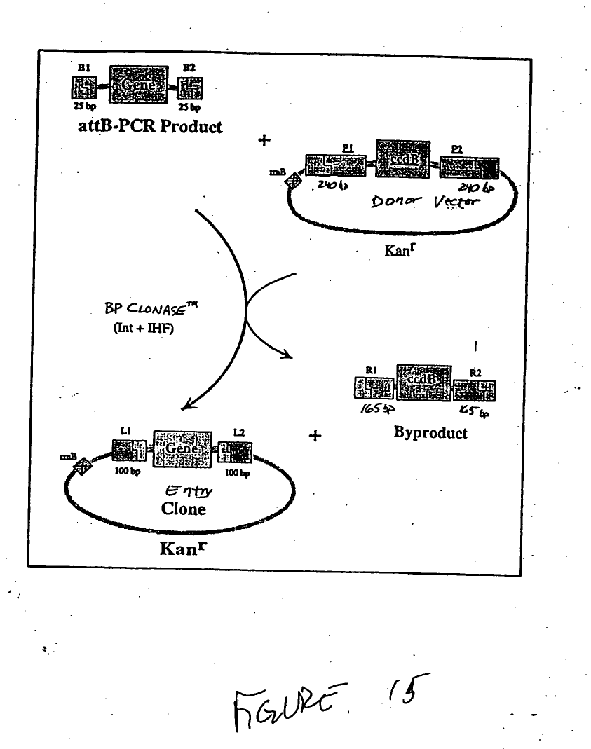

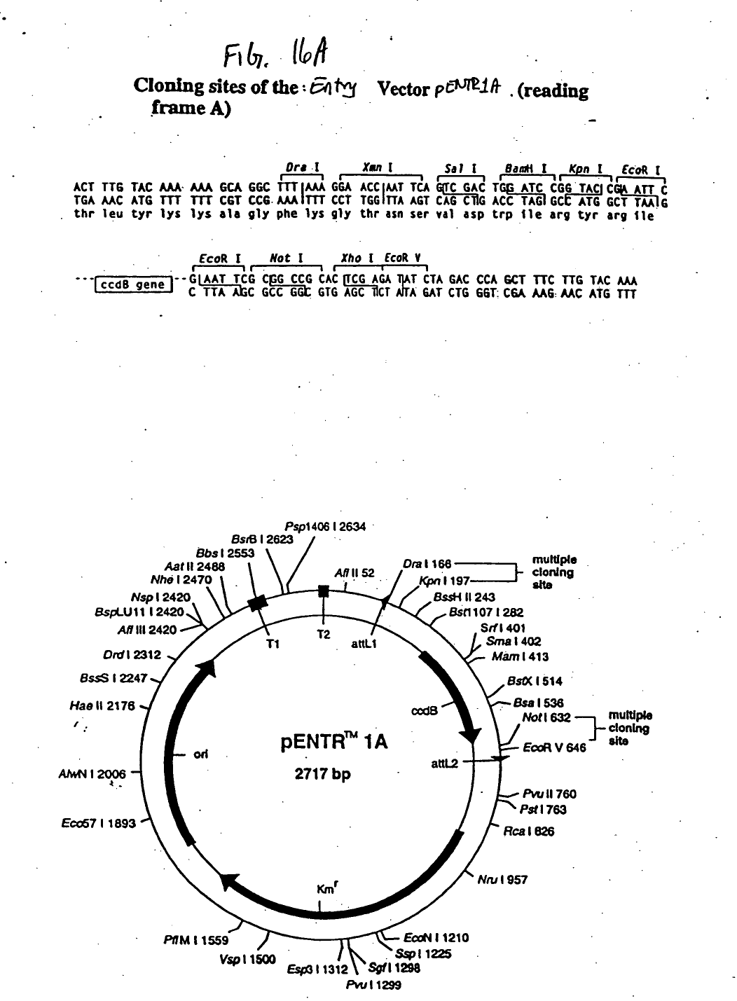

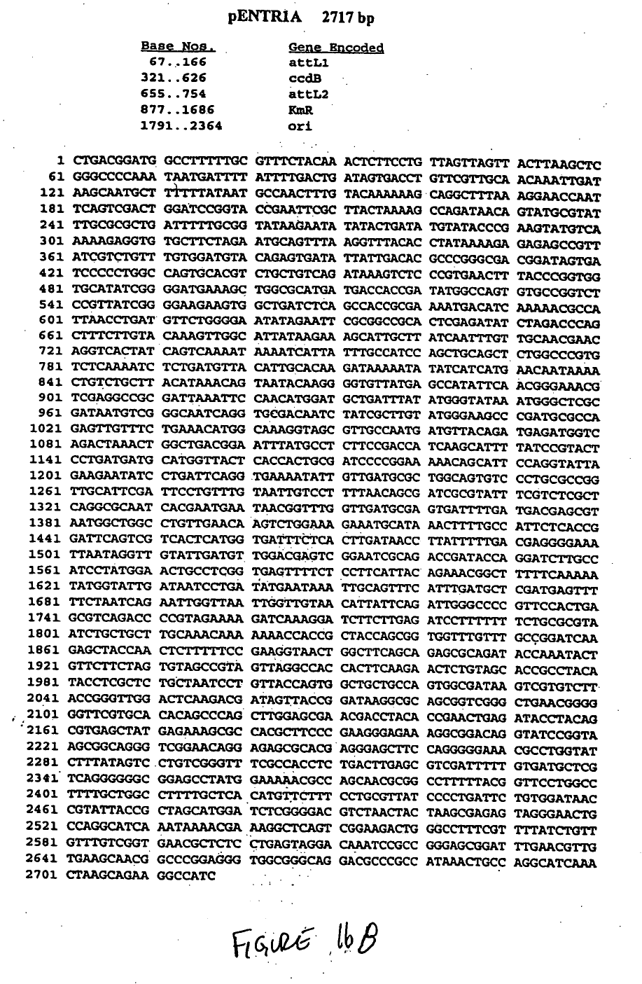

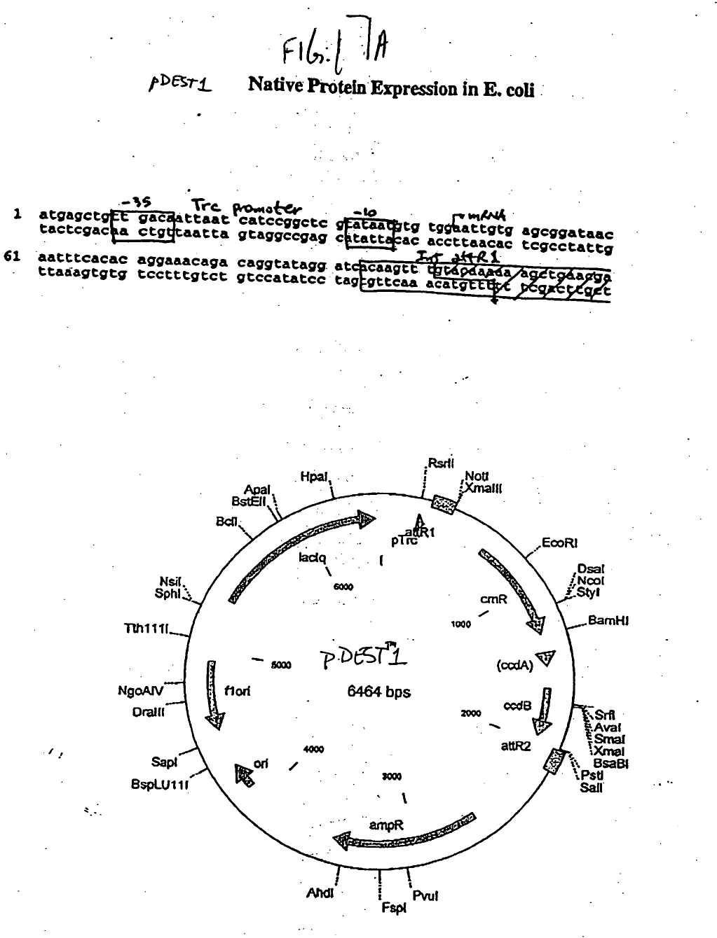

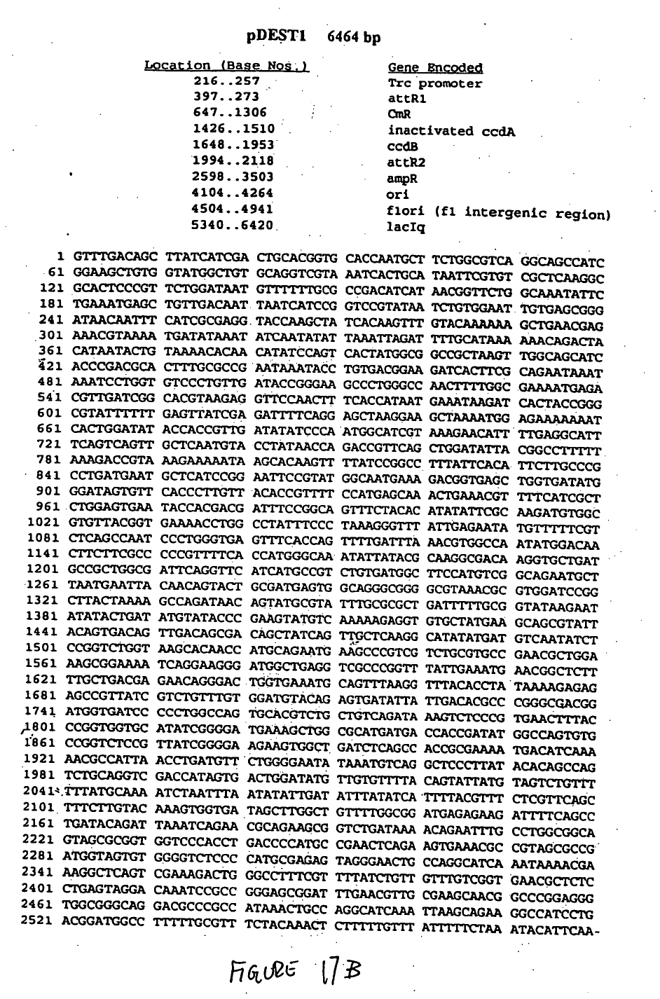

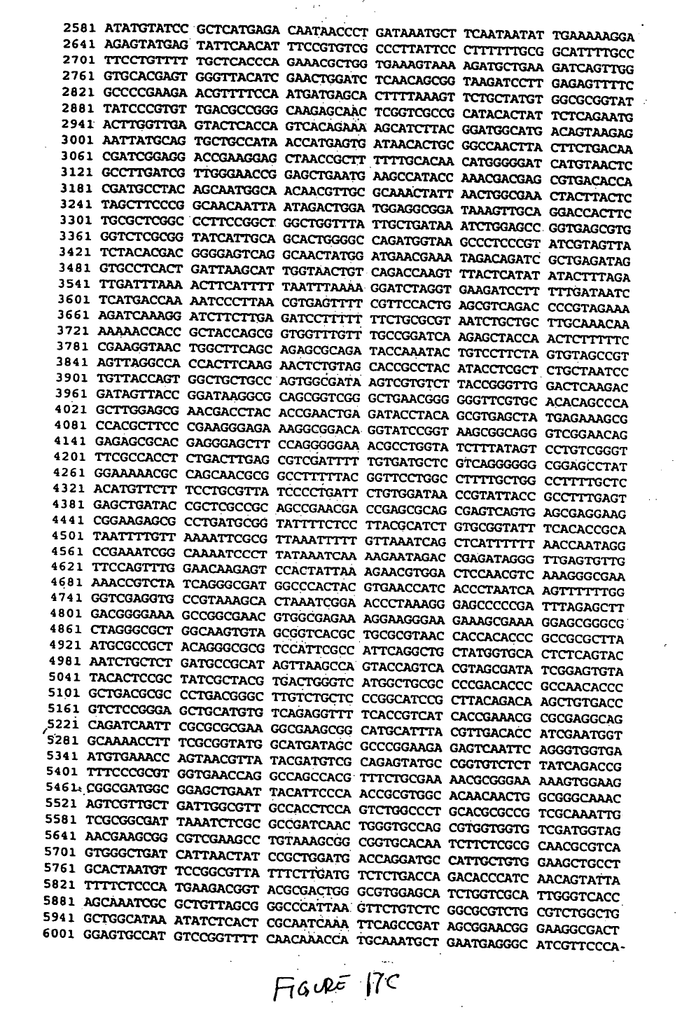

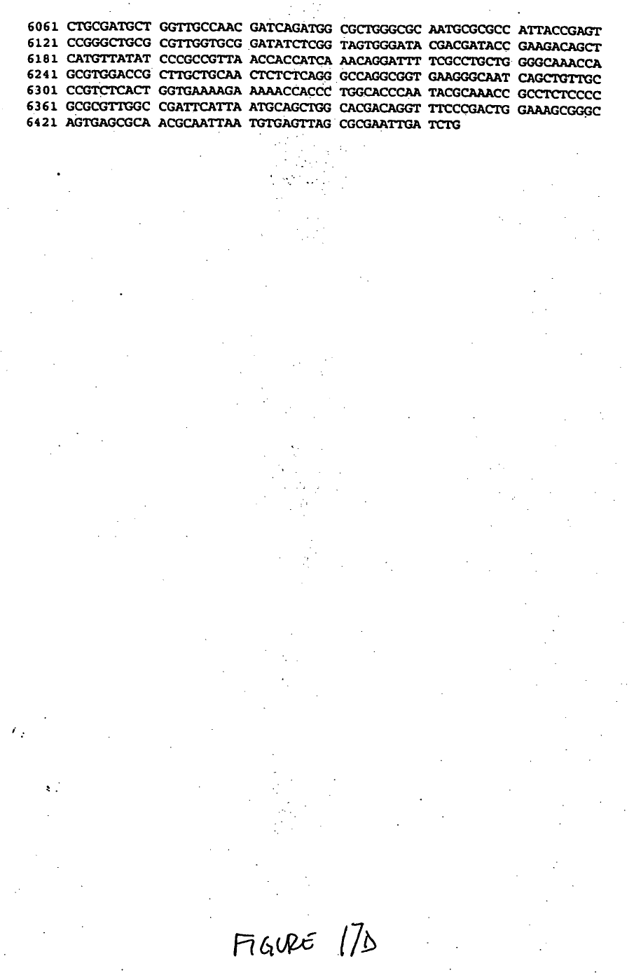

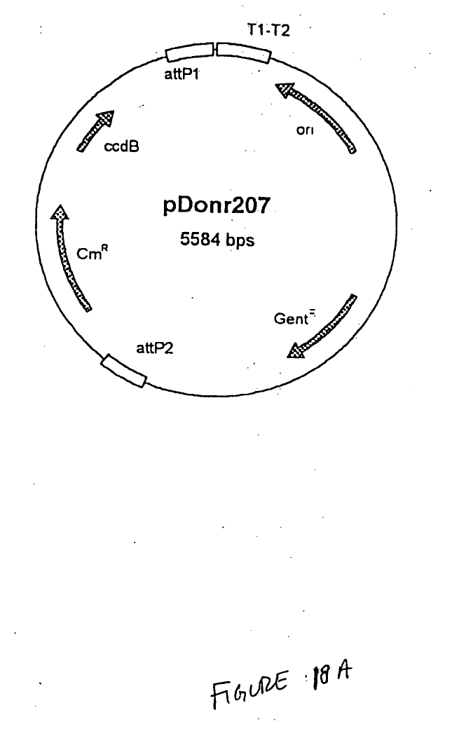

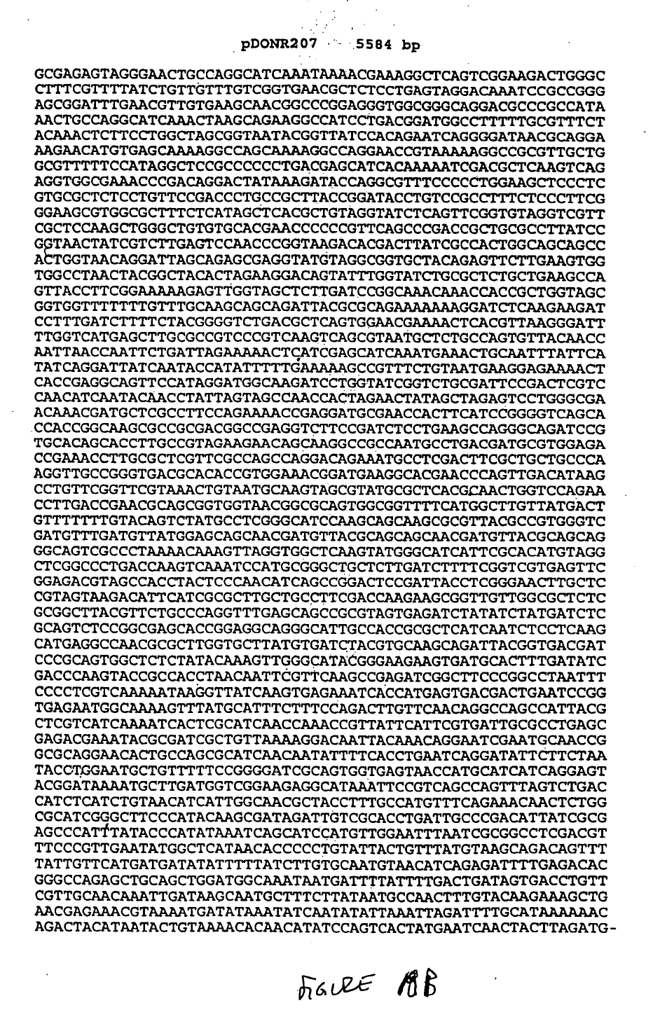

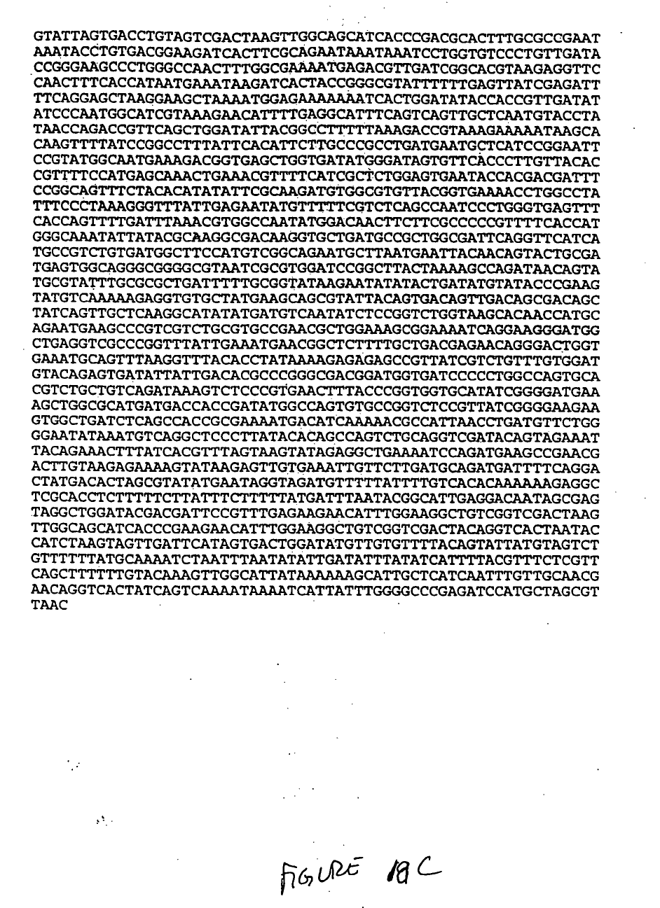

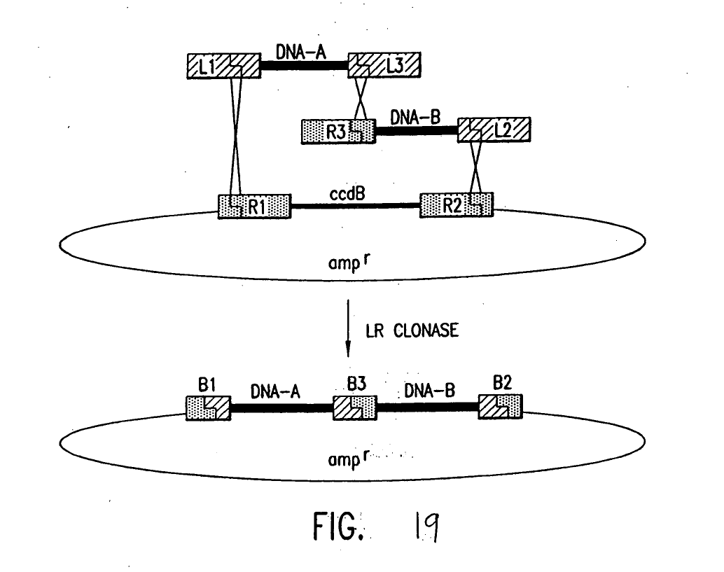

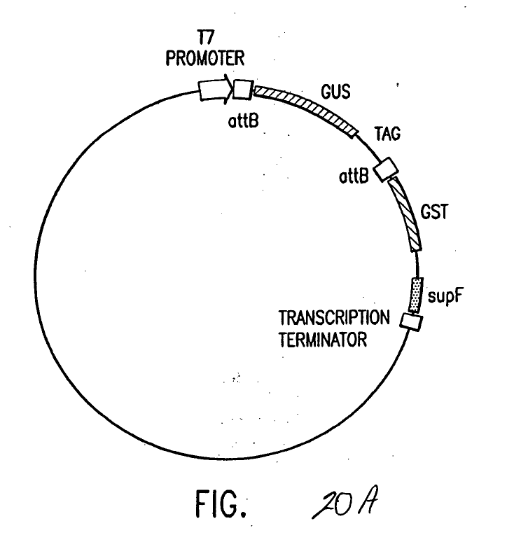

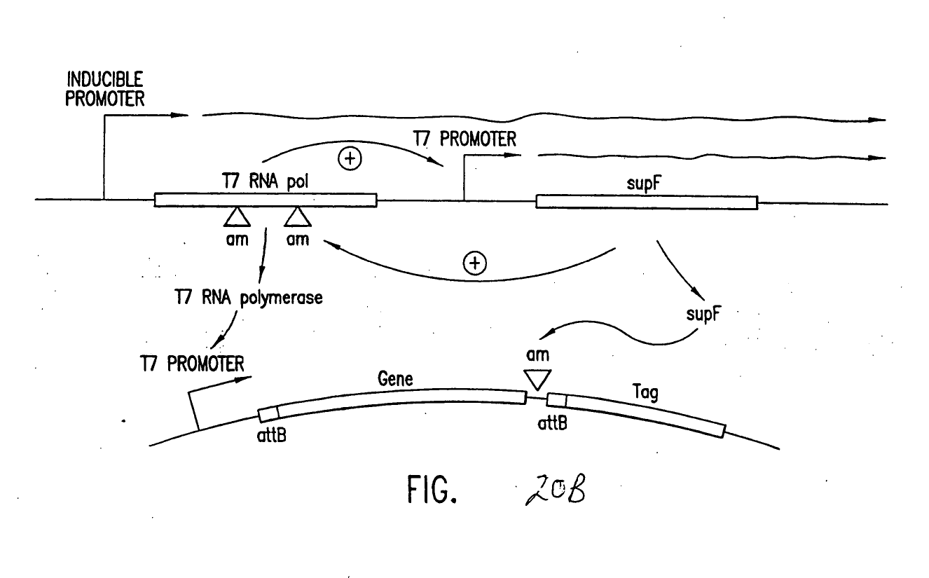

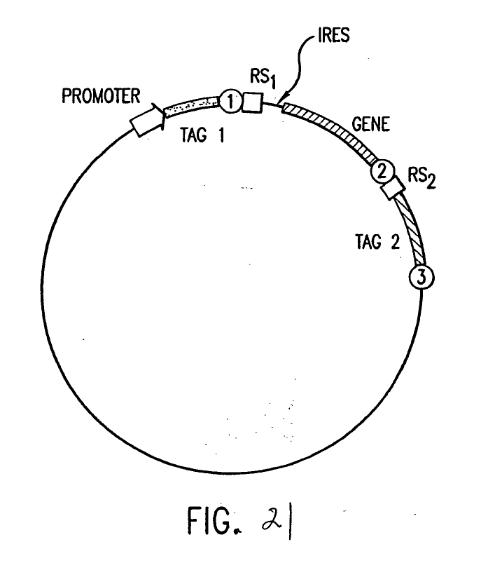

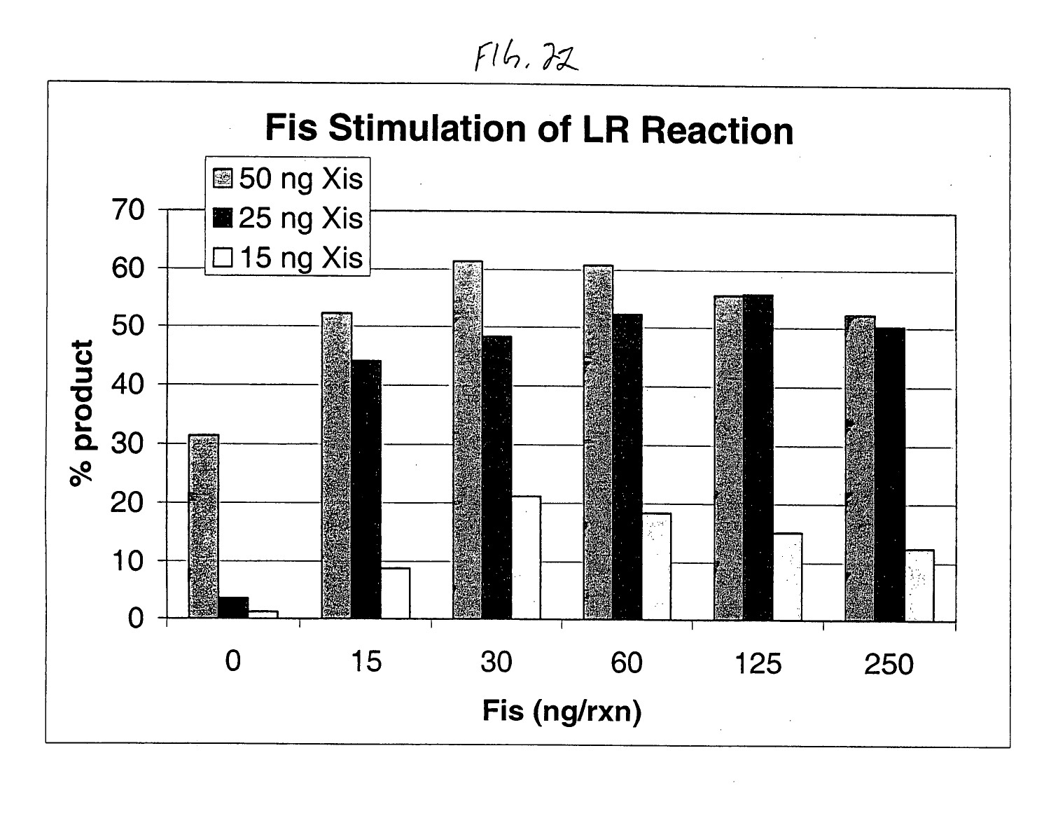

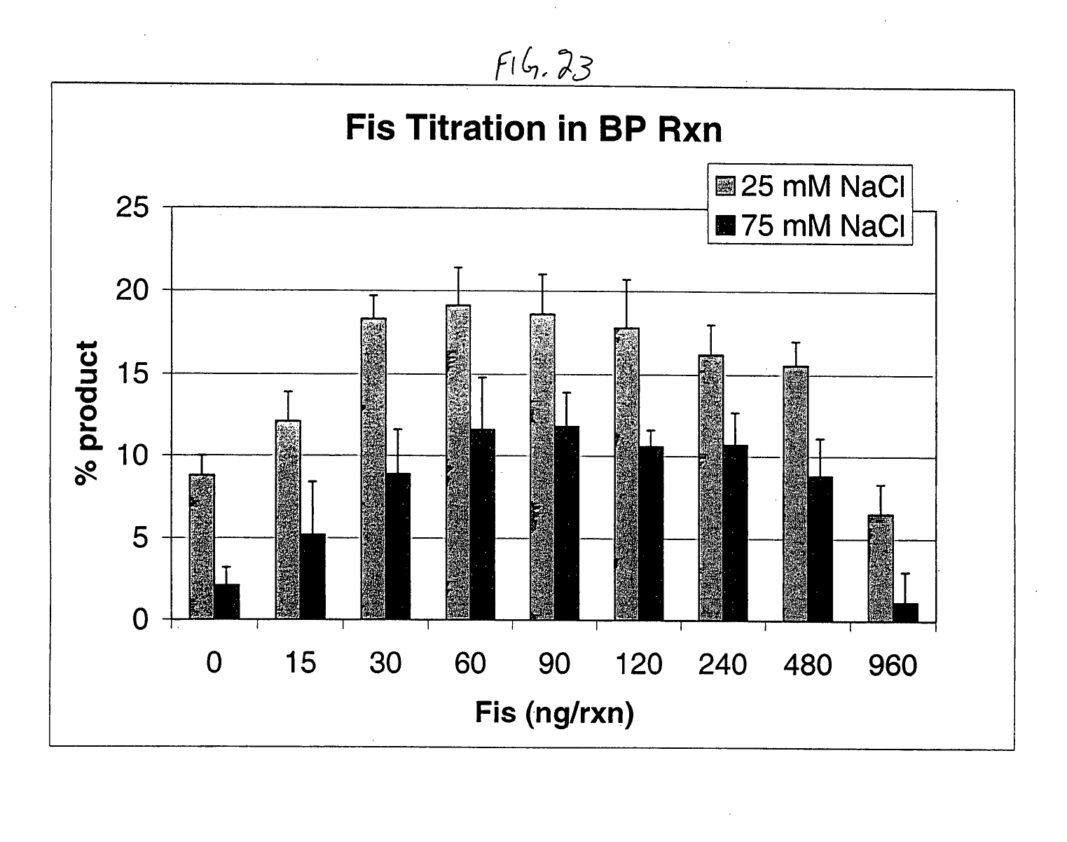

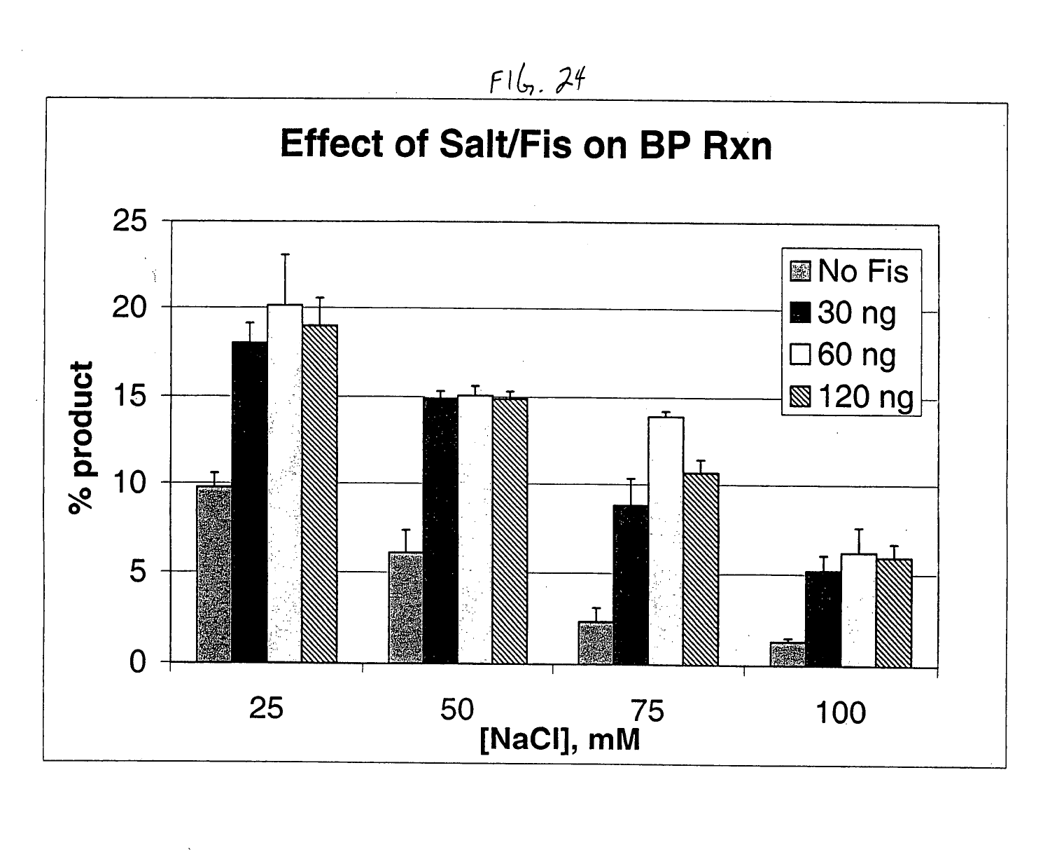

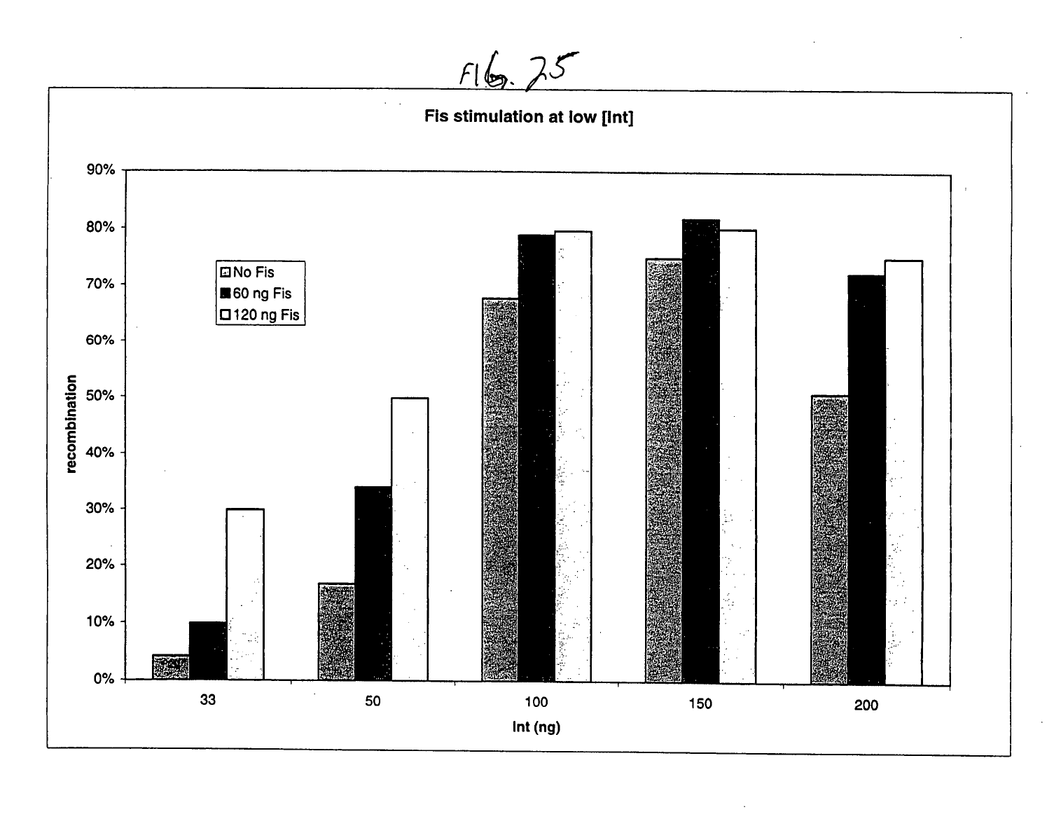

Insert(s): As used herein, the term “insert,” which, for the most part, is used interchangeably with the plural term “inserts,” refers to a nucleic acid segment or a population of nucleic acid segments (segment A of FIG. (9) which may be manipulated by the methods of the present invention. While the sizes of inserts and nucleic acid molecules into which inserts are introduced may vary considerably and are not critical, in many instances, insert will be introduced into larger nucleic acid molecules (e.g., vectors, chromosomes, etc.). For example, the nucleic acid segment labeled “cDNA” in FIG. 2 and the nucleic acid segment labeled “Insert” in FIG. 9 are nucleic acid inserts with respect to the larger nucleic acid molecules (i.e., vectors) into which they are introduced. In most instances, inserts will be flanked by recombination sites (e.g., at least one recombination site at each end). In certain embodiments, however, the insert will only contain a recombination site on one end. Further, the insert may be linear or circular.

Insert Donor: As used herein, the phrase “Insert Donor” refers to one of the two parental nucleic acid molecules (e.g., RNA or DNA) of the present invention which carries the insert. In most instances, the Insert Donor molecule comprises the insert flanked on both sides with recombination sites. The Insert Donor can be linear or circular. In one embodiment of the invention, the Insert Donor is a circular DNA molecule and further comprises nucleic acid of a cloning vector outside of the recombination signals (see FIG. 9). When a population of inserts or population of nucleic acid segments are used to make Insert Donors, a population of Insert Donors results which may be used in accordance with the invention. Examples of such Insert Donor molecules include, but are not limited to, GATEWAY™ Entry Vectors, such as the Entry Vectors depicted in FIGS. 16A-16B, as well as other vectors comprising a gene of interest flanked by one or more attL sites (e.g., attL1, attL2, etc.) for the production of library clones. Insert Donors may be linear or circular and may contain one or more recombination site.

Product: As used herein, the term “Product” refers to one of the desired daughter molecules comprising the A and D segments which is produced after the second recombination event during a recombinational cloning process (see lower portion of FIG. 9). The Product contains the nucleic acid which was to be cloned or subcloned. In accordance with the invention, when a population of Insert Donors are used, the resulting population of Product molecules will contain either all or a portion of the population of inserts of the Insert Donors. Further, the Insert Donors will generally contain a representative population of the original inserts of the Insert Donors. Product molecules may be linear or circular and may contain one or more recombination site.

Target Nucleic Acid Molecule: As used herein, the phrase “target nucleic acid molecule” refers to a nucleic acid molecule which is joined by recombination to a nucleic acid molecule of interest (e.g., a cDNA molecule of a library). Examples of target nucleic acid molecules include, but are not limited to, synthetic nucleic acid molecules, cDNAs, chromosomes, phage genomes, plasmids (e.g., Destination Vectors, Donor Plasmids, etc.), non-nucleic acid molecules containing one or more recombination sites, sub-portions of any of the above, etc. Target nucleic acid molecules will generally contain at least one (e.g., one, two, three, four, five, etc.) recombination site.

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of one or more (e.g., two, three, four, five, seven, ten, etc.) nucleic acid segments into (1) one or more messenger RNAs or (2) one or more untranslated RNAs. Examples of transcriptional regulatory sequences include, but are not limited to, promoters, internal ribosome entry sites (IRES), enhancers, repressors, and the like.

Promoter: A promoter is an example of a transcriptional regulatory sequence. Promoters are nucleic acid are generally located in the 5′-region of a gene, proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Protein which enhances the efficiency of recombination reactions: refers to a protein or peptide which either (1) increases the rate of a recombination reaction or (2) increases the amount of end product resulting from a recombination reaction. Examples of such proteins include Fis proteins and Escherchia coli ribosomal proteins S10, S14, S15, S16, S17, S18, S19, S20, S21, L14, L21, L23, L24, L25, L27, L28, L29, L30, L31, L32, L33 and L34. Further examples are protein fragments (e.g., Fis protein fragments) which enhance the efficiency of one or more recombination reactions. Additional examples are proteins and protein fragments which bind to nucleic acid molecules that Fis binds to (e.g., nucleic acid molecules comprising the nucleotide sequence shown in SEQ ID NO:45 or SEQ ID NO:46) and enhance the efficiency of one or more recombination reactions.

An amount effective for enhancing the efficiency of recombinational cloning: refers to amounts of proteins or protein fragments which enhance the efficiency of recombination reactions. Methods for determining such amounts are set out below in Example 9. In general, proteins or protein fragments which enhance the efficiency of recombination reactions will be included in amounts which result in measurable increases (e.g., increases of at least 5%, at least 10%, at least 15%, at least 20%, at least 25%, at least 30%, at least 35%, at least 50%, etc.) in the efficiency of one or more recombination reactions in comparison to recombination reactions performed in the absence of the proteins or protein fragments. One example of an assay which can be used to measure Fis activity, as well as whether a composition enhances the efficiency of recombination reactions, is the “Recombination assays” section set out below in Example 9.

Ribosomal protein: is a protein, or a mutant or derivative thereof, that is a constituent of a subunit of a ribosome. According to the invention, the ribosome may be a prokaryotic or eukaryotic ribosome. One example of a ribosome is an E. coli ribosome, which comprises a 30S and a 50S subunit.

Ribosomal protein fragment: is a fragment of a protein that is a constituent of a subunit of a ribosome. Generally, ribosomal protein fragments used in the practice of the invention will be functional fragments. By a “functional” fragment is meant a fragment of a native ribosomal protein, or a mutant or derivative of such a fragment, that has substantially the same biological activity as the corresponding native ribosomal protein in stimulating one or more recombination reactions (e.g., a recombination reaction of the λ Int recombination system).

Purified: As used herein, the term purified means that the molecule which is subjected to purification has been separated from at least some surrounding contaminants (e.g., protein, nucleic acids, carbohydrates, etc.). Thus, the term purified is a relative term, with respect to the amount of surrounding contaminants both before and after a desired molecule is subjected to a purification process. Generally, salts, water, buffers and the like are not considered to be contaminants for the purposes of this definition. Thus, the removal of salt from a desired nucleic acid using, for example, a desalting column does not result in purification of the nucleic acid molecule. The term “substantially purified”, as used herein, refers to the removal of at least 90% of original contaminants from the molecules subjected to a purification process.

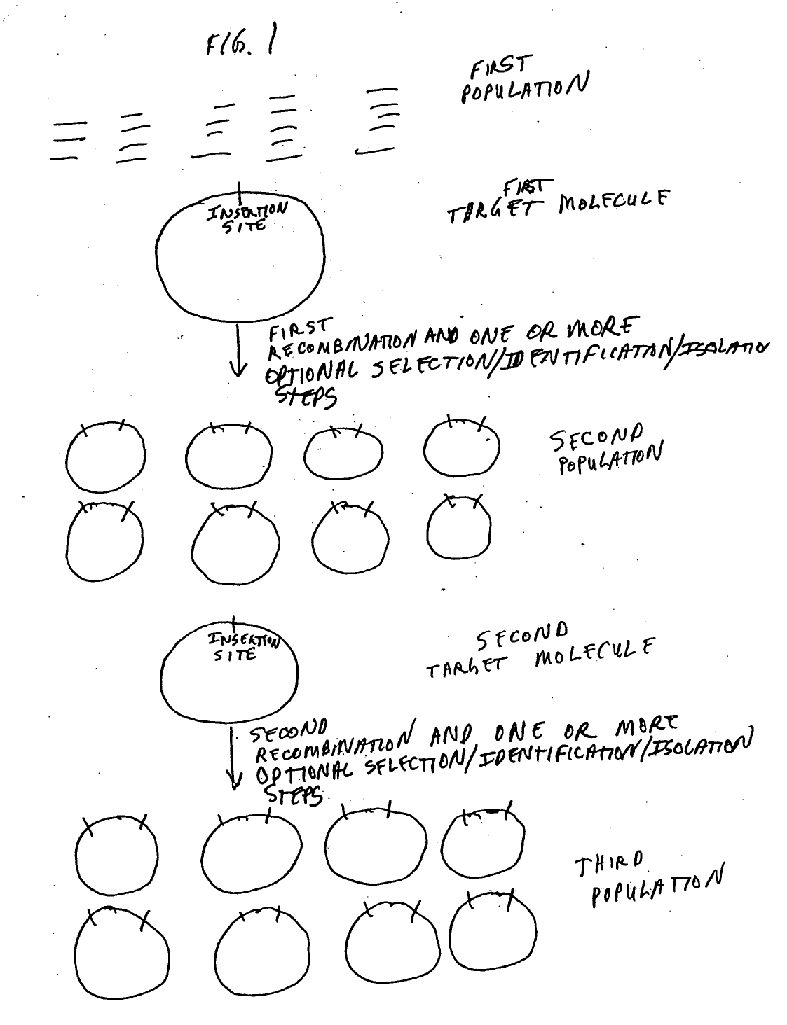

Recognition Sequence: As used herein, the phrase “recognition sequence” refers to a particular sequence to which a protein, chemical compound, DNA, or RNA molecule (e.g., restriction endonuclease, a modification methylase, or a recombinase) recognizes and binds. In the present invention, a recognition sequence will usually refer to a recombination site. For example, the recognition sequence for Cre recombinase is loxP which is a 34 base pair sequence comprising two 13 base pair inverted repeats (serving as the recombinase binding sites) flanking an 8 base pair core sequence. (See FIG. 1 of Sauer, B., Current Opinion in Biotechnology 5:521-527 (1994).) Other examples of recognition sequences are the attB, attP, attL, and attR sequences which are recognized by the recombinase enzyme λ Integrase. AttB is an approximately 25 base pair sequence containing two 9 base pair core-type Int binding sites and a 7 base pair overlap region. AttP is an approximately 240 base pair sequence containing core-type Int binding sites and arm-type Int binding sites as well as sites for auxiliary proteins integration host factor (IHF), Fis, and excisionase (Xis). (See Landy, Current Opinion in Biotechnology 3:699-707 (1993).) Such sites may also be engineered according to the present invention to enhance production of products in the methods of the invention. For example, when such engineered sites lack the P1 or H1 domains to make the recombination reactions irreversible (e.g., attR or attP), such sites may be designated attR′ or attP′ to show that the domains of these sites have been modified in some way.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, φC31, Cin, Tn3 resolvase, TndX, XerC, XerD, Tn7, TnpX, Hjc, Gin, SpCCE1, and ParA. Additional examples of recombination proteins also include Vibrio fischeri super-integron InVfi site-specific recombinase IntIA (intIA) (see, e.g., GenBank Accession No. AY014400), Xanthomonas campestris pv. campestris super-integron InXca site-specific recombinase IntIA (intIA) (see, e.g., GenBank Accession No. AF324483), Salmonella typhimurium recombinase, transposase (tnpA) (see, e.g., GenBank Accession No. AF117344), Bacteriophage mv4 ORFI2, recombinase (int) (see, e.g., GenBank Accession No. U15564), Neisseria gonorrhoeae site-specific recombinase (gcr) (see, e.g., GenBank Accession No. U82253), Clostridium perfringens transposon Tn4451 site-specific recombinase (tnpX) (see, e.g., GenBank Accession No. U15027), Bacillus thuringiensis morrisoni EG2158 transposon Tn5401 site-specific recombinase (tnpI) (see, e.g., GenBank Accession No. U03554), and Anabaena sp. developmentally-regulated site specific recombinase (xisF) (see, e.g., GenBank Accession No. L23220).

Recombination Site: As used herein, the phrase “recombination site” refers to a recognition sequence on a nucleic acid molecule which participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences described herein, and mutants, fragments, variants and derivatives thereof, which are recognized by the recombination protein λ Int and by the auxiliary proteins integration host factor (IHF), Fis and excisionase (Xis). (See Landy, Curr. Opin. Biotech. 3:699-707 (1993).)

Recombination sites may be added to molecules by any number of known methods. For example, recombination sites can be added to nucleic acid molecules by blunt end ligation, PCR performed with fully or partially random primers, inserting the nucleic acid molecules into an vector using a restriction site which flanked by recombination sites or by the use of topoisomerase cloning (see Shuman, J. Biol. Chem. 269:32678-32684 (1994)), which describes molecular cloning and polynucleotide synthesis using Vaccinia DNA topoisomerase; see also Invitrogen 2001 Catalog, pages 6-12 (Invitrogen Corp., Carlsbad, Calif.)).

Recombinational Cloning: As used herein, the phrase “recombinational cloning” refers to a method described herein, whereby segments of nucleic acid molecules or populations of such molecules are exchanged, inserted, replaced, substituted or modified, in vitro or in vivo. By “in vitro” and “in vivo” herein is meant recombinational cloning that is carried out outside of host cells (e.g., in cell-free systems) or inside of host cells (e.g., using recombination proteins expressed by host cells), respectively.

Repression Cassette: As used herein, the phrase “repression cassette” refers to a nucleic acid segment that contains a repressor or a selectable marker present in the subcloning vector.

Selectable Marker: As used herein, the phrase “selectable marker” refers to a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics such as ampicillin, tetracycline, kanamycin, neomycin, hygromycin, zeocin, blastomycin, phleomycin, and G-418); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as (β-galactosidase, green fluorescent protein (GFP), yellow fluorescent protein (YFP), red fluorescent protein (RFP), cyan fluorescent protein (CFP), cell surface proteins, and receptor proteins and other cell surface markers); (5) nucleic acid segments that bind products which are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence which can be otherwise non-functional (e.g., for PCR amplification of subpopulations of molecules); (10) nucleic acid segments, which when absent, directly or indirectly confer resistance or sensitivity to particular compounds; and/or (11) nucleic acid segments that encode products which either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Thus, the phrase “selectable marker” also includes nucleic acid segments which can be used to identify cells having particular characteristics that are not necessarily associated with cell viability (e.g., phenotypic markers such as (β-galactosidase, green fluorescent protein (GFP), yellow fluorescent protein (YFP), red fluorescent protein (RFP), cyan fluorescent protein (CFP), and cell surface proteins).

Further, selection can occur in vitro or in vivo. In vitro selection can be used to select for or identify nucleic acid molecules having particular properties, features, or activities (e.g., bind to particular proteins, encoding proteins with particular properties, features, or activities). In vivo selection can be performed using any number of organisms including bacteria, fingi, plants, and animals. When metazoan organisms are used in selection processes, selection can be based on phenotypic expression exhibited by particular cells of the organisms (e.g., cells of an organ) or all of the cells of the organism.

Selection Scheme: As used herein, the phrase “selection scheme” refers to any method which allows selection, enrichment, or identification of a desired nucleic acid molecules or host cells contacting them (in particular Product or Product(s) from a mixture containing an Entry Clone or Vector, a Destination Vector, a Donor Vector, an Expression Clone or Vector, any intermediates (e.g., a Cointegrate or a replicon), and/or By-products). In one aspect, selection schemes of the invention rely on one or more selectable markers. The selection schemes of some embodiments have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some embodiments, the selection scheme results in selection of or enrichment for only one or more desired nucleic acid molecules (such as Products). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one embodiment, the selection schemes (which can be carried out in reverse) will take one of three forms, which will be discussed in terms of FIG. 9. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, apoptosis-related genes (e.g., ASK1 or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from φX174 or bacteriophage T4; genes which confer metabolite sensitivity such as sacB; antibiotic sensitivity genes such as rpsL; antimicrobial sensitivity genes such as pheS; plasmid killer genes; eukaryotic transcriptional vector genes that produce a gene product toxic to bacteria, such as GATA-1; genes that kill hosts in the absence of a suppressing function, e.g., kicB, ccdB, φX174 E (Liu, Q. et al., Curr. Biol. 8:1300-1309 (1998)); and other genes that negatively affect replicon stability and/or replication. A toxic gene can alternatively be selectable in vitro, e.g., a restriction site.

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

The phrase “selection scheme” also includes methods for screening cells to identify cells having particular characteristics that are not necessarily associated with cell viability (e.g., phenotypic markers such as (β-galactosidase, green fluorescent protein (GFP), yellow fluorescent protein (YFP), red fluorescent protein (RFP), cyan fluorescent protein (CFP), and cell surface proteins). Once such cells have been identified, they may be separated from other cells in a population. Methods which may be used to identify cells having particular characteristics that are not necessarily associated with cell viability include fluorescent detection methods (e.g., FACS cell sorting).

In vitro selection of nucleic acid molecules can be accomplished by any number of means. One example of such a means is by amplification of molecules which hybridize to primers having specified sequences.

Site-Specific Recombinase: As used herein, the phrase “site-specific recombinase” refers to a type of recombinase which typically has at least the following four activities (or combinations thereof): (1) recognition of specific nucleic acid sequences; (2) cleavage of these sequences; (3) topoisomerase-like or transferase activity involved in strand exchange; and (4) ligase activity to reseal the cleaved strands of nucleic acid. (See Sauer, B., Current Opinions in Biotechnology 5:521-527 (1994).) The strand exchange mechanism involves the cleavage and rejoining of specific nucleic acid sequences in the absence of DNA synthesis (Landy, A. (1989) Ann. Rev. Biochem. 58:913-949).

Homologous Recombination: As used herein, the phrase “homologous recombination” refers to the process in which nucleic acid molecules with similar nucleotide sequences associate and exchange nucleotide strands. A nucleotide sequence of a first nucleic acid molecule which is effective for engaging in homologous recombination at a predefined position of a second nucleic acid molecule will therefore have a nucleotide sequence which facilitates the exchange of nucleotide strands between the first nucleic acid molecule and a defined position of the second nucleic acid molecule. Thus, the first nucleic acid will generally have a nucleotide sequence which is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination which occurs, for example, at recombination sites such as att sites, is not considered to be “homologous recombination,” as the phrase is used herein. However, homologous recombination may be used to introduce one or more recombination sites into nucleic acid molecules. Further, due to sequence similarity, nucleic acid molecules which contain recombination sites may undergo homologous recombination.

Subcloning Vector: As used herein, the phrase “subcloning vector” refers to a cloning vector comprising a circular or linear nucleic acid molecule which normally includes an appropriate replicon. In the present invention, the subcloning vector (segment D in FIG. 9) can also contain functional and/or regulatory elements that are desired to be incorporated into the final product to act upon or with the cloned DNA Insert (segment A in FIG. 9). The subcloning vector can also contain a selectable marker and/or may be a nucleic acid segment having a particular property feature, or activity (e.g., promoter activity, hybridizes with another nucleic acid segment, etc.).

Vector: As used herein, the term “vector” refers to a nucleic acid molecule (e.g., DNA) that provides a useful biological or biochemical property to an insert. Examples include plasmids, viruses, phages, autonomously replicating sequences (ARS), centromeres, and other sequences which are able to replicate or be replicated in vitro or in a host cell, or to convey a desired nucleic acid segment to a desired location within a host cell (e.g., by retroviral integration). A vector can have one or more restriction endonuclease recognition sites or recombination sites at which the sequences can be cut in a determinable fashion without loss of an essential biological function of the vector, and into which a nucleic acid fragment can be spliced in order to bring about its replication and cloning. Vectors can further provide primer sites, e.g., for PCR, transcriptional and/or translational initiation and/or regulation sites, recombinational signals, replicons, selectable markers, etc. Thus, methods of inserting a desired nucleic acid fragment which do not require the use of homologous recombination, transpositions or restriction enzymes (such as, but not limited to, UDG cloning of PCR fragments (U.S. Pat. No. 5,334,575, entirely incorporated herein by reference), T:A cloning, and the like) can also be applied to clone a fragment into a cloning vector to be used according to the present invention. The cloning vector can further contain one or more selectable markers suitable for use in the identification of cells transformed with the cloning vector.

Vector Donor: As used herein, the phrase “Vector Donor” refers to one of the two parental nucleic acid molecules (e.g., RNA or DNA) which carries the nucleic acid segments comprising the nucleic acid vector which is to become part of the desired Product(s). The Vector Donor comprises a subcloning vector D (or it can be called the cloning vector if the Insert Donor does not already contain a cloning vector) and a segment C flanked by recombination sites (see FIG. 9). Segments C and/or D can contain elements which contribute to selection for the desired Product daughter molecule, as described above for selection schemes. The recombination signals can be the same or different, and can be acted upon by the same or different recombinases. In addition, the Vector Donor can be linear or circular. Examples of such Vector Donor molecules include GATEWAY™ Destination Vectors, which include but are not limited to the Destination Vectors such as that depicted in FIGS. 17A-17D.

Vector Donors, as well as other vectors of the invention, may contain one or more elements derived from adenoviruses, retroviruses, baculoviruses, alphaviruses, lentiviruses, bacteria, or eukaryotic cells (e.g., yeast cells, plants cells animal cells). Examples of such elements include promoters, packaging signals, coding regions, and nucleic acid which allows for integration into host cell chromosomes. Vector Donors, as well as other vectors of the invention, may be linear or circular.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof. Portions of recombination sites comprise at least 2 bases (or base pairs), at least 5-200 bases, at least 10-100 bases, at least 15-75 bases, at least 15-50 bases, at least 15-25 bases, or at least 16-25 bases, of the recombination sites of interest. When using primers comprising portions of recombination sites, the missing portion of the recombination site may be provided as a template by the newly synthesized nucleic acid molecule. Such recombination sites may be located within and/or at one or both termini of the primer. In many instances, additional sequences are added to the primer adjacent to the recombination site(s) to enhance or improve recombination and/or to stabilize the recombination site during recombination. Such stabilization sequences may be any sequences (e.g., G/C rich sequences) of any length. Such sequences may have a wide range of sizes, such as from about 3 to about 1000 bases, from about 3 to about 500 bases, from about 3 to about 100 bases, from about 3 to about 60 bases, from about 3 to about 25, from about 3 to about 10, from about 3 to about 10, and from about 3 to about 4 bases.

Template: As used herein, the term “template” refers to a double stranded or single stranded nucleic acid molecule which is to be amplified, synthesized or sequenced. In the case of a double-stranded DNA molecule, denaturation of its strands to form a first and a second strand can occur before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Adapter: As used herein, the term “adapter” refers to an oligonucleotide or nucleic acid fragment or segment (e.g., DNA) which comprises one or more recombination sites (or portions of such recombination sites) which in accordance with the invention can be added to a circular or linear Insert Donor molecule, as well as other nucleic acid molecules described herein. When using portions of recombination sites, the missing portion may be provided by the Insert Donor molecule. Such adapters may be added at any location within a circular or linear molecule, although the adapters may be added at or near one or both termini of a linear molecule. Further, adapters may be positioned to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule which contains the adapter(s) at the site of cleavage. In other aspects, adapters may be added by homologous recombination, by integration of RNA molecules, and the like. Alternatively, adapters may be ligated directly to one, more and/or both termini of a linear molecule thereby resulting in linear molecule(s) having adapters at one or both termini. In one aspect of the invention, adapters may be added to a population of linear molecules (e.g., a cDNA library or genomic DNA which has been cleaved or digested) to form a population of linear molecules containing adapters at one or both termini of all or substantial portion of said population.

Adapter-Primer: As used herein, the phrase “adapter-primer” refers to primer molecule which comprises one or more recombination sites (or portions of such recombination sites) which in accordance with the invention can be added to a circular or linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers may be added at or near one or both termini of a linear molecule. Adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

Library: As used herein, the term “library” refers to a collection of nucleic acid molecules (circular or linear) which differ in nucleotide sequence (e.g., a population of nucleic acid molecules in which at least 75, 85, 96, 100, 192, 288, 384, 480, 500, 576, 672, 768, 864, 960, 1,000, 1056, 1152, 1248, 1344, 1440, 1536, 1632, 1728, 1824, 2,000, 3,000, 5,000, 10,000, 15,000, 20,000, 30,000, 50,000, 70,000, 80,000, etc. of the individual nucleic acid molecules comprise different sequences and share no regions of sequence identify which are greater than 100 nucleotides). In one embodiment, a library is representative of all or a portion or a significant portion of the nucleic acid content of an organism (a “genomic” library), or a set of nucleic acid molecules representative of all, a portion or a significant portion (e.g., about 50%, about 60%, about 70%, about 80%, about 90%, about 95%, etc.) of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one vector or two or more (e.g., two, three, four, five, seven, ten, twelve, fifteen, twenty, thirty, fifty, etc.) different vectors. Libraries used in the practice of the invention may be normalized libraries. Further, these libraries may comprise molecules which are linear or circular.

In addition, libraries of the invention may comprise (1) multiple nucleic acid molecules which differ in sequence but are not vectors (e.g., cDNA molecules, genomic DNA molecules, synthetic nucleic acid molecules), which may or may not be inserted into a vector, or (2) multiple vectors which differ in nucleotide sequence, which may or may not contain one or a small number (e.g., two, three, four, etc.) of nucleic acid molecules but are not vectors.

Normalized Libraries: As used herein, the phrase “normalized libraries” refers to libraries where the number of nucleic acid molecules originally present in relatively high/higher copy numbers are reduced with respect to the number of nucleic acid molecules which are present in low/lower copy numbers. Normalization of libraries is often done to reduce the number of cDNA molecules in a library which represent highly expressed genes. In other words, libraries are often normalized to reduce the number of nucleic acid molecules which represent abundant RNAs. Methods for preparing normalized libraries are known in the art and are described, for example, in U.S. Pat. Nos. 6,001,574, 5,637,685, 5,846,721, and 5,763,239, the entire disclosures of which are incorporated herein by reference.

One methods for normalizing libraries is described in Patanjali et al., Proc. Natl. Acad. Sci. USA 88:1943-1947 (1991) (the entire disclosure of which is incorporated herein by reference). This method employs a kinetic approach to construct cDNA libraries containing roughly equal representations of all molecules in a preparation of poly(A)+ RNA. According to this method, randomly primed cDNA fragments of a selected size range are cloned in a vector, inserts are then amplified by PCR, denatured, and self-annealed under optimized conditions. Upon extensive but incomplete reannealing, single-stranded fractions become depleted of more abundant species of cDNA.

Rubenstein et al., Nucleic Acids Res. 18:4833-4842 (1990) (the entire disclosure of which is incorporated herein by reference), for example, describes a subtractive hybridization protocol which permits subtractions between cDNA libraries. The method uses single-stranded phagemids with directional inserts as both the driver and the target. Using a model system, Rubenstein et al. found that one round of subtractive hybridization resulted in a 5,000-fold specific subtraction of abundant molecules. A number of similar processes are also known in the art. Subtractive hybridization may be used to normalize libraries of the invention.

“Normalized” libraries may also be generated by the introduction of mutations in a fixed number of nucleic acid molecules (e.g., one, two, three, four, five, ten, twenty, etc.). For example, a normalized library may be generated by the introduction of random mutations in one nucleic acid molecule. Upon amplification after completion of mutagenesis, the individual mutagenized nucleic acid molecules should be represented in roughly equal proportions. Further, mutations may be introduced into only part of one or more nucleic acid molecules. For example, random mutations may be introduced into a region of a nucleic acid molecule which encodes a domain of a protein. Such a normalized libraries may be normalized with respect to sequences represented by the mutagenized portion of the nucleic acid molecule.

Amplification: Depending on the context, as used herein, the term “amplification” refers to any in vitro method for increasing the number of copies of a nucleic acid with the use of a polymerase. Nucleic acid amplification results in the incorporation of nucleotides into a DNA and/or RNA molecule or primer thereby forming a new molecule complementary to a template. The formed nucleic acid molecule and its template can be used as templates to synthesize additional nucleic acid molecules. As used herein, one amplification reaction may consist of many rounds of replication. DNA amplification reactions include, for example, polymerase chain reaction (PCR), ligase chain reaction, and rolling circle amplification. (See PCT Publication Nos. WO 93/00447 and WO 00/15779, the entire disclosures of which are incorporated herein by reference.) Further, one PCR reaction may consist of 5-100 “cycles” of denaturation and synthesis of a DNA molecule.

The term “amplification” can also refer to the production of nucleic acid molecules in vivo, which often occurs after introduction into a cell. Thus, a plasmid, for example, may be amplified by transformation of cells in which the plasmid is capable of replicating. These cells may then be cultured and the “amplified” plasmid can then be isolated.

Oligonucleotide: As used herein, the term “oligonucleotide” refers to refers to a synthetic or natural molecule comprising a covalently linked sequence of nucleotides which are joined by a phosphodiester bond between the 3′ position of the deoxyribose or ribose of one nucleotide and the 5′ position of the deoxyribose or ribose of the adjacent nucleotide. This term may be used interchangeably herein with the terms “nucleic acid molecule” and “polynucleotide,” without any of these terms necessarily indicating any particular length of the nucleic acid molecule to which the term specifically refers.

Nucleotide: As used herein, the term “nucleotide” refers to refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [γS]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Other terms used in the fields of recombinant DNA technology and molecular and cell biology as used herein will be generally understood by one of ordinary skill in the applicable arts.

Overview

In one general aspect, the invention relates to methods for inserting one or more (e.g., one, two, three, four, five, six, seven, eight, nine, ten, fifteen, twenty, thirty, fifty, one hundred, five hundred, one thousand, two thousand, five thousand, ten thousand, twenty thousand, fifty thousand, one hundred thousand, etc.) nucleic acid molecules into one or more other nucleic acid molecules (e.g., a “target nucleic acid molecule”), methods for transferring one or more nucleic acid molecules which reside in a first nucleic acid molecule (e.g., a “target nucleic acid molecule”) into a second nucleic acid molecule (e.g., a “target nucleic acid molecule”), and selection and/or screening methods for identifying nucleic acids and proteins having particular properties, features, activities, and/or characteristics. In many embodiments, methods of the invention involve the use and/or transfer of populations of nucleic acid molecules (e.g., cDNA libraries). The invention further relates to populations of nucleic acid molecules prepared by methods of the invention and individual nucleic acid molecules prepared and/or isolated by methods of the invention.

The invention further relates, in part, to methods for inserting nucleic acid molecules into one or more target nucleic acid molecules (e.g., vectors, chromosomes, etc.), methods for transferring nucleic acid molecules between target nucleic acid molecules, and screening and selection methods for identifying nucleic acid molecules and proteins having particular features, activities, characteristics and/or properties.

In addition, the invention relates, in part, to methods and compositions for the identification and/or isolation of one or more populations or subpopulations of nucleic acid molecules. In specific embodiments, methods and compositions of the invention employ recombinational cloning systems, such as the GATEWAY™ Cloning System described in detail in U.S. Pat. No. 5,888,732; PCT Publication No. WO 00/52027; U.S. application Ser. No. 09/177,387, filed Oct. 23, 1998; U.S. application Ser. No. 09/438,358, filed Nov. 12, 1999; U.S. application Ser. No. 09/517,466, filed Mar. 2, 2000, and U.S. application Ser. No. 09/732,914, filed Dec. 11, 2000 (the disclosures of all of which are incorporated herein by reference in their entireties) to rapidly and efficient (1) transfer nucleic acid molecules (e.g., cDNA molecules) from a nucleic acid molecule (e.g., vector) in which they are contained into a target nucleic acid molecule or (2) insert nucleic acid molecules (e.g., cDNA molecules) into a target nucleic acid molecule. Since different target nucleic acid molecules provide different properties, features, or activities to nucleic acid molecules which are inserted into them (and vice versa), populations and subpopulations of nucleic acid molecules can be selected for based on these different properties, features, or activities in a reiterative (e.g., sequential) manner using methods of the invention.

In one specific aspect, the invention is directed to methods for transferring populations of nucleic acid molecules between target nucleic acid molecules. In particular, populations of nucleic acid molecules are transferred from one target nucleic acid molecule to another target nucleic acid molecule using at least one (e.g., one, two, three, four, five, etc.) recombination reaction. Further, the populations of nucleic acid molecules which are transferred between target nucleic acid molecules will generally contain at least one (e.g., one, two, three, four, five, etc.) recombination site generally located at at least one terminus of the individual members of the population. In addition, populations of nucleic acid molecules which are transferred between target nucleic acid molecules may contain two recombination sites, one located at each end of the individual members of the population. The invention further includes populations of nucleic acid molecules produced by methods of the invention, as well as individual members of these populations.

In specific embodiments, the invention is directed to methods for improving the efficiency of processes for transferring nucleic acid molecules (e.g., the nucleic acid molecules of a cDNA or genomic library) which reside in a first nucleic acid molecule (e.g., a vector, a chromosome, etc.) into a target nucleic acid molecule. As one skilled in the art would recognize, how the efficiency of transfer is determined depends on the conditions of the specific transfer process. For example, transfer efficiency may be quite different when comparing the percentage of an initial population of nucleic acid molecules (e.g., cDNA molecules) which are inserted into a first target molecules, as compared to the efficiency of transfer of insert between target molecules or the efficiency of transfer of one insert between populations of different vector molecules.

Thus, in one aspect, the invention provides methods for transferring nucleic acid molecules of a population of nucleic acid molecules into a first target nucleic acid molecule (e.g., a vector, a chromosome, etc.) such that a substantial percentage (e.g., greater than about 10%, greater than about 20%, greater than about 30%, greater than about 40%, greater than about 50%, greater than about 60%, greater than about 70%, greater than about 80%, greater than about 90%, greater than about 95%, greater than about 98%, greater than about 99%, etc.) of the first target nucleic acid molecules contain inserts. In a related aspect, the first target nucleic acid molecules may comprise a mixed population of molecules which differ in nucleotide sequence. Of course, the percentage of target molecules which contain inserts will vary with the relative concentrations of the nucleic acid molecules which undergo recombination. For example, when the nucleic acid molecules of a population of nucleic acid molecules are in excess with respect to the first target nucleic acid molecules, then a relatively high percentage of the first target nucleic acid molecules will generally contain inserts after recombination.

In another aspect, the invention provides methods for transferring nucleic acid molecules of a population of nucleic acid molecules contained in a first target nucleic acid molecule (e.g., a vector, a chromosome, etc.) into a second target nucleic acid molecule such that a substantial percentage (e.g., greater than about 10%, greater than about 20%, greater than about 30%, greater than about 40%, greater than about 50%, greater than about 60%, greater than about 70%, greater than about 80%, greater than about 90%, greater than about 95%, greater than about 98%, greater than about 99%, etc.) of the nucleic acid molecules intended for transfer are transferred into the second target nucleic acid molecule. In a related aspect, the invention provides methods for transferring nucleic acid molecules of a population of nucleic acid molecules contained in a first target nucleic acid molecule (e.g., a vector, a chromosome, etc.) into a second target nucleic acid molecule such that a substantial percentage (e.g., greater than about 10%, greater than about 20%, greater than about 30%, greater than about 40%, greater than about 50%, greater than about 60%, greater than about 70%, greater than about 80%, greater than about 90%, greater than about 95%, greater than about 98%, greater than about 99%, etc.) of the second target nucleic acid molecule contain inserts. In other words, the invention provides methods for the efficient transfer of nucleic acid molecules (e.g., the molecules of a cDNA library) from nucleic acid molecule in which they reside (e.g., a vector, a chromosome, etc.) into target nucleic acid molecules (e.g., a vector, a chromosome, etc.).

The invention further provides methods for transferring multiple copies of one or a small number of nucleic acid molecules, which are not target nucleic acid molecules, from a first target nucleic acid molecule into a population of second target nucleic acid molecules, such that a substantial percentage (e.g., greater than about 10%, greater than about 20%, greater than about 30%, greater than about 40%, greater than about 50%, greater than about 60%, greater than about 70%, greater than about 80%, greater than about 90%, greater than about 95%, greater than about 98%, greater than about 99%, etc.) of the second target nucleic acid molecules undergo recombination which results in the insertion of the one or a small number of nucleic acid molecules.

Nucleic acid transfer methods of the invention may result in nucleic acid molecules (e.g., cDNAs) being sequentially transferred to more than one (e.g., two, three, four, five, six, seven, eight, nine, ten, etc.) target nucleic acid molecules. For example, nucleic acid molecules being sequentially transferred from one target nucleic acid molecule to another target nucleic acid molecule may be transferred to one or more (e.g., two, three, four, five, six, seven, eight, nine, ten, etc.) intermediary target nucleic acid molecules. These intermediary target nucleic acid molecules may be used for any number of purposes. For example, intermediary target nucleic acid molecules may be used to amplify the nucleic acid molecules being transferred or to add or remove particular nucleotide sequences (e.g., recombination sites; restriction sites; nucleotide sequences which encode signal peptides, epitope tags, polypeptides having one or more enzymatic activities; etc.) to/from the molecules being transferred.

Using the process shown in FIG. 1 for illustration, a first population of nucleic acid molecules (e.g., a cDNA library), each of the individual molecules of which contain recombination sites at one or both termini, are inserted into a first target nucleic acid molecule (e.g., a vector, a chromosome, etc.) by a first recombination reaction to produce a second population of nucleic acid molecules. In this instance, the first target nucleic acid molecule is an intermediary target nucleic acid molecule since individual members of the population of nucleic acid molecules which have been inserted into the first target nucleic acid molecule are then transferred to a second target nucleic acid molecule by a second recombination reaction to form a third population of nucleic acid molecules. Thus, methods of the invention include the transfer of nucleic acid molecules, using one or more recombination reactions (e.g., reactions of the Cre/loxP and/or the Flp/FRT recombination systems), from one target nucleic acid molecule to another target nucleic acid molecule, either directly or through one or more intermediary target nucleic acid molecules.

As one skilled in the art would recognize, numerous variations of the general process show in FIG. 1, many of which are set out herein are possible, are included within the scope of the invention.

In one general aspect, the invention is directed to methods for inserting populations of nucleic acid molecules into target molecules. In specific embodiments, these methods comprise:
(a) mixing at least one first population of nucleic acid molecules (e.g., a cDNA library) comprising one or more (e.g., one, two, three, four, five, six, eight, ten, etc.) recombination sites with at least one (e.g., one, two, three, four, five, six, eight, ten, etc.) first target nucleic acid molecule comprising one or more (e.g., one, two, three, four, five, six, eight, ten, etc.) recombination sites;
(b) causing some or all of the nucleic acid molecules of the at least one first population to recombine with some or all of the first target nucleic acid molecules, thereby forming a second population of nucleic acid molecules;
(c) mixing at least the second population of nucleic acid molecules with at least one second target nucleic acid molecule comprising one or more (e.g., one, two, three, four, five, six, eight, ten, etc.) recombination sites; and
(d) causing some or all of the nucleic acid molecules of the at least second population to recombine with some or all of the second target nucleic acid molecules, thereby forming a third population of nucleic acid molecules.

Further, steps (c) and (d) referred to above may be repeated, resulting in the transfer of individual members of the first population of nucleic acid molecules through a series of target nucleic acid molecules, referred to herein as intermediary target nucleic acid molecules. Thus, according to methods of the invention, individual members of the first population of nucleic acid molecules may be transferred from one target nucleic acid molecule to one or more other target nucleic acid molecules. Further, with each transfer, new populations of nucleic acid molecules are formed.

As discussed below, either one or both of the nucleic acid molecules (e.g., the individual members of the first population of nucleic acid molecules, the first target nucleic acid molecule, etc.) which participate in recombination reactions performed during the practice of the invention may be linear or closed, circular. Further, closed, circular nucleic acid molecules may be relaxed, negatively supercoiled, or positively supercoiled.

In addition, sites suitable for linearizing nucleic acid molecules may be present in one or both of the molecules undergoing recombination (e.g., the individual members of the first population of nucleic acid molecules, the first target nucleic acid molecule, etc.). Examples of such sites include recombination sites and restriction enzyme recognition sites. Further, linear nucleic acid molecules may be generated by amplification across a population of molecules to generate a linear population.

Generally, sites suitable for linearizing nucleic acid molecules will be designed to linearize the nucleic acid molecule in which they are present while having little or no effect on nucleic acid molecules being transferred (e.g., cDNA molecules) or nucleic acid which confers functional properties, features, or activities used for molecular cloning (e.g., selection markers, origins of replication, etc.). As noted above, examples of such sites include recombination sites and restriction sites which recognize rare sequences. These sites may be used to cleave nucleic acid molecules such that in almost all instances, nucleic acid is cleaved only at desired locations. Thus, when a population of molecules which contains a genomic library, for example, is linearized, the nucleic acid molecules which make up the library will be cleaved in only extremely rare instances. Further, limit digests may be used in instances where there is a concern that the linearization method used results in the exclusion of particular nucleic acid molecules from the transfer process. Recombination sites which can be used with this aspect of the invention are described elsewhere herein.

Restriction sites which both recognize rare sequences and can be used with the invention include ISceI (see Kirik et al., EMBO J. 19:5562-5566 (2000)), NotI, SfiI (see Caccio et al., Gene 219:73-79 (1998)) sgfI (Kappelman et al., Gene 160:55-58 (1995)), and the HO nuclease of Saccharomyces cerevisiae (see Kostriken and Heffron, Cold Spring Harb. Symp. Quant. Biol. 49:89-96 (1984), Nickoloff et al., Proc. Natl. Acad. Sci. USA 83:7831-5 (1986)). Homing endonucleases, which are rare-cutting enzymes encoded by introns and inteins (see Belfort and Roberts, Nucleic Acids Res. 25:3379-3388 (1997), may also be used with the invention.

In many instances, it will be desirable for recombination reactions to occur at particular nucleic acid concentrations of the population of nucleic acid molecules and target nucleic acid molecules. For example, nucleic acid molecules of the population of nucleic acid molecules (e.g., a cDNA library/Expression Clones) may be present at a variety of concentrations including about 0.1 ng/μl, about 0.5 ng/μl, about 1.0 ng/μl, about 1.5 ng/μl, about 2.0 ng/μl, about 2.5 ng/μl, about 3.0 ng/μl, about 4.0 ng/μl, about 5.0 ng/μl, about 6.0 ng/μl, about 7.0 ng/μl, about 8.0 ng/μl, about 9.0 ng/μl, about 10 ng/μl, about 12 ng/μl, about 13 ng/μl, about 15 ng/μl, about 20 ng/μl, about 25 ng/μl, about 40 ng/μl, about 50 ng/μl, about 70 ng/μl, about 100 ng/l, about 150 ng/μl, about 200 ng/μl, about 250 ng/μl, about 300 ng/μl, about 350 ng/μl, about 400 ng/μl, about 500 ng/μl, about 600 ng/μl, about 700 ng/μl, about 800 ng/μl, about 900 ng/μl, or about 1000 ng/μl.

Further, the target nucleic acid molecule (e.g., a pDONR plasmid, a Destination Vector) may be present at a variety of concentrations including about 0.1 ng/μl, about 0.5 ng/μl, about 1.0 ng/μl, about 1.5 ng/μl, about 2.0 ng/μl, about 2.5 ng/μl, about 3.0 ng/μl, about 4.0 ng/μl, about 5.0 ng/μl, about 6.0 ng/μl, about 7.0 ng/μl, about 8.0 ng/μl, about 9.0 ng/μl, about 10 ng/μl, about 12 ng/μl, about 13 ng/μl, about 15 ng/μl, about 20 ng/μl, about 25 ng/μl, about 40 ng/μl, about 50 ng/μl, about 70 ng/μl, about 100 ng/μl, about 150 ng/μl, about 200 ng/μl, about 250 ng/μl, about 300 ng/μl, about 350 ng/μl, about 400 ng/μl, about 500 ng/μl, about 600 ng/μl, about 700 ng/μl, about 800 ng/μl, about 900 ng/μl, or about 1000 ng/μl.

As discussed below, in many instances, it will be desirable for the population of nucleic acid molecules to be a limiting component of a recombination reaction. In such instances, the target nucleic acid molecule will normally be present in excess with respect to the population of nucleic acid molecules. The ratio of target nucleic acid molecule to the population of nucleic acid molecules may vary considerable but can be, for example, about 0.1:1, about 0.2:1, about 0.4:1, about 0.5:1, about 1.0:1, about 1.5:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 11:1, about 12:1, about 13:1, about 14:1, about 15:1, about 17:1, about 20:1, about 22:1, about 25:1, about 27:1, about 30:1, about 35:1, about 40:1, about 45:1, about 50:1, about 60:1, about 70:1, about 80:1, about 90:1, or about 100:1.

In instances where the initial nucleic acid molecules involved in one recombination reaction (e.g., the first population of nucleic acid molecules, the first target nucleic acid molecule, etc.), or other nucleic acid molecules which are present, either will not substantially interfere with later recombination reactions or can be eliminated (e.g., removed, degraded, substantially diluted, etc.), the entire transfer process can be efficiently performed in a single tube. Using the depiction in FIG. 2 for purposes of illustration, if the Expression Clones or pDONR plasmid will not (1) substantially interfere with the LR CLONASE™ catalyzed recombination reaction or (2) interfere with the identification of Expression Clone products of this reaction, then amplification of the Entry Clones (i.e., the second population of nucleic acid molecules), for example, would not be necessary and the transfer of libraries of nucleic acids can be accomplished in a single tube.

One way that nucleic acid molecules involved in one recombination reaction can interfere with later events in processes of the invention is by co-transformation of cells along with the individual members of later formed populations of nucleic acid molecules. Again using the process set out in FIG. 2 for purposes of illustration, the initial Expression Clones and the product Expression Clones each contain an ampicillin resistance marker. Thus, if substantial quantities of the initial Expression Clones are present and remain capable of transforming cells, then the initial Expression Clones could co-transform cells along with product Expression Clones, thereby decreasing the efficiency of the overall process.

Conjugative transfer may also be employed to facilitate the transfer of particular nucleic acid molecules between cells. Using the process shown in FIG. 7 for purposes of illustration, the pDONR vector shown in this figure contains an origin of CDT (oriT) which results in the transfer of the vector from a donor cell to a recipient cell during conjugation. Essentially only vectors which contain the oriT will be transferred during conjugation. Conjugative transfer methods are described in Schafer et al., U.S. Pat. No. 5,346,818, the entire disclosure of which is incorporated herein by reference. Thus, nucleic acid molecules, as well as the use of such molecules in processes of the invention, which contain components which result in the selective transfer of these molecules between cells are included within the scope of the invention.

Potential problems related to interference from initial nucleic acid molecules can be reduced or prevented in a number of ways. For example, the concentration of populations of nucleic acid molecules which undergo recombination can be kept low, as compared to the concentration of target nucleic acid molecules. Thus, the populations of nucleic acid molecules will be a limiting participant in recombination reactions. Further, recombination proteins can be included in reaction mixtures in relatively high concentrations to drive first recombination reactions as far to completion as possible. Also, the products of the recombination reactions which might interfere with later steps can be linearized and then treated with one or more nucleases which digest nucleic acid molecules having one or more free ends. Examples of such enzymes include λ Exonuclease, Exonuclease I, Exonuclease III, and Exonuclease V, and U70 (i.e., an alkaline exonuclease of Human herpesvirus 6, see GenBank Accession No. NP_{—}042963). Thus, the invention includes methods in which the products of recombination reactions are treated with exonucleases. Further, nucleic acid molecules may be removed by subtractive hybridization, as described for the preparation of normalized libraries. In other words, the invention provides both negative and positive selection systems for isolating nucleic acid molecules.

Further, potential problems related to interference from initial nucleic acid molecules can be reduced or prevented by the use of subtractive hybridization, as described above for the preparation of normalized libraries.

Another method which can be used to favor the amplification of one nucleic acid molecule over another in cellular systems is by the use of genetic components which only function under particular conditions (e.g., temperature sensitive genetic components, conditional origins of replication). Thus, in one aspect, the invention provides nucleic acid molecules which can be amplified intracellularly only under certain conditions. Example of components which can be used to prepare such nucleic acid molecules are illustrated in FIGS. 6A-6D. In particular, as discussed below in Example 11, the inventors have found that when a kanamycin resistance gene (e.g., kanamycin resistance genes contained in pDONR212 or pDONR212(F), illustrated, respectively, in FIGS. 27A-27C and 28A-28C) is located on a nucleic acid molecule in close proximity to an origin of replication (e.g., an origin of replication contained in pDONR212 or pDONR212(F), illustrated, respectively, in FIGS. 27A-27C and 28A-28C), either the kanamycin resistance gene or the origin of replication cease to function under particular conditions. For example, when a kanamycin resistance gene is located in a nucleic acid molecule at a distance of about 165 base pairs from an Escherchia coli origin of replication and the directions of function of these components face away from each other (see the orientation shown in FIG. 6A), at least one of these two genetic elements does not function in E. coli cells at temperatures between 25° C. and 30° C., referred to herein as “restrictive temperatures”. However, both of these two genetic elements do function at 37° C., referred to herein as a “permissive temperature”.

Thus, in one general aspect, the invention provides compositions comprising combinations of genetic elements which confer upon cells a temperature sensitive phenotype. These combinations of genetic elements may exhibit “cold” (i.e., permissive temperatures are higher than restrictive temperatures) or “hot” (i.e., permissive temperatures are lower than restrictive temperatures) sensitivity. Further, the combinations of genetic elements may comprise two or more (e.g., two, three, four, five, six, seven, eight, etc.) selectable markers, transcriptional regulatory sequences, origins of replication (e.g., origins of conjugative DNA transfer; conditional origins of replication, such as those of plasmids RK2 and R6K (see Easter et al., J. Bacteriol. 179:6472-6479 (1997)), etc.), and replication terminator alleles (e.g., tus and ter (see Anderson et al., Mol. Microbiol. 36:1327-1335 (2000))). The invention further provides methods for using temperature sensitive combinations of genetic elements in methods of the invention, as well as host cells which contain these combinations of genetic elements.

The invention further includes methods which are performed in multiple (e.g., two, three, four, five, six, eight, ten, etc.) steps and/or reaction tubes in which transfer of nucleic acid molecules either into a target nucleic acid molecule or between target nucleic acid molecules occurs at different times or in different reaction mixtures or tubes. One exmaple of such a precess is set out below in Example 6.

In specific embodiments, as noted above and below in Example 11, the invention provides temperature sensitive combinations of at least two genetic elements, wherein one of the at least two genetic elements is an antibiotic resistance marker (e.g., a kanamycin resistance marker, an ampicillin resistance marker, a gentamycin resistance marker, etc.) and one of the at least two genetic elements is an origin of replication. In additional specific embodiments, the antibiotic resistance marker and origin of replication are situated with respect to each other such that they confer a temperature sensitive phenotype. In particular, these genetic elements, as well as other genetic elements used in compositions and methods of the invention, have directions of function shown in FIGS. 6A-6D. In specific embodiments, these directions of functionalities correspond to that shown in FIG. 6A (i.e., their directions of function face away from each other).

Using the schematic shown in FIG. 6A and FIGS. 27A-27C for purposes of illustration, the positioning a kanamycin resistance marker and an origin of replication about 162 base pairs from each other, wherein the marker and origin have directions of function which are directed away from each other results in exhibition of a “cold” sensitive phenotype. More specifically, E. coli cells which contain a vector (e.g., pDONR212 and pDONR212(F)) having these elements in such positions that fewer colonies form on plates containing kanamycin at 25° C. and 30° C. than at 37° C.

Thus, in specific embodiments, the invention provides compositions comprising temperature sensitive combinations of genetic elements, wherein the genetic elements comprise at least one antibiotic resistance marker and at least one origin of replication. Further, the directions of function of these elements may be directed away from each other (see FIG. 6A), towards each other (see FIG. 6C), or in the same direction (see FIGS. 6B and 6D).

In general, genetic elements which confer the temperature sensitive phenotype will be on the same nucleic acid molecules (i.e., are in a cis format). Further, these elements may be located at various distances from each other. For example, the elements may be separated by about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 90 nucleotides, about 100 nucleotides, about 120 nucleotides, about 140 nucleotides, about 160 nucleotides, about 180 nucleotides, about 200 nucleotides, about 230 nucleotides, or about 250 nucleotides of intervening nucleic acid.

The temperature sensitive phenotype of combinations of genetic elements may be exhibited at various temperatures. Further, the particular restrictive and permissive temperatures will vary with the particular genetic elements and the cells which exhibit phenotypes conferred by these elements. For combinations of genetic components which confer cold sensitive phenotypes, examples of restrictive temperatures include 10° C., 15° C., 20° C., 21° C., 22° C., 23° C., 24° C., 25° C., 26° C., 27° C., 28° C., 29° C., 30° C., 31° C., and 32° C., and examples of permissive temperatures include 35° C., 36° C., 37° C., 38° C., 39° C., 40° C., 41° C., and 42° C. For combinations of genetic components which confer cold sensitive phenotypes, examples of permissive temperatures include 10° C., 15° C., 20° C., 21° C., 22° C., 23° C., 24° C., 25° C., 26° C., 27° C., 28° C., 29° C., 30° C., 31° C., and 32° C., and examples of restrictive temperatures include 35° C., 36° C., 37° C., 38° C., 39° C., 40° C., 41° C., and 42° C.

Assays which may be used to determine whether particular combinations of genetic elements confers a temperature sensitive phenotype include assays involving culturing cells which contain the genetic elements at various temperatures. Such assays would be readily apparent to one skilled in the art.

A wide variety of genetic elements, in addition to temperature sensitive elements, and systems may be used to favor the amplification of one nucleic acid molecule over another. One example is an origin of replication which functions in bacterial cells but not yeast cells. Thus, when nucleic acid molecules of a mixed population of vectors are introduced into yeast cells, molecules which contain origins of replication which function in yeast will be preferentially amplified over those which do not contain such an origin. Additional elements include drug sensitivity markers such as Herpes simplex thymidine kinase, which can be used to select against cells which express this protein, and IPTG inducible promoters, which can be used to select for or against cells in which this promoter activates transcription.

As noted above, FIG. 2 illustrates specific embodiments of the invention. In particular, FIG. 2 shows a process for the transfer of nucleic acid molecules of a cDNA library from Expression Clones (a population of nucleic acid molecules) to a Destination Vector (a target nucleic acid molecule), through a pDONR plasmid intermediate (an intermediary target nucleic acid molecule), to generate additional Expression Clones (a population of nucleic acid molecules).

The first step in the process shown in FIG. 2 involves a BP CLONASE™ catalyzed recombination reaction between Expression Clones (a population of nucleic acid molecules), which comprise the nucleic acid molecules of a cDNA library, and a pDONR plasmid (a target nucleic acid molecule) to generate Entry Clones (a population of nucleic acid molecules). The Expression Clone (a population of nucleic acid molecules) or the pDONR plasmid (a target nucleic acid molecule) may be linear or closed, circular. Further, closed, circular nucleic acid molecules may be relaxed, negatively supercoiled, or positively supercoiled. Supercoiled molecules may each have any number (e.g., one, two, three, four five, six, seven, eight, nine, ten, etc.) of supercoils.

The BP CLONASE™ catalyzed recombination reaction shown in FIG. 2 (a first recombination reaction) occurs in the presence of a protein referred to as Fis. Fis, as well as a number of other proteins (e.g., E. coli ribosomal proteins S10, S14, S15, S16, S17, S18, S19, S20, S21, L14, L21, L23, L24, L25, L27, L28, L29, L30, L31, L32, L33 and L34; U.S. application Ser. No. 09/438,358, filed Nov. 12, 1999, the entire disclosure of which is incorporated herein by reference), enhances the efficiency of recombination reactions (e.g., BP CLONASE™ catalyzed recombination reactions). Thus, the invention further provides methods which employ proteins that enhance recombination reactions (e.g., Fis; E. coli ribosomal proteins S10, S14, S15, S16, S17, S18, S19, S20, S21, L14, L21, L23, L24, L25, L27, L28, L29, L30, L31, L32, L33 and L34; etc.)

Specific parameters and conditions related to the optimization of recombination reactions performed in the presence of Fis are set out below in Example 9. Proteins which enhance recombination reactions (e.g., Fis) may be included in BP CLONASE™ catalyzed recombination reactions, as well as other recombination reactions, in a variety of concentrations, including about 0.5 ng/μl, about 1.0 ng/μl, about 1.5 ng/μl, about 2.0 ng/μl, about 2.5 ng/μl, about 3.0 ng/μl, about 3.5 ng/μl, about 4.0 ng/μl, about 4.5 ng/μl, about 5.0 ng/μl, about 5.5 ng/μl, about 6.0 ng/μl, about 6.5 ng/μl, about 7.0 ng/μl, about 7.5 ng/μl, about 8.0 ng/μl, about 8.5 ng/μl, about 9.0 ng/μl, about 9.5 ng/μl, about 10.0 ng/μl, about 10.5 ng/μl, about 11.0 ng/μl, about 11.5 ng/μl, about 12.0 ng/μl, about 12.5 ng/μl, about 13.0 ng/μl, about 13.5 ng/μl, about 14.0 ng/μl, about 14.5 ng/μl, about 15.0 ng/μl, about 16.0 ng/μl, about 17.0 ng/μl, about 18.0 ng/μl, about 19.0 ng/μl, about 20.0 ng/μl, about 22.0 ng/μl, about 25.0 ng/μl, about 27.0 ng/μl, about 30.0 ng/μl, about 35.0 ng/μl, or about 40.0 ng/μl. Thus, the invention further includes methods which employ proteins that enhance the efficiency of recombination reactions.

As noted above, the concentrations of reagents involved in the first step of the process shown in FIG. 2 can vary considerably. For example, the BP CLONASE™, which contains 25-50 ng/μl Int and 20 ng/μl IHF, may be used in various amounts to catalyze recombination reactions. Using the Int protein of the BP CLONASE™ as a point of reference, the BP CLONASE™ may be used in recombination reactions of the invention such that Int is present at concentrations such as 3 ng/μl, 5 ng/μl, 10 ng/μl, 50 ng/μl, 100 ng/μl, 200 ng/μl, 300 ng/μl, 400 ng/μl, 500 ng/μl, 700 ng/μl, 900 ng/μl, 1000 ng/μl, 1200 ng/μl, 1500 ng/μl, 1700 ng/μl, 1900 ng/μl, or 2000 ng/μl.

The second step in the process shown in FIG. 2 involves an LR CLONASE™ catalyzed recombination reaction between Entry Clones, which comprise the nucleic acid molecules of a cDNA library, and a Destination Vector to re-generate Expression Clones. The Entry Clones or the Destination Vector may be linear or closed, circular. Further, closed, circular nucleic acid molecules may be relaxed, negatively supercoiled, or positively supercoiled. Supercoiled molecules may have any number (e.g., one, two, three, four five, six, seven, eight, nine, ten, etc.) of supercoils.

In many embodiments, the Destination Vector will be linearized before undergoing recombination. Thus, the Destination Vector will generally contain a site which can be used for linearization.

The invention also includes processes for recombining populations of nucleic acid molecules which contain at least one recombination site and the insertion of the recombination products into vectors. Further, the populations of nucleic acid molecules which are inserted into vectors may then be transferred to other vectors.

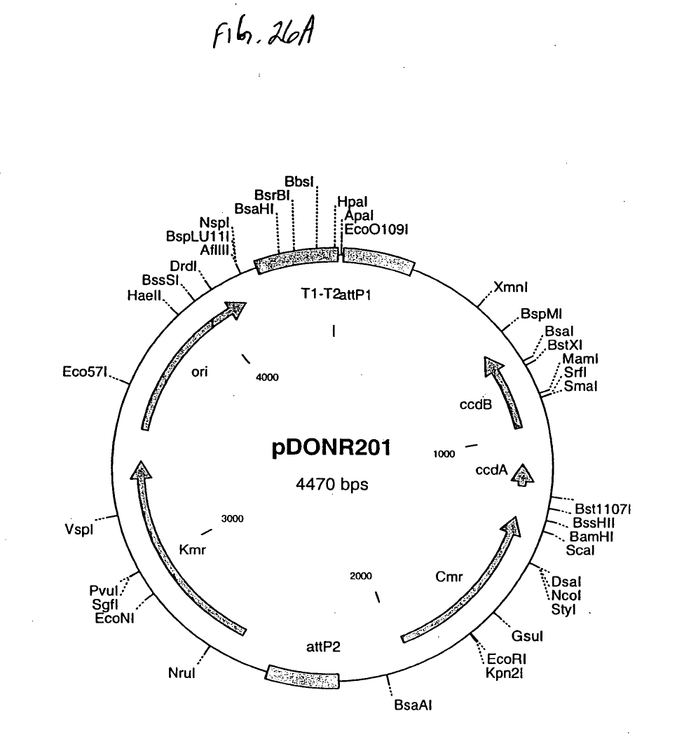

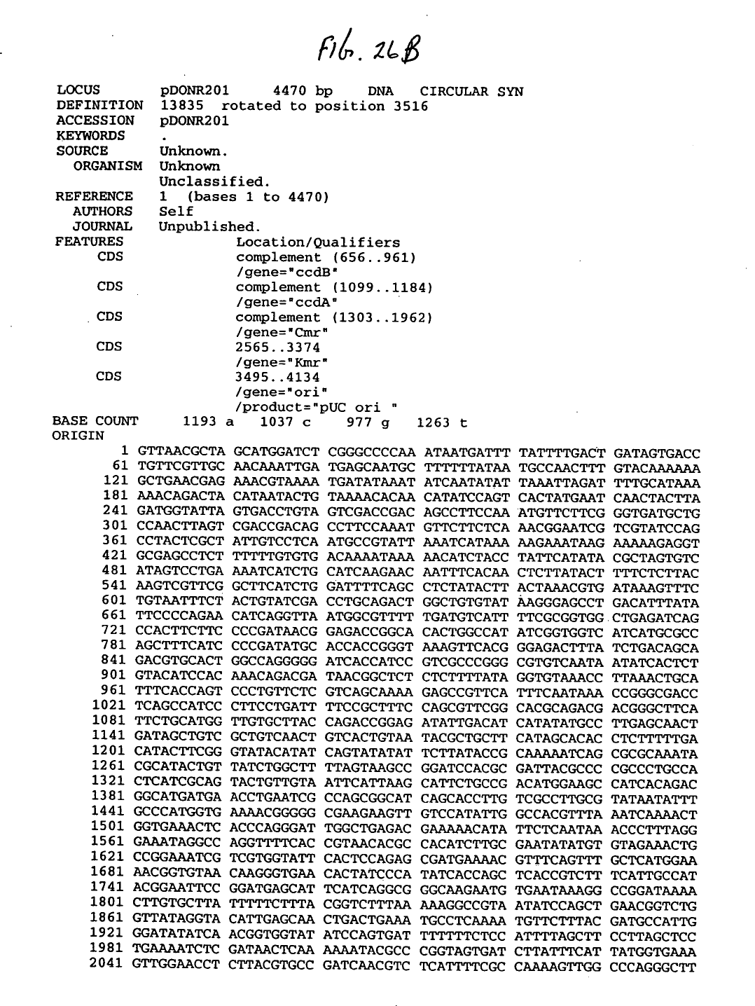

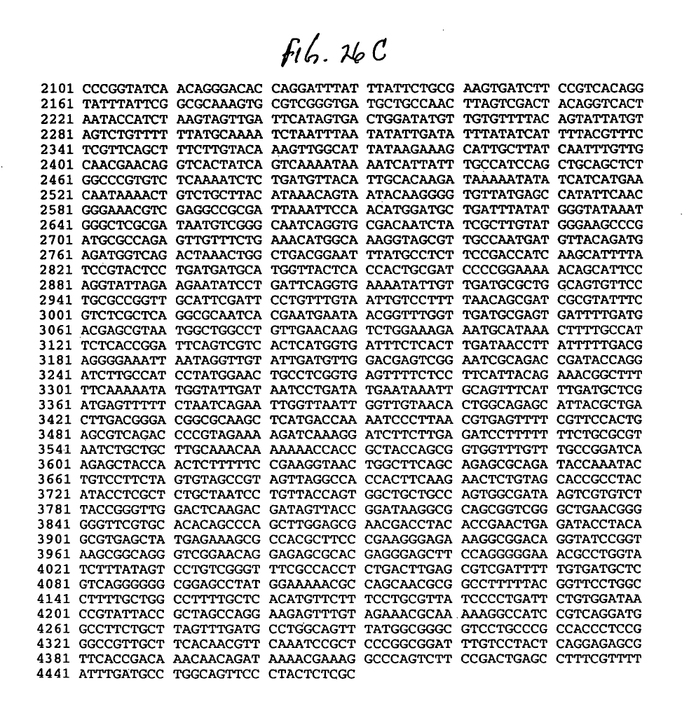

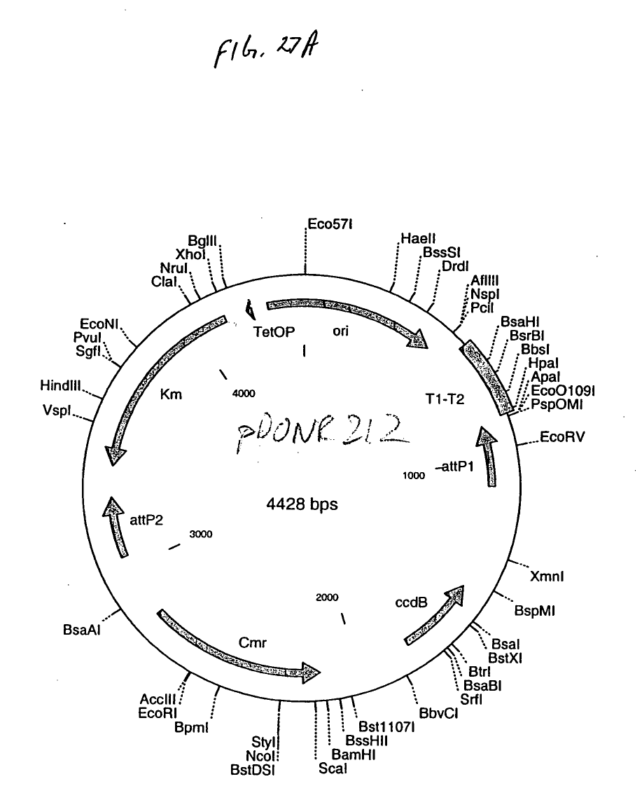

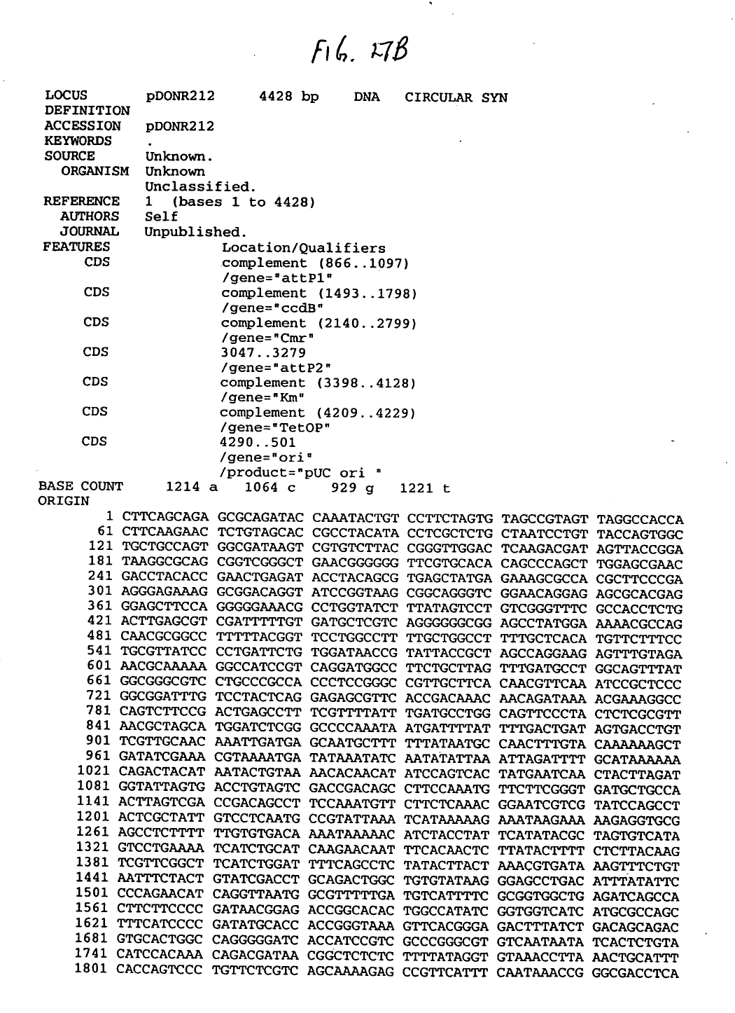

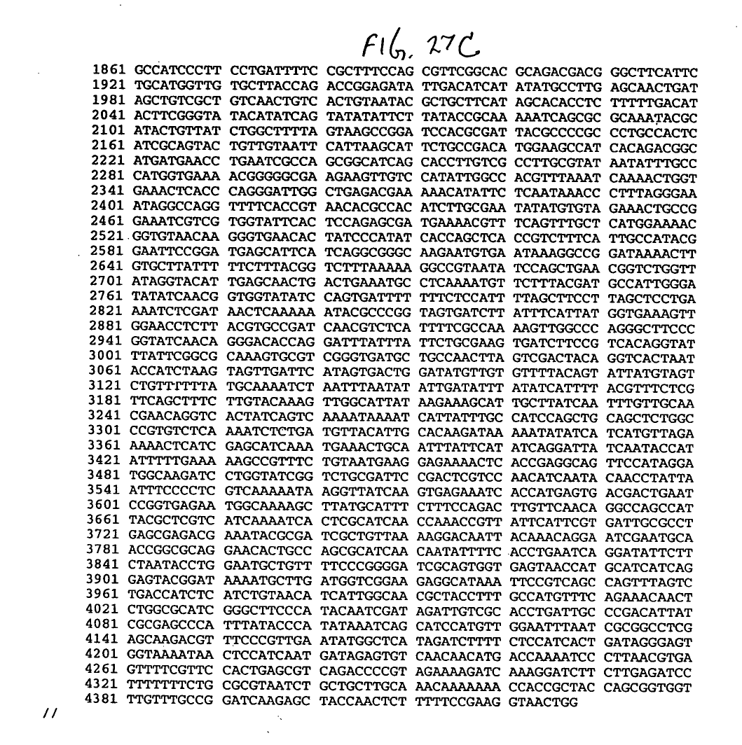

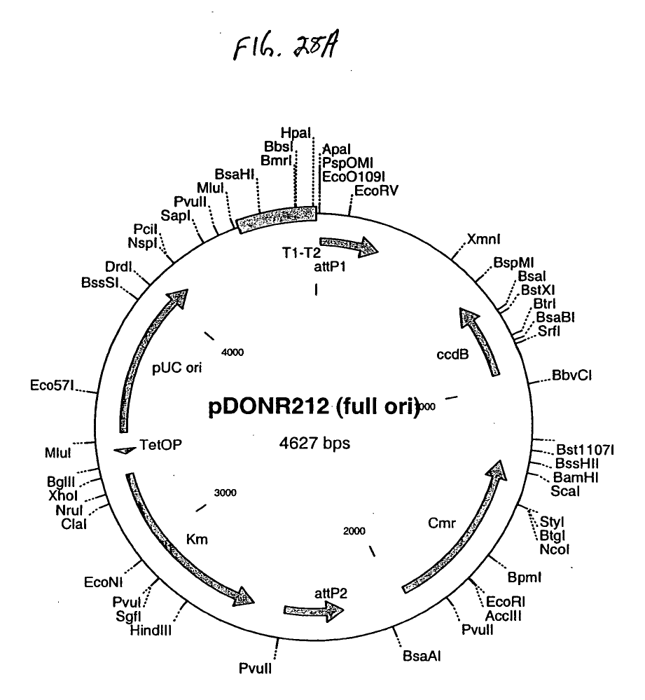

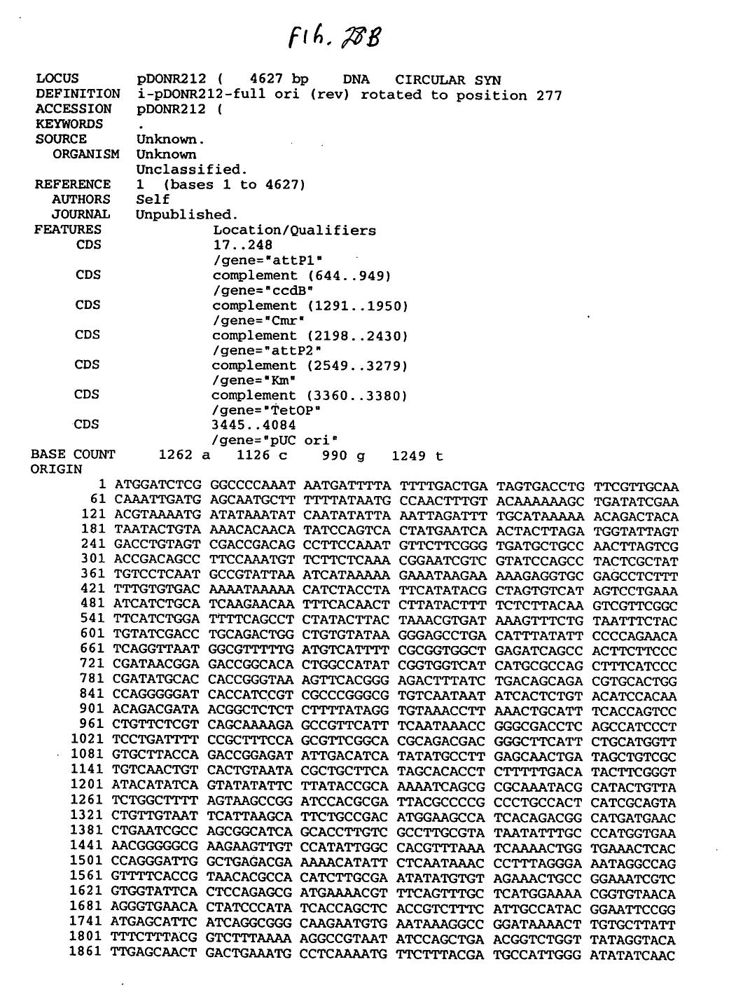

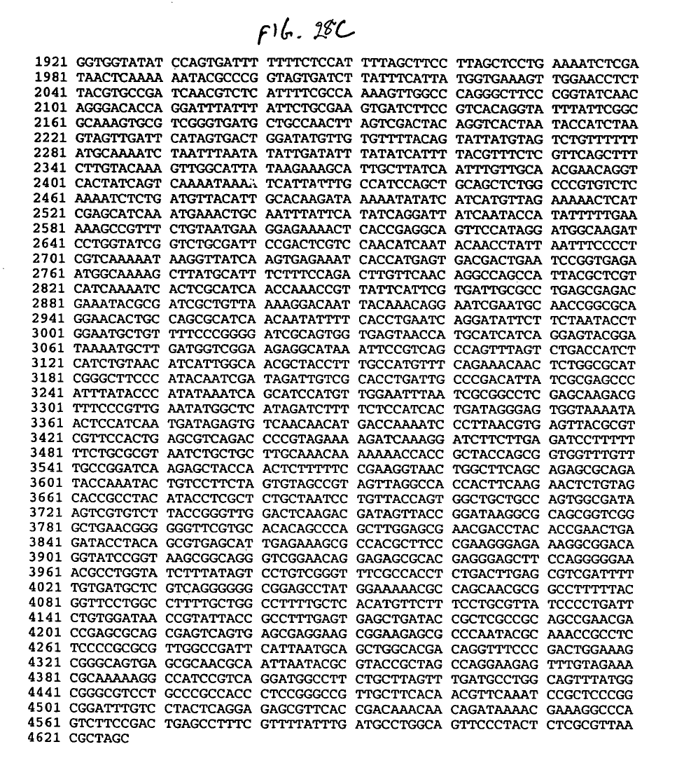

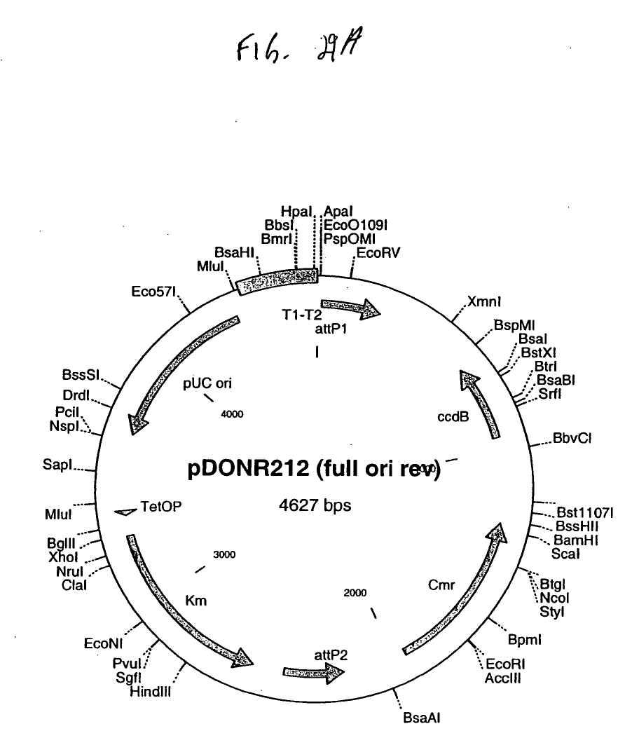

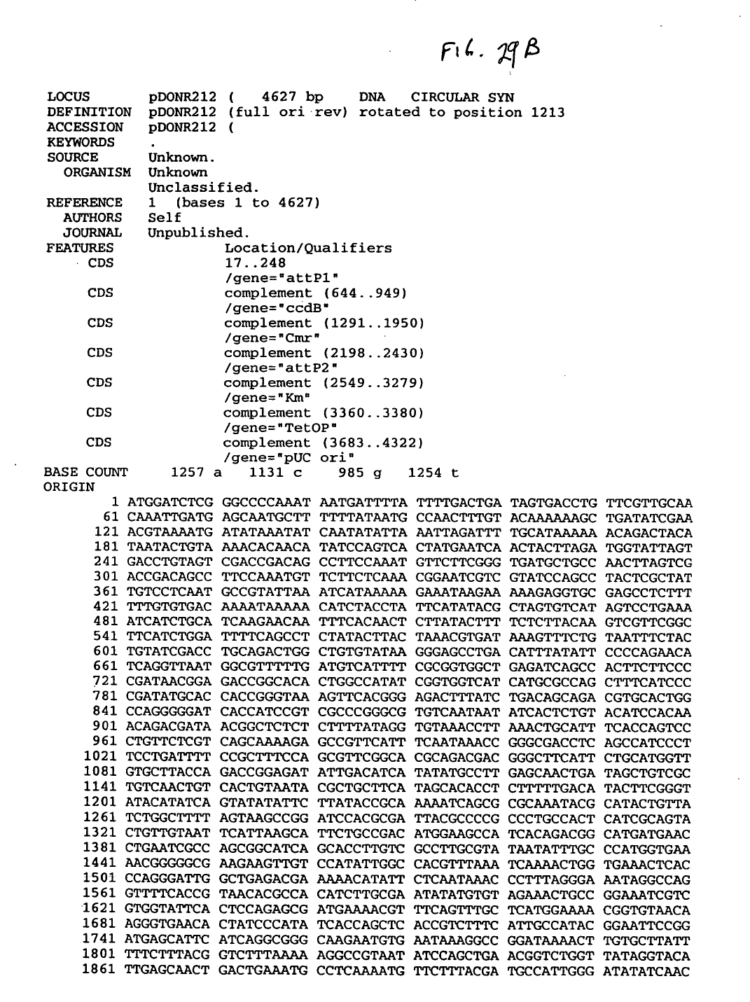

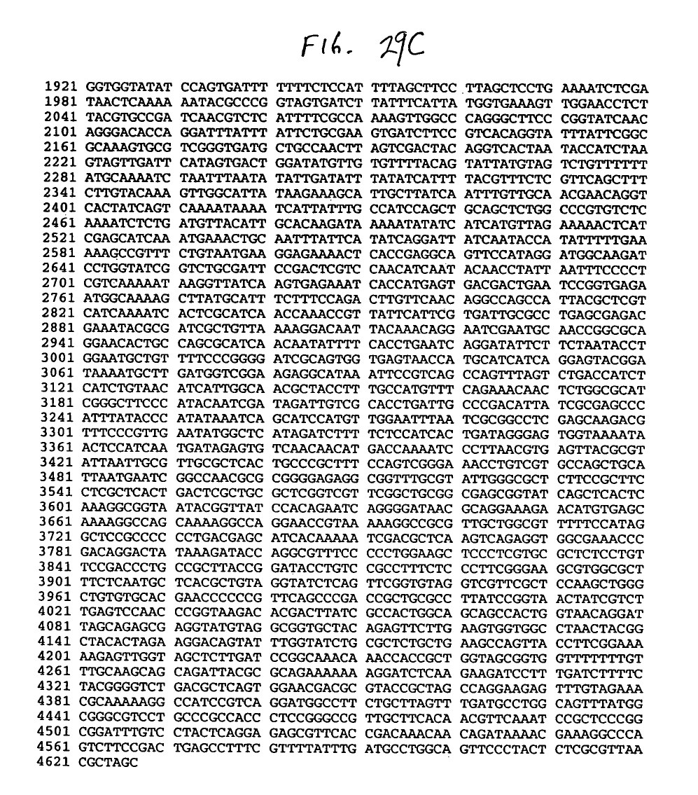

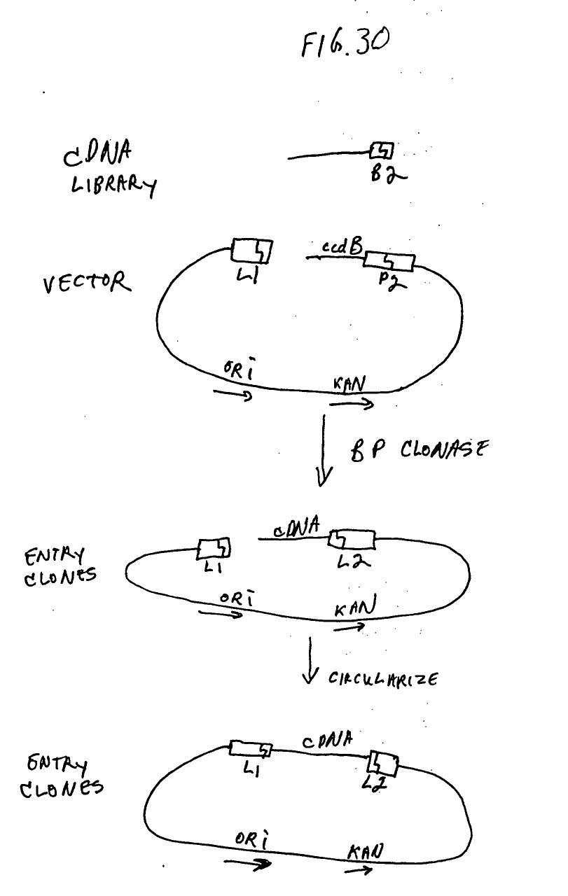

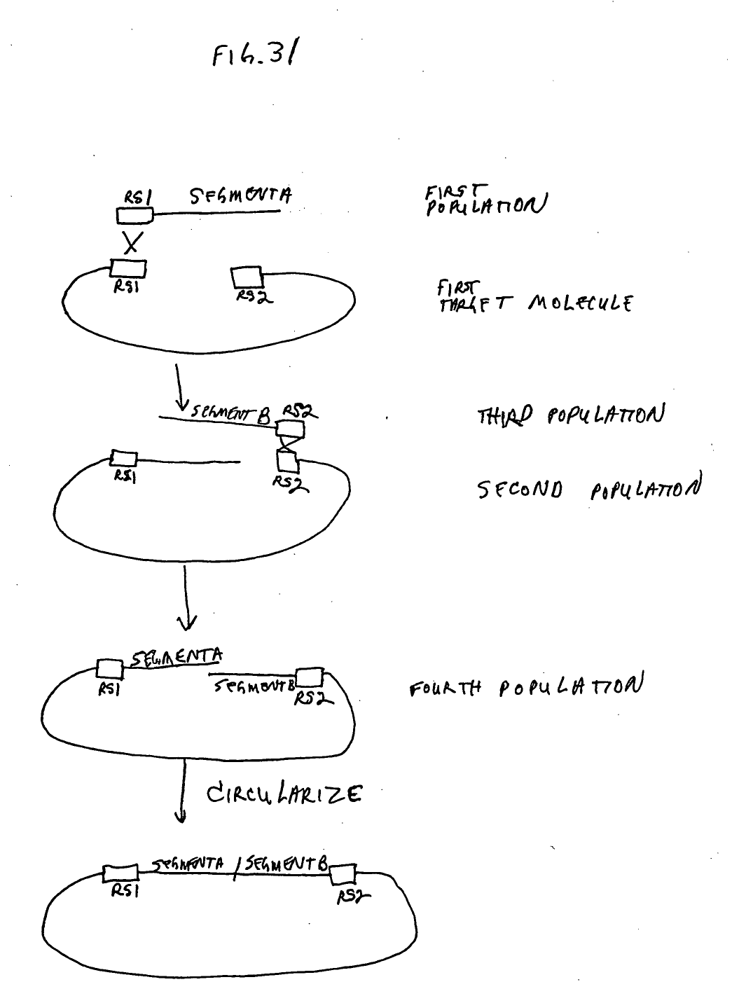

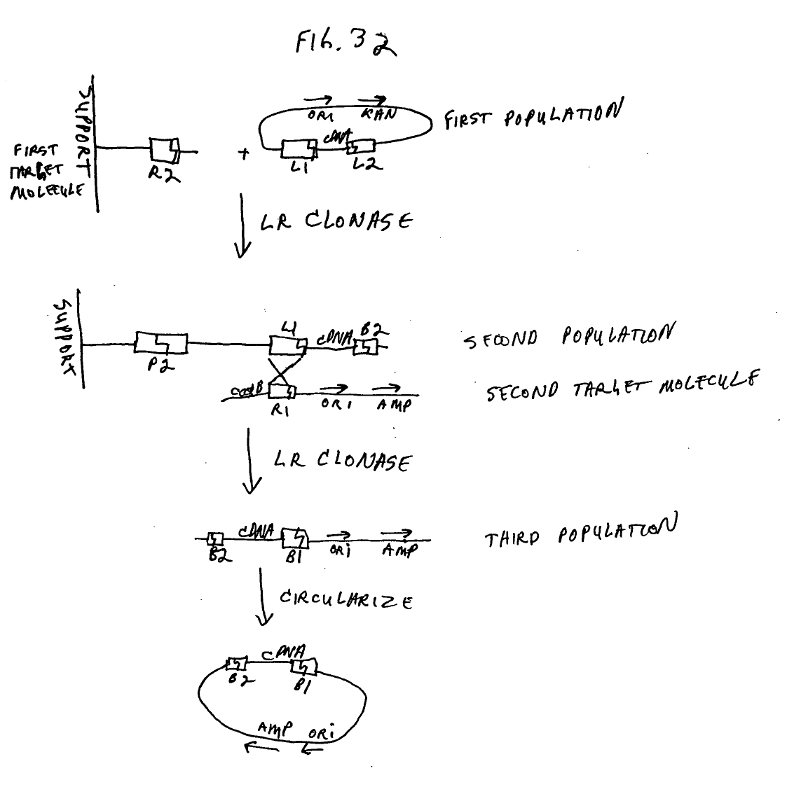

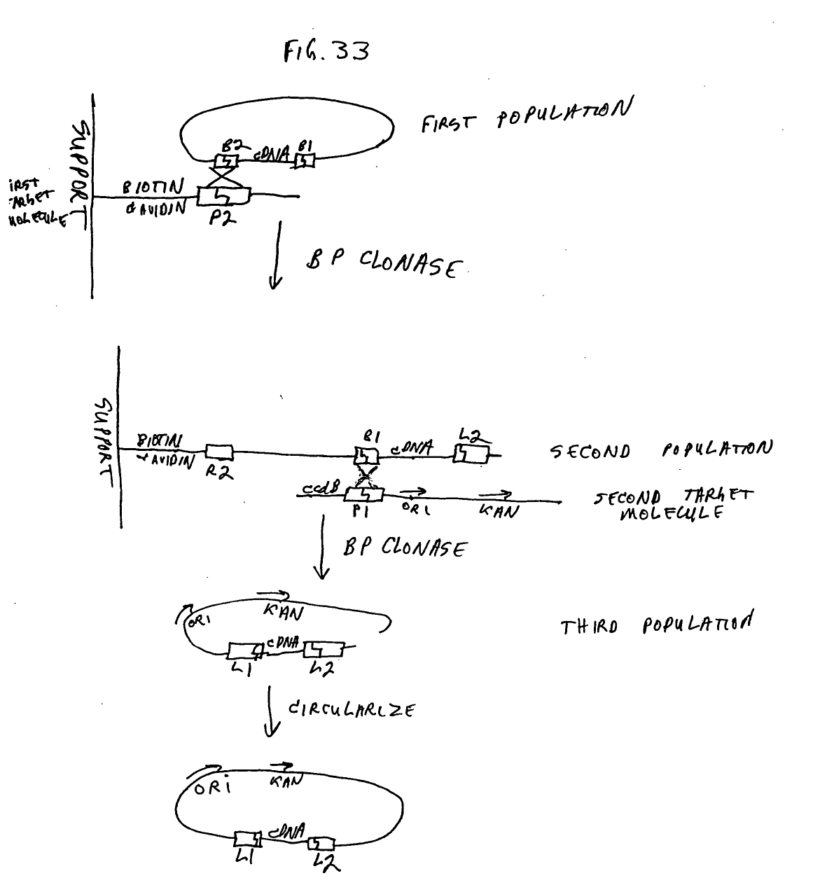

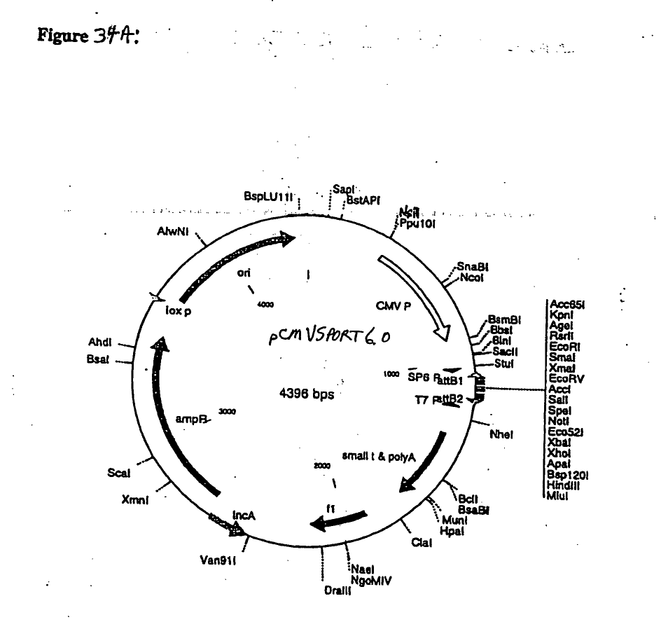

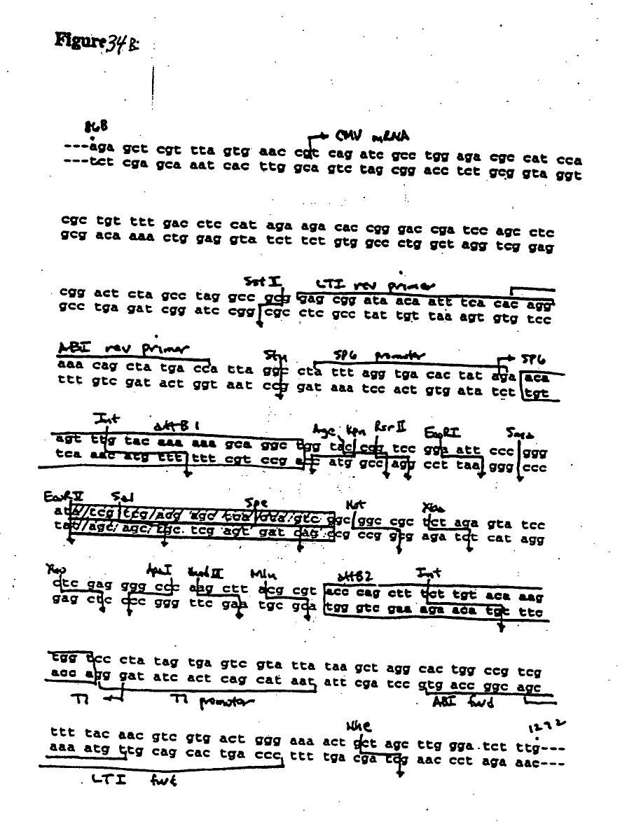

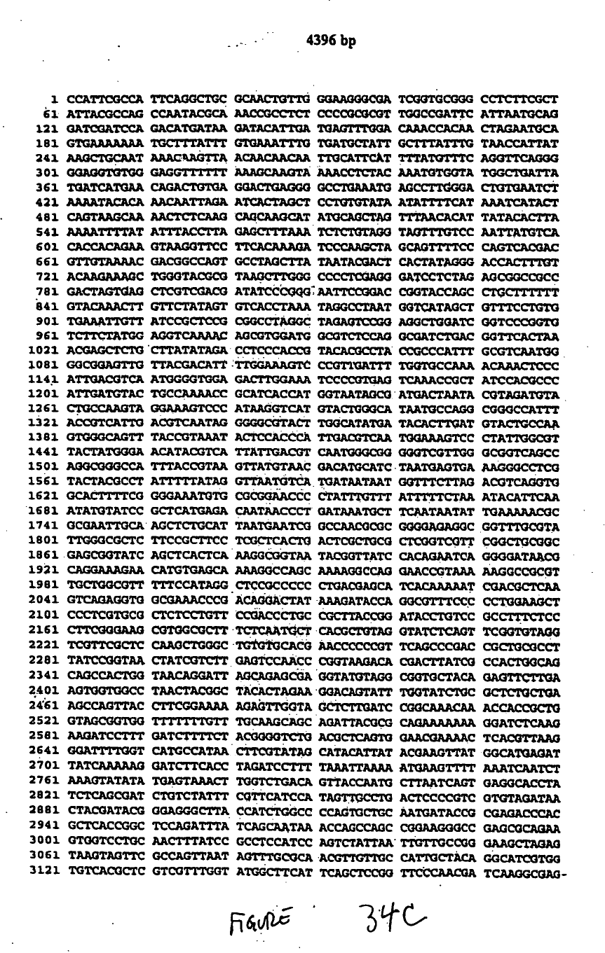

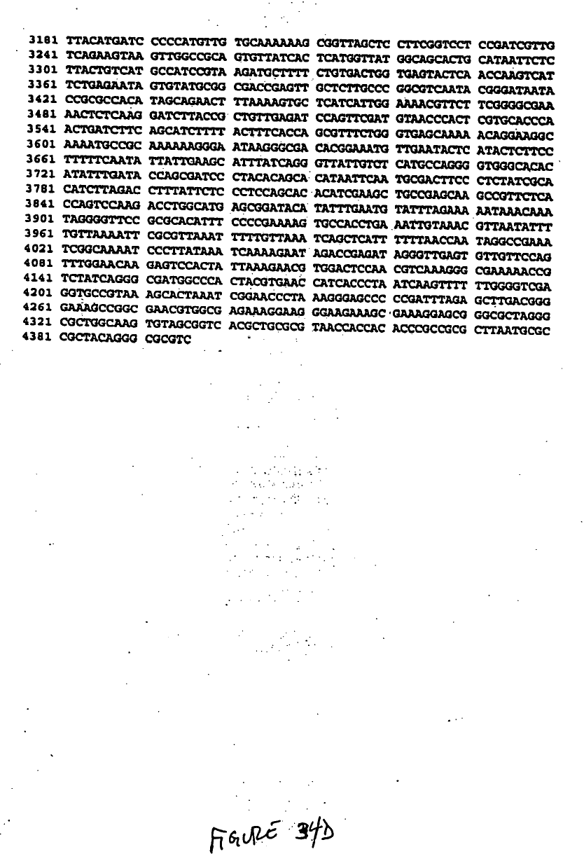

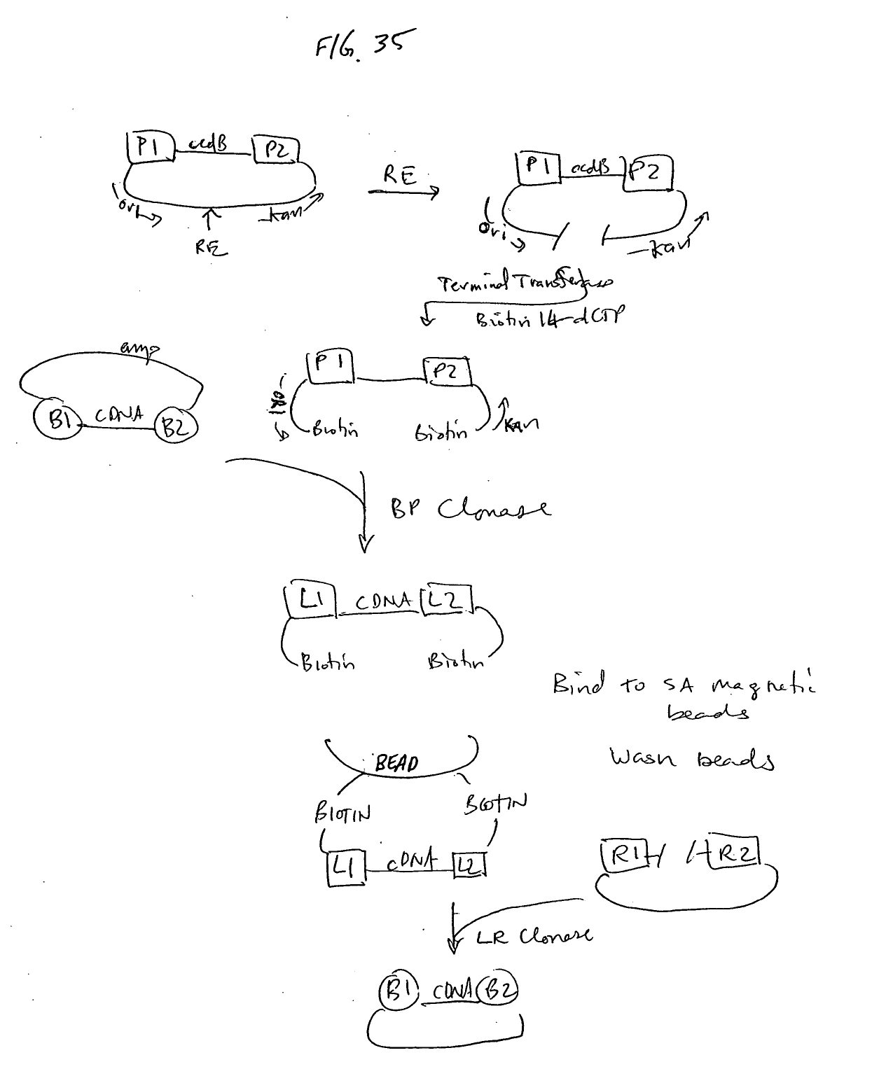

With respect to methods for recombining populations of linear nucleic acid molecules (e.g., molecules of a cDNA library), the invention provides methods for generating populations of nucleic acid molecules which contain one or more recombination sites and methods for recombining theses molecules to alter one or more of these recombination sites (e.g., the conversion of attB sites to attL sites, as shown in FIG. 3). The resulting molecules, which comprise one or more altered recombination sites, may then be recombined with a target nucleic acid molecule to form hybrid nucleic acid molecules.

Using the process shown in FIG. 3 for purposes of illustration, linear molecules of a cDNA library which contain attB sites at each terminus are recombined with linear attP molecules (i.e., a target nucleic acid molecule) to generate a population of cDNA molecules which contain attL sites or attR sites at each terminus (a population of nucleic acid molecules). The resulting population of cDNA molecules is then recombined with a Destination Vector (a target nucleic acid molecule) to generate Expression Clones (a population of nucleic acid molecules).

As one skilled in the art would recognize, numerous variations of the process shown in FIG. 3 are possible and within the scope of the invention. For example, the starting population of cDNA molecules may instead comprise genomic or synthetic nucleic acid molecules. Further, the starting population of nucleic acid molecules, the target nucleic acid molecule, or both may contain additional nucleic acid (1) 5′ to the 5′ end of the 5′ recombination site, (2) 3′ to the 3′ end of the 3′ recombination site, or (3) both 5′ to the 5′ end of the 5′ recombination site and 3′ to the 3′ end of the 3′ recombination site. In addition, the starting population of nucleic acid molecules, the target nucleic acid molecule, or both may be closed, circular. Further, such closed, circular nucleic acid molecules may be relaxed, positively supercoiled, or negatively supercoiled.

Nucleic acid segments may be added to individual members of the populations of nucleic acid molecules which are used to practice methods of the invention. One method for adding nucleic acid segments involves the insertion of individual members of populations of nucleic acid molecules into other nucleic acid molecules (e.g., a vector) which contain the nucleic acid segment to be added. One example of a Destination Vector which may be used in such a process in shown in FIG. 4. A cDNA library, for example, may be inserted into a Destination Vector (i.e., a first target nucleic acid molecule) using recombination between attL1, attR1, attL2 and attR2 sites, to generate a nucleic acid molecule which contains three separate nucleic acid segments (four if the vector is counted) which are separated by attB sites. Recombination between various combinations of attB1, attP1, attB2 and attP2, attB3, attP3, attB4 and attP4 sites, can be used to (1) effect transfer of the resulting population of nucleic acid molecules to a second target nucleic acid molecule or (2) replaced a nucleic acid segment located between two recombination sites. For example, when the second target molecules have been linearized between the recombination sites (see, for example, the pDONOR molecule in the upper left hand corner which is linearized between attP3 and attP1), nucleic acid molecules may be designed such that transfer of the population of nucleic acid molecules of the second population to the second target nucleic acid molecule occurs during recombination to generate Entry Clones.

Further, when the second target molecules have been linearized between in the backbone of the vector (e.g., between kan and ori in the pDONOR molecules shown in FIG. 4), nucleic acid molecules may be designed such that Destination Vectors are either generated/regenerated. For example, using the process shown in FIG. 4 for purposes of illustration, a ccdB coding region from second target molecules may be inserted into members of the second population of nucleic acid molecules, replacing nucleic acids which reside between one or more recombination sites.

Depending on the recombination sites present on the pDONOR molecules (i.e., second target nucleic acid molecules), the population of cDNA molecules may be transferred to the pDONOR vectors with or without additional flanking nucleic acid segments. As one skilled in the art would recognize, any possible number of combinations of the above is included within the scope of the invention. Further, the pDONOR molecules may contain additional recombination sites and nucleic acid segments (e.g., nucleic acid segments having promoter activities) which may be joined to the individual members of the populations of nucleic acid molecules which are transferred. Thus, the invention also provides methods for connecting nucleic acid molecules to other nucleic acid molecules, as well as nucleic acid molecules produced by these methods. This aspect of the invention is particularly useful when combined with screening methods designed to identify nucleic acid molecules which either have specific properties, features, or activities or encode expression products having particular properties, features, or activities.

The invention further allows for the addition of nucleic acid segments to individual members of the populations of nucleic acid molecules used to practice methods of the invention. The invention also allows for the deletion or substitution of nucleic acid segments associated with members of these populations. For example, individual members of the populations of nucleic acid molecules may be introduced into a vector which has multiple recombination sites (e.g., attP sites) having different specificities (e.g., two, three, four five, six, seven, eight, nine, ten, etc. specificities). Nucleic acid segments which confer particular properties, features, or activities upon individual members of the population may be contained between different recombination sites, and may even extend across recombination sites. In the latter instance, under particular circumstances (e.g., when the nucleic acid encode an expression product) recombination can be used, for example, to disrupt properties, features, or activities conferred by nucleic acid segments. As noted above, representative examples of nucleic acid molecules and processes described above are set out in FIG. 4.

The invention also provides methods for constructing nucleic acid molecules in which nucleic acid segments are connected (see, e.g., FIG. 4). Again using the process set out in FIG. 4 for purposes of illustration, once a second population of nucleic acid molecules has been produced, other associated nucleic acid segments may be replaced with members of a library. For example, once a second population of nucleic acid molecules has been generated, these molecules may be screened to identify molecules (e.g., members of the population with cDNA inserts) which have one or more properties, features, or activities. Once nucleic acid molecules containing these inserts have been identified, a nucleic acid library may be inserted into a different region of the second population of nucleic acid molecules. For example, the promoter, shown between attB3 and attB1 sites, in the second population of nucleic acid molecules shown in FIG. 4 may be replaced with members a library of nucleic acid molecules (e.g., a genomic library). Optionally, the resulting new population of nucleic acid molecules may then be screened for promoter activities which result in the expression of the inserted cDNA. Numerous variations of the above are possible. Thus, in certain embodiments, the invention provides methods for the construction of libraries, followed by a first round of screening to identify library members having one or more specified properties, features, or activities, followed by insertion of nucleic acid molecules into the library members identified by the above screening step, followed by second round of screening to identify library members having one or more specified properties, features, or activities. As one skilled in the art would recognize, the above processes of nucleic acid insertion followed by screening may be repeated numerous times (e.g., three, four, five, six, seven, eight, nine, ten, etc.) to arrive at one or more nucleic acid molecules which have one or more desired properties, features, or activities.

In specific embodiment, the final target nucleic acid molecule may be a viral vector (e.g., a Herpes viral vector, an Adenoviral vector, etc.). Such vectors are particularly useful for gene therapy applications, which are discussed below.

Populations of Nucleic Acid Molecules

Virtually any population of nucleic acid molecules may be used in the practice of the invention. Examples of such populations include genomic nucleic acid libraries, cDNA libraries, libraries of variable regions of antibody molecules, and synthetic nucleic acid molecules (e.g., synthetic nucleic acid molecules which encode peptides), as well as modified forms of these libraries.

Populations of nucleic acid molecules used in the practice of the invention may be obtained from virtually any source and may be either purchased for a commercial supplier or prepared by methods well known in the art. For example, libraries prepared from a wide array of biological entities (e.g., viruses, bacterial cells, human cells, etc.) can be obtained from sources such as the American Type Culture Collection (ATCC), 10801 University Boulevard, Manassas, Va. 20110-2209, USA.

Sources from which populations of nucleic acid molecules suitable for use with the invention may be obtained include viruses (e.g., HIV-1, HIV-2, Hepatitis A, Hepatitis B, Hepatitis C, Hepatitis D, Hepatitis E, Hepatitis F, etc.), bacteria (e.g., Escherichia coli, Salmonella typhimurium, Yersinia pestis, Vibrio cholera, Borellia burgdoferi, Thermus aquaticus, Methanococcus janaschii, Thermococcus aegaeicus, Staphylothermus hellenicus, Aquifex pyrophilis, Thermotoga marina, etc.), fungi (e.g., Cryptococcus neoformans, Candida albicans, Tinea corporis, Tinea pedis, Tinea capitis, Saccharomyces cerevisiae, Pichia pastoris, Schizosaccharomyces pombe, etc.), plants (e.g., Lepidium sativum, Brassica juncea, Brassica oleracea, Brassica rapa, Acena sativa, Triticum aestivum, Helianthus annuus, Colonial bentgrass, Kentucky bluegrass, perennial ryegrass, creeping bentgrass, Bermudagrass, Buffalograss, centipedegrass, switch grass, Japanese lawngrass, coastal panicgrass, spinach, sorghum, tobacco, corn, etc.), and animals (e.g., Drosophila melanogaster, mice, rats, rabbits, hamsters, guinea pigs, pigs, goats, sheep, cows, baboons, monkeys, chimpanzees, human, etc.).

The populations of nucleic acid molecules of the invention may contain coding regions, non-coding regions (e.g., promoters), or both coding regions and non-coding regions. Further, coding regions, when present, may encode either polypeptide expression products or functional RNA molecules. As explained below in more detail, non-coding regions include nucleic acids which control the transcription of nucleic acid molecules when present on the molecules undergoing transcription (i.e., when present in cis and in operable linkage with nucleic acid which may be expressed).

In specific embodiments, the nucleic acid libraries used in the practice of the invention are not libraries wherein a high percentage (e.g., at least 20%, at least 30%, at least 40%, at least 50%, at least 70%, at least 80%, at least 90%, etc.) of the nucleic acid molecules encode variable regions of antibody molecules.

The populations of nucleic acid molecules used in the practice of the invention may be combinatorial libraries. Numerous examples of the preparation and use of combinatorial libraries are known in the art. (See, e.g., Waterhouse et al., Nucleic Acids Res. 21:2265-2266 (1993), Tsurushita et al., Gene 172:59-63 (1996), Persson, Int. Rev. Immunol. 10:2-3 153-163 (1993), Chanock et al., Infect. Agents Dis. 2:118-131 (1993), Burioni et al., Res. Virol. 148:161-4 (1997), Leung, Thromb. Haemost. 74:373-376 (1995), Sandhu, Crit. Rev. Biotechnol. 12:5-6 437-62 (1992), and U.S. Pat. Nos. 5,733,743, 5,871,907 and 5,858,657, all of which are specifically incorporated herein by reference.)

Libraries used in the practice of the invention may comprise, for example, normalized cDNA or genomic libraries.

Libraries used in the practice of the invention may also comprise, for example, nucleic acid molecules corresponding to permutations of an original library of nucleic acid molecules prepared by mutagenesis, referred to herein as a “mutagenized library”. Nucleic acid molecules in a mutagenized library may encode, for example, polypeptides or functional RNAs. Further, such libraries may contain nucleic acids which have functions other than encoding expression products (e.g., nucleic acids which have promoter activity). The nucleic acid molecules of mutagenized libraries can be joined to other nucleic acid segments consisting of (1) one or more nucleic acid molecules which are the same or different with respect to sequence or (2) a library of nucleic acid molecules. The nucleic acid molecules of the mutagenized library may be linked to other nucleic acid segments either contiguously or non-contiguously (e.g., intervening nucleic acid may be present). Further, one or more (e.g., one, two, three, four, five, six, seven, eight, nine, ten, etc.) nucleic acid molecules of a mutagenized library may be linked to one or more (e.g., one, two, three, four, five, six, seven, eight, nine, ten, etc.) members of the same library or of a different library, the members of which may or may not have been subjected to mutagenesis.

Mutagenized libraries may be prepared by any number of art known means, including synthesis of the library members by low fidelity polymerases and/or reverse transcriptases. Thus, mutagenized libraries suitable for use with the invention may be prepared using, for example, PCR.

When one or more nucleic acid molecules used in methods and compositions of the invention are subjected to mutagenesis, these molecules may contain either (1) a particular number of mutations or (2) an average number of mutations. Further, mutations may be scored with reference to the nucleic acid molecules themselves or the expression products (e.g., polypeptides encoded by the nucleic acid molecules). For example, nucleic acid molecules of a library may be mutated to produce populations of nucleic acid molecules which are, on average, at least 50%, at least 55%, at least 60%, at least 65%, at least 70%, at least 75%, at least 80%, at least 85%, at least 90%, at least 95%, at least 96%, at least 97%, at least 98%, or at least 99% identical to corresponding nucleic acid molecules of the original library. Further, nucleic acid molecules of a library may be mutated to produce populations of nucleic acid molecules which are, on average, between 50% and 60%, between 55% and 65%, between 60% and 70%, between 65% and 75%, between 70% and 80%, between 75% and 85%, between 80% and 90%, between 85% and 95%, or between 90% and 99% identical to corresponding nucleic acid molecules of the original library.

Similarly, nucleic acid molecules of a library may be mutated to produce populations of nucleic acid molecules which encode polypeptides that are, on average, at least 50%, at least 55%, at least 60%, at least 65%, at least 70%, at least 75%, at least 80%, at least 85%, at least 90%, at least 95%, at least 96%, at least 97%, at least 98%, or at least 99% identical to polypeptides encoded by corresponding nucleic acid molecules of the original library. Further, nucleic acid molecules of a library may be mutated to produce populations of nucleic acid molecules which encode polypeptides that are, on average, between 50% and 60%, between 55% and 65%, between 60% and 70%, between 65% and 75%, between 70% and 80%, between 75% and 85%, between 80% and 90%, between 85% and 95%, or between 90% and 99% identical to polypeptides encoded by corresponding nucleic acid molecules of the original library.

Mutagenesis of nucleic acid molecules has been utilized to generate proteins with altered functions (e.g., binding specificity). Often, the mutagenesis is site-directed, and therefore laborious depending on the systematic choice of mutation to induce in the protein. For example Corey et al., J. Amer. Chem. Soc. 114:1784-1790 (1992), modified rat trypsins by site-directed mutagenesis. Partial randomization of selected codons in the thymidine kinase (TK) gene has also been used as a mutagenesis procedure to develop variant TK proteins. (Munir et al., J. Biol. Chem. 267:6584-6589 (1992).) Mutagenesis may also be performed using methods such as error-prone PCR (see, e.g., Leung et al., Technique, 1:11-15 (1989) and Caldwell and Joyce, PCR Methods Applic., 2:28-33 (1992)) and saturation mutagenesis (see, e.g., Short, U.S. Pat. No. 6,171,820). Thus, methods for introducing specific mutations into nucleic acid sequences are known in the art. A number of such methods are described in Ausubel, F. M. et al., Current Protocols in Molecular Biology, Wiley Interscience, New York (1989-1996). Mutations can be designed into oligonucleotides, which can be used to modify existing cloned sequences, or in amplification reactions. Random mutagenesis can also be employed if appropriate selection methods are available to isolate the desired mutant DNA or RNA. The presence of the desired mutations can be confirmed by sequencing the nucleic acid by well known methods.

In one aspect, the invention allows controlled expression of fusion proteins by suppression of one or more stop codons. According to the invention, one or more nucleic acid molecules (e.g., one, two, three, four, five, seven, ten, twelve, etc.) joined by methods of the invention may comprise one or more stop codons which may be suppressed to allow expression from a first starting molecule through the next joined starting molecule. For example, a nucleic acid molecules comprising a first-second-third segment joined together (when each of such first and second molecules contains a stop codon) can express a tripartite fusion protein encoded by the joined molecules by suppressing each of the stop codons of the first and second segments. Moreover, the invention allows selective or controlled fusion protein expression by varying the suppression of selected stop codons. Thus, by suppressing the stop codon between the first and second molecules but not between the second and third molecules of the first-second-third molecule, a fusion protein encoded by the first and second molecule may be produced rather than the tripartite fusion. Thus, use of different stop codons and variable control of suppression allows production of various fusion proteins or portions thereof encoded by all or different portions of the joined starting nucleic acid molecules of interest.

In one aspect, one or more stop codons may be included anywhere within one or more of the starting nucleic acid molecules (e.g., a member of a mutagenized library) or within a recombination site contained by one or more of the starting molecules. Such stop codons may be located, for example, at or near the termini of any of the joined nucleic acid segments, although such stop codons may be included internally within the molecule. In instances where all or part of a coding sequence is followed by a stop codon, the stop codon may then be followed by a recombination site allowing joining of another nucleic acid molecule. In some embodiments of this type, the stop codon may be optionally suppressed by a suppressor tRNA molecule. The genes coding for the suppressor tRNA molecule may be provided on the same nucleic acid molecule (see FIGS. 20A-20B), on a different nucleic acid molecule, or in the chromosome of the host cell into which a nucleic acid molecule comprising the coding sequence is inserted. In some embodiments, more than one copy (e.g., two, three, four, five, seven, ten, twelve, fifteen, twenty, thirty, fifty, etc. copies) of the suppressor tRNA may be provided. Further, in some embodiments, the transcription of the suppressor tRNA may be under the control of a regulatable (e.g., inducible or repressible) promoter.

When a library used in methods of the invention is a cDNA library, this library may be enriched for nucleic acid molecules which correspond to either the 5′ or 3′ termini of RNA molecules used to generate the library. Methods for making such libraries are known in the art. For example, oligo dT columns can be used to isolate nucleic acid molecules having polyA regions, which are normally associated with the 3′ terminus of RNA molecules. cDNA may then be generated from these RNA molecules. Thus, oligo dT purification of nucleic acids can be used to generate populations of molecules which are enriched for nucleic acid molecules corresponding to the 3′ termini of RNAs. Further, processes such as the “5′ Race System for Rapid Amplification of cDNA Ends” (available from Invitrogen Corp., Carlsbad, Calif., Cat No. 18374-058) may be used to generate libraries which are enriched for nucleic acid molecules which correspond to the 5′ termini of RNAs. Methods for generating cDNA libraries enriched for molecules corresponding to 5′ and/or 3′ of RNA molecules are also discussed in PCT Publication No. WO 00/66722, the entire disclosure of which is incorporated herein by reference.

Properties, Features, and Activities Identified by Methods of the Invention

The invention further provides methods for identifying nucleic acid molecules which either have at least one identifiable property, feature, or activity (e.g., one, two, three, four, five, six, seven, eight, nine, ten, etc.) or encode one or more (e.g., one, two, three, four, five, six, seven, eight, nine, ten, etc.) expression products having at least one (e.g., one, two, three, four, five, six, seven, eight, nine, ten, etc.) identifiable property, feature, or activity. In specific aspects, the invention provides iterative screening methods for identifying nucleic acid molecules which either have particular properties, features, or activities (e.g., encode a polypeptide which is in-frame with a polypeptide encoded by a first target nucleic acid molecule) or encode expression products which have particular properties, features, or activities. For example, nucleic acid molecules may be screened to identify those having one property, feature, or activity (e.g., a property, feature, or activity described below), then nucleic acid molecules identified by the initial screening step may be re-screened to identify those which have either the same or another property, feature, or activity. In many instances, nucleic acid molecules which either have the particular property, feature, or activity for which it is screened or encode an expression property, feature, or activity having this property, feature, or activity will be either inserted into a target nucleic acid molecule or transferred from a first target nucleic acid molecule to a second target nucleic acid molecule between screening steps. Such screening steps may be repeated any number of times (e.g., two, three, four, five, six, seven, etc.). Further, nucleic acid molecules which are subjected to screening steps may be inserted into different target molecules before each screening step.

Processes similar to those described above may be used to screen populations of target nucleic acid molecules which differ in nucleotide sequence but contain one or a small number of inserted nucleic acid molecules. For example, target nucleic acid molecules can be screened for the ability to express an inserted open reading frame in particular cell types (e.g., hepatocytes, leukocytes, etc.).

As one skilled in the art would recognize, nucleic acid molecules have functions and activities which are separate from their ability to encode genetic information. Further, functions and activities identified by methods of the invention are not directed solely to properties, features, or activities exhibited in nature or, when the nucleic acid molecule has been modified, to properties, features, or activities exhibited by the unmodified molecule (e.g., a nucleic acid molecule of a cDNA library).

Examples of properties, features, and activities of nucleic acid molecules which can be assayed in the practice of the invention include (1) the ability to hybridize to other nucleic acid molecules under stringent conditions, (2) the ability to activate gene expression (e.g., the ability to activate gene expression either constitutively in cells of an organism or in a tissue-specific manner), (3) the ability to bind molecules (e.g., proteins, carbohydrates, metal ions, organic compounds, etc.) which exhibit binding affinity for nucleic acid molecules (e.g., proteins which activate transcription), (4) the ability to initiate nucleic acid replication (e.g., origins of replication, autonomously replicating sequences, transcriptional regulatory elements), (5) the ability to segregate nucleic acid molecules during cell divisional (e.g., centromeres), (6) the ability to integrate into other nucleic acid molecules by homologous recombination, (7) the ability to be joined to another nucleic acid molecule by topoisomerase, (8) the ability to be ligated to another nucleic acid molecule, (9) the ability to be digested by particular restriction endonucleases, (10) the ability to anneal to another nucleic acid molecule, (11) the ability to serve as a template for PCR, (12) the ability to participate in transposition, (13) the ability to form secondary structures (e.g., hairpin turns, tRNA-like structures), (14) the ability to participate in recombination reactions (e.g., site-specific recombination and homologous recombination), (15) the ability to direct the “packaging” of nucleic acid molecules (e.g., packaging signals) into viral particles, and (16)the ability to recombine with another nucleic acid molecule by site specific recombination.

Genomic libraries, as well as other libraries (e.g., synthetic libraries), may be screened to identify properties, features, or activities associated with genomic nucleic acids. Examples of such properties, features, and activities include (1) promoter activity and (2) the ability to bind to molecules (e.g., proteins) which bind either specifically or non-specifically to nucleic acids. Genomic libraries of the invention may be used, for example, to identify nucleic acids which exhibit tissue-specific and/or species-specific promoter activity. One example of a system which could be used to identify tissue-specific promoter elements is one where nucleic acid of genomic library is inserted into a vector 5′ to a nucleic acid region which encodes green fluorescent protein (GFP). This vector may then be inserted into cells of particular tissues (e.g., hepatocytes, chondrocytes, leukocytes, etc.) or species (e.g., Escherichia coli, Saccharomyces cerevisiae, Neurospra crassa, Amoeba proteus, etc.) and the cells may then be screened to identify those in which expression of GFP occurs. Numerous other expression detection methods may also be used, including positive and negative selection systems which result in either increased or decreased cell viability.

Genomic libraries, as well as other libraries of the invention, may be screened to identify peptides which bind nucleic acids either specifically or non-specifically. For example, random peptide libraries may be screened to identify peptides which bind genomic nucleic acids. Further, libraries of the invention may also be prepared which express large numbers of peptides. These peptide libraries may then be screened to identify nucleic acid molecules which encode peptides that bind to nucleic acid molecules having a particular nucleotide sequence. Methods for preparing and screening such peptide libraries (e.g., using phage display systems) are described elsewhere herein.

Nucleic acid molecules may also be identified by the identification of properties, features, or activities of their expression products (e.g., RNAs, proteins, etc.). RNA molecules, for example, have a number of functions and activities which are not directly related their ability to encode polypeptides. Examples of activities associated with RNA include ribozyme activity, tRNA activities, and the ability to hybridize to nucleic acids which have complementary nucleotides sequences (e.g., antisense activity, RNAi activity).

Methods of the invention may also be used to identify nucleic acid molecules which allow for silencing of genes in vivo. One method of silencing genes involves the production of double-stranded RNA, termed RNA interference (RNAi). (See, e.g., Mette et al., EMBO J., 19:5194-5201 (2000)). Another method of silencing genes involves the production of antisense RNA/ribozymes fusions which comprise (1) antisense RNA corresponding to a target gene and (2) one or more ribozymes which cleave RNA (e.g., hammerhead ribozyme, hairpin ribozyme, delta ribozyme, Tetrahymena L-21 ribozyme, etc.). Thus, expression products of nucleic acid molecules of the invention can be used to silence gene expression and nucleic acid molecules can be screened to identify those with activities related to gene silencing.

Nucleic acid molecules can also be screened to identify those with functions or activities related to encoded polypeptides expression products. One example of such a function or activity is that the reading frame of the nucleic acid is “in-frame” with nucleic acid of a nucleic acid molecule to which it is connected. Further examples of functions or activities of nucleic acids include encoding polypeptides which (1) induce immunological or other cellular responses (e.g., activate transcription, induce apoptosis, effect the stability of one or more intracellular proteins, etc.), (2) have binding affinity for particular ligands (e.g., small molecules, nucleic acids, functions as a ligand, cell surface receptors, soluble proteins, metal ions, structural elements, protein interaction domains, antibodies, antigens, SH₃ domains, etc.), (3) target proteins to particular locations in cells (e.g., mitochondria, chloroplasts, nuclei, endoplasmic reticulum, cell membranes, etc.), (4) target proteins for export from cells, (5) contain sequences involved in post-translational modifications (e.g., glycosylation sites, ribosylation sites, etc.), (6) have varying degrees of solubility in aqueous solutions, (7) target proteins to specific locations (e.g., endoplasmic reticulum, nucleus, etc.) within a cell or target proteins for export from the cell, (8) alter the infectivity of viruses, (9) alter (e.g., increase or decrease) the solubility of proteins, (10) the ability to co-immune precipitated along with another molecule (e.g., a protein), and (11) have enzymatic activities (e.g., kinase activity, phosphorylase activity, phosphatase activity, reductase activity, oxidase activity, superoxide dismutase activity, catalase activity, etc.).

Using FIG. 8 for purposes of illustration, selection is used in a first step to identify members of a cDNA library which encode proteins that associate with a “bait” protein in a two-hybrid assay. Two-hybrid assays are been described in Yavuzer and Goding, Gene 165:93-96 (1995); Vidal et al., U.S. Pat. No. 5,955,280; and Fields et al., U.S. Pat. No. 5,283,173, and in Example 3 below. In most instances, two-hybrid assays are used to identify proteins which associate with known proteins. For example, a nucleic acid molecule may be constructed which encodes a polypeptide ligand linked to a DNA binding domain (e.g., Gal 4 Binding Domain (Gal4 BD), lexA, etc.). Using the Gal4 system for purposes of illustration, an expression library (e.g., a cDNA library (full-length or partial), a library of mutagenized nucleic acid molecules which encode protein domains, a library which encode random peptides, etc.) may then be constructed which expresses a mixed population of proteins linked to a DNA activation domain (e.g., Gal4 Activation Domain (Gal4 AD), VP22, B42, etc.). Both of these nucleic acids are then introduced into a yeast cell which requires Gal4 promoter gene activation for growth under particular conditions. Thus, because Gal4 AD and Gal4 BD lack protein:protein interaction domains and function to activate transcription when brought into close proximity to each other, yeast cells will only grow when Gal4 AD and Gal4 BD are fused to proteins which associate with each other. As a result, the first step of the process shown in FIG. 8 leads to nucleic acid molecules which are in the same reading frame as the Gal4 AD coding sequences and encode polypeptides which associate with a “bait” protein.

The screening of cDNA libraries enriched for molecules which correspond to 5′ and 3′ regions of RNAs may be used to map domains of proteins which associate with other protein domains. For example, multiple cDNA molecules which encode an interaction domains may be identified using a particular “bait” protein in two-hybrid assays. The sequences of these cDNA molecules may then be compared to identify consensus coding regions. In many instances, these consensus coding regions will encode a domain which interacts with the bait domain employed. Processes of this type are discussed in PCT Publication No. WO 00/66722, the entire disclosure of which is incorporated herein by reference.

In many instances (e.g., when a fusion protein is to be generated as in FIG. 8), it will be desirable to identify or prepare nucleic acid molecules which are in-frame with coding sequences of another nucleic acid molecules (e.g., a vector). Nucleic acid molecules have six potential open reading frames: three forward and three reverse. In many instances, recombination sites can be added (e.g., by the use of PCR with suitable primers) such that the reading frame of all, or substantially all (e.g., at least 95%), of the nucleic acid molecules in the population are in either forward or reverse orientation upon insertion into a target nucleic acid molecule. Methods for preparing directional cDNA libraries are described, for example, in Ohara and Temple, Nucleic Acids Res. 29:E22 (2001), the entire disclosure of which is incorporated herein by reference.

Again using FIG. 8 for illustration, the members of the cDNA library in the initial Expression Clones are flanked by attB1 and attB2 sites. Thus, directionality of these nucleic acid molecules will be maintained upon recombination with, for example, a nucleic acid molecules containing attP1 and attP2 sites, as well as in subsequent recombination reactions.

One method for directionally cloning nucleic acid molecules is to introduce recombination sites the 3′ ends of the molecules by reverse transcription using primers which contain recombination site sequences and sequences which will hybridize to polyA “tails.” The nucleic acid molecules may then be introduced into target nucleic acid molecules, as described elsewhere herein, by single site recombination, followed by attachment (e.g., by ligation) of the 5′ end of the nucleic acid molecules to the target nucleic acid molecules.

In the second step of the process shown in FIG. 8, the nucleic acid molecules identified in the first step are inserted into a vector in-frame with a nucleotide sequence that encodes an epitope tag (i.e., a HIS6 tag) to generate a fusion protein. Thus, the resulting fusion protein may be precipitated with antibody having binding affinity for the epitope tag. All of the cDNA inserts inserted to the vector containing nucleic acid encoding the HIS6 tag, should be in-frame with the nucleotide sequences encoding the tag. However, due to factor such as steric hindrance and conformation properties, features, or activities specific for each fusion protein, all of the expression products of the nucleic acid molecules produced in the second step may not precipitate with antibodies having binding affinity for the epitope tag.

As noted above, expressed proteins may be screened to identify those which have particular biological activities. Examples of such activities include binding affinity for nucleic acid molecules (e.g., DNA or RNA) or other proteins. In particular, expressed proteins may be screened to identify those with binding affinity for either other proteins or themselves. Proteins which have binding affinities for themselves will generally be capable of forming multimers or aggregates. Proteins which have binding affinities for themselves and/or other proteins will often be capable of forming or participating in the formation of multi-protein complexes such as antibodies, splicesomes, multi-subunit enzymes, multi-subunit enzymes, ribosomes, etc. Further included within the scope of the invention are the expressed proteins described above, nucleic acid molecules which encodes these proteins, methods for making these nucleic acid molecules, methods for producing recombinant host cells which contain these nucleic acid molecules, recombinant host cells produced by these methods, and methods for producing the expressed proteins.

One example of a protein characteristic which is readily assayable is solubility. For example, fluorescence generated by GFP is quenched when an insoluble GFP fusion protein is produced. Further, alterations in a relatively small number of amino acid residues of a protein (e.g., one, two, three, four, etc.), when appropriately positioned, can alter the solubility of that protein. Thus, libraries which express GFP fusion proteins can be used to isolate proteins and protein variants which have altered solubility. In one specific example, a combinatorial library designed to express GFP fused with variants of a single, insoluble polypeptide can be used to isolate nucleic acid molecules which encode soluble variants of the polypeptide.

In addition, the nucleic acid molecules of these libraries may encode variable domains of antibody molecules (e.g., variable domains of antibody light and heavy chains). In specific embodiments, the invention provides screening methods for identifying nucleic acid molecules which encode proteins having binding specificity for one or more antigens.

In certain specific embodiments, the one or more libraries referred to above comprise polynucleotides which encode variable domains of antibody light and heavy chains. In related embodiments, at least one nucleic acid segment is located between nucleic acid which encodes the variable domains. This intervening nucleic acid encodes a polypeptide linker for connecting variable domains of antibody molecules. In specific embodiments, the protein complex identified by methods of the invention comprises an antibody molecule or multivalent antigen-binding protein comprising at least two single-chain antigen-binding protein.

A number of methods have been developed for preparing combinatorial libraries of antibody molecules. For example, large libraries of wholly or partially synthetic antibody combining sites, or paratopes, have been constructed utilizing filamentous phage display vectors, referred to as phagemids, yielding large libraries of monoclonal antibodies having diverse and novel immunospecificities. This technology uses a filamentous phage coat protein membrane anchor domain as a means for linking gene-product and gene during the assembly stage of filamentous phage replication, and has been used for the cloning and expression of antibodies from combinatorial libraries. (Kang et al., Proc. Natl. Acad. Sci., USA, 88:4363-4366 (1991).) Combinatorial libraries of antibodies have been produced using both the cpVIII membrane anchor (Kang et al., Proc. Natl. Acad. Sci., USA, 88:4363-4366 (1991)) and the cpIII membrane anchor (Barbas et al., Proc. Natl. Acad. Sci., USA, 88:7978-7982 (1991)).

The diversity of a filamentous phage-based combinatorial antibody library can be increased, for example, by shuffling of the heavy and light chain genes (Kang et al., Proc. Natl. Acad. Sci., USA, 88:11120-11123 (1991)), by altering the complementarity determining region 3 (CDR3) of the cloned heavy chain genes of the library (Barbas et al., Proc. Natl. Acad. Sci., USA, 89:4457-4461 (1992)), and by introducing random mutations into the library by error-prone polymerase chain reactions (PCR) (Gram et al., Proc. Natl. Acad. Sci., USA, 89:3576-3580 (1992)). Further, various cloning systems for producing combinatorial libraries have been described by others. The preparation of combinatorial antibody libraries on phagemids are described, for example, in Kang et al., Proc. Natl. Acad. Sci., USA, 88:4363-4366 (1991); Barbas et al., Proc. Natl. Acad. Sci., USA, 88:7978-7982 (1991); Zebedee et al., Proc. Natl. Acad. Sci., USA, 89:3175-3179 (1992); Kang et al., Proc. Natl. Acad. Sci., USA, 88:11120-11123 (1991); Barbas et al., Proc. Natl. Acad. Sci., USA, 89:4457-4461 (1992); and Gram et al., Proc. Natl. Acad. Sci., USA, 89:3576-3580 (1992), the disclosures of each of which are hereby incorporated by reference.

The present invention relates generally to methods for producing novel antibody molecules and single-chain antigen-binding proteins by the preparation of diverse libraries of antibody domain (e.g., variable light and variable heavy immunoglobin domains), and subsequent screening of such libraries to identify molecules having particular binding specificities. Such antibody molecules may be obtained by screening for expression products which demonstrate binding affinity for one or more antigens. For example, protein expression products encoded by a library and displayed on the surface of a filamentous phage (e.g., gIII phage) may be screened to identify those which bind to one or more preselected antigens.

Furthermore, libraries of variable light and variable heavy immunoglobin domains (i.e., the variable regions of light and heavy chains) may be combined to form random pairings of species of variable heavy and variable light chains, yielding unique heterodimers. Such combinations can be conducted in a variety of ways, as described further herein, including (1) combining a single variable heavy domain to a library of variable light domains, (2) combining a single variable light domain to a library of variable heavy domains, (3) combining a randomized variable light or variable heavy domain against a single variable heavy or variable light domain, respectively, (4) combining a randomized variable light or variable heavy domain against a variable heavy or variable light domain library, respectively, and (5) combining a randomized variable light or variable heavy domain against a randomized variable heavy or variable light domain, respectively. Other permutations are also apparent. The variable light and heavy domains referred to above may be on the same or different protein chains. Single-chain antigen-binding proteins are one example of where variable light and heavy domains may be on a single protein chain.

By randomized is meant generally to connote the preparation of a library of nucleic acid molecules encoding variable light and variable heavy immunoglobin domains by mutagenesis.

One permutation of the above methods to produce an antibody repertoire is by the use of randomized nucleic acid molecules encoding variable light domain nucleic acids combined with a variable heavy domain library, and particularly combined with a randomized variable heavy domain library. Other embodiments of the invention involve methods which employ a “universal light chain”, or a variable light domain thereof. Immunoglobulin light chains which have the ability to complex into a functional heterodimer with any of a variety of heavy chains, and therefore are referred to as “universal light chains” to connote their ability to be used with a variety of heavy chains are described in Barbas et al., U.S. Pat. No. 6,096,551 and may be used in methods of the invention. In one embodiment, a randomized universal light chain against a heavy chain or heavy chain library is screened to identify antigen-binding proteins having specificity for one or more antigens.

Nucleic acid molecules of the invention can also be screened to identify those which complement a cellular gene upon expression in a host cell (e.g., an animal cell) or confer a phenotypic property, feature, or activity upon a host cell. Thus, nucleic acid molecules of the invention can be used, for example, to prepare gene therapy vectors designed to replace genes which reside in the genome of a cell, to delete such genes, or to insert a heterologous gene or groups of genes. When nucleic acid molecules of the invention function to delete or replace a gene or genes, the gene or genes being deleted or replaced may lead to the expression of either a “normal” phenotype or an aberrant phenotype (e.g., the disease cystic fibrosis). Further, the gene therapy vectors may be either stably maintained (e.g., integrate into cellular nucleic acid by homologous recombination) or non-stably maintained in cells.

Nucleic acid molecules of the invention may also be used to suppress “abnormal” phenotypes or complement or supplement “normal” phenotypes which result from the expression of endogenous genes. One example of a nucleic acid molecule of the invention designed to suppress an abnormal phenotype would be where an expression product of the nucleic acid molecule has dominant/negative activity. An example of a nucleic acid molecule of the invention designed to supplement a normal phenotype would be where introduction of the nucleic acid molecule effectively results in the amplification of a gene resident in the cell.

As an example, protocols similar to the following may be used to design and produce gene therapy vectors. Nucleic acid molecules of a cDNA library may be screened to identify nucleic acid molecules which encode a product (e.g., CFTR) which can alleviate manifestations resulting from a genetic defect (e.g., cystic fibrosis). These nucleic acid molecules may be identified, for example, by screening for nucleic acid molecules which encode expression products which can complement cellular effects resulting from the particular genetic defect or by the ability to hybridize to a primer having a sequence derived from a gene known to be associated with the particular defect. Further, processes of the invention may also be used to identify promoter elements which function in the cells in which the genetic defect is manifested. Such promoters may be constitutive or tissue-specific.

Once the nucleic acid molecules described above have been identified and isolated, nucleic acid molecules which encode a product may be operably linked to the promoter element. Further, the operably linked nucleic acid conjugate may then be placed in a vector suitable for gene therapy (e.g., an adenoviral vectors), as described elsewhere herein.

Thus, in related aspects, the invention provides gene therapy vectors which express one or more expression products (e.g., one or more fusion proteins), methods for producing such vectors, methods for performing gene therapy using vectors of the invention, expression products of such vector (e.g., encoded RNA and/or proteins), and host cells which contain vectors of the invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In another specific embodiment, viral vectors that contains nucleic acid sequences encoding an antibody or other antigen-binding protein of the invention are used. For example, a retroviral vector can be used (see Miller et al., Meth. Enzymol. 217:581-599 (1993)). These retroviral vectors have been used to delete retroviral sequences that are not necessary for packaging of the viral genome and integration into host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia and the use of such vectors are included within the scope of the invention. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication Nos. WO94/12649 and WO 96/17053; U.S. Pat. No. 5,998,205;
and Wang et al., 1995, Gene Therapy 2:775-783, the disclosures of all of which are incorporated herein by reference in their entireties. In a one embodiment, adenovirus vectors are used.

Adeno-associated virus (AAV) and Herpes viruses, as well as vectors prepared from these viruses have also been proposed for use in gene therapy (Walsh et al., 1993, Proc. Soc. Exp. Biol. Med. 204:289-300; U.S. Pat. No. 5,436,146; Wagstaff et al., Gene Ther. 5:1566-70 (1998)). Herpes viral vectors are particularly useful for applications where gene expression is desired in nerve cells.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and, optionally, heritable and expressible by its cell progeny.

In a specific embodiment, nucleic acid molecules to be introduced for purposes of gene therapy comprises an inducible promoter operably linked to the coding region, such that expression of the nucleic acid molecules are controllable by controlling the presence or absence of the appropriate inducer of transcription.

In brief, each target nucleic acid molecule may comprise, in addition to one or more recombination sites (e.g., two, three, four, five, seven, ten, twelve, fifteen, twenty, thirty, fifty, etc.), a variety of sequences (or combinations thereof) including, but not limited to sequences suitable for use as primer sites (e.g., sequences which a primer such as a sequencing primer or amplification primer may hybridize to initiate nucleic acid synthesis, amplification or sequencing), transcription or translation signals or regulatory sequences such as promoters or enhancers, ribosomal binding sites, Kozak sequences, start codons, transcription and/or translation termination signals such as stop codons (which may be optimally suppressed by one or more suppressor tRNA molecules), origins of replication, selectable markers, and coding regions which may be used to create protein fusions (e.g., N-terminal or carboxy terminal) such as glutathione S-transferase (GST), β-glucuronidase (GUS), the Fc portion of an immunoglobin, an antibody, histidine tags (HIS6), green fluorescent protein (GFP), yellow fluorescent protein (YFP), cyan fluorescent protein (CFP), open reading frame (ORF) sequences a transcription activation domain, a protein or domain involved in translation, protein localization tag, a protease cleavage site, a protein stabilization or destabalization sequence, a protein interaction domains, a binding domain for DNA, a protein substrate, a purification tag (e.g., an epitope tag, maltose binding protein, a six histidine tag, glutathione S-transferase, etc.), and any other sequence of interest which may be desired or used in various molecular biology techniques including sequences for use in homologous recombination (e.g., for use in gene targeting).

Recombination Systems and Recombination Sites

Recombination sites for use in the invention may be any nucleic acid that can serve as a substrate in a recombination reaction. Such recombination sites may be wild-type or naturally occurring recombination sites, or modified, variant, derivative, or mutant recombination sites. Examples of recombination sites for use in the invention include, but are not limited to, λ phage recombination sites (such as attP, attB, attL, and attR and mutants or derivatives thereof) and recombination sites from other bacteriophage such as HP1, S2, phi80, P22, P2, 186, P4 and P1 (including lox sites such as loxP, loxP511, and variants thereof). Mutated att sites (e.g., attB1-10, attP1-10, attR1-10 and attL1-10) are described in U.S. Appl. No. 60/136,744, filed May 28, 1999; U.S. application Ser. No. 09/517,466, filed Mar. 2, 2000; and PCT Publication No. WO 00/52027, each of which are specifically incorporated herein by reference. Different site specificities allow directional cloning or linkage of desired molecules thus providing desired orientation of the cloned molecules. Other recombination sites having unique specificity (i.e., a first site will recombine with its corresponding site and will not recombine with a second site having a different specificity) are known to those skilled in the art and may be used to practice the present invention. Corresponding recombination proteins for these systems may be used in accordance with the invention with the indicated recombination sites.

Other systems providing recombination sites and recombination proteins for use in the invention include the FLP/FRT system from Saccharomyces cerevisiae, the resolvase family (e.g., RuvC, γΔ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in Escherchia coli. Other suitable recombination sites may be found in U.S. Pat. No. 5,851,808 issued to Elledge and Liu which is specifically incorporated herein by reference. Recombination proteins and mutant, modified, variant, or derivative recombination sites for use in the invention include those described in U.S. Pat. Nos. 5,888,732 and 6,143,557, and in U.S. application Ser. No. 09/438,358 (filed Nov. 12, 1999), U.S. Appl. No. 60/108,324 (filed Nov. 13, 1998), U.S. application Ser. No. 09/732,914 (filed Dec. 11, 2000), U.S. application Ser. No. 09/517,466 (filed Mar. 2, 2000), and U.S. Appl. No. 60/136,744 (filed May 28, 1999), as well as those associated with the GATEWAY™ Cloning Technology available from Invitrogen Corp., Carlsbad, Calif., the entire disclosure of each of which is specifically incorporated herein by reference. Recombination cloning methods are also described in Esposito et al., “Compositions and Methods for Recombinational Cloning of Nucleic Acid Molecules,” filed in the U.S. Patent & Trademark Office on Mar. _{—}, 2001, the entire disclosure of which is incorporated herein by reference.

In certain embodiments, recombination sites used in compositions and methods of the invention do not include loxP and/or loxP511 sites.

Two primary reactions constitute the GATEWAY™ Cloning System, as depicted generally in FIG. 9. The first of these reactions, the LR Reaction (FIG. 10A), which may also be referred to interchangeably herein as the Destination Reaction, is the main pathway of this system. The LR Reaction is a recombination reaction between an Entry vector or clone and a Destination Vector, mediated by a cocktail of recombination proteins such as the GATEWAY™ LR CLONASE™ Enzyme Mix described herein. In the embodiment shown in FIG. 10A, this reaction transfers nucleic acid molecules of interest (which may be genes, cDNAs, cDNA libraries, or fragments thereof) from the Entry Clone to an Expression Vector, to create an Expression Clone.

The sites labeled L, R, B, and P in FIGS. 10A and 10B are respectively the attL, attR, attB, and attP recombination sites for the bacteriophage λ recombination proteins that constitute the CLONASE™ cocktail (referred to herein variously as “CLONASE™” or “GATEWAY™ LR CLONASE™ Enzyme Mix” (for recombination protein mixtures mediating attL×attR recombination reactions, as described herein) (Invitrogen Corp., Carlsbad, Calif., catalog number 11791-019) or “GATEWAY™ BP CLONASE™ Enzyme Mix” (for recombination protein mixtures mediating attB×attP recombination reactions, as described herein) (Invitrogen Corp., Carlsbad, Calif., catalog number 11789-013)). The recombinational cloning reactions are equivalent to concerted, highly specific, cutting and ligation reactions. Viewed in this way, the recombination proteins cut, for example, to the left and right of the nucleic acid molecule of interest in the Entry Clone and ligate it into the Destination vector, creating a new Expression Clone.

The nucleic acid insert in an Expression Clone is generally flanked by the small attB1 and attB2 sites. The orientation and reading frame of the nucleic acid insert are maintained throughout the subcloning, because attL1 reacts only with attR1, and attL2 reacts only with attR2. Likewise, attB1 reacts only with attP1, and attB2 reacts only with attP2. Thus, the invention also relates to methods of controlled or directional cloning using the recombination sites of the invention (or portions thereof), including variants, fragments, mutants and derivatives thereof which may have altered or enhanced specificity. The invention also relates more generally to any number of recombination site partners or pairs (where each recombination site is specific for and interacts with its corresponding recombination site). Such recombination sites may be made by mutating or modifying the recombination site to provide any number of necessary specificities, non-limiting examples of which are described in FIG. 13A-13C.

Using embodiments shown in FIG. 10A-10B for purposes of illustration, when an aliquot from the recombination reaction is transformed into host cells (e.g., E. coli) and spread on plates containing an appropriate selection agent (e.g., an antibiotic such as ampicillin), cells that take up the desired clone form colonies. The unreacted Destination Vector does not give ampicillin-resistant colonies, even though it carries the ampicillin-resistance gene, because it contains a toxic gene (e.g., ccdB). Thus, selection for ampicillin resistance selects for E. coli cells that carry the desired product, which usually comprise >90% of the colonies on the ampicillin plate.

To participate in the recombinational cloning reaction, a nucleic acid insert (e.g., an individual member of a cDNA library) first may be cloned into an Entry Vector, creating an Entry Clone. Multiple options are available for creating Entry Clones, including: cloning of PCR sequences with terminal attB recombination sites into Entry Vectors; using the GATEWAY™ Cloning System recombination reaction; transfer of genes from libraries prepared in GATEWAY™ Cloning System vectors by recombination into Entry Vectors; cloning of restriction enzyme-generated fragments and PCR fragments into Entry Vectors by standard recombinant DNA methods, and topoisomerase cloning. These approaches are discussed in further detail herein.

A key advantage of the GATEWAY™ Cloning System is that a nucleic acid molecule of interest (or even a population of nucleic acid molecules of interest) present as an Entry Clone can be subcloned in parallel into one or more Destination Vectors in a simple reactions for anywhere from about 30 seconds to about 60 minutes (e.g., about 1-60 minutes, about 1-45 minutes, about 1-30 minutes, about 2-60 minutes, about 2-45 minutes, about 2-30 minutes, about 1-2 minutes, about 30-60 minutes, about 45-60 minutes, or about 30-45 minutes). Longer reaction times (e.g., 2-24 hours, or overnight) may increase recombination efficiency, particularly where larger nucleic acid molecules are used. Moreover, a high percentage of the colonies obtained carry the desired Expression Clone. This process is illustrated schematically in FIG. 11, which shows an advantage of the invention in which the molecule of interest can be moved simultaneously or separately into multiple Destination Vectors. In the LR Reaction, one or both of the nucleic acid molecules to be recombined may have any topology (e.g., linear, relaxed circular, nicked circular, supercoiled, etc.).

The second major pathway of the GATEWAY™ Cloning System is the BP Reaction (FIG. 10B), which may also be referred to interchangeably herein as the Entry Reaction or the Entry Reaction. The BP Reaction may recombine an Expression Clone with a Donor Plasmid (the counterpart of the by-product in FIG. 9). This reaction transfers the nucleic acid molecule of interest (which may have any of a variety of topologies, including linear, coiled, supercoiled, etc.) in the Expression Clone into an Entry Vector, to produce a new Entry Clone. Once this nucleic acid molecule of interest is cloned into an Entry Vector, it can be transferred into new Expression Vectors, through the LR Reaction as described above. In the BP Reaction, one or both of the nucleic acid molecules to be recombined may have any topology (e.g., linear, relaxed circular, nicked circular, supercoiled, etc.).

One variation of the BP Reaction permits rapid cloning and expression of products of amplification (e.g., PCR) or nucleic acid synthesis. Amplification (e.g., PCR) products synthesized with primers containing terminal 25 base pair attB sites serve as efficient substrates for the Entry Cloning reaction. Such amplification products may be recombined with a Donor Vector to produce an Entry Clone (see FIG. 10B). The result is an Entry Clone containing the amplification fragment. Such Entry Clones can then be recombined with Destination Vectors—through the LR Reaction—to yield Expression Clones of the PCR product.

Additional details of the LR Reaction are shown in FIG. 10A. The GATEWAY™ LR CLONASE™ Enzyme Mix that mediates this reaction contains lambda recombination proteins Int (Integrase), Xis (Excisionase), and IHF (Integration Host Factor). In contrast, the GATEWAY™ BP CLONASE™ Enzyme Mix, which mediates the BP Reaction (FIG. 10B), comprises Int and IHF alone.

The recombination (att) sites of each vector comprise two distinct segments, donated by the parental vectors. The staggered lines dividing the two portions of each att site, depicted in FIGS. 10A and 10B, represent the seven-base staggered cut produced by Int during the recombination reactions. This structure is seen in greater detail in FIG. 12, which displays attB recombination site sequences of an Expression Clone, generated by recombination between the attL1 and attL2 sites of an Entry Clone and the attR1 and attR2 sites of a Destination Vector.

In one embodiment, a nucleic acid molecule of interest in an Expression Clone is flanked by attB sites: attB1 to the left (amino terminus) and attB2 to the right (carboxy terminus). The bases in attB1 to the left of the seven-base staggered cut produced by Int are derived from the Destination vector, and the bases to the right of the staggered cut are derived from the Entry Vector (see FIG. 12). Note that the sequence is displayed in triplets corresponding to an open reading frame. If the reading frame of the nucleic acid molecule of interest cloned in the Entry Vector is in phase with the reading frame shown for attB1, amino-terminal protein fusions can be made between the nucleic acid molecule of interest and any GATEWAY™ Cloning System Destination Vector encoding an amino-terminal fusion domain. Entry Vectors and Destination Vectors that enable cloning in all three reading frames.

The LR Reaction allows the transfer of a desired nucleic acid molecule of interest into new Expression Vectors by recombining a Entry Clone with various Destination Vectors. To participate in the LR or Destination Reaction, however, a nucleic acid molecule of interest may first be inserted into a vector to generate an Entry Clone. Entry Clones can be made in a number of ways, as shown in FIG. 14.

One approach is to clone the nucleic acid molecule of interest into one or more of the Entry Vectors, using standard recombinant DNA methods, with restriction enzymes and ligase. The starting DNA fragment can be generated by restriction enzyme digestion or as a PCR product. The fragment is cloned between the attL1 and attL2 recombination sites in the Entry Vector. Note that a toxic or “death” gene (e.g., ccdB), provided to minimize background colonies from incompletely digested Entry Vector, must be excised and replaced by the nucleic acid molecule of interest.

A second approach to making an Entry Clone (FIG. 14) is to make a library (e.g., genomic library, cDNA library, synthetic nucleic acid library, etc.) in an Entry Vector, as described in detail herein. Such libraries may then be transferred into Destination Vectors for expression screening, for example, in appropriate host cells such as yeast cells or mammalian cells.

A third approach to making Entry Clones (FIG. 14) is to use Expression Clones obtained from cDNA molecules or libraries prepared in Expression Vectors. Such cDNAs or libraries, flanked by attB sites, can be introduced into a Entry Vector by recombination with a Donor Vector via the BP Reaction. If desired, an entire Expression Clone library can be transferred into the Entry Vector through the BP Reaction. Expression Clone cDNA libraries may also be constructed in a variety of prokaryotic and eukaryotic GATEWAY™-modified vectors (e.g., pDEST1 (see, e.g., FIGS. 17A-17D)).

A fourth, and potentially most versatile, approach to making an Entry Clone (FIG. 14) is to introduce a sequence for a nucleic acid molecule of interest into an Entry Vector by amplification (e.g., PCR) fragment cloning. The DNA sequence first is amplified (for example, with PCR) using primers comprising two or more (e.g., two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five nucleotides of the attB nucleotide sequences (such as, but not limited to, those depicted in FIG. 12 or FIG. 13A-13C). Optionally one or more, two or more, three or more, four or more, or four or five or more additional terminal nucleotide bases may be guanines. The PCR product then may be converted to a Entry Clone by performing a BP Reaction, in which the attB-PCR product recombines with a Donor Vector containing one or more attP sites and, optionally, one or more topoisomerase cloning sites.

A variety of Entry Clones may be produced by these methods, providing a wide array of cloning options; a number of specific Entry Vectors are also available commercially from Invitrogen Corp., Carlsbad, Calif.

Entry Vectors and Destination Vectors will often be constructed so that the amino-terminal region of a nucleic acid insert (e.g., a member of a cDNA library) will be positioned next to the attL1 site. Entry Vectors may contain the mmB transcriptional terminator upstream of the attL1 site. This sequence ensures that expression of cloned nucleic acid molecules of interest is reliably “off” in E. coli, so that even toxic genes can be successfully cloned. Thus, Entry Clones may be designed to be transcriptionally silent. Note also that Entry Vectors, and hence Entry Clones, may contain the kanamycin antibiotic resistance (kan^{r}) gene to facilitate selection of host cells containing Entry Clones after transformation. In certain applications, however, Entry Clones may contain other selection markers, including but not limited to a gentamycin resistance (gen^{r}) or tetracycline resistance (tet^{r}) gene, to facilitate selection of host cells containing Entry Clones after transformation.

Once a nucleic acid molecule of interest has been cloned into an Entry Vector, it may be moved into a Destination Vector. The upper right portion of FIG. 10A shows a schematic of a Destination Vector. The thick arrow represents some function (often transcription or translation) that will act on the nucleic acid molecule of interest in the clone. In this example, during the recombination reaction, the region between the attR1 and attR2 sites, including a gene which encodes a product which either is toxic (e.g., ccdB) or inhibits growth, is replaced by the DNA segment from the Entry Clone. Selection for recombinants that have acquired the ampicillin resistance (amp^{r}) gene (carried on the Destination Vector) and that have also lost the gene which encodes the toxic or growth inhibitory product ensures that a high percentage (usually >90%) of the resulting colonies will contain the correct insert.

To move a nucleic acid molecule of interest into a Destination Vector, the Destination Vector is mixed with the Entry Clone comprising the desired nucleic acid molecule of interest, a cocktail of recombination proteins (e.g., GATEWAY™ LR CLONASE™ Enzyme Mix) is added, the mixture is incubated (e.g., at about 25° C. for about 15 minutes, or longer under certain circumstances, e.g., for transfer of large nucleic acid molecules, as described below) and any standard host cell (including bacterial cells such as E. coli; animal cells such as insect cells, mammalian cells, nematode cells and the like; plant cells; and yeast cells) strain is transformed with the reaction mixture. The host cell used will be determined by the desired selection (e.g., E. coli DB3.1, available commercially from Invitrogen Corp., Carlsbad, Calif., allows survival of clones containing the ccdB death gene, and thus can be used to select for cointegrate molecules—i.e., molecules that are hybrids between the Entry Clone and Destination Vector). The Examples below provide further details and protocols for use of Entry and Destination Vectors in transferring nucleic acid molecules of interest.

The cloning system of the invention therefore offers multiple advantages:

Once a nucleic acid molecule of interest is cloned into the GATEWAY™ Cloning System, it can be moved into and out of other vectors with complete fidelity of reading frame and orientation. That is, since the reactions proceed whereby attL1 on the Entry Clone recombines with attR1 on the Destination Vector, the directionality of the nucleic acid molecule of interest is maintained or may be controlled upon transfer from the Entry Clone into the Destination Vector. Hence, the GATEWAY™ Cloning System provides a powerful and easy method of directional cloning of nucleic acid molecule of interest.

One-step cloning or subcloning: Entry Clones and the Destination Vectors can be mixed with LR CLONASE™, incubated, and used to transform cells.

PCR products can be readily cloned by adding attB sites to PCR primers, followed by in vitro recombination. The cloned products can then be directly transfer from resulting Entry Clones into Destination Vectors. This process may also be carried out in one step.

Powerful selections give high reliability: >90% (and often >99%) of the colonies contain the desired DNA in its new vector.

Conversion of existing standard vectors into GATEWAY™ Cloning System vectors can be done in one step. Such processes are ideal for large vectors or those with few cloning sites. Further, recombination sites are short (25 base pairs), and may be engineered to contain no stop codons or secondary structures.

Reactions may be automated, for high-throughput applications (e.g., for diagnostic purposes or for therapeutic candidate screening).

The reactions are economical: 0.3 μg of each DNA may be used and no restriction enzymes, phosphatase, ligase, or gel purification are necessary. Further, the reactions work well with miniprep DNA.

Multiple clones, and even libraries, may be transferred into one or more Destination Vectors, in a single experiment.

A variety of Destination Vectors may be produced, for applications including, but not limited to:
a). Protein expression in E. coli. For example, native proteins or fusion proteins (e.g., fusions with GST, His6, thioredoxin, etc. for protein purification, or with one or more epitope tags) may be expressed. Further, any promoter useful in expressing proteins in E. coli may be used. Examples of such promoters include lac, trp, ptrc, and T7 promoters.
b). Protein expression in eukaryotic cells. For example, native proteins or fusion proteins, as set out above, may be expressed. Further, any promoter useful in expressing proteins in eukaryotic cells may be used. Examples of such promoters include the baculovirus polyhedrin, SP6, metallothionein I, Autographa californica nuclear polyhidrosis virus, Semliki Forest virus, Tet, CMV, Gal1, Gal10, and T7 promoters.
c). DNA sequencing (e.g., using lac primers, RNA probes, phagemids, etc.).
d). Gene therapy.
e). Expression cloning.
f). Bacterial artificial chromosome (BAC) production.
g). Yeast artificial chromosome (YAC) production.
h). Human artificial chromosome (HAC) production.
i). P1-based replicon artificial chromosome (PAC) production.

A variety of Entry Vectors (for recombinational cloning entry by standard recombinant DNA methods) may be produced:
a). Strong transcription stop just upstream, for genes toxic to E. coli.
b). Three reading frames.
c). With or without TEV protease cleavage site.
d). Motifs for prokaryotic and/or eukaryotic translation.
e). Compatible with commercial cDNA libraries.

Expression Clone cDNA (attB) libraries, for expression screening, including two-hybrid libraries and phage display libraries, may also be constructed.

The transfer reactions described herein may be accomplished using the described recombinational cloning process in a single step or in multiple steps. For example, an initial population flanked by attB recombination sites, mixed with an appropriate attP vector (e.g., pDONR201 (Invitrogen Corp., Carlsbad, Calif., Cat. No. 11798-014)) and BP CLONASE™ to generate Entry Clones flanked by attL sites. This population may be isolated (in vivo or in vitro) and used subsequently for additional future transfer reactions. Alternatively, the desired second vector background (Destination Vector) may be added directly to the first in vitro transferred population, along with LR CLONASE™, to generate a further population of molecules in a new vector background (flanked by attB sites in an Expression Clone) upon which the next selection may be applied.

In one embodiment, the initial and/or resulting population is flanked by attB1 and attB2 sites. In another embodiment, the initial and/or resulting population is flanked by attL1 and attL2 sites. Such an organization maintains orientation of the transferring population. Other site-specific recombination systems (other lambdoid or lambdoid-like systems, Cre/loxP, Flp/FRT, and those described broadly elsewhere as mediating site-specific recombination or transposition, etc.) can be designed to perform this process in an analogous manner. Examples of lox sites which differ in recombination specificity are disclosed in PCT Publication No. WO 01/11058, the entire disclosure of which is incorporated herein by reference.

It should be noted that not all selection schemes require that orientation be maintained. In cases where maintenance of orientation is not required, the DNA segment of interest might be flanked by a single recombination site (e.g., attB1-DNA segment-attB1). Here also, other recombination systems can be applied, and in some cases may be preferable. These approaches may or may not be supplemented with additional selection schemes (e.g., site-DNA segment-selection marker-site) to facilitate the identification or removal of starting or product populations or members thereof.

It will be appreciated that just as a population or subpopulation can be identified or selected for as a result of functions supplied by the vector (or the Insert Clone or the vector and insert combination), so might a population or subpopulation be selected against or removed from a population prior to subsequent transfers. Moreover, that selection may include inhibiting the transfer itself, such that a particular population is sequestered or inhibited from participating in the transfer reaction, thereby resulting in a population of transferred molecules not thereby inhibited.

Representative examples of recombination sites which can be used in the practice of the invention include att sites referred to above, as well as modified forms of these sites. For example, att sites which specifically recombine with other att sites can be constructed by altering nucleotides in and near the 7 base pair overlap region. Thus, recombination sites suitable for use in the methods, compositions, and vectors of the invention include, but are not limited to, those with insertions, deletions or substitutions of one, two, three, four, or more nucleotide bases within the 15 base pair core region (GCTTTTTTATACTAA (SEQ ID NO:47)), which is identical in all four wild-type lambda att sites, attB, attP, attL and attR (see U.S. application Ser. No. 08/663,002, filed Jun. 7, 1996 (now U.S. Pat. No. 5,888,732) and Ser. No. 09/177,387, filed Oct. 23, 1998, which describes the core region in further detail, and the disclosures of which are incorporated herein by reference in their entireties). Recombination sites suitable for use in the methods, compositions, and vectors of the invention also include those with insertions, deletions or substitutions of one, two, three, four, or more nucleotide bases within the 15 base pair core region (GCTTTTTTATACTAA (SEQ ID NO:47)) which are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

Analogously, the core regions in attB1, attP1, attL1 and attR1 are identical to one another, as are the core regions in attB2, attP2, attL2 and attR2. Nucleic acid molecules suitable for use with the invention also include those which comprising insertions, deletions or substitutions of one, two, three, four, or more nucleotides within the seven base pair overlap region (TTTATAC, which is defined by the cut sites for the integrase protein and is the region where strand exchange takes place) that occurs within this 15 base pair core region (GCTTTTTTATACTAA (SEQ ID NO:47)). Examples of such mutants, fragments, variants and derivatives include, but are not limited to, nucleic acid molecules in which (1) the thymine at position 1 of the seven base pair overlap region has been deleted or substituted with a guanine, cytosine, or adenine; (2) the thymine at position 2 of the seven base pair overlap region has been deleted or substituted with a guanine, cytosine, or adenine; (3) the thymine at position 3 of the seven base pair overlap region has been deleted or substituted with a guanine, cytosine, or adenine; (4) the adenine at position 4 of the seven base pair overlap region has been deleted or substituted with a guanine, cytosine, or thymine; (5) the thymine at position 5 of the seven base pair overlap region has been deleted or substituted with a guanine, cytosine, or adenine; (6) the adenine at position 6 of the seven base pair overlap region has been deleted or substituted with a guanine, cytosine, or thymine; and (7) the cytosine at position 7 of the seven base pair overlap region has been deleted or substituted with a guanine, thymine, or adenine; or any combination of one or more such deletions and/or substitutions within this seven base pair overlap region. The nucleotide sequences of the above described seven base pair core region are set out below in Table 1.

The following non-limiting methods can be used to modify or mutate a given nucleic acid molecule encoding a particular recombination site to provide mutated sites that can be used in the present invention:
1. By recombination of two parental DNA sequences by site-specific (e.g., attL and attR to give attp) or other (e.g., homologous) recombination mechanisms where the parental DNA segments contain one or more base alterations resulting in the final mutated nucleic acid molecule;
2. By mutation or mutagenesis (site-specific, PCR, random, spontaneous, etc) directly of the desired nucleic acid molecule;
3. By mutagenesis (site-specific, PCR, random, spontaneous, etc) of parental DNA sequences, which are recombined to generate a desired nucleic acid molecule;
4. By reverse transcription of an RNA encoding the desired core sequence; and
5. By de novo synthesis (chemical synthesis) of a sequence having the desired base changes, or random base changes followed by sequencing or functional analysis according to methods that are routine in the art.

The functionality of the mutant recombination sites can be demonstrated in ways that depend on the particular characteristic that is desired, or on the property, feature, or activity upon which selection is based. For example, the lack of translation stop codons in a recombination site can be demonstrated by expressing the appropriate fusion proteins. Specificity of recombination between homologous partners can be demonstrated by introducing the appropriate molecules into in vitro reactions, and assaying for recombination products as described herein or known in the art. Other desired mutations in recombination sites might include the presence or absence of restriction sites, translation or transcription start signals, protein binding sites, one or more protease cleavage sites, particular coding sequences, and other known functionalities of nucleic acid base sequences. Genetic selection schemes for particular functional attributes in the recombination sites can be used according to known method steps. For example, the modification of sites to provide (from a pair of sites that do not interact) partners that do interact could be achieved by requiring deletion, via recombination between the sites, of a DNA sequence encoding a toxic substance. Similarly, selection for sites that remove translation stop sequences, the presence or absence of protein binding sites, etc., can be easily devised by those skilled in the art.

Altered att sites have been constructed which demonstrate that (1) substitutions made within the first three positions of the seven base pair overlap (TTTATAC) strongly affect the specificity of recombination, (2) substitutions made in the last four positions (TTTATAC) only partially alter recombination specificity, and (3) nucleotide substitutions outside of the seven base pair overlap, but elsewhere within the 15 base pair core region, do not affect specificity of recombination but do influence the efficiency of recombination. Thus, nucleic acid molecules and methods of the invention include those which comprising or employ one, two, three, four, five, six, eight, ten, or more recombination sites which affect recombination specificity, particularly one or more (e.g., one, two, three, four, five, six, eight, ten, twenty, thirty, forty, fifty, etc.) different recombination sites that may correspond substantially to the seven base pair overlap within the 15 base pair core region, having one or more mutations that affect recombination specificity. Further, such molecules may comprise a consensus sequence such as NNNATAC, wherein “N” refers to any nucleotide (i.e., may be A, G, T/U or C). In general, if one of the first three nucleotides in the consensus sequence is a T/U, then at least one of the other two of the first three nucleotides is not a T/U.

The core sequence of each att site (attB, attP, attL and attR) can be divided into functional units consisting of integrase binding sites, integrase cleavage sites and sequences that determine specificity. Specificity determinants are defined by the first three positions following the integrase top strand cleavage site. These three positions are shown with underlining in the following reference sequence: CAACTTTTTTATACAAAGTTG (SEQ ID NO:48). Modification of these three positions (64 possible combinations) which can be used to generate att sites which recombine with high specificity with other att sites having the same sequence for the first three nucleotides of the seven base pair overlap region are shown in Table 1.

TABLE 1

Modifications of the First Three Nucleotides of
the att Site Seven Base Pair Overlap Region which
Alter Recombination Specificity.

| | | | |
|---|---|---|---|
| AAA | CAA | GAA | TAA |
| AAC | CAC | GAC | TAC |
| AAG | CAG | GAG | TAG |
| AAT | CAT | GAT | TAT |
| ACA | CCA | GCA | TCA |
| ACC | CCC | GCC | TCC |
| ACG | CCG | GCG | TCG |
| ACT | CCT | GCT | TCT |
| AGA | CGA | GGA | TGA |
| AGC | CGC | GGC | TGC |
| AGG | CGG | GGG | TGG |
| AGT | CGT | GGT | TGT |
| ATA | CTA | GTA | TTA |
| ATC | CTC | GTC | TTC |
| ATG | CTG | GTG | TTG |
| ATT | CTT | GTT | TTT |

Representative examples of seven base pair att site overlap regions suitable for in methods, compositions and vectors of the invention are shown in Table 2. The invention further includes nucleic acid molecules comprising one or more (e.g., one, two, three, four, five, six, eight, ten, twenty, thirty, forty, fifty, etc.) nucleotides sequences set out in Table 2. Thus, for example, in one aspect, the invention provides nucleic acid molecules comprising the nucleotide sequence GAAATAC, GATATAC, ACAATAC, or TGCATAC. However, in certain embodiments, the invention will not include nucleic acid molecules which comprise att site core regions set out herein in FIGS. 13A-13C.

TABLE 2

Representative Examples of Seven Base Pair att
Site Overlap Regions Suitable for Use with the
Invention.

| | | | |
|---|---|---|---|
| AAAATAC | CAAATAC | GAAATAC | TAAATAC |
| AACATAC | CACATAC | GACATAC | TACATAC |
| AAGATAC | CAGATAC | GAGATAC | TAGATAC |
| AATATAC | CATATAC | GATATAC | TATATAC |
| ACAATAC | CCAATAC | GCAATAC | TCAATAC |
| ACCATAC | CCCATAC | GCCATAC | TCCATAC |
| ACGATAC | CCGATAC | GCGATAC | TCGATAC |
| ACTATAC | CCTATAC | GCTATAC | TCTATAC |
| AGAATAC | CGAATAC | GGAATAC | TGAATAC |
| AGCATAC | CGCATAC | GGCATAC | TGCATAC |
| AGGATAC | CGGATAC | GGGATAC | TGGATAC |
| AGTATAC | CGTATAC | GGTATAC | TGTATAC |
| ATAATAC | CTAATAC | GTAATAC | TTAATAC |
| ATCATAC | CTCATAC | GTCATAC | TTCATAC |
| ATGATAC | CTGATAC | GTGATAC | TTGATAC |
| ATTATAC | CTTATAC | GTTATAC | TTTATAC |

As noted above, alterations of nucleotides located 3′ to the three base pair region discussed above can also affect recombination specificity. For example, alterations within the last four positions of the seven base pair overlap can also affect recombination specificity.

The invention thus provides recombination sites which recombine with a cognate partner, as well as molecules which contain these recombination sites and methods for generating, identifying, and using these sites. Methods which can be used to identify such sites are set out in U.S. application Ser. No. 09/732,914, filed Dec. 11, 2000, the entire disclosure of which is incorporated herein by reference. Examples of such recombination sites include att sites which contain 7 base pairs overlap regions which associate and recombine with cognate partners. The nucleotide sequences of specific examples of such 7 base pair overlap regions are set out above in Table 2.

Further embodiments of the invention include isolated nucleic acid molecules comprising a nucleotide sequence at least 50% identical, at least 60% identical, at least 70% identical, at least 75% identical, at least 80% identical, at least 85% identical, at least 90% identical, or at least 95% identical to the nucleotide sequences of the seven base pair overlap regions set out above in Table 2 or the 15 base pair core region shown in SEQ ID NO:47, as well as a nucleotide sequence complementary to any of these nucleotide sequences or fragments, variants, mutants, and derivatives thereof. Additional embodiments of the invention include compositions and vectors which contain these nucleic acid molecules, as well as methods for using these nucleic acid molecules.

In specific embodiments, recombination sites having nucleotide sequences set out below in FIGS. 13A-13C, as well as recombination sites comprising a nucleotide sequence at least 50% identical, at least 60% identical, at least 70% identical, at least 75% identical, at least 80% identical, at least 85% identical, at least 90% identical, or at least 95% identical to the nucleotide sequences set out in FIGS. 13A-13C, may also be used in the practice of the invention.

Recombinant host cells comprising a nucleic acid molecule (the attP vector pDONR201 (Invitrogen Corp., Carlsbad, Calif., Cat. No. 11798-014), containing attP1 and attP2 sites, E. coli DB3.1 (also called E. coli DB3.1 (pAHKan)), were deposited on Feb. 27, 1999, with the Collection, Agricultural Research Culture Collection (NRRL), 1815 North University Street, Peoria, Ill. 61604 USA, as Deposit No. NRRL B-30099. The attP1 and attP2 sites within the deposited nucleic acid molecule are contained in nucleic acid cassettes in association with one or more additional functional sequences as described in more detail elsewhere herein.

Further, recombinant host cell strains containing attR1 sites apposed to cloning sites in reading frame A, reading frame B, and reading frame C, E. coli DB3.1 (pEZC15101) (reading frame A), E. coli DB3.1 (pEZC15102) (reading frame B), and E. coli DB3.1 (pEZC15103) (reading frame C), and containing corresponding attR2 sites, were deposited on Feb. 27, 1999, with the Collection, Agricultural Research Culture Collection (NRRL), 1815 North University Street, Peoria, Ill. 61604 USA, as Deposit Nos. NRRL B-30103, NRRL B-30104, and NRRL B-30105, respectively. The attR1 and attR2 sites within the deposited nucleic acid molecules are contained in nucleic acid cassettes in association with one or more additional functional sequences as described in more detail elsewhere herein. Variations of these vectors may or may not contain stop codons just after the attR2 site.

In addition, recombinant host cell strains containing attL1 sites apposed to cloning sites in reading frame A, reading frame B, and reading frame C, E. coli DB3.1(pENTR1A) (reading frame A), E. coli DB3.1(pENTR2B) (reading frame B), and E. coli DB3.1(pENTR3C) (reading frame C), and containing corresponding attL2 sites, were deposited on Feb. 27, 1999, with the Collection, Agricultural Research Culture Collection (NRRL), 1815 North University Street, Peoria, Ill. 61604 USA, as Deposit Nos. NRRL B-30100, NRRL B-30101, and NRRL B-30102, respectively. The attL1 and attL2 sites within the deposited nucleic acid molecules are contained in nucleic acid cassettes in association with one or more additional functional sequences as described in more detail elsewhere herein.

By a polynucleotide having a nucleotide sequence at least, for example, 95% “identical” to a reference nucleotide sequence encoding a particular recombination site or portion thereof is intended that the nucleotide sequence of the polynucleotide is identical to the reference sequence except that the polynucleotide sequence may include up to five point mutations (e.g., insertions, substitutions, or deletions) per each 100 nucleotides of the reference nucleotide sequence encoding the recombination site. For example, to obtain a polynucleotide having a nucleotide sequence at least 95% identical to a reference attB1 nucleotide sequence (SEQ ID NO:5), up to 5% of the nucleotides in the attB1 reference sequence may be deleted or substituted with another nucleotide, or a number of nucleotides up to 5% of the total nucleotides in the attB1 reference sequence may be inserted into the attB1 reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 50%, 60%, 70%, 75%, 80%, 85%, 90%, 95%, 96%, 97%, 98% or 99% identical to, for instance, a given recombination site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2:482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

Unless otherwise indicated, each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). Thus, the invention relates to sequences of the invention in the form of DNA or RNA molecules, or hybrid DNA/RNA molecules, and their corresponding complementary DNA, RNA, or DNA/RNA strands.

In a related aspect, the present invention also relates to nucleic acid molecules comprising one or more recombination site nucleotide sequences that enhance recombination efficiency, particularly one or more nucleotide sequences that may correspond substantially to the core region and having one or more mutations that enhance recombination efficiency. By sequences or mutations that “enhance recombination efficiency” is meant a sequence or mutation in a recombination site, often in the core region (e.g., the 15 base pair core region of att recombination sites), that results in an increase in cloning efficiency (typically measured by determining successful cloning of a test sequence, e.g., by determining CFU/ml for a given cloning mixture) when recombining molecules comprising the mutated sequence or core region as compared to molecules that do not comprise the mutated sequence or core region (e.g., those comprising a wild-type recombination site core region sequence). More specifically, whether or not a given sequence or mutation enhances recombination efficiency may be determined using the sequence or mutation in recombinational cloning as described herein, and determining whether the sequence or mutation provides enhanced recombinational cloning efficiency when compared to a non-mutated (e.g., wild-type) sequence.

Using the information provided herein, such as the nucleotide sequences for the recombination site sequences described herein, an isolated nucleic acid molecule to be used in the present invention encoding one or more recombination sites or portions thereof may be obtained using standard cloning and screening procedures, such as those for cloning cDNAs using mRNA as starting material. Such methods include PCR-based cloning methods, such as reverse transcriptase-PCR (RT-PCR). Alternatively, vectors comprising the cassettes containing the recombination site sequences described herein are available commercially from Invitrogen Corp., Carlsbad, Calif.

The invention also relates to nucleic acid molecules comprising one or more of the recombination site sequences or portions thereof and one or more additional nucleotide sequences, which may encode functional or structural sites such as one or more multiple cloning sites, one or more transcription termination sites, one or more transcriptional regulatory sequences (which may be promoters, enhancers, repressors, and the like), one or more translational signals (e.g., secretion signal sequences), one or more origins of replication, one or more fusion partner peptides (particularly thioredoxin (Trx), glutathione S-transferase (GST), maltose binding protein (MBP), epitopes, defined amino acid sequences such as epitopes, haptens, six histidines (HIS6), and the like), one or more selection markers or modules, one or more nucleotide sequences encoding localization signals such as nuclear localization signals or secretion signals, one or more origins of replication, one or more protease cleavage sites, one or more genes or portions of genes encoding a protein or polypeptide of interest, and one or more 5′ polynucleotide extensions (particularly an extension of nucleotides (e.g., guanine residues) ranging in length from about 1 to about 20, from about 2 to about 15, from about 3 to about 10, from about 4 to about 10, or an extension of 4 or 5 nucleotides (e.g., guanine, cytosine, adenine, or thymine residues) at the 5′ end of the recombination site). The one or more additional functional or structural sequences may or may not flank one or more of the recombination site sequences contained on the nucleic acid molecules used in the invention.

In some nucleic acid molecules used in the invention, the one or more nucleotide sequences encoding one or more additional functional or structural sites may be operably linked to the nucleotide sequence encoding the recombination site. For example, certain nucleic acid molecules used in the invention may have a promoter sequence operably linked to a nucleotide sequence encoding a recombination site or portion thereof of the invention, such as a T7 promoter, a phage lambda PL promoter, an E. coli lac, trp or tac promoter, and other suitable promoters which will be familiar to the skilled artisan.

Nucleic acid molecules used in the present invention, which may be isolated nucleic acid molecules, may be in the form of RNA, such as mRNA, or in the form of DNA, including, for instance, cDNA and genomic DNA obtained by cloning or produced synthetically, or in the form of DNA-RNA hybrids. The nucleic acid molecules used in the invention may be double-stranded or single-stranded. Single-stranded DNA or RNA may be the coding strand, also known as the sense strand, or it may be the non-coding strand, also referred to as the anti-sense strand. The nucleic acid molecules used in the invention may also have a number of topologies, including linear, circular, coiled, or supercoiled.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells, and those DNA molecules purified (partially or substantially) from a solution whether produced by recombinant DNA or synthetic chemistry techniques. Isolated RNA molecules include in vivo or in vitro RNA transcripts of the DNA molecules of the present invention.

Mutations can also be introduced into the recombination site nucleotide sequences for enhancing site specific recombination or altering the specificities of the reactants, etc. Such mutations include, but are not limited to: recombination sites without translation stop codons that allow fusion proteins to be encoded, recombination sites recognized by the same proteins but differing in base sequence such that they react largely or exclusively with their homologous partners allowing multiple reactions to be contemplated, and mutations that prevent hairpin formation of recombination sites. Which particular reactions take place can be specified by which particular partners are present in the reaction mixture.

Recombination Reaction Enhancers

The invention further provides methods for enhancing the efficiency of recombination reactions used in processes of the invention, as well as compositions which enhance the efficiency of recombination reactions.

In one aspect, the invention provides methods for enhancing the efficiency of recombination reactions. These methods involve the addition of one or more (e.g., one, two, three, four, five, six, seven, eight, nine, ten, etc.) proteins which enhance recombination efficiency to recombination reactions. Examples of proteins which enhance the efficiency of recombination reactions include E. coli ribosomal proteins S10, S14, S15, S16, S17, S18, S19, S20, S21, L14, L21, L23, L24, L25, L27, L28, L29, L30, L31, L32, L33 and L34, as well as fragments of these proteins comprising at least fifteen, at least twenty, at least thirty, at least forty, at least fifty, at least sixty, etc. amino acid residues. Additional examples include ribosomal proteins from organisms other than E. coli. Further examples include Fis proteins and Fis protein fragments.

Fis proteins or Fis protein fragments used in compositions and/or methods of the invention may be obtained from a wide variety of organisms (e.g., bacteria including, but not limited to, those of the genera Escherichia, Serratia, Salmonella, Pseudomonas, Haemophilus, Bacillus, Streptomyces, Staphylococcus, Streptococcus, or other gram positive or gram negative bacteria).

Generally, Fis proteins and Fis protein fragments used with the invention will have molecular weights which are below 14 kiloDaltons (kDa). Further, in many instances, between about 2% and about 40%, about 5% and about 35%, about 10% and about 35%, about 10% and about 30%, about 15% and about 30%, or about 15% and about 25% of the amino acid residues of these proteins will be basic amino acid residues. By “basic amino acid residues” is meant amino acid residues which have pKₐs above 7.0 (e.g., arginine, lysine, histidine, etc.). Thus, the invention includes compositions which contain the above described Fis proteins and Fis protein fragments, as well as methods for using these compositions in methods of the invention.

One example of a Fis protein is the 98 amino acid Fis protein of E. coli, which has the following amino acid sequence:

| | | | | | | | |
|---|---|---|---|---|---|---|---|
| 1 | MFEQRVNSDV | LTVSTVNSQD | QVTQKPLRDS | VKQALKNYFA | QLNGQDVNDL | YELVLAEVEQ | (SEQ ID NO:49) |
| 61 | PLLDMVMAYT | RGNQTRAALM | MGINRGTLRK | KLKKYGMN | | | |

Another example of a Fis protein is the 93 amino acid Fis protein of Klebsiella pneumoniae, which has the following amino acid sequence:

| | | | | | | | |
|---|---|---|---|---|---|---|---|
| 1 | MFEQRVNSDV | LTVSTVNSQD | QVTQKPLRDS | VKQALKNYFA | QLNGQDVNDL | YELVLAEVEQ | (SEQ ID NO:50) |
| 61 | PLLDMVMQYT | RGNQTRAALM | MGINRGTLRK | KLK | | | |

Yet another example of a Fis protein is the 98 amino acid Fis protein of Vibrio cholera, which has the following amino acid sequence:

| | | | | | | | |
|---|---|---|---|---|---|---|---|
| 1 | MFEQNLTSEA | LTVTTVTSQD | QITQKPLRDS | VKASLKNYLA | QLNGQEVTEL | YELVLAEVEQ | (SEQ ID NO:51) |
| 61 | PLLDTIMQYT | RGNQTRAATM | MGINRGTLRK | KLKKYGMN | | | |

Another example of a Fis protein is the 99 amino acid Fis protein of Haemophilus influenzae, which has the following amino acid sequence:

| | | | | | | | |
|---|---|---|---|---|---|---|---|
| 1 | MLEQQRNSAD | ALTVSVLNAQ | SQVTSKPLRD | SVKQALRNYL | AQLDGQDVND | LYELVLAEVE | (SEQ ID NO:52) |
| 61 | HPMLDMIMQY | TRGNQTRAAN | MLGINRGTLR | KKLKKYGMG | | | |

A further example of a Fis protein is the 107 amino acid Fis protein of Pseudomonas aeruginosa, which has the following amino acid sequence:

| | | | | | | | |
|---|---|---|---|---|---|---|---|
| 1 | MTTMTTETLV | SGTTPVSDNA | NLKQHLTTPT | QEGQTLRDSV | EKALHNYFAH | LEGQPVTDVY | (SEQ ID NO:53) |
| 61 | NMVLCEVEAP | LLETVMNHVK | GNQTKASELL | GLNRGTLRKK | LKQYDLL | | |

A yet further example of a Fis protein is the 98 amino acid Fis protein of Salmonella typhimurium, which has the following amino acid sequence:

| | | | | | | | |
|---|---|---|---|---|---|---|---|
| 1 | MFEQRVNSDV | LTVSTVNSQD | QVTQKPLRDS | VKQALKNYFA | QLNGQDVNDL | YELVLAEVEQ | (SEQ ID NO:54) |
| 61 | PLLDMVMQYT | RGNQTRAALM | MGINRGTLRK | KLKKYGMN | | | |

Methods of the invention employ Fis proteins and Fis protein fragments, as well as variants, derivatives and mutants of Fis proteins and Fis protein fragments which enhance the efficiency of recombination reactions. Fis protein fragments suitable for use with the invention include fragments which comprise at least 10 amino acids, at least 15 amino acids, at least 20 amino acids, at least 30 amino acids, at least 35 amino acids, at least 40 amino acids, at least 45 amino acids, at least 50 amino acids, at least 55 amino acids, at least 60 amino acids, at least 70 amino acids, at least 75 amino acids, at least 80 amino acids, at least 85 amino acids, etc. Fis protein fragments suitable for use with the invention also include fragments which comprise between about 10-20 amino acids, about 20-30 amino acids, about 30-40 amino acids, about 50-60 amino acids, about 60-70 amino acids, about 70-80 amino acids, about 90-100 amino acids, etc.

Proteins which may also be used with the invention include variants, derivatives and mutants which comprise amino acid sequences at least 65%, 70%, 75%, 80%, 85%, 90%, 95%, 98%, or 99% identical to a reference Fis protein (e.g., a Fis protein having an amino acid sequence set out above) or Fis protein fragment.

By a protein or protein fragment having an amino acid sequence at least, for example, 65% “identical” to a reference amino acid sequence is intended that the amino acid sequence of the protein is identical to the reference sequence except that the protein sequence may include up to 35 amino acid alterations per each 100 amino acids of the amino acid sequence of the reference protein. In other words, to obtain a protein having an amino acid sequence at least 65% identical to a reference amino acid sequence, up to 35% of the amino acid residues in the reference sequence may be deleted or substituted with another amino acid, or a number of amino acids up to 35% of the total amino acid residues in the reference sequence may be inserted into the reference sequence. These alterations of the reference sequence may occur at the amino (N—) or carboxy (C—) terminal positions of the reference amino acid sequence or anywhere between those terminal positions, interspersed either individually among residues in the reference sequence or in one or more contiguous groups within the reference sequence. As a practical matter, whether a given amino acid sequence is, for example, at least 65% identical to the amino acid sequence of a reference protein can be determined conventionally using known computer programs such as those described above for nucleic acid sequence identity determinations, or using the CLUSTAL W program (Thompson, J. D., et al., Nucleic Acids Res. 22:4673-4680 (1994)).

Fis protein fragments which may be used in the practice of the invention also comprise N-terminal and C-terminal deletion mutants of Fis proteins (e.g., a Fis protein having an amino acid sequence set out in any of SEQ ID NOs:49-54). Such Fis protein fragments include those in which at least 5 amino acids, at least 10 amino acids, at least 15 amino acids, at least 20 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, or at least 75 amino acids have been deleted from the N-terminus. Such Fis protein fragments also include those in which at least 1 amino acid, at least 2 amino acids, at least 3 amino acids, at least 4 amino acids, at least 5 amino acids, at least 6 amino acids, at least 7 amino acids, at least 8 amino acids, at least 9 amino acids, or at least 10 amino acids have been deleted from the C-terminus. Further, such Fis protein fragments include proteins comprising both the N-terminal and C-terminal deletions set out above.

Specific examples of Fis deletion mutants which may be used in the practice of the invention include Fis protein fragments comprising amino acids 75-98 of SEQ ID NO:49, amino acid 76-97 of SEQ ID NO:49, amino acid 77-96 of SEQ ID NO:49, amino acid 78-95 of SEQ ID NO:49, amino acid 79-93 of SEQ ID NO:49, or amino acid 80-92 of SEQ ID NO:49, as well as corresponding regions of other Fis proteins.

The invention also includes nucleic acid molecules which encode the Fis proteins referred to herein, as well as the use of these nucleic acid molecules in processes of the invention.

Compositions of the invention may also comprise proteins and protein fragments which bind to nucleic acids that Fis specifically binds to and enhance the efficiency of recombination reactions. For example, Fis has been shown to bind to nucleic acids having the following nucleotide sequence:

GNTYAAWWWTTRANC (SEQ ID NO:45), where R = A
or G, W = A or T, and Y = C or T.

Fis also binds to nucleic acids having the following nucleotide sequence:

| | |
|---|---|
| AGTCTGTTTTTTATGCAAAA. | (SEQ ID NO:46) |

Thus, in certain embodiments, the invention includes methods for enhancing recombination reactions which employ proteins and peptides that (1) bind to nucleic acids having the nucleotide sequence shown in SEQ ID NO:45 or SEQ ID NO:46, or proteins and peptides that bind to nucleic acids having a nucleotide sequence shown in SEQ ID NO:45 or SEQ ID NO:46 with one, two, three, or four substitutions, deletions or insertions, and (2) enhance the efficiency of recombination reactions.

Fis proteins and Fis protein fragments of the invention, as well as proteins and peptides which bind nucleic acids that Fis specifically binds to, may be prepared and used as fusion proteins. Fis is believed to form dimers. Thus, examples of fusion proteins which may be used in methods of the invention are fusion proteins which comprises (1) a Fis protein, a Fis protein fragment, or a peptide which binds to nucleic acid comprising the nucleotide sequence shown in SEQ ID NO:45 or SEQ ID NO:46 and (2) a protein or protein domain which facilitates the formation of multimers (e.g., homodimers). Examples of such proteins and protein domains include SH2 domains, protein DnaA of Streptomyces, AraC, heat shock protein 90, etc. Thus, the invention includes fusion proteins described above, nucleic acid molecules which encode these fusion proteins, and methods for using these fusion proteins and nucleic acid molecules to enhance the efficiency of recombination reactions.

Specific parameters and conditions related to the optimization of recombination reactions performed in the presence of Fis are set out below in Example 9 and can also be determined using known assays. For example, a titration assay may be used to determine the appropriate amount of a purified Fis protein, or the appropriate amount of an extract. Such assays are described in detail in the Examples below.

Fis proteins and Fis protein fragments, as well as other proteins and protein fragments which enhance the efficiency of recombination reactions, may be included in recombination reactions (e.g., BP CLONASE™ catalyzed recombination reactions) in a variety of concentrations, including about 0.5 ng/μl, about 1.0 ng/μl, about 1.5 ng/μl, about 2.0 ng/μl, about 2.5 ng/μl, about 3.0 ng/μl, about 3.5 ng/μl, about 4.0 ng/μl, about 4.5 ng/μl, about 5.0 ng/μl, about 5.5 ng/μl, about 6.0 ng/μl, about 6.5 ng/lμl, about 7.0 ng/μl, about 7.5 ng/μl, about 8.0 ng/μl, about 8.5 ng/μl, about 9.0 ng/μl, about 9.5 ng/μl, about 10.0 ng/μl, about 10.5 ng/μl, about 11.0 ng/μl, about 11.5 ng/μl, about 12.0 ng/μl, about 12.5 ng/μl, about 13.0 ng/μl, about 13.5 ng/μl, about 14.0 ng/μl, about 14.5 ng/μl, about 15.0 ng/μl, about 16.0 ng/μl, about 17.0 ng/μl, about 18.0 ng/μl, about 19.0 ng/μl, about 20.0 ng/μl, about 22.0 ng/μl, about 25.0 ng/μl, about 27.0 ng/μl, about 30.0 ng/μl, about 35.0 ng/μl, or about 40.0 ng/μl. Similarly, Fis may be included in recombination reactions in a variety of ranges, including from about 0.5 ng/μl to about 40.0 ng/μl, from about 0.5 ng/μl to about 30.0 ng/μl, from about 0.5 ng/μl to about 15.0 ng/μl, from about 1.0 ng/μl to about 14.0 ng/μl, from about 5.0 ng/μl to about 10.0 ng/μl, from about 7.0 ng/μl to about 15.0 ng/μl, from about 10.0 ng/μl to about 15.0 ng/μl, from about 5.0 ng/μl to about 30.0 ng/μl, from about 10.0 ng/μl to about 30.0 ng/μl, from about 20 ng/μl to about 30.0 ng/μl, from about 20 ng/μl to about 35.0 ng/μl, or from about 20 ng/μl to about 40.0 ng/μl. Of course, other concentrations and ranges suitable for use in methods of the invention may be determined by one of ordinary skill without undue experimentation by carrying out a titration assay as noted above and as described in detail in the Examples below. Concentrations and ranges set out above of ribosomal proteins which enhance recombination efficiency may also be included in recombination reactions to enhance efficiency. Thus, the invention further includes methods described herein which employ proteins that enhance the efficiency of recombination reactions.

Vectors

The invention also relates to vectors comprising one or more of the nucleic acid molecules used in the invention and/or used in methods of the invention. In accordance with the invention, any vector may be used to construct the vectors of invention. In particular, vectors known in the art and those commercially available (and variants or derivatives thereof) may in accordance with the invention be engineered to include one or more nucleic acid molecules encoding one or more recombination sites (or portions thereof), or mutants, fragments, or derivatives thereof, for use in the methods of the invention. Such vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, vector suitable for use for gene therapy applications (e.g., viral vectors), vectors for receiving large inserts, and the like.

Other vectors of interest include viral origin vectors (M13 vectors, bacterial phage λ vectors, bacteriophage P1 vectors, adenovirus vectors, herpesvirus vectors, retrovirus vectors, phage display vectors, combinatorial library vectors), high, low, and adjustable copy number vectors, vectors which have compatible replicons for use in combination in a single host (pACYC184 and pBR322) and eukaryotic episomal replication vectors (pCDM8).

Particular vectors of interest include prokaryotic Expression Vectors such as pcDNA II, pSL301, pSE280, pSE380, pSE420, pTrcHisA, B, and C, pRSET A, B, and C (Invitrogen Corp., Carlsbad, Calif.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen Corp., Carlsbad, Calif.) and variants and derivatives thereof. Destination Vectors can also be made from eukaryotic Expression Vectors such as pFastBac, pFastBac HT, pFastBac DUAL, pSFV, and pTet-Splice (Invitrogen Corp., Carlsbad, Calif.), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXT1, pSG5, pPbac, pMbac, pMC1neo, and pOG44 (Stratagene, Inc.), and pYES2, pAC360, pBlueBacHis A, B, and C, pVL1392, pBsueBacIII, pCDM8, pcDNA1, pZeoSV, pcDNA3 pREP4, pCEP4, and pEBVHis (Invitrogen Corp., Carlsbad, Calif.) and variants or derivatives thereof.

Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), pQE70, pQE60, pQE9 (Quiagen), pBS vectors, PhageScript vectors, BlueScript vectors, pNH8A, pNH16A, pNH18A, pNH46A (Stratagene), pcDNA3 (Invitrogen, Carlsbad, Calif.), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen Corp., Carlsbad, Calif.) and variants or derivatives thereof.

Additional vectors of interest include pTrxFus, pThioHis, pLEX, pTrcHis, pTrcHis2, pRSET, pBlueBacHis2, pcDNA3.1/His, pcDNA3.1(−)/Myc-His, pSecTag, pEBVHis, pPIC9K, pPIC3.5K, pAO815, pPICZ, pGAPZ, pBlueBac4.5, pBlueBacHis2, pMelBac, pSinRep5, pSinHis, pIND, pIND(SP1), pVgRXR, pcDNA2.1. pYES2, pZErO1.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP10, pCEP4, pEBVHis, pCR3.1, pCR2.1, pCR3.1-Uni, and pCRBac from Invitrogen; λgt11, pTrc99A, pKK223-3, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4abc(+), pOCUS-2, pTAg, pET-32 LIC, pET-30 LIC, pBAC-2 cp LIC, pBACgus-2 cp LIC, pT7Blue-2 LIC, pT7Blue-2, pET-3abcd, pET-7abc, pET9abcd, pET11abcd, pET12abc, pET-14b, pET-15b, pET-16b, pET-17b-pET-17xb, pET-19b, pET-20b(+), pET-21 abcd(+), pET-22b(+), pET-23abcd(+), pET-24abcd(+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, plg, Signal plg, pYX, Selecta Vecta-Neo, Selecta Vecta—Hyg, and Selecta Vecta—Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFP-N, pEGFP-C, pEBFP, pGFPuv, pGFP, p6xHis-GFP, pSEAP2-Basic, pSEAP2-Contral, pSEAP2-Promoter, pSEAP2-Enhancer, pβgal-Basic, pβgal-Control, pβgal-Promoter, ppgal-Enhancer, pTet-Off, pTet-On, pTK-Hyg, pRetro-Off, pRetro-On, pIRES1neo, pIRES1hyg, pLXSN, pLNCX, pLAPSN, pMAMneo, pMAMneo-CAT, pMAMneo-LUC, pPUR, pSV2neo, pYEX 4T-1/2/3, pYEX-S1, pBacPAK-His, pBacPAK8/9, pAcUW31, BacPAK6, pTriplEx, λgt10, λgt11, and pWE15, and from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS +/−, pBC KS+/−, pBC SK+/−, Phagescript, pCAL-n-EK, pCAL-n, pCAL-c, pCAL-kc, pET-3abcd, pET-1 labcd, pSPUTK, pESP-1, pCMVLacI, pOPRSVI/MCS, pOPI3 CAT, pXT1, pSG5, pPbac, pMbac, pMClneo, pMC1neo Poly A, pOG44, pOG45, pFRTβGAL, pNEOβGAL, pRS403, pRS404, pRS405, pRS406, pRS413, pRS414, pRS415, and pRS416 from Stratagene.

Two-hybrid and reverse two-hybrid vectors of particular interest include pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp and variants or derivatives thereof.

Yeast Expression Vectors of particular interest include pESP-1, pESP-2, pESC-His, pESC-Trp, pESC-URA, pESC-Leu (Stratagene), pRS401, pRS402, pRS411, pRS412, pRS421, pRS422, and variants or derivatives thereof.

According to the invention, vectors comprising one or more nucleic acid molecules encoding one or more recombination sites, or mutants, variants, fragments, or derivatives thereof, may be produced by one of ordinary skill in the art without resorting to undue experimentation using standard molecular biology methods. For example, vectors of the invention, as well as vector suitable for use in methods of the invention, may be produced by introducing one or more of the nucleic acid molecules encoding one or more recombination sites (or mutants, fragments, variants or derivatives thereof) into one or more of the vectors described herein, according to the methods described, for example, in Maniatis et al., Molecular Cloning: A Laboratory Manual, Cold Spring Harbor Laboratory, Cold Spring Harbor, N.Y. (1982). In a related aspect of the invention, the vectors may be engineered to contain, in addition to one or more nucleic acid molecules encoding one or more recombination sites (or portions thereof), one or more additional physical or functional nucleotide sequences, such as those encoding one or more multiple cloning sites, one or more transcription termination sites, one or more transcriptional regulatory sequences (e.g., one or more promoters, enhancers, or repressors), one or more selection markers or modules, one or more genes or portions of genes encoding a protein or polypeptide of interest, one or more translational signal sequences, one or more nucleotide sequences encoding a fusion partner protein or peptide (e.g., GST, His₆ or thioredoxin), one or more origins of replication, and one or more 5′ or 3′ polynucleotide tails (particularly a poly-G tail). According to this aspect of the invention, the one or more recombination site nucleotide sequences (or portions thereof) may optionally be operably linked to the one or more additional physical or functional nucleotide sequences described herein.

Vectors according to this aspect of the invention include, but are not limited to: pENTR1A, pENTR2B, pENTR3C, pENTR4, pENTR5, pENTR6, pENTR7, pENTR8, pENTR9, pENTR10, pENTR11, pDEST1, pDEST2, pDEST3, pDEST4, pDEST5, pDEST6, pDEST7, pDEST8, pDEST9, pDEST10, pDEST11, pDEST12.2 (also known as pDEST12), pDEST13, pDEST14, pDEST15, pDEST16, pDEST17, pDEST18, pDEST19, pDEST20, pDEST21, pDEST22, pDEST23, pDEST24, pDEST25, pDEST26, pDEST27, pEXP501 (also known as pCMVSPORT6.0, FIG. 34A-34D), pDONR201 (FIGS. 26A-26C), pDONR202, pDONR203, pDONR204, pDONR205, pDONR206, pDONR212 (FIGS. 27A-27C), pDONR212(F) (FIGS. 28A-28C), pDONR212(R) (FIGS. 29A-29C), pMAB58, pMAB62, pDEST28, pDEST29, pDEST30, pDEST31, pDEST32, pDEST33, pDEST34, pDONR207 (FIGS. 18A-18C), pMAB85, pMAB86, a number of which are described in PCT Publication WO 00/52027 (the entire disclosure of which is incorporated herein by reference), and fragments, mutants, variants, and derivatives of each of these vectors. However, it will be understood by one of ordinary skill that the present invention also encompasses other vectors not specifically designated herein, which comprise one or more of the isolated nucleic acid molecules used in the invention encoding one or more recombination sites or portions thereof (or mutants, fragments, variants or derivatives thereof), and which may further comprise one or more additional physical or functional nucleotide sequences described herein which may optionally be operably linked to the one or more nucleic acid molecules encoding one or more recombination sites or portions thereof. Such additional vectors may be produced by one of ordinary skill according to the guidance provided in the present specification.

Additional vectors which can be used with the invention include vectors suitable for use in gene therapy applications. Adenoviruses are especially attractive vehicles for delivering genes to respiratory epithelia and the use of such vectors are included within the scope of the invention. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication Nos. WO 94/12649 and WO 96/17053; U.S. Pat. No. 6,190,907; U.S. Pat. No. 6,140,087; U.S. Pat. No. 6,204,060; U.S. Pat. No. 5,998,205; and Wang et al., 1995, Gene Therapy 2:775-783, the disclosures of all of which are incorporated herein by reference in their entireties. In certain embodiments, adenovirus vectors are used.

Adeno-associated virus (AAV), retroviruses, lentiviruses, and Herpes viruses, as well as vectors prepared from these viruses have also been proposed for use in gene therapy (see Walsh et al., 1993, Proc. Soc. Exp. Biol. Med. 204:289-300; Steinberg et al., Gene Ther. 7:1392-1400 (2000); Kordower et al., Science 290:767-773 (2000); U.S. Pat. No. 5,436,146; Wagstaff et al., Gene Ther. 5:1566-1570 (1998), the entire disclosures of each of which are incorporated herein by reference). Herpes viral vectors are particularly useful for applications where gene expression is desired in nerve cells.

Polymerases

Polypeptides having reverse transcriptase activity (i.e., those polypeptides able to catalyze the synthesis of a DNA molecule from an RNA template) for use in accordance with the present invention include, but are not limited to Moloney Murine Leukemia Virus (M-MLV) reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase, Human Immunodeficiency Virus (HIV) reverse transcriptase, retroviral reverse transcriptase, retrotransposon reverse transcriptase, hepatitis B reverse transcriptase, cauliflower mosaic virus reverse transcriptase and bacterial reverse transcriptase. These polypeptides having reverse transcriptase activity may further have substantially reduced RNAse H activity (i.e., “RNAse H-” polypeptides). By polypeptides that “have substantially reduced RNAse H activity” is meant that the polypeptides, or an individual polypeptide, have less than about 20%, less than about 15%, less than about 10%, less than about 5%, or less than about 2%, of the RNase H activity of a wild-type or RNase H⁺ enzyme such as wild-type M-MLV reverse transcriptase. The RNase H activity may be determined by a variety of assays, such as those described, for example, in U.S. Pat. No. 5,244,797, in Kotewicz, M. L. et al., Nucl. Acids Res. 16:265 (1988) and in Gerard, G. F., et al., FOCUS 14(5):91 (1992), the disclosures of all of which are fully incorporated herein by reference. Suitable RNAse H⁻ polypeptides for use in the present invention include, but are not limited to, M-MLV H⁻ reverse transcriptase, RSV H⁻ reverse transcriptase, AMV H⁻ reverse transcriptase, RAV H⁻ reverse transcriptase, MAV H⁻ reverse transcriptase, HIV H⁻ reverse transcriptase, THERMOSCRIPT™ reverse transcriptase and THERMOSCRIPT™ II reverse transcriptase, and SUPERSCRIPT™ I reverse transcriptase and SUPERSCRIPT™ II reverse transcriptase, which are obtainable, for example, from Invitrogen Corp., Carlsbad, Calif. (See generally PCT Publication No. WO 98/47912.)

Other polypeptides having nucleic acid polymerase activity suitable for use in the present methods include thermophilic DNA polymerases such as DNA polymerase I, DNA polymerase III, Klenow fragment, T7 polymerase, and T5 polymerase, and thermostable DNA polymerases including, but not limited to, Thermus thermophilus (Tth) DNA polymerase, Thermus aquaticus (Taq) DNA polymerase, Thermotoga neopolitana (Tne) DNA polymerase, Thermotoga maritima (Tma) DNA polymerase, Thermococcus litoralis (Tli or VENT®) DNA polymerase, Pyrococcus furiosus (Pfu) DNA polymerase, Pyrococcus species GB-D (or DEEPVENT®) DNA polymerase, Pyrococcus woosii (Pwo) DNA polymerase, Bacillus sterothermophilus (Bst) DNA polymerase, Sulfolobus acidocaldarius (Sac) DNA polymerase, Thermoplasma acidophilum (Tac) DNA polymerase, Thermus flavus (Tfl/Tub) DNA polymerase, Thermus ruber (Tru) DNA polymerase, Thermus brockianus (DYNAZYME®) DNA polymerase, Methanobacterium thermoautotrophicum (Mth) DNA polymerase, and mutants, variants and derivatives thereof. Such polypeptides are available commercially, for example from Invitrogen Corp., Carlsbad, Calif., New England BioLabs (Beverly, Mass.), and Sigma/Aldrich (St. Louis, Mo.).

Host Cells

The invention also relates to host cells comprising one or more of the nucleic acid molecules or vectors used in, selected and/or isolated by the invention, particularly those nucleic acid molecules and vectors described in detail herein. Representative host cells that may be used according to this aspect of the invention include, but are not limited to, bacterial cells, yeast cells, plant cells and animal cells. Bacterial host cells suitable for use with the invention include Escherichia spp. cells (particularly E. coli cells and most particularly E. coli strains DH10B, Stbl2, DH5α, DB3, DB3.1 (e.g., E. coli LIBRARY EFFICIENCY® DB3.1™ Competent Cells; Invitrogen Corp., Carlsbad, Calif.), DB4 and DB5; see U.S. application Ser. No. 09/518,188, filed on Mar. 2, 2000, the disclosure of which is incorporated by reference herein in its entirety), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Animal host cells suitable for use with the invention include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly CHO, COS, VERO, BHK and human cells). Yeast host cells suitable for use with the invention include Saccharomyces cerevisiae cells and Pichia pastoris cells. These and other suitable host cells are available commercially, for example from Invitrogen Corp., Carlsbad, Calif., American Type Culture Collection (Manassas, Va.), and Agricultural Research Culture Collection (NRRL; Peoria, Ill.).

Methods of the invention may also be used in cell free systems. Examples of cell free systems which can be used with the invention include in vitro transcription and translation systems.

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, transfection, and transformation. The nucleic acid molecules and/or vectors of the invention may be introduced alone or in conjunction with other the nucleic acid molecules and/or vectors. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Hence, a wide variety of techniques suitable for introducing the nucleic acid molecules and/or vectors of the invention into cells (e.g., ballistic bombardment, electroporation, lipofection, etc.) in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E., From Genes to Clones, New York: VCH Publishers (1987), which are illustrative of the many laboratory manuals that detail these techniques and which are incorporated by reference herein in their entireties for their relevant disclosures.

Polypeptides

In another aspect, the invention relates to polypeptides encoded by the nucleic acid molecules selected and/or isolated by the invention (including polypeptides and amino acid sequences encoded by all possible reading frames of the nucleic acid molecules used in the invention), and to methods of producing such polypeptides. Polypeptides of the present invention include purified or isolated natural products, products of chemical synthetic procedures, and products produced by recombinant techniques from a prokaryotic or eukaryotic host, including, for example, bacterial, yeast, insect, mammalian, avian and higher plant cells.

The polypeptides of the invention may be produced by methods such as those involving synthetic organic chemistry or by recombinant methods (e.g., methods employing one or more of the host cells of the invention comprising the vectors or isolated nucleic acid molecules used in the invention). According to the invention, polypeptides may be produced by cultivating the host cells of the invention (which comprise one or more of the nucleic acid molecules used in the invention that may contained within an Expression Vector) under conditions favoring the expression of the nucleotide sequence contained on the nucleic acid molecule of the invention, such that the polypeptide encoded by the nucleic acid molecule of the invention is produced by the host cell. As used herein, “conditions favoring the expression of the nucleotide sequence” or “conditions favoring the production of a polypeptide” include optimal physical (e.g., temperature, humidity, etc.) and nutritional (e.g., culture medium, ionic) conditions required for production of a recombinant polypeptide by a given host cell. Such optimal conditions for a variety of host cells, including prokaryotic (bacterial), mammalian, insect, yeast, and plant cells will be familiar to one of ordinary skill in the art, and may be found, for example, in Sambrook, J., et al., Molecular Cloning, A Laboratory Manual, 2nd Ed., Cold Spring Harbor, N.Y.: Cold Spring Harbor Laboratory Press, (1989), Watson, J. D., et al., Recombinant DNA, 2nd Ed., New York: W.H. Freeman and Co., and Winnacker, E.-L., From Genes to Clones, New York: VCH Publishers (1987).

In some aspects, it may be desirable to isolate or purify the polypeptides of the invention (e.g., for production of antibodies as described below), resulting in the production of the polypeptides of the invention in isolated form. The polypeptides of the invention can be recovered and purified from recombinant cell cultures by well-known methods of protein purification that are routine in the art, including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. For example, HIS6 or GST fusion tags on polypeptides made by the methods of the invention may be isolated using appropriate affinity chromatography matrices which bind polypeptides bearing His6 or GST tags, as will be familiar to one of ordinary skill in the art. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Isolated polypeptides of the invention include those comprising the amino acid sequences encoded by one or more of the reading frames of the polynucleotides comprising one or more of the recombination site-encoding nucleic acid molecules used in the invention, including those encoding attB1, attB2, attP1, attP2, attL1, attL2, attR1 and attR2 having the nucleotide sequences set forth in FIGS. 13A-13C (or nucleotide sequences complementary thereto), or fragments, variants, mutants and derivatives thereof; the complete amino acid sequences encoded by the polynucleotides contained in the deposited clones described herein; the amino acid sequences encoded by polynucleotides which hybridize under stringent hybridization conditions to polynucleotides having the nucleotide sequences encoding the recombination site sequences of the invention as set forth in FIGS. 13A-13C (or a nucleotide sequence complementary thereto); or a peptide or polypeptide comprising a portion or a fragment of the above polypeptides. The invention also relates to additional polypeptides having one or more additional amino acids linked (typically by peptidyl bonds to form a nascent polypeptide) to the polypeptides encoded by the recombination site nucleotide sequences or the deposited clones. Such additional amino acid residues may comprise one or more functional peptide sequences, for example one or more fusion partner peptides (e.g., GST, HIS6, Trx, etc.) and the like.

As used herein, the terms “protein,” “peptide,” “oligopeptide” and “polypeptide” are considered synonymous (as is commonly recognized) and each term can be used interchangeably as the context requires to indicate a chain of two or more amino acids, five or more amino acids, or ten or more amino acids, coupled by (a) peptidyl linkage(s), unless otherwise defined in the specific contexts below. As is commonly recognized in the art, all polypeptide formulas or sequences herein are written from left to right and in the direction from amino terminus to carboxy terminus.

By “isolated” polypeptide or protein is intended a polypeptide or protein removed from its native environment. For example, recombinantly produced polypeptides and proteins expressed in host cells are considered isolated for purposes of the invention, as are native or recombinant polypeptides which have been substantially purified by any suitable technique such as, for example, the single-step purification method disclosed in Smith and Johnson, Gene 67:31-40 (1988).

It will be recognized by those of ordinary skill in the art that some amino acid sequences of the polypeptides of the invention can be varied without significant effect on the structure or function of the polypeptides. If such differences in sequence are contemplated, it should be remembered that there will be critical areas on the protein which determine structure and activity. In general, it is possible to replace residues which form the tertiary structure, provided that residues performing a similar function are used. In other instances, the type of residue may be completely unimportant if the alteration occurs at a non-critical region of the polypeptide.

Thus, the invention further relates to variants of the polypeptides of the invention, including allelic variants, which show substantial structural homology to the polypeptides described herein, or which include specific regions of these polypeptides such as the portions discussed below. Such mutants may include deletions, insertions, inversions, repeats, and type substitutions (for example, substituting one hydrophilic residue for another, but not strongly hydrophilic for strongly hydrophobic as a rule). Small changes or such “neutral” or “conservative” amino acid substitutions will generally have little effect on activity.

Typical conservative substitutions are the replacements, one for another, among the aliphatic amino acids Ala, Val, Leu and Ile; interchange of the hydroxylated residues Ser and Thr; exchange of the acidic residues Asp and Glu; substitution between the amidated residues Asn and Gln; exchange of the basic residues Lys and Arg; and replacements among the aromatic residues Phe and Tyr.

Thus, the fragment, derivative or analog of the polypeptides of the invention, such as those comprising peptides encoded by the recombination site nucleotide sequences described herein, may be (i) one in which one or more of the amino acid residues are substituted with a conservative or non-conservative amino acid residue, and such substituted amino acid residue may be encoded by the genetic code or may be an amino acid (e.g., desmosine, citrulline, ornithine, etc.) that is not encoded by the genetic code; (ii) one in which one or more of the amino acid residues includes a substituent group (e.g., a phosphate, hydroxyl, sulfate or other group) in addition to the normal “R” group of the amino acid; (iii) one in which the mature polypeptide is fused with another compound, such as a compound to increase the half-life of the polypeptide (for example, polyethylene glycol), or (iv) one in which additional amino acids are fused to the mature polypeptide, such as an immunoglobulin Fc region peptide, a leader or secretory sequence, a sequence which is employed for purification of the mature polypeptide (such as GST) or a proprotein sequence. Such fragments, derivatives and analogs are intended to be encompassed by the present invention, and are within the scope of those skilled in the art from the teachings herein and the state of the art at the time of invention.

The polypeptides of the present invention may be provided in an isolated form, and may be substantially purified. Recombinantly produced versions of the polypeptides of the invention can be substantially purified by the one-step method described in Smith and Johnson, Gene 67:31-40 (1988). As used herein, the term “substantially purified” means a preparation of an individual polypeptide of the invention wherein at least 50%, at least 60%, at least 70%, at least 75%, at least 80%, at least 85%, at least 90%, at least 91%, at least 92%, at least 93%, at least 94%, at least 95%, at least 96%, at least 97%, at least 98% or at least 99% (by mass) of contaminating proteins (i.e., those that are not the individual polypeptides described herein or fragments, variants, mutants or derivatives thereof) have been removed from the preparation.

The polypeptides of the present invention include those which are at least about 50% identical, at least 60% identical, at least 65% identical, at least about 70%, at least about 75%, at least about 80%, at least about 85%, at least about 90%, at least about 95%, at least about 96%, at least about 97%, at least about 98% or at least about 99% identical, to the polypeptides described herein. For example, attB1-containing polypeptides of the invention include those that are at least about 50% identical, at least 60% identical, at least 65% identical, at least about 70%, at least about 75%, at least about 80%, at least about 85%, at least about 90%, at least about 95%, at least about 96%, at least about 97%, at least about 98% or at least about 99% identical, to the polypeptide(s) encoded by the three reading frames of a polynucleotide comprising a nucleotide sequence of attB1 having a nucleic acid sequence as set forth in FIGS. 13A-13C (or a nucleic acid sequence complementary thereto), to a polypeptide encoded by a polynucleotide contained in the deposited cDNA clones described herein, or to a polypeptide encoded by a polynucleotide hybridizing under stringent conditions to a polynucleotide comprising a nucleotide sequence of attB1 having a nucleic acid sequence as set forth in FIGS. 13A-13C (or a nucleic acid sequence complementary thereto). Analogous polypeptides may be prepared that are at least about 65% identical, more at least about 70%, at least about 75%, at least about 80%, at least about 85%, at least about 90%, at least about 95%, at least about 96%, at least about 97%, at least about 98% or at least about 99% identical, to the attB2, attP1, attP2, attL1, attL2, attR1 and attR2 polypeptides of the invention as depicted in FIGS. 13A-13C. The present polypeptides also include portions or fragments of the above-described polypeptides with at least 5, 10, 15, 20, or 25 amino acids.

By a polypeptide having an amino acid sequence at least, for example, 65% “identical” to a reference amino acid sequence of a given polypeptide of the invention is intended that the amino acid sequence of the polypeptide is identical to the reference sequence except that the polypeptide sequence may include up to 35 amino acid alterations per each 100 amino acids of the reference amino acid sequence of a given polypeptide of the invention. In other words, to obtain a polypeptide having an amino acid sequence at least 65% identical to a reference amino acid sequence, up to 35% of the amino acid residues in the reference sequence may be deleted or substituted with another amino acid, or a number of amino acids up to 35% of the total amino acid residues in the reference sequence may be inserted into the reference sequence. These alterations of the reference sequence may occur at the amino (N-) or carboxy (C-) terminal positions of the reference amino acid sequence or anywhere between those terminal positions, interspersed either individually among residues in the reference sequence or in one or more contiguous groups within the reference sequence. As a practical matter, whether a given amino acid sequence is, for example, at least 65% identical to the amino acid sequence of a given polypeptide of the invention can be determined conventionally using known computer programs such as those described above for nucleic acid sequence identity determinations, or using the CLUSTAL W program (Thompson, J. D., et al., Nucleic Acids Res. 22:4673-4680 (1994)).

In another aspect, the present invention provides a peptide or polypeptide comprising an epitope-bearing portion of a polypeptide of the invention, which may be used to raise antibodies, particularly monoclonal antibodies, that bind specifically to a one or more of the polypeptides of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes (see, e.g., Geysen et al., Proc. Natl. Acad. Sci. USA 81:3998-4002 (1983)).

As to the selection of peptides or polypeptides bearing an antigenic epitope (i.e., that contain a region of a protein molecule to which an antibody can bind), it is well-known in the art that relatively short synthetic peptides that mimic part of a protein sequence are routinely capable of eliciting an antiserum that reacts with the partially mimicked protein (see, e.g., Sutcliffe, J. G., et al., Science 219:660-666 (1983)). Peptides capable of eliciting protein-reactive sera are frequently represented in the primary sequence of a protein, can be characterized by a set of simple chemical rules, and are not confined to the immunodominant regions of intact proteins (i.e., immunogenic epitopes) or to the amino or carboxy termini. Peptides that are extremely hydrophobic and those of six or fewer residues generally are ineffective at inducing antibodies that bind to the mimicked protein; longer peptides, especially those containing proline residues, usually are effective (Sutcliffe, J. G., et al., Science 219:660-666 (1983)).

Epitope-bearing peptides and polypeptides of the invention designed according to the above guidelines will often contain a sequence of at least five amino acids, at least seven amino acids, at least ten amino acids, at least fifteen amino acids, at least twenty amino acids, at least twenty-five amino acids contained within the amino acid sequence of a polypeptide of the invention. However, peptides or polypeptides comprising a larger portion of an amino acid sequence of a polypeptide of the invention, containing at least about 30 to at least about 50 amino acids, or any length up to and including the entire amino acid sequence of a given polypeptide of the invention, also are considered epitope-bearing peptides or polypeptides of the invention and also are useful for inducing antibodies that react with the mimicked protein.

As one of skill in the art will also appreciate, the polypeptides of the present invention and the epitope-bearing fragments thereof described herein can be combined with one or more fusion partner proteins or peptides, or portions thereof, including but not limited to GST, His₆, Trx, and portions of the constant domain of immunoglobulins (Ig), resulting in chimeric or fusion polypeptides. These fusion polypeptides facilitate purification of the polypeptides of the invention (EP 0 394 827; Traunecker et al., Nature 331:84-86 (1988)) for use in analytical or diagnostic (including high-throughput) format.

Antibodies

In another aspect, the invention relates to antibodies and other antigen-binding proteins (e.g., single-chain antigen-binding proteins) produced by methods of the invention. In a related aspect, the invention relates to antibodies that recognize and bind to one or more polypeptides encoded by all reading frames of one or more recombination site nucleic acid sequences or portions thereof, or to one or more nucleic acid molecules comprising one or more recombination site nucleic acid sequences or portions thereof, including but not limited to att sites (including attB1, attB2, attP1, attP2, attL1, attL2, attR1, attR2 and the like), lox sites (e.g., loxP, loxP511, and the like), FRT, and the like, or mutants, fragments, variants and derivatives thereof. See generally U.S. Pat. No. 5,888,732, which is incorporated herein by reference in its entirety. The antibodies of the present invention may be polyclonal, monoclonal, or synthetic and may be prepared by any of a variety of methods and in a variety of species according to methods that are well-known in the art. See, for instance, U.S. Pat. No. 5,587,287; Sutcliffe, J. G., et al., Science 219:660-666 (1983); Wilson et al., Cell 37: 767 (1984); and Bittle, F. J., et al., J. Gen. Virol. 66:2347-2354 (1985). Antibodies specific for any of the polypeptides or nucleic acid molecules described herein, such as antibodies specifically binding to one or more of the polypeptides encoded by the recombination site nucleotide sequences, or one or more nucleic acid molecules, described herein or contained in the deposited clones, antibodies against fusion polypeptides (e.g., binding to fusion polypeptides between one or more of the fusion partner proteins and one or more of the recombination site polypeptides of the invention, as described herein), and the like, can be raised against the intact polypeptides or polynucleotides of the invention or one or more antigenic polypeptide fragments thereof.

As used herein, the term “antibody” (Ab) may be used interchangeably with the terms “polyclonal antibody” or “monoclonal antibody” (mAb), except in specific contexts as described below. These terms, as used herein, are meant to include intact molecules as well as antibody fragments (such as, for example, Fab and F(ab′)₂ fragments) which are capable of specifically binding to a polypeptide or nucleic acid molecule of the invention or a portion thereof. It will therefore be appreciated that, in addition to the intact antibodies of the invention, Fab, F(ab′)₂ and other fragments of the antibodies described herein, and other peptides and peptide fragments that bind one or more polypeptides or polynucleotides of the invention, are also encompassed within the scope of the invention. Such antibody fragments are typically produced by proteolytic cleavage of intact antibodies, using enzymes such as papain (to produce Fab fragments) or pepsin (to produce F(ab′)₂ fragments). Antibody fragments, and peptides or peptide fragments, may also be produced through the application of recombinant DNA technology or through synthetic chemistry.

Polyclonal antibodies according to this aspect of the invention may be made by immunizing an animal with one or more of the polypeptides or nucleic acid molecules of the invention described herein or portions thereof according to standard techniques (see, e.g., Harlow, E., and Lane, D., Antibodies: A Laboratory Manual, Cold Spring Harbor, N.Y.: Cold Spring Harbor Laboratory Press (1988); Kaufman, P. B., et al., In: Handbook of Molecular and Cellular Methods in Biology and Medicine, Boca Raton, Fla.: CRC Press, pp. 468-469 (1995)).

Monoclonal antibodies (or fragments thereof which bind to one or more of the polypeptides of the invention) according to this aspect of the invention may be made using hybridoma technology (Kohler et al., Nature 256:495 (1975); Köhler et al., Eur. J. Immunol. 6:511 (1976); Köhler et al., Eur. J. Immunol. 6:292 (1976); Hammerling et al., In: Monoclonal Antibodies and T-Cell Hybridomas, Elsevier, N.Y., pp. 563-681 (1981)).

Phage display technology may be used to represent polypeptides on the surface of phage (see U.S. Pat. No. 6,190,908; U.S. Pat. No. 6,194,183). Further, phage display systems may be used in the practice of the invention to modify polypeptides and then screen the modified polypeptides for functional activities. For example, phage displayed libraries may be screened to identify those which bind antibody molecules.

It will be appreciated by one of ordinary skill that the antibodies of the present invention may alternatively be coupled to a solid support, to facilitate, for example, chromatographic and other immunological procedures using such solid phase-immobilized antibodies. Included among such procedures are the use of the antibodies of the invention to isolate or purify polypeptides comprising one or more epitopes encoded by the nucleic acid molecules used in the invention (which may be fusion polypeptides or other polypeptides of the invention described herein), or to isolate or purify polynucleotides comprising one or more recombination site sequences of the invention or portions thereof. Methods for isolation and purification of polypeptides (and, by analogy, polynucleotides) by affinity chromatography, for example using the antibodies of the invention coupled to a solid phase support, are well-known in the art and will be familiar to one of ordinary skill.

Supports

In one aspect, the invention provides methods for connecting populations of nucleic acid molecules to target nucleic acid molecules, wherein (1) the target nucleic acid molecules, (2) nucleic acid molecules which each contain at least one recombination site, or (3) individual members of the populations of nucleic acid molecules are bound to a support. The invention further provides methods for releasing nucleic acid molecules from support. Nucleic acid release may be effected by any number of means, including recombination and digestion with one or more restriction endonucleases.

Using the process set out in FIG. 32 for purposes of illustration, a nucleic acid molecule which contains a recombination site (e.g., an attR2 site) may be bound to a solid support (e.g., a bead). A population of nucleic acid molecules (e.g., cDNA molecules or cDNA molecules contained within a vector) in which the individual members of the population contain at least one recombination site (e.g., an attL2 site) may then undergo recombination with recombination sites (e.g., attR2 sites) of nucleic acid molecules attached to the support resulting in the attachment of members of the population to the support through new recombination sites (e.g., attP2 sites). A second recombination reaction may then used to release the nucleic acid molecules from the support and to incorporate these molecules into another vector. The recombined vectors may then be circularized, if desired, using art known means (e.g., ligation, homologous recombination, topoisomerase cloning, etc.).

A process similar to that discussed above is shown in FIG. 33 where biotin and avidin are used to attach nucleic acid molecules which contain recombination sites to the support. These recombination sites are these employed to attach other nucleic acid molecules to the support.

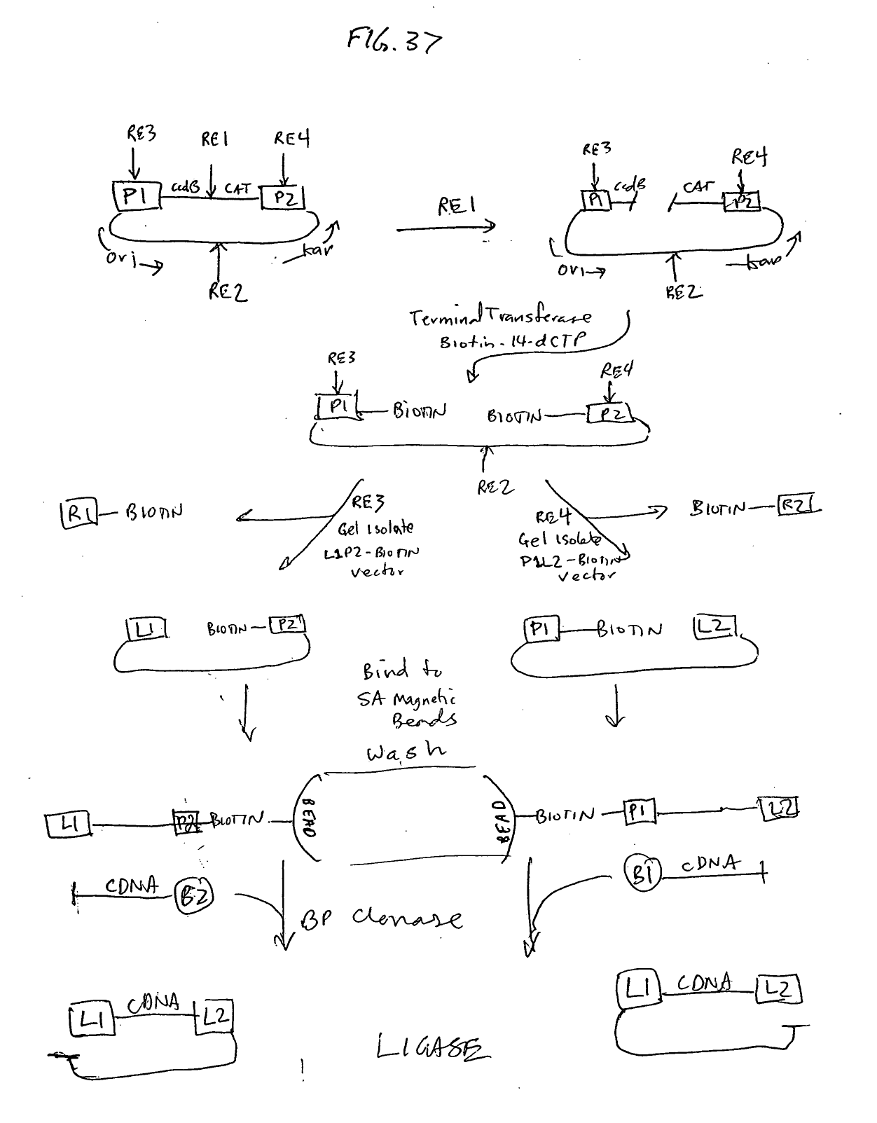

As would be recognized by those skilled in the art, any number of means may be used in the practice of the invention to attach nucleic acid molecules to supports. A number of such means are set out in more detail below. Further, any number or variations of the above may be practiced. For example, one or more initial recombination reactions may be performed before recombined nucleic acid molecules are attached to a support. Further, if two nucleic acid molecules are joined by a recombination reaction and one of the molecules contains a biotin moiety, for example, these molecules may then be attached to the support by association with avidin, which could be bound directly to the support (see FIG. 35-37). As one skilled in the art would recognize, any number of other means could be used to attach such nucleic acid molecules to supports. Further, in certain instances, processes similar to those described above could be used to purify nucleic acid molecules in the absence of recombination which occurs while the nucleic acid molecules are attached to a support. For example, nucleic acid molecules could be generated by recombination prior to attachment to the support. Further, after attachment to the support, nucleic acid molecules could be released by digestion with one or more restriction endonuclease.

The attachment of nucleic acid molecules of the invention to supports has the advantage that the support can be washed to remove unbound reagents. Again using the processes shown in FIGS. 32 and 33 for illustration, once cDNA molecules, or other nucleic acid molecules of a population, are attached to a solid support, unreacted reagents may be removed by washing. Thus, unbound/unreacted molecules (e.g., vectors and cDNA molecules) and reagents may be removed prior to release of nucleic acid molecules from the support. Thus, the invention provides methods for separating members of populations of nucleic acid molecules from contaminants such as proteins, salts, carbohydrates, detergents, other nucleic acid molecules (e.g., RNA, vectors, primers, etc.), etc.

Further, as noted above, release of cDNA molecules from supports may be effected by any number of means. FIGS. 32 and 33 show the release of these molecules by the use of a recombination reaction, but release may be effectuated by, for example, digestion with a restriction endonuclease.

Additional embodiments of the invention in which recombination occurs on supports are shown in FIGS. 35-37. In each of these instances, nucleic acid molecules are attached to supports (i.e., beads) via interaction between biotin and avidin. Nucleic acid segments which contain the individual members of populations of nucleic acid molecules are then released from the supports by recombination.

Thus, in one aspect, the invention provides methods for recombining populations of nucleic acid molecules on supports. In specific related embodiments, the invention further provides methods for purifying nucleic acid molecules by attaching them to support and washing away undesired materials (i.e., contaminants). Thus, in one general aspect, the invention provides methods for purifying nucleic acid molecules by connecting these molecules to supports, followed by the removal of unbound materials and release of the nucleic acid molecules from the supports. The invention further provides populations of nucleic acid molecules purified by methods of the invention and supports which contain these populations of nucleic acid molecules.

Supports suitable for use in accordance with the invention may be any support or matrix suitable for attaching nucleic acid molecules comprising one or more recombination sites or portions thereof. These nucleic acid molecules may be added or bound (covalently or non-covalently) to the supports of the invention by any technique or any combination of techniques well known in the art. Supports of the invention may comprise nitrocellulose, diazocellulose, glass, polystyrene (including microtiter plates), polyvinylchloride, polypropylene, polyethylene, polyvinylidenedifluoride (PVDF), dextran, Sepharose, agar, starch and nylon. Supports of the invention may be in any form or configuration including beads, filters, membranes, sheets, frits, plugs, columns and the like. Supports may also include multi-well tubes (such as microtiter plates) such as 12-well plates, 24-well plates, 48-well plates, 96-well plates, and 384-well plates. Beads may be made, for example, of glass, latex or a magnetic material (magnetic, paramagnetic or superparamagnetic beads).

Methods for the attachment of nucleic acids to supports have been described (see, e.g., U.S. Pat. No. 5,436,327, U.S. Pat. No. 5,800,992, U.S. Pat. No. 5,445,934, U.S. Pat. No. 5,763,170, U.S. Pat. No. 5,599,695 and U.S. Pat. No. 5,837,832). For example, disulfide-modified oligonucleotides can be covalently attached to supports using disulfide bonds. (See Rogers et al., Anal. Biochem. 266:23-30 (1999).) Further, disulfide-modified oligonucleotides can be peptide nucleic acid (PNA) using solid-phase synthesis. (See Aldrian-Herrada et al., J. Pept. Sci. 4:266-281 (1998).) Thus, nucleic acid molecules comprising one or more recombination sites or portions thereof can be added to one or more supports and nucleic acids, proteins or other molecules and/or compounds can be added to such supports through recombination methods of the invention. Conjugation of nucleic acids to a molecule of interest are known in the art and thus one of ordinary skill can produce molecules and/or compounds comprising recombination sites (or portions thereof) for attachment to supports according to the invention.

Essentially, any conceivable support may be employed in the invention. The support may be biological, non-biological, organic, inorganic, or a combination of any of these, existing as particles, strands, precipitates, gels, sheets, tubing, spheres, containers, capillaries, pads, slices, films, plates, slides, etc. The support may have any convenient shape, such as a disc, square, sphere, circle, etc. The support is preferably flat but may take on a variety of alternative surface configurations. For example, the support may contain raised or depressed regions which may be used for synthesis or other reactions. The support and its surface preferably form a rigid support on which to carry out the reactions described herein. The support and its surface are also chosen to provide appropriate light-absorbing characteristics. For instance, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. Other support materials will be readily apparent to those of skill in the art upon review of this disclosure. In a preferred embodiment the support is flat glass or single-crystal silicon.

Thus, the invention provides methods for preparing supports to which nucleic acid molecules are attached. In some embodiments, these nucleic acid molecules will have recombination sites at one or more (e.g., one, two, three or four) of their termini. In some additional embodiments, one nucleic acid molecule will be attached directly to the support, or to a specific section of the support, and one or more additional nucleic acid molecules will be indirectly attached to the support via attachment to the nucleic acid molecule which is attached directly to the support. In such cases, the nucleic acid molecule which is attached directly to the support provides a site of nucleation around which larger nucleic acid molecules may be constructed.

The invention further provides methods for screening populations of nucleic acid molecules (e.g., nucleic acid libraries) to identifying molecules having particular properties, features, or activities. Examples of compositions which can be formed by binding nucleic acid molecules to supports and used in such screening methods are “gene chips,” often referred to in the art as “DNA microarrays” or “genome chips” (see U.S. Pat. Nos. 5,412,087 and 5,889,165, and PCT Publication Nos. WO 97/02357, WO 97/43450, WO 98/20967, WO 99/05574, WO 99/05591, and WO 99/40105, the disclosures of which are incorporated by reference herein in their entireties). For purposes of illustration, nucleic acid molecules, each of which contain a recombination site having the same specificity (e.g., attP1, attP2, attP3, attP4 sites) may be positioned on a gene chip, for example, at specified locations (i.e., addresses) to generate a chip in which nucleic acid molecules having recombination sites with the same specificity are grouped together. Such a chip would have locations where nucleic acid molecules having recombination sites (e.g., attB1, attB2, attB3, attB4 sites) which will recombine with recombination sites (e.g., attP1, attP2, attP3, attP4 sites) associated with the chip can be attached to the chip by recombination.

Once a chip such as that described above has been prepared, one or more populations of nucleic acid molecules which contain recombination sites (e.g., attB1, attB2, attB3, attB4 site) capable of recombining with the recombination sites of the molecules bound to the chip may be contacted with the chip under conditions which facilitate recombination. Recombination between recombination sites of the nucleic acid molecules bound to the gene chip and those of the individual members of the population(s) will result in individual members of the population(s) being attached to the chip. Further, due to the specificity of the recombination reaction(s), the chip may be contacted with numerous different nucleic acid molecules (e.g., nucleic acid molecules which have recombination sites with different specificities) at one time to generate a chip having nucleic acid molecules with the same sequence or closely related sequences (e.g., sequences which are greater than 95% identical to each other) clustered at particular locations. The nucleic acid molecules attached to the chip may then be used in art known processes.

To increased the number of specificities which can be used to generate chips such as those described above, components of multiple recombination systems may be used. For example, a chip could contain nucleic acid molecules with attP sites and lox sites. As noted above, lox sites having various recombination specificities are disclosed in PCT Publication No. WO 01/11058, the entire disclosure of which is incorporated herein by reference. Thus, the invention provides gene chips in which nucleic acid molecules having the same recombination specificity are placed together in specific locations (e.g., 5, 10, 15, 20, 25, 30, 35, 40, 45, 50, 55, 60, 65, 70, 80, 85, 90, 95, 100, 120, 140, 160, 180, 200, 240, 280, 300, 240, 380, 400, 450, 500, 550, 600, 650, 700, 75, 800, 850, 900, 950, 1,000, etc. addresses). These “generic” gene chips may then be used to prepare chips in which nucleic acid molecules having cognate recombination sites are attached via recombination.

In other embodiments, nucleic acid molecules having recombination sites of the same or differing recombinational specificities may be positioned randomly at locations on a gene chip or subportion thereof. The chip may then be contacted with one or more populations of nucleic acid molecules which contain recombination sites capable of recombining with the recombination sites of molecules bound to the chip under conditions which facilitate recombination. As an alternative, populations of nucleic acid molecules may be contacted with only portions of the gene chip to which nucleic acid molecules having cognate sites are attached.

The invention thus provides methods for attaching nucleic acid molecules to supports by recombination, as well as supports prepared by methods of the invention and methods for using these supports for identifying nucleic acid molecules having particular properties, features, or activities.

Gene chips of the invention may also be used to identify recombination sites which differ in specificity. For example, nucleic acid molecules comprising a recombination site may be subjected to mutagenesis (e.g., random mutagenesis), mutagenized nucleic acid molecules may then be placed at various positions on a chip and screened to identify those which undergo recombination with one or more additional recombination sites. For example, a nucleic acid molecule comprising a recombination site (e.g., an attL1 site) may be subjected to random mutagenesis. The resulting individual, nucleic acid molecules may then be amplified and placed at particular locations on the chip. The chip may then be exposed to nucleic acid molecules which comprise either (1) different recombination sites or (2) the same recombination site (e.g., an attR1 site) under conditions which facilitate recombination and scored to identify positions where recombination has occurred. Nucleic acid molecules which participate in the recombination reaction may then be sequenced to determine the nucleotide sequence of the recombination site. The invention further include recombination sites identified by processes such as those described above.

The addressability of nucleic acid arrays of the invention means that molecules or compounds which bind to nucleic acid molecules comprising specific nucleotide sequences can be attached to the arrays. Thus, components such as proteins and other nucleic acids may be attached to specific, addressable locations in nucleic acid arrays of the invention.

The invention thus provides methods for preparing nucleic acid arrays in which nucleic acid molecules having particular recombination specificities are located in particular regions. The invention further provides arrays prepared by methods of the invention, methods for attaching nucleic acid molecules to such arrays using recombination reactions, methods for screening such arrays to identify nucleic acid molecules having particular properties, features, or activities, and nucleic acid molecules identified by methods of the invention.

Kits

The invention also provides kits which may be used in producing nucleic acid molecules, polypeptides, vectors, host cells, and antibodies of the invention. The invention further provides kits which may be used for the insertion of nucleic acid molecules into target nucleic acid molecules, for the transfer of nucleic acid molecules between target nucleic acid molecules, and in sequential selection methods of the invention.

Kits according to this aspect of the invention may comprise one or more containers, which may contain one or more of the nucleic acid molecules, primers, polypeptides, vectors, host cells, or antibodies of the invention. In particular, kits of the invention may comprise one or more components (or combinations thereof) selected from the group consisting of one or more recombination proteins (e.g., Int) or auxiliary factors (e.g., IHF and/or Xis) or combinations thereof, one or more compositions comprising one or more recombination proteins or auxiliary factors or combinations thereof (for example, GATEWAY™ LR CLONASE™ Enzyme Mix or GATEWAY™ BP CLONASE™ Enzyme Mix) one or more Destination Vector molecules (including those described herein), one or more Entry Clone or Entry Vector molecules (including those described herein), one or more primer nucleic acid molecules (particularly those described herein), one or more host cells (e.g., competent cells, such as E. coli cells, yeast cells, animal cells (including mammalian cells, insect cells, nematode cells, avian cells, fish cells, etc.), plant cells, and most particularly E. coli DB3, DB3.1 (e.g., E. coli LIBRARY EFFICIENCY® DB3.1™ Competent Cells; Invitrogen Corp., Carlsbad, Calif.), DB4 and DB5; see U.S. application Ser. No. 09/518,188, filed on Mar. 2, 2000, the disclosure of which is incorporated by reference herein in its entirety), and the like.

In related aspects, kits of the invention may comprise one or more nucleic acid molecules encoding one or more recombination sites or portions thereof, such as one or more nucleic acid molecules comprising a nucleotide sequence encoding the one or more recombination sites (or portions thereof) of the invention, and particularly one or more of the nucleic acid molecules contained in the deposited clones described herein. Kits according to this aspect of the invention may also comprise one or more isolated nucleic acid molecules used in the invention, one or more vectors of the invention, one or more primer nucleic acid molecules used in the invention, and/or one or more antibodies of the invention.

Kits of the invention may further comprise one or more additional containers containing one or more additional components useful in combination with the nucleic acid molecules, polypeptides, vectors, host cells, or antibodies of the invention, such as one or more buffers, one or more detergents, one or more polypeptides having nucleic acid polymerase activity, one or more polypeptides having reverse transcriptase activity, one or more transfection reagents, one or more nucleotides, and the like. In a related aspect the kits of the invention may comprise one or more reagents for selection such as enzymes, substrates, ligands, inhibitors, labels, antibodies, probes or primers. Such kits may be used in any process advantageously using the nucleic acid molecules, primers, vectors, host cells, polypeptides, antibodies and other compositions used in or selected by the invention, for example in methods of synthesizing nucleic acid molecules (e.g., via amplification such as via PCR), in methods of cloning nucleic acid molecules (e.g., via recombinational cloning as described herein), and the like.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

The entire disclosures of U.S. application Ser. No. 09/732,914, filed Dec. 11, 2000; U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/233,492, filed Jan. 20, 1999; U.S. Pat. No. 6,143,557; U.S. Appl. No. 60/065,930, filed Oct. 24, 1997; U.S. application Ser. No. 09/177,387 filed Oct. 23, 1998; U.S. application Ser. No. 09/296,280, filed Apr. 22, 1999; U.S. application Ser. No. 09/296,281, filed Apr. 22, 1999; U.S. Appl. No. 60/108,324, filed Nov. 13, 1998; U.S. application Ser. No. 09/438,358, filed Nov. 12, 1999; U.S. application Ser. No. 09/695,065, filed Oct. 25, 2000; U.S. application Ser. No. 09/432,085 filed Nov. 2, 1999; U.S. Appl. No. 60/122,389, filed Mar. 2, 1999; U.S. Appl. No. 60/126,049, filed Mar. 23, 1999; U.S. Appl. No. 60/136,744, filed May 28, 1999; U.S. Appl. No. 60/122,392, filed Mar. 2, 1999; and U.S. Appl. No. 60/161,403, filed Oct. 25, 1999, are herein incorporated by reference.

### EXAMPLES

### Example 1

Simultaneous Cloning of Two Nucleic Acid Segments Using an LR Reaction

Two nucleic acid segments (either or both of which may be individual members of one or more population of nucleic acid molecules) may be cloned in a single reaction using methods of the present invention. Methods of the present invention may comprise the steps of providing a first nucleic acid segment (e.g., nucleic acid encoding a HIS6 tag) flanked by a first and a second recombination site, providing a second nucleic acid segment (e.g., a member of a cDNA library) flanked by a third and a fourth recombination site, wherein either the first or the second recombination site is capable of recombining with either the third or the fourth recombination site, conducting a recombination reaction such that the two nucleic acid segments are recombined into a single nucleic acid molecule and cloning the single nucleic acid molecule.

With reference to FIG. 19, two nucleic acid segments flanked by recombination sites may be provided. Those skilled in the art will appreciate that the nucleic acid segments may be provided either as discrete fragments or as part of a larger nucleic acid molecule and may be circular and optionally supercoiled or linear. The sites can be selected such that one member of a reactive pair of sites flanks each of the two segments.

By “reactive pair of sites,” what is meant is two recombination sites that can, in the presence of the appropriate enzymes and cofactors, recombine. For example, in some embodiments, one nucleic acid molecule may comprise an attR site while the other comprises an attL site that reacts with the attR site. As the products of an LR reaction are two molecules, one of which comprises an attB site and one of which comprises an attP site, it is possible to arrange the orientation of the starting attL and attR sites such that, after joining, the two starting nucleic acid segments are separated by a nucleic acid sequence that comprises either an attB site or an attP site.

In some embodiments, the sites may be arranged such that the two starting nucleic acid segments are separated by an attB site after the recombination reaction. In other embodiments, recombination sites from other recombination systems may be used. For example, in some embodiments one or more of the recombination sites may be a lox site or derivative. In some embodiments, recombination sites from more than one recombination system may be used in the same construct. For example, one or more of the recombination sites may be an att site while others may be lox sites. Various combinations of sites from different recombination systems (e.g., Flp sites, Flp site derivatives, etc.) may occur to those skilled in the art and such combinations are deemed to be within the scope of the present invention.

As shown in FIG. 19, nucleic acid segment A (DNA-A) may be flanked by recombination sites having unique specificity, for example attL1 and attL3 sites and nucleic acid segment B (DNA-B) may be flanked by recombination sites attR3 and attL2. For illustrative purposes, the segments are indicated as DNA. This should not be construed as limiting the nucleic acids used in the practice of the present invention to DNA to the exclusion of other nucleic acids. In addition, in this and the subsequent examples, the designation of the recombination sites (i.e., L1, L3, R1, R3, etc.) is merely intend to convey that the recombination sites used have different specificities and should not be construed as limiting the invention to the use of the specifically recited sites. One skilled in the art could readily substitute other pairs of sites for those specifically exemplified.

The attR3 and attL3 sites comprise a reactive pair of sites. Other pairs of unique recombination sites may be used to flank the nucleic acid segments. For example, lox sites could be used as one reactive pair while another reactive pair may be att sites and suitable recombination proteins included in the reaction. Likewise, the recombination sites discussed above can be used in various combinations. In this embodiment, the only critical feature is that, of the recombination sites flanking each segment, one member of a reactive pair of sites, in this example an LR pair L3 and R3, is present on one nucleic acid segment and the other member of the reactive pair is present on the other nucleic acid segment.

The two segments may be contacted with the appropriate enzymes and a Destination Vector.

The Destination Vector comprises a suitable selectable marker flanked by two recombination sites. In some embodiments, the selectable marker may be a negative selectable marker (such as a toxic gene, e.g., ccdB). One site in the Destination Vector will be compatible with one site present on one of the nucleic acid segments while the other compatible site present in the Destination Vector will be present on the other nucleic acid segment.

Absent a recombination between the two starting nucleic acid segments, neither starting nucleic acid segment has recombination sites compatible with both the sites in the Destination Vector. Thus, neither starting nucleic acid segment can replace the selectable marker present in the Destination Vector.

The reaction mixture may be incubated at about 25° C. for from about 5 minutes to about 48 hours. All or a portion of the reaction mixture will be used to transform competent microorganisms and the microorganisms screened for the presence of the desired construct.

In some embodiments, the Destination Vector comprises a negative selectable marker and the microorganisms transformed are susceptible to the negative selectable marker present on the Destination Vector. The transformed microorganisms will be grown under conditions permitting the negative selection against microorganisms not containing the desired recombination product.

In FIG. 19, the resulting desired product consists of DNA-A and DNA-B separated by an attB3 site and cloned into the Destination Vector backbone. In this embodiment, the same type of reaction (i.e., an LR reaction) may be used to combine the two fragments and insert the combined fragments into a Destination Vector.

In some embodiments, it may not be necessary to control the orientation of one or more of the nucleic acid segments and recombination sites of the same specificity can be used on both ends of the segment.

With reference to FIG. 19, if the orientation of segment A with respect to segment B were not critical, segment A could be flanked by L1 sites on both ends oriented as inverted repeats and the end of segment B to be joined to segment A could be equipped with an R1 site. This might be useful in generating additional complexity in the formation of combinatorial libraries between segments A and B. That is, the joining of the segments can occur in various orientations and given that one or both segments joined may be derived from one or more libraries, a new population or library comprising hybrid molecules in random orientations may be constructed according to the invention.

Although, in the present examples, the recombination between the two starting nucleic acid segments is shown as occurring before the recombination reactions with the Destination Vector, the order of the recombination reactions is not important. Thus, in some embodiments, it may be desirable to conduct the recombination reaction between the segments and isolate the combined segments. The combined segments can be used directly, for example, may be amplified, sequenced or used as linear expression elements as taught by Sykes et al. (Nature Biotechnology 17:355-359 (1999)). In some embodiments, the joined segments may be encapsulated as taught by Tawfik et al. (Nature Biotechnology 16:652-656 (1998)) and subsequently assayed for one or more desirable properties, features, or activities. In some embodiments, the combined segments may be used for in vitro expression of RNA by, for example, including a promoter such as the T7 promoter or SP6 promoter on one of the segments. Such in vitro expressed RNA may optionally be translated in an in vitro translation system such as rabbit reticulocyte lysate. Thus, in certain embodiments, nucleic acid molecules of the invention may not be inserted into a Destination Vector. Further, nucleic acid segments which each contain recombination sites at one terminus, may be joined at the termini which do not contain recombination sites by methods such as topoisomerase cloning.

Optionally, the joined segments may be further reacted with a Destination Vector resulting in the insertion of the combined segments into the vector. In some instances, it may be desirable to isolate an intermediate comprising one of the segments and the vector. For insertion of the segments into a vector, it is not critical to the practice of the present invention whether the recombination reaction joining the two segments occurs before or after the recombination reaction between the segments and the Destination Vector.

According to the invention, all three recombination reactions may occur (i.e., the reaction between segment A and the Destination Vector, the reaction between segment B and the Destination Vector, and the reaction between segment A and segment B) in order to produce a nucleic acid molecule in which both of the two starting nucleic acid segments are now joined in a single molecule. In some embodiments, recombination sites may be selected such that, after insertion into the vector, the recombination sites flanking the joined segments form a reactive pair of sites and the joined segments may be excised from the vector by reaction of the flanking sites with suitable recombination proteins. In other embodiments, segments A and B may each have a recombination sites at only one end. The “free” ends of these segments may then be joined by any number of methods. For example, one or both of the ends may be covalently linked to a topoisomerase molecule, which is then used to join the two segments. Cloning methods employing topoisomerases are described, for example, in Invitrogen 2001 Catalog, pages 6-12 (Invitrogen Corp., Carlsbad, Calif.).

With reference to FIG. 19, if the L2 site on segment B were replaced by an L1 site in the opposite orientation with respect to segment B (i.e., the long portion of the box indicating the recombination site was not adjacent to the segment) and the R2 site in the vector were replaced by an R1 site in opposite orientation, the recombination reaction would produce an attP1 site in the vector. The attP1 site would then be capable of reaction with the attB1 site on the other end of the joined segments. Thus, the joined segments could be excised using the recombination proteins appropriate for a BP reaction.

This embodiment of the invention is particularly suited for the construction of combinatorial libraries. In some embodiments, each of the nucleic acid segments in FIG. 19 may represent libraries, each of which may have a known or unknown nucleic acid sequence to be screened. In some embodiments, one or more of the segments may have a sequence encoding one or more permutations of the amino acid sequence of a given peptide, polypeptide or protein. In some embodiments, each segment may have a sequence that encodes a protein domain or a library representing various permutations of the sequence of protein domain. For example, one segment may represent a library of mutated forms of the variable domain of an antibody light chain while the other segment represents a library of mutated forms of an antibody heavy chain. Thus, recombination would generate a population of molecules (e.g., antibodies, single-chain antigen-binding proteins, etc.) each potentially containing a unique combination of sequences and, therefore, a unique binding specificity.

In other embodiments, one of the segments may represent a single nucleic acid sequence while the other represents a library. The result of recombination will be a population of sequences all of which have one portion in common and are varied in the other portion. Embodiments of this type will be useful for the generation of a library of fusion constructs. For example, DNA-A may comprise a regulatory sequence for directing expression (i.e., a promoter) and a sequence encoding a purification tag. Suitable purification tags include, but are not limited to, glutathione S-transferase (GST), maltose binding protein (MBP), epitopes, defined amino acid sequences such as epitopes, haptens, six histidines (HIS6), and the like. DNA-B may comprise a library of mutated forms of a protein of interest. The resultant constructs could be assayed for a desired characteristic such as enzymatic activity or ligand binding.

Alternatively, DNA-B might comprise the common portion of the resulting fusion molecule. In some embodiments, the above described methods may be used to facilitate the fusion of promoter regions or transcription termination signals to the 5′-end or 3′-end of structural genes, respectively, to create expression cassettes designed for expression in different cellular contexts, for example, by adding a tissue-specific promoter to a structural gene.

In some embodiments, one or more of the segments may represent a sequence encoding members of a random peptide library. This approach might be used, for example, to generate a population of molecules with a certain desirable characteristic. For example, one segment might contain a sequence coding for a DNA binding domain while the other segment represents a random protein library. The resulting population might be screened for the ability to modulate the expression of a target gene of interest. In other embodiments, both segments may represent sequences encoding members of a random protein library and the resultant synthetic proteins (e.g., fusion proteins) could be assayed for any desirable characteristic such as, for example, binding a specific ligand or receptor or possessing some enzymatic activity.

As suggested above, regions of proteins, referred to as domains, generally confer upon proteins various functional activities. A considerable number of domains which confer activities upon proteins are known in the art (e.g., SH2 domains, zinc finger domains, NADPH binding domains, apoptosis-induction domains, eIF4A-binding domains, IGF binding domain, DNA binding domains, UBX domains, zona pellucida domains, p53 core domains, Src homology 2 domains, etc.). Methods of the invention can be used to generate and screen mutagenized nucleic acid molecules which encode such domains to identify those which encode polypeptides having particular properties, features, or activities.

It is not necessary that the nucleic acid segments encode an amino acid sequence. For example, both of the segments may direct the transcription of an RNA molecule that is not translated into protein. This will be useful for the construction of tRNA molecules, ribozymes and anti-sense molecules. Alternatively, one segment may direct the transcription of an untranslated RNA molecule while the other codes for a protein. For example, DNA-A may direct the transcription of an untranslated leader sequence that enhances protein expression such as the encephalomyocarditis virus leader sequence (EMC leader) while DNA-B encodes a peptide, polypeptide or protein of interest. In some embodiments, a segment comprising a leader sequence might further comprise a sequence encoding an amino acid sequence. For example, DNA-A might have a nucleic acid sequence corresponding to an EMC leader sequence and a purification tag while DNA-B has a nucleic acid sequence encoding a peptide, polypeptide or protein of interest.

The above process is especially useful for the preparation of combinatorial libraries of single-chain antigen-binding proteins. Methods for preparing single-chain antigen-binding proteins are known in the art. (See, e.g., PCT Publication No. WO 94/07921, the entire disclosure of which is incorporated herein by reference.) DNA-A could encode, for example, mutated forms of the variable domain of an antibody light chain and DNA-B could encode, for example, mutated forms of the variable domain of an antibody light chain. Further, intervening nucleic acid between DNA-A and DNA-B could encode a peptide linker for connecting the light and heavy chains. Cells which express the single-chain antigen-binding proteins can then be screened to identify those which produce molecules that bind to a particular antigen.

Numerous variation of the above are possible. For example, instead of using a construct illustrated above, a construct similar to that illustrated in FIG. 19 could be used with the linker peptide coding region being embedded in the recombination site. This is one example of recombination site embedded functionality discussed above, which is included within the scope of the invention.

As another example, single-chain antigen-binding proteins each composed of two antibody light chains or two antibody heavy chains can also be produced. These single-chain antigen-binding proteins can be designed to associate and form multivalent antigen binding complexes. Using the constructs shown in FIG. 19 again for illustration, DNA-A and DNA-B could each encode, for example, mutated forms of the variable domain of an antibody light chain. At the same site in a similar vector or at another site in a vector which is designed for the insertion of four nucleic acid inserts, DNA-A and DNA-B could each encode, for example, mutated forms of the variable domain of an antibody heavy chain. Cells which express both single-chain antigen-binding proteins could then be screened to identify, for example, those which produce multivalent antigen-binding complexes having specificity for a particular antigen.

Thus, the methods of the invention can be used, for example, to prepare and screen combinatorial libraries to identify cells which produce antigen-binding proteins (e.g., antibodies and/or antibody fragments or antibody fragment complexes comprising variable heavy or variable light domains) having specificities for particular epitopes. The methods of the invention also methods for preparing antigen-binding proteins and antigen-binding proteins prepared by the methods of the invention.

Further, an iterative approach may be followed to prepare and identify nucleic acid molecules which encode antigen-binding proteins that exhibit high affinity for one or more antigens. For example, combinatorial libraries may be screened to identify nucleic acid molecules which encode antigen-binding proteins which exhibit affinity for a particular antigen. Further, once nucleic acid which encodes a variable light or a variable heavy domain which forms one component of antigen-binding proteins having affinity for a particular antigen, any number of steps may be taken to obtain antigen-binding proteins which exhibit increased affinity for the antigen. For example, antigen-binding proteins encoded for by the following nucleic acids may be screened to identify those which encode proteins with increased affinity:

Nucleic acid encoding one domain (i.e., the variable light or variable heavy domain) may be left unaltered and nucleic acid encoding the other domain may be subjected to one or more rounds of mutagenesis.

Nucleic acid encoding one domain (i.e., the variable light or variable heavy domain) may be left unaltered and nucleic acid molecules of a library which encodes variable domains may be combined with nucleic acid encoding the unaltered domain.

Nucleic acid encoding both domains may be subjected to mutagenesis.

Antigen-binding proteins prepared from nucleic acid molecules generated by the above process may then be screened to identify proteins having desired properties, features, or activities (e.g., binding affinities for the particular antigen). Further, multiple rounds of selection (e.g., mutagenesis followed by screening) may be used to generate antigen-binding proteins having desired properties, features, or activities.

Using FIG. 19 to illustrate additional variations of the invention, one or more nucleic acid segment which forms recombination sites shown in this figure may be omitted and nucleic acid which confers other properties, features, or activities upon molecules may be included. For example, either one or both of the regions on DNA-A and DNA-B labeled “L3” and “R3” in FIG. 19 may be replaced with nucleic acids which do not recombine with each other but still allow for the joining of the two segments. Examples of such nucleic acids include (1) nucleic acids which allow for topoisomerase mediated cloning, (2) “sticky ends” which anneal to each other, (3) restriction endonuclease recognition sites which can be used to generate “sticky ends,” and (4) nucleic acids which are capable of engaging in homologous recombination. Thus, the invention includes methods for cloning multiple nucleic acid molecules which involve recombination at specific sites and connection of nucleic acid segments by means other than recombination at other sites.

Further, as an extension of the representation shown in FIG. 19, any number of nucleic acid segments may be joined by methods of the invention, inserted into a target molecules, and/or then transferred to additional target molecules. In addition, as noted above, when multiple nucleic acid molecules are connected to each other, all of these molecules need not be connected to each other through recombination. For example, three nucleic acid segments may be connected to each other in the following 5′ to 3′ order: 1-2-3. Segment 1 may have recombination sites at both the 5′ and 3′ ends. Further, the 5′ recombination site may be capable of recombining with a first recombination site of a target nucleic acid molecule and the 3′ recombination site may be capable of recombining with the recombination site at the 5′ end of segment 2. Segment 2 may have a first recombination site at the 5′ end and a second recombination site which is internal. The 5′ recombination site may be capable of recombining with the 3′ recombination site of segment 1. Segment 3 may have a 3′ recombination site which is capable of recombining with a second recombination site of the target nucleic acid molecule. Thus, upon recombination, segments 1, 2, and 3 may be inserted into the target nucleic acid molecule. Further, segments 2 and 3 may be connected using processes such as ligation.

### Example 2

### Use of Suppressor tRNAs to Generate Fusion Proteins

The recombinational cloning techniques described above permit the rapid movement of nucleic acids (e.g., a member of a cDNA library) flanked by recombination sites from one vector to one or more other vector. Because the recombination event is site specific, the orientation and reading frame of the nucleic acid can be controlled with respect to the vector. This control makes the construction of fusions between sequences present on the nucleic acid inserts and sequences present on the vector a simple matter.

Site specificity also allows for the joining of multiple nucleic acid segments to form contiguous nucleic acid molecules, and the subsequent insertion of such contiguous molecules into vectors, as well as the transfer of such contiguous molecules between vectors.

In general terms, nucleic acid may be expressed in four forms: native at both amino and carboxy termini, modified at either end, or modified at both ends. A construct containing the nucleic acid molecules being transferred (e.g., members of a cDNA library) may include the N-terminal methionine ATG codon, and a stop codon at the carboxy end, of the open reading frame, or ORF, thus ATG-ORF-stop. Frequently, the expressible nucleic acid construct will include translation initiation sequences, tis, that may be located upstream of the ATG that allow expression of the gene, thus tis-ATG-ORF-stop. Constructs of this sort allow expression of a nucleic acid which encodes a protein that contains the same amino and carboxy amino acids as in the native, uncloned, protein. When such a construct is fused in-frame with an amino-terminal tag, e.g., GST, the tag will have its own tis, thus tis-ATG-segment-tis-ATG-ORF-stop, and the bases comprising the tis of the ORF will be translated into amino acids between the tag and the ORF. In addition, some level of translation initiation may be expected in the interior of the mRNA (i.e., at the ORF's ATG and not the tag's ATG) resulting in a certain amount of native protein expression contaminating the desired protein.

DNA (lower case):
tis1-atg-tag-tis2-atg-orf-stop
RNA (lower case, italics):
tis1-atg-tag-tis2-atg-orf-stop

Protein (upper case): ATG-TAG-TIS2-ATG-ORF (tis1 and stop are not translated)+contaminating ATG-ORF (translation of ORF beginning at tis2).

Using recombinational cloning, it is a simple matter for those skilled in the art to construct a vector containing nucleic acid which encodes a tag adjacent to a recombination site permitting the in-frame fusion of the nucleic acid to the C- and/or N-terminus of the ORF of interest.

Given the ability to rapidly create a number of clones in a variety of vectors, there is a need in the art to maximize the number of ways a single cloned nucleic acid can be expressed without the need to manipulate the construct itself. The present invention meets this need by providing materials and methods for the controlled expression of a C- and/or N-terminal fusion to the expression product of a nucleic acid insert using one or more suppressor tRNAs to suppress the termination of translation at a stop codon. Thus, the present invention provides materials and methods in which nucleic acid molecules are prepared flanked with recombination sites.

The construct is prepared with a sequence coding for a stop codon optionally at the C-terminus of the nucleic acid encoding the protein of interest. In some embodiments, a stop codon can be located adjacent to the gene, for example, within the recombination site flanking the expressible nucleic acid. The nucleic acid inserts can be transferred through recombination to various vectors which can provide various C-terminal or N-terminal tags (e.g., GFP, GST, His Tag, GUS, etc.) to the final expression product. When the stop codon is located at the carboxy terminus of the expression product, expression of a product with a “native” carboxy end amino acid sequence occurs under non-suppressing conditions (i.e., when the suppressor tRNA is not expressed) while expression of a product having a carboxy fusion protein occurs under suppressing conditions. The present invention is exemplified using an amber suppressor supF, which is a particular tyrosine tRNA gene (tyrT) mutated to recognize the UAG stop codon. Those skilled in the art will recognize that other suppressors and other stop codons could be used in the practice of the present invention. Those skilled in the art will also recognize that it may be necessary to charge suppressor tRNA molecules with an appropriate amino acid residue. This may be accomplished in vivo by modulating the activity an amino acyl-tRNA synthetase.

In the present example, the gene coding for the suppressing tRNA has been incorporated into the vector from which the nucleic acid inserts are to be expressed. In other embodiments, the gene for the suppressor tRNA may be in the genome of the host cell. In still other embodiments, the gene for the suppressor may be located on a separate vector and provided in trans. In embodiments of this type, the vector containing the suppressor gene may have an origin of replication selected so as to be compatible with the vector containing the expressible nucleic acid. The selection and preparation of such compatible vectors is within ordinary skill in the art. Those skilled in the art will appreciate that the selection of an appropriate vector for providing the suppressor tRNA in trans may include the selection of an appropriate antibiotic resistance marker. For example, if the vector expressing the expression products of the nucleic acid inserts contains an antibiotic resistance marker for one antibiotic, a vector used to provide a suppressor tRNA may encode resistance to a second antibiotic. This permits the selection for host cells containing both vectors.

In some embodiments, more than one copy of a suppressor tRNA may be provided in all of the embodiments described above. For example, a host cell may be provided that contains multiple copies of a gene encoding the suppressor tRNA. Alternatively, multiple copies of the suppressor tRNA coding sequences under the same or different promoters may be provided in the same vector as the nucleic acid inserts. In some embodiments, multiple copies of a suppressor tRNA may be provided in a different vector than the one use to contain the nucleic acid inserts. In other embodiments, one or more copies of the suppressor tRNA gene may be provided on the vector containing the nucleic acid encoding the protein of interest and/or on another vector and/or in the genome of the host cell or in combinations of the above. When more than one copy of a suppressor tRNA gene is provided, the genes may be expressed from the same or different promoters which may be the same or different as the promoter used to express the nucleic acid encoding the protein of interest.

In some embodiments, two or more different suppressor tRNA genes may be provided. In embodiments of this type one or more of the individual suppressors may be provided in multiple copies and the number of copies of a particular suppressor tRNA gene may be the same or different as the number of copies of another suppressor tRNA gene. Each suppressor tRNA gene, independently of any other suppressor tRNA gene, may be provided on the vector used to express the nucleic acid of interest and/or on a different vector and/or in the genome of the host cell. A given tRNA gene may be provided in more than one place in some embodiments. For example, a copy of the suppressor tRNA may be provided on the vector containing the nucleic acid of interest while one or more additional copies may be provided on an additional vector and/or in the genome of the host cell. When more than one copy of a suppressor tRNA gene is provided, the genes may be expressed from the same or different promoters which may be the same or different as the promoter used to express the nucleic acid encoding the protein of interest and may be the same or different as a promoter used to express a different tRNA gene.

With reference to FIGS. 20A-20B, the GUS gene was cloned in frame with a GST gene separated by the TAG codon. The plasmid also contained a supF gene expressing a suppressor tRNA. The plasmid was introduced into a host cell where approximately 60 percent of the GUS gene was expressed as a fusion protein containing the GST tag. In control experiments, a plasmid containing the same GUS-stop codon-GST construct did not express a detectable amount of a fusion protein when expressed from a vector lacking the supF gene. In this example, the supF gene was expressed as part of the mRNA containing the GUS-GST fusion. Since tRNAs are generally processed from larger RNA molecules, constructs of this sort can be used to express the suppressor tRNAs of the present invention. In other embodiments, the RNA containing the tRNA sequence may be expressed separately from the mRNA containing the gene of interest.

In some embodiments of the present invention, the nucleic acid inserts and the gene expressing the suppressor tRNA may be controlled by the same promoter. In other embodiments, the nucleic acid inserts may be expressed from a different promoter than the suppressor tRNA. Those skilled in the art will appreciate that, under certain circumstances, it may be desirable to control the expression of the suppressor tRNA and/or the nucleic acid inserts using a regulatable promoter. For example, either the nucleic acid inserts and/or the gene expressing the suppressor tRNA may be controlled by a promoter such as the lac promoter or derivatives thereof such as the tac promoter. In the embodiment shown, both the nucleic acid inserts and the suppressor tRNA gene are expressed from the T7 RNA polymerase promoter. Induction of the T7 RNA polymerase turns on expression of both the expressible nucleic acid of interest (GUS in this case) and the supF gene expressing the suppressor tRNA as part of one RNA molecule.

In some embodiments, the expression of the suppressor tRNA gene may be under the control of a different promoter from that of the expressible nucleic acid of interest. In some embodiments, it may be possible to express the suppressor gene before the expression of the nucleic acid inserts. This would allow levels of suppressor to build up to a high level, before they are needed to allow expression of a fusion protein by suppression of a the stop codon. For example, in embodiments of the invention where the suppressor gene is controlled by a promoter inducible with IPTG, the nucleic acid inserts are controlled by the T7 RNA polymerase promoter and the expression of the T7 RNA polymerase is controlled by a promoter inducible with an inducing signal other than IPTG, e.g., NaCl, one could turn on expression of the suppressor tRNA gene with IPTG prior to the induction of the T7 RNA polymerase gene and subsequent expression of the expressible nucleic acid of interest. In some embodiments, the expression of the suppressor tRNA might be induced about 15 minutes to about one hour before the induction of the T7 RNA polymerase gene. In a embodiment, the expression of the suppressor tRNA may be induced from about 15 minutes to about 30 minutes before induction of the T7 RNA polymerase gene. In the specific example shown, the expression of the T7 RNA polymerase gene is under the control of a salt inducible promoter. A cell line having an inducible copy of the T7 RNA polymerase gene under the control of a salt inducible promoter is commercially available from Invitrogen Corp., Carlsbad, Calif. under the designation of the BL21SI strain.

In some embodiments, the expression of the nucleic acid inserts and the suppressor tRNA can be arranged in the form of a feedback loop. For example, the nucleic acid inserts may be placed under the control of the T7 RNA polymerase promoter while the suppressor gene is under the control of both the T7 promoter and the lac promoter, and the T7 RNA polymerase gene itself is transcribed by both the T7 promoter and the lac promoter, and the T7 RNA polymerase gene has an amber stop mutation replacing a normal tyrosine stop codon, e.g., the 28^{th} codon (out of 883). No active T7 RNA polymerase can be made before levels of suppressor are high enough to give significant suppression. Then expression of the polymerase rapidly rises, because the T7 polymerase expresses the suppressor gene as well as itself. In other embodiments, only the suppressor gene is expressed from the T7 RNA polymerase promoter. Embodiments of this type would give a high level of suppressor without producing an excess amount of T7 RNA polymerase. In other embodiments, the T7 RNA polymerase gene has more than one amber stop mutation (see, e.g., FIG. 20B). This will require higher levels of suppressor before active T7 RNA polymerase is produced.

In some embodiments of the present invention it may be desirable to have more than one stop codon suppressible by more than one suppressor tRNA. With reference to FIG. 21, a vector may be constructed so as to permit the regulatable expression of N- and/or C-terminal fusions of a protein of interest from the same construct. A first tag sequence, TAG1 in FIG. 21, is expressed from a promoter represented by an arrow in the figure. The tag sequence includes a stop codon in the same reading frame as the tag. The stop codon 1, may be located anywhere in the tag sequence and may be located at or near the C-terminal of the tag sequence. The stop codon may also be located in the recombination site RS₁ or in the internal ribosome entry sequence (IRES). The construct also includes an expressible nucleic acid of interest (GENE) which includes a stop codon 2. The first tag and the nucleic acid insert may be in the same reading frame although inclusion of a sequence that causes frame shifting to bring the first tag into the same reading frame as the expressible nucleic acid of interest is within the scope of the present invention. Stop codon 2 is in the same reading frame as the expressible nucleic acid of interest and may be located at or near the end of the coding sequence for the gene. Stop codon 2 may optionally be located within the recombination site RS₂. The construct also includes a second tag sequence in the same reading frame as the expressible nucleic acid of interest indicated by TAG2 in FIG. 21 and the second tag sequence may optionally include a stop codon 3 in the same reading frame as the second tag. A transcription terminator may be included in the construct after the coding sequence of the second tag (not shown in FIG. 21). Stop codons 1, 2 and 3 may be the same or different. In some embodiments, stop codons 1, 2 and 3 are different. In embodiments where 1 and 2 are different, the same construct may be used to express an N-terminal fusion, a C-terminal fusion and the native protein by varying the expression of the appropriate suppressor tRNA. For example, to express the native protein, no suppressor tRNAs are expressed and protein translation is controlled by the IRES. When an N-terminal fusion is desired, a suppressor tRNA that suppresses stop codon 1 is expressed while a suppressor tRNA that suppresses stop codon 2 is expressed in order to produce a C-terminal fusion. In some instances it may be desirable to express a doubly tagged protein of interest in which case suppressor tRNAs that suppress both stop codon 1 and stop codon 2 may be expressed.

### Example 3

### Identification of Proteins which Interact with a Known Target Protein

The DPI protein is known to interact with co-transcription factors of the E2F family, many members of which are known. (See, e.g., Harbour and Dean, Nat. Cell. Biol. 2:E65 (2000); Muller and Helin, Biochim. Biophys. Acta 14:1470 (2000); Ohtani K, Front. Biosci. 1:4 (1999)). The vector pMAB32, which is a derivative of pDBLeu (a yeast two-hybrid vector), contains DNA encoding the full length human DP1 coding region fused at the N-terminus of DP1 to the GAL4 DNA binding domain (Gal4 DB).

A cDNA library derived from mouse brain RNA was constructed in vector pMAB58. This vector is an RC-compatible E. coli/yeast two-hybrid shuttle vector which contains the Activation Domain of GAL4 (Gal4 AD). The resulting library fuses the GAL4 AD to the 5′ end of the cDNA population such that the cDNA is flanked by attB sites (attB1 and attB2: GAL4AD-attB1-cDNA-attB2). It should be noted that because this library contains random 5′ ends, only ⅓ of the library is in the correct reading frame for the GAL4 AD fusions. The attB1 site is situated such that the AD fusion domain and attB1 site are in the same reading frame.

Yeast strain MaV203 contains three GAL4-responsive reporter genes for use in two-hybrid analysis. As a first selection, a population of cDNAs fused to the GAL4 AD region was screened against a fusion of human DP1 protein fused to GAL4 DB. Approximately 1.5×10⁶ total transformants were analyzed of which approximately 106 colonies were found to induce the HIS3 reporter gene. These colonies represent a subpopulation which presumably encode proteins that interact with DP1. PCR analysis indicated that at least some of these candidate interactors represented E2F factors and were therefore valid interacting proteins. Based on these preliminary results, a subpopulation representing candidates of E2F 1, E2F4 and E2F5 were isolated from yeast and introduced into E. coli. Note that because the initial selection was developed to identify interacting proteins (as Activation Domain-cDNA protein fusions), the resulting subset contains cDNAs that are in frame with GAL4 AD. Consequently, this cDNA is also expected to be in frame with attB1.

A second selection was applied to this subpopulation in which the clones interacting with DP1 were further selected to identify those also able to express protein in E. coli when fused to either a HIS6 fusion tag or a GST fusion tag. For this, the above selected DNAs were isolated from E. coli, incubated in vitro with an appropriate attP vector (pDONR201) and BP CLONASE™. After overnight incubation, Destination Vector (attR5) DNAs which encoded a T7 RNA Polymerase promoter and N-terminal His6 tag or an N-terminal GST-fusion tag and LR CLONASE™ was added. Resulting clones contained the DNA segment encoding a protein that interacted with DP1, now in a His6 fusion vector in E. coli strain BL21SI, which encoded the T7 RNA polymerase under control of a salt inducible promoter.

Two random colonies from each reaction were grown in liquid media then induced to express protein by addition of NaCl. After an expression period, the cells were lysed and samples loaded onto an SDS-Polyacrylamide gel for identification of coomassie-staining protein bands corresponding to the induced proteins. Novel bands were observed in induced samples (but not in uninduced samples) for both GST and HIS fusions for E2F1 and E2F4. DNA sequence analysis revealed that the 5′ ends of the cDNAs encoding these proteins were in the appropriate reading frame with the attB1 and AD. The predicted molecular weights of these fusion proteins were consistent with the induced bands on the protein gel. In contrast, no protein expression was observed for GST or HIS fusions of the EF5 clones tested. DNA sequence analysis of these clones showed that like E2F1 and E2F4 clones, the E2F5 clones were in the expected reading frame to allow expression. Similar results were observed for additional independent clones of E2F5 assayed. Hence, selection for proteins that interacted with DP 1 provided representatives E2F 1, E2F4 and E2F5, while imposing a second selection (protein expression in E. coli as GST or HIS fusions) generated the subset E2F1 and E2F5.

### Example 4

### In Vitro Selection by Hybridization

The vector pCMVSPORT6.0 (FIG. 34A-34D) contains attB1 and attB2 sites flanking a multiple cloning site. A cDNA library of high complexity (>106 individuals) constructed in this vector is used to identify potential members that encode 7-transmembrane helix proteins. First, a degenerate oligonucleotide is designed that corresponds to domains largely conserved in such protein types. A representative protein may resemble the human beta-2 adrenergic receptor (see, e.g., GenBank Accession No. M15169). A liquid hybridization with this oligonucleotide is performed according to methods previously described (see, e.g., U.S. Pat. No. 5,759,778) and cDNAs that hybridize to the probe are isolated, made double stranded and introduced into E. coli by transformation. Resulting clones are pooled, cultivated and DNA is prepared. The resulting mix represents a subpopulation of the original library that potentially encode authentic 7-transmembrane helix proteins. The mixture further contains other proteins with DNA sequence homology to the probe that are not 7-transmembrane helix proteins, and false positives. Plasmid DNA from this population is prepared and reacted with a vector containing attP sites (e.g., pDONR201, Invitrogen Corp., Carlsbad, Calif., Cat. No. 11798-014) in the presence of buffer and BP CLONASE™ to generate a population of ENTRY clones, which can be recovered in E. coli.

Alternatively, a sample of this in vitro mixture can be reacted directly with a Destination Vector (containing attR sites) in buffer and LR CLONASE™, to generate Expression Clones (containing attB sites) that harbor the cDNA in vectors encoding an N-terminal fusion to Green Fluorescent Protein (GFP). This population is subsequently introduced into E. coli by transformation, and DNA from the resulting pool of transformants is prepared and introduced into mammalian cells. Resulting transfected cells are examined for those clones in which GFP is localized to the membrane. This selection identifies individuals originating from a cDNA library that were isolated due to hybridization with a degenerate oligonucleotide probe, and that further generated a functional N-terminal fusion with GFP (i.e., was in the proper reading frame with GFP and attB1) and that localized to the cell membrane. Individuals from this population could be analyzed by DNA sequence determination (either directly, or following transfer via recombinational cloning into a more desirable vector). Alternatively, clones possessing the desired properties, features, or activities could be subjected to further selections: DNA from the subpopulation of cells in which the GFP-cDNA fusion is localized to the membrane is recovered and introduced into E. coli. DNA from the resulting pool of transformants is transferred into Adenoviral-based vectors (this can be done either by first isolating a pool of ENTRY Clones following reaction with pDONR201 (Invitrogen Corp., Carlsbad, Calif., Cat. No. 11798-014) in a BP CLONASE™ reaction, or in a single reaction in which a portion of this reaction is transferred directly into a mixture of buffer, Adenovirus-Destination Vector and LR CLONASE™) for in vivo infection of mice with selection for those clones that complement a defect in a presumed 7-transmembrane receptor protein or provide a phenotype of interest. DNA from the resulting mice is isolated and recovered in E. coli, or the cDNA insert is amplified using PCR and primers known to flank the cDNA from vector sequences. Because the resulting PCR product is flanked by attB1 and attB2 sites, the PCR product can be cloned using pDONR201 and BP CLONASE™ and used for further selections, or characterized directly.

### Example 5

### Screening of a PCR Generated Library

A collection of four hundred genes are amplified using PCR and oligonucleotides containing attB1 (5′ oligo) and attB2 (3′ oligo). The open reading frames extend from the translational start signal ATG, to the translational stop codon, with the wild-type stop codon altered to insert an amino acid, thereby allowing C-terminal protein fusions. The resulting PCR products are transferred using recombinational cloning into pDONR201 in a reaction with BP CLONASE™ to generate a collection of Entry Clones in E. coli. The resulting Entry Clones are combined into 8 pools of approximately 50 Entry Clones each, and DNA from the pools is prepared.

Each pool is transferred, using recombinational cloning (in a reaction containing LR CLONASE™) into a retroviral Destination vector in which the ccdB counterselection marker for use in E. coli is replaced by a marker allowing direct selection in mammalian cells (e.g., Herpes simplex thymidine kinase). The in vitro reaction mixture is transfected into packaging cell lines, and infectious virus (containing the population of cDNAs derived from the Entry Clones) is used to infect a recipient cell line designed to express a reporter gene in response to induction of the activation of particular transcription factors. As a result, cells expressing the reporter identify cDNAs that possess the ability to activate any of a number of signal transduction pathways. Cells showing a positive signal for induction of the reporter gene are pooled, genomic DNA is prepared, and the cDNA harbored by the retrovirus is rescued using PCR amplification from retroviral sequences. The resulting PCR products contain attB1 and attB2 flanking the cDNA, and are cloned using recombinational cloning in a reaction with BP CLONASE™ and pDONR201 (Invitrogen Corp., Carlsbad, Calif., Cat. No. 11798-014). Entry Clones from this mixture are pooled and represent subpopulations that encode proteins able to activate certain signal transduction pathways.

This population of Entry Clones is transferred using LR CLONASE™into a Destination Vector that contains a T7 RNA Polymerase responsive promoter, and the resulting reaction mixture is added to an in vitro transcription/translation reaction containing T7 RNA polymerase. Samples from the extract are assayed for the presence of proteins that possess kinase activity by their ability to utilize radio-labeled NTPs and phosphorylate known substrates. Hence, this process has provided selection of a subset of ORFs that induce specific signal transduction pathways and possess kinase activity.

### Example 6

### Transfer of a Library Between Vectors

Part I: Preparation of Library for Transfer

An Expression Clone library DNA derived from human brain tissue cloned in pCMVSPORT6.0 (FIG. 34A-34D) was diluted to 25 ng/μl based on an O.D. value at 260 nm. Samples containing 50 ng, 100 ng and 200 ng (2 μl, 4 μl, and 8 μl, respectively) of DNA were then respectively run on a 1% ethidium bromide (EtBr)-stained agarose gel to determine the quality of the library DNA. Depending on the type of library, the DNA generally ran as a 5-8 kb supercoiled smear with the major intensity at about 6 kb. The majority of the DNA generally ran as a supercoiled plasmid monomer and contained little or no non-recombinant vector DNA.

In instances were the library DNA appeared less concentrated than calculated from the O.D. readings, aliquots of the original library stock were PEG precipitated by adding 0.4 volumes of 30% PEG 8000/1.8M NaCl solution, mixing well and spinning at 13,000 rpm for 15 minutes at room temperature. The DNA was then dissolved in 10 mM Tris-HCl, at pH 7.5, 1.0 mM EDTA (TE), after which the DNA was again diluted with TE to 25 ng/μl based on an O.D. value at 260 nm. The diluted DNA was then rerun on a EtBr-stained agarose gel as described above to again to determine the quality of the library DNA.

Two aliquots of the 25 ng/μl library DNA was diluted 1/10 and 1/100 to 2.5 ng/μl and 0.25 ng/μl, respectively. One μl of each tube (25 ng, 2.5 ng and 0.25 ng total DNA) was then electroplated into DH10B Electromax cells. Two ml of S.O.C. medium (Invitrogen Corp., Carlsbad, Calif., Catalog No. 15544-034) was added to each of the transformations, after which the mixtures were shaken at 37° C. for 1 hour. One hundred μl of these diluted transformations (10⁻⁴ and 10⁻⁵ for 25 ng, 10⁻³ and 10⁻⁴ for 2.5 ng and 10⁻² and 10⁻³ for 0.25 ng) were then plated on amp plates to determine the total amount of DNA in terms of colony forming units/ng (CFU/ng). Generally, approximately 3×10⁶ CFU/ng were present based upon a transformation efficiency of 10¹⁰ CFU/μg of pUC DNA. In instances where the colony output of the library DNA did not appear to be accurate, the concentration of the library DNA was adjusted to approximately 75×10⁶ CFU/μl.

Part II: One Tube Reaction

BP reactions were set up as follows:

TABLE 3

| Component | Rxn 1 | | Rxn 2 | | Rxn 3 | | Rxn 4 | | Rxn 5 | |
|---|---|---|---|---|---|---|---|---|---|---|
| TE | 7 | μl | 5 | μl | 3 | μl | 1 | μl | 1 | μl |
| Linear pDONR plasmid | 3 | μl | 3 | μl | 3 | μl | 3 | μl | 3 | μl |
| (250 ng/μl) | | | | | | | | | | |
| cDNA library (25 ng/μl | 2 | μl | 4 | μl | 6 | μl | 8 | μl | 8 | μl |
| or 75 × 10⁶ CFU/μl) | | | | | | | | | | |
| BP Buffer | 4.5 | μl | 4.5 | μl | 4.5 | μl | 4.5 | μl | 4.5 | μl |
| Fis (1/4 dilution in H₂0 | 1.5 | μl | 1.5 | μl | 1.5 | μl | 1.5 | μl | 1.5 | μl |
| of 0.38 mg/ml) | | | | | | | | | | |
| BP CLONASE ™ | — | | — | | — | | — | | 12 | μl |
| Storage Buffer | | | | | | | | | | |
| BP CLoNASE ™ | 12 | μl | 12 | μl | 12 | μl | 12 | μl | — | |
| Final BP reaction volume | 30 | μl | 30 | μl | 30 | μl | 30 | μl | 30 | μl |

The tubes containing the above reaction mixtures were incubated at 25° C. overnight. Three μl of Proteinase-K (2 mg/ml) was then added to each reaction tube, after which the tubes were mixed well and incubated at 37° C. for 10 minutes. The Proteinase K was then heat inactivated by incubating the reaction tubes at 75° C. for 10 minutes. Five μl of each sample was then run on a 1% Sybr Gold gel, after which the efficiency of the BP reaction was determined whether a linear 6.5 kb by-product band was present. The linear 12-14 kb co-integrate molecules could generally also be identified on this gel. Further, in most instances, there was a shift of the library DNA down in size from 6-8 kb to 4-6 kb.

The following reaction mixtures were then set up for exonuclease treatment as follows:

TABLE 4

| Component | Volume | |
|---|---|---|
| H₂0 | 54 | μl |
| BP reaction | 28 | μl |
| 25 mM ATP | 4 | μl |
| 10x Exo buffer | 10 | μl |
| Exonuclease I (20 units/μl) | 2 | μl |
| Exonuclease V (10 u/μl) | 2 | μl |
| Total volume | 100 | μl |

The reaction tubes were incubated at 42° C. for 30 minutes. After which, the exonuclease reactions were stopped by incubation at 80° C. for 15 minutes. DNA was then ethanol precipitate by adding 100 μl of TE and 600 μl of ethanol/Na acetate solution and centrifugation at room temperature for 15 minutes at 13,000×rpm. The resulting DNA precipitate was dissolved in 30 μl of TE, 1 μl of which was used to electroporate Electromax DH10B cells. Two ml of S.O.C. medium was then added to each transformation and shaken at 37° C. for 1 hour. For reaction 5, 100 μl of undiluted transformations was plated on kan and 100 μl of 10⁻³ and 10⁻⁻⁴ dilutions on amp. For reactions 1, 2, 3, and 4, 100 μl of 10⁻³ and 10⁻⁴ dilutions was plated on kan plates and 100 μl of 10⁻² and 10⁻³ dilutions was plated on amp plates.

Two LR reactions were set up for the exonuclease treated BP reactions 1, 2, 3, and 4, as shown in Table 5.

TABLE 5

| | No LR | Plus LR |
|---|---|---|
| Component | CLONASE ™ | CLONASE ™ |
| Exo treated BP reaction | 5 μl | 15 μl |
| pDEST linearized (150 ng/μl) | 1 μl | 3 μl |
| LR4 buffer | 2 μl | 6 μl |
| LR storage buffer | 2 μl | — |
| LR CLONASE ™ | — | 6 μl |
| Total reaction volume | 10 μl | 30 μl |

The tubes containing the above reaction mixtures were incubated at 25° C. overnight. One μl of Proteinase K solution was added to the no CLONASE™ reactions and 3 μl of Proteinase K solution was added to the plus CLONASE™ reactions. The reaction tubes were then mixed and incubated at 37° C. for 10 minutes. Five μl of each reaction mixture was then run on a 1% Sybr Gold gel to assess the efficiency of each reaction. Two μl of each reaction mixture was electroporated into Electromax DH10B cells. The cells were then shaken at 37° C. for 1 hour in 2 ml of S.O.C. medium. For the no CLONASE™ reactions, 100 μl of 10⁻³ and 10⁻⁴ dilutions was plated on kan plates and 100 μl of 10⁻² and 10⁻³ dilutions was plated on amp plates. For the Plus CLONASE™ reactions, 100 μl of 10⁻² and 10⁻³ dilutions was plated on kan plates and 100 μl 10⁻³ and 10⁻⁴ dilutions was plated on amp plates. Optionally, nucleic acid in the reaction mixtures can be ethanol precipitated and concentrated prior to electroporation.

After overnight incubation, colonies were counted. The number of amp CFUs, as determined by the number of colonies on the amp plates, in the no CLONASE™ LR reaction was compared to the number of amp CFUs in the Plus CLONASE™ LR reaction. Clone checker analysis and colony PCR were performed to confirm (1) the ratio of new Expression clones to starting Expression Clones and (2) average size of the inserts.

Part III: Two Step/Tube Reaction and Alternative One Tube Reaction

Nucleic acid of a cDNA library was purified from E. coli using the Concert High Purity Plasmid Maxiprep System (Invitrogen Corp. Carlsbad, Calif., Catalog Series No. 11451). Ten μg of the library DNA was precipitated by adding 0.8 volumes of 15% PEG 8000/0.9M NaCl solution. The resulting solution was mixed well and centrifuged at 13,000 rpm in a microfuge for 15 minutes at room temperature. The supernatant was carefully removed and the DNA in the pellet was dissolved in 100 μl of TE. The DNA concentration was estimated by reading the OD 260 value. After which, the library DNA was diluted to about 25 ng/μl.

A. BP Reactions

BP reaction mixtures were prepared as follows:

BP CLONASE™ was thawed on ice and mixed well before use. A Supermix of following component was prepared at room temperature:

| | |
|---|---|
| Linear pDONR plasmid (250 ng/μl) | 10 μl |
| BP Buffer | 15 μl |
| Fis solution (80 ng/μl) | 5 μl |

TABLE 6

| | Titration of the amount of the | | | | Control library | |
|---|---|---|---|---|---|---|
| | starting library in BP reaction | | | | transfer | |
| | | | | Neg- | | Neg- |
| | | | | ative | | ative |
| | Titration | | | control | Positive | control |
| | Rxn | Rxn | Rxn | Rxn | Rxn | Rxn |
| Component | 1 | 2 | 3 | 4 | 5 | 6 |
| Water | 5 μl | 4 μl | 2 μl | 12 μl | 2 μl | 6 μl |
| Supermix | 6 μl | 6 μl | 6 μl | 6 μl | 3 μl | 3 μl |
| cDNA library | 1 μl | 2 μl | 4 μl | 2 μl | — | — |
| (25 ng/μl) | | | | | | |
| Positive control | — | — | — | — | 1 μl | 1 μl |
| library | | | | | | |
| (25 ng/μl) | | | | | | |
| saBP | 8 μl | 8 μl | 8 μl | — | 4 μl | — |
| CLONASE ™ | | | | | | |
| Final BP | 20 μl | 20 μl | 20 μl | 20 μl | 10 μl | 10 μl |
| reaction | | | | | | |
| volume | | | | | | |

The reactions tubes were mixed at room temperature and incubated at 25° C. for 48 hours. Two μl of Proteinase-K (2 mg/ml) was then added to reaction tubes 1, 2, 3 and 4 and 1 μl of Proteinase-K (2 mg/ml) was added to reaction tubes 5 and 6. All of the tubes were mixed well by pipeting and incubated at 37° C. for 10 minutes. One μl of each sample was electroporated into 25 μl Electromax DH10B cells (Invitrogen Corp., Cat. No. 18290-015) using the Cell-Porator Electroporation System (Invitrogen Corp.) and the remaining 21 μl in reaction tubes 1, 2, 3 and 4 were stored at −20° C. One ml of S.O.C. was added to each transformation mixture and shaken at 37° C. for 1 hour.

A series of dilutions of 100 μl of the transformation mixtures of reaction tubes 4 and 6 (10⁻³, 10⁻⁴ and 10⁻⁵) were made in S.O.C. These dilutions were then plated on LB amp (100 μg/ml) plates to determine the number of clones in the starting library. A series of dilutions of the transformation mixtures of reaction tubes 1, 2, 3 and 5 (10⁻¹,10⁻², 10⁻³ and 10⁻⁴) were also made in S.O.C. and plated on LB amp (100 μg/ml) and LB kan (50 μg/ml) plates to determine the number of clones in the Entry library and the residual starting library. These plates were incubated at 37° C. overnight.

Successful transfer generally demonstrated >50% conversion and <2% of residual starting library. The following formulas were used to determine the % conversion and the % residual:
% converted=[#KAN colonies (rxn 1, 2, 3, 5) with CLONASE™ rxn (×) dilution factor]/[(# AMP colonies (rxn 4, 6) no CLONASE™ rxn (×) dilution factor] (×) [μg of starting library (rxn 4, 6)/μg of starting library (rxn 1, 2, 3, 5)]
% residual starting library=#AMP colonies (rxn 1, 2, 3, 5) with CLONASE™rxn (×) dilution factor/(# Kan colonies (rxn 1, 2, 3, 5) (×) dilution factor)

Reactions with the highest entry clone titer and lowest residual starting library were chosen for use in the steps set out below.

B. Construction of an Entry Library

Enough DNA from the BP reaction to generate at least 10 million entry clones was electroporated into cells. One ml of S.O.C. was added to 25 μl of electroporated ElectroMax DH10B cells, which were then shaken at 37° C. for 1 hour. Fifty μl of the resulting transformation mix was removed and diluted 10⁻², 10⁻³, 10⁻⁴ and 10⁻⁵ in S.O.C. 100 μl of the resulting mixtures were then plated on LB amp and LB kan plates and incubated at 37° C. overnight. Sterile glycerol was added to the remaining undiluted transformation reaction (Entry library) to a final concentration of 15% and the mixture was stored at −80° C. for further use.

The titer of the Entry library was calculated by counting the number of colonies formed on LB kan plates as described above. 10 million colony forming units (CFU) from the frozen stock was then innoculated into 50 ml of LB containing kanamycin (50 μg/ml). The mixture was then shaken at 37° C. until the OD₆₀₀ reached 1.0 (approximately 6 hours). The culture was then centrifuged and the pellet was stored for later use at −80° C.

The pellet, which contains the Entry library, was thawed at room temperature and DNA was isolated using the Concert High Purity Plasmid Midiprep System (Invitrogen Corp. Carlsbad, Calif., Catalog Series No. 11451). The DNA was then resuspended in TE and the O.D. at 260 nm was read to estimate the DNA concentration.

Five μg of the Entry library DNA was precipitated by adding 0.8 volumes of a 15% PEG 8000/0.9M NaCl solution. The resulting solution was mixed well and centrifuged in a microfuge (13,000 rpm) for 15 minutes at room temperature. The supernatant was carefully removed and the DNA in the pellet was dissolved in 50 μl of TE. The O.D. at 260 nm was again read to estimate the DNA concentration.

C. LR Reaction to Transfer the Entry Library to the Expression Library

0.5 μg of Entry library DNA was diluted to 25 ng/μl and the remaining portion of the Entry library was stored at −20° C.

LR reaction mixtures were prepared as follows:

A Supermix of following components was prepared at room temperature:

| | |
|---|---|
| Linear Destination vector (150 ng/μl) | 12 μl |
| LR Buffer | 14 μl |
| Water | 22 μl |

TABLE 7

| | Library Transfer | | Control Library | |
|---|---|---|---|---|
| | Reactions | | Transfer | |
| | Negative | | Negative | |
| | control | Positive | control | Positive |
| Component | Rxn 1 | Rxn 2 | Rxn 3 | Rxn 4 |
| Water | 6 μl | — | 6 μl | — |
| Supermix | 12 μl | 12 μl | 12 μl | 12 μl |
| Entry cDNA library (25 ng/μl) | 2 μl | 2 μl | — | — |
| Positive control Entry library | — | — | 2 μl | 2 μl |
| (25 ng/μl) | | | | |
| LR CLONASE ™ | — | 6 μl | — | 6 μl |
| Final LR reaction volume | 20 μl | 20 μl | 20 μl | 20 μl |

The reaction mixtures were mixed gently at room temperature and incubated at 25° C. overnight. The samples were then treated with 2 μl Proteinase K at 37° C. for 10 minutes.

One μl of reaction tubes 1, 2, 3, and 4 was electroporated into 25 μl Electromax DH10B cells. One ml of S.O.C. was also added to reaction tubes 1, 2, 3, and 4 and the tubes were shaken at 37° C. for 1 hour. 100 μl of each transformation mix were removed and 10⁻², 10⁻³, 10⁻⁴ and 10⁻⁵ diltuions were prepared in S.O.C. 100 μl of the dilutions were then plated on LB amp and LB kan plates. The remaining 21 μl in reaction tubes 1, 2, 3 and 4 were stored at −20° C.

Successful LR transfer will generally demonstrate >50% conversion and ˜10% of residual Entry library. The following formulas were used to determine the % conversion and the % residual:
% converted=#AMP colonies (rxn 2, 4) with CLONASE™ rxn (×) dilution factor/(# KAN colonies (rxn 1, 3) no CLONASE™ rxn (×) dilution factor).
% residual starting library=#KAN colonies (rxn 2, 4) with CLONASE™ rxn (×) dilution factor/(# AMP colonies (rxn 2, 4) (×) dilution factor).

Enough DNA from reaction tube 2 to generate at least 10 million Expression clones was electroporated into cells. One ml of S.O.C. was added to 25 μl of electroporated ElectroMax DH10B cells, which were then shaken at 37° C. for 1 hour. Fifty μl of the transformation mix was removed and used to prepare dilutions of 10⁻², 10⁻³, 10⁻⁴ and 10⁻⁵ in S.O.C. 100 μl was then plated on LB amp and LB kan plates, which were incubated at 37° C. overnight. Sterile glycerol was added to the remaining undiluted transformation reaction mixtures (Expression library) to final concentration of 15%. These mixtures were then stored at −80° C. for further use.

D. Expression Library Analysis

Analysis of the expression libraries was performed as follows.

Titer analysis: Colonies on LB amp and LB kan plates were counted to determine the efficiency of conversion and the total colony output, also referred to as the number of colony forming units (CFU).

Sizing: Forty-four colonies on LB amp plates were randomly chosen and picked to confirm the ratio of new Expression library clones to starting cDNA library clones and to insure that the average size of the inserts did not change.

Methods which can be used for insert sizing include PCR amplification of the cDNA inserts with primers that hybridize to the Expression vector and miniprep preparation of plasmid DNA followed by digestion with EcoRI and NotI restriction endonucleases.

### Example 7

### Transfer of Libraries Between Plasmids

When transferring libraries or populations of DNA fragments from one plasmid backbone to another, it is generally advantageous for the transfer reactions to occur with an efficiency such that the representation of the original population of molecules remains essentially the same after transfer as it was before the transfer reaction. It is advantageous to transfer highly complex populations of molecules with the highest possible level of reaction efficiency (approaching 100 percent efficiency or the complete transfer of every molecule in the population).

The GATEWAY™ system is ideally suited to facilitate the transfer of complex populations of molecules. There presently exists many cDNA libraries already established as GATEWAY™ Expression Clones. These Expression Clones contain attB sites flanking their cDNA inserts. Thus, the first step in the transfer of an Expression Clone library would require a BP reaction. The subsequent Entry Clone products would then be used in an LR reaction with a Destination vector of choice.

The efficiency of BP reactions are highest when the DNA substrates consist of a supercoiled attP molecule reacted with a linear attB molecule. One common way to linearize a molecule at specific sites is to digest the plasmid with restriction endonucleases. However, not all Expression Clone libraries may contain the appropriate restriction sites and there will be insert molecules that would also be cut by the enzyme and thus could not be transferred by this method. It would be advantageous to optimize the BP reaction such that supercoiled attB molecules could be used as the substrate for the reaction. This would simplify the reaction and be generally applicable to all Expression Clone libraries.

Experiment 1: Test of DNA Topologies in BP Reactions

Expression Clones (linear and supercoiled) were reacted with attP Donor vectors (linear and supercoiled) in BP reactions. The cloning efficiency of two different Expression Clone DNAs (containing the lacZ alpha fragment and tetR inserts) at two different concentrations (25 fmoles and 50 fmoles) were compared in standard BP reaction conditions (300 ng attP plasmid, 4 μl of BP CLONASE™ in 20 μl reaction volume). Reaction efficiency was assessed following overnight incubation by gel electrophoresis and transformation (see data in Table 8).

TABLE 8
Colony output from BP reactions expressed in colonies/
transformation.

| Expression | | sc B × | sc B × | | |
|---|---|---|---|---|---|
| Clone | fmoles | sc P | lin P | linB × sc P | lin B × lin P |
| lacZ alpha | 25 | 4,700 | 29,000 | 65,000 | 33,400 |
| | 50 | 6,700 | 34,500 | 92,000 | 45,000 |
| Tet | 25 | 13,000 | 30,700 | 64,000 | 39,000 |
| | 50 | 19,500 | 42,900 | 99,000 | 82,000 |

This experiment shows that supercoiled attB Expression Clones can be most efficiently reacted with linear attP Donor plasmid.

Experiment 2: Inclusion of Fis in a Recombination Reaction

It has been shown that the Fis protein can enhance the output of the BP reaction. The effect of Fis protein was thus tested in BP reactions with the Tet Expression Clone DNA. Reactions were prepared with 300 ng of supercoiled or linear attP Donor plasmid reacted with 200 ng of supercoiled or linear Tet Expression Clone DNA in the presence (24 ng in a 20 μl reaction) and absence of Fis protein. The results are summarized in Table 9.

TABLE 9
The effect of Fis protein in BP reactions.

| Reaction | | | | lin B × |
|---|---|---|---|---|
| time | sc B × lin P | sc B × lin P + Fis | linB × sc P | sc P + Fis |
| 1 hour | 3,700 | 37,250 | 86,000 | 129,500 |
| overnight | 280,500 | 900,000 | 835,555 | 935,000 |

The experiment shows that linear attP Donor vectors are much less efficient in cloning than supercoiled vectors after 1 hour reactions but given enough time this difference can be minimized. Fis protein stimulates reactions with both linear and supercoiled attP Donor plasmids but the greatest effect of Fis is seen with linear attP plasmid.

### Example 8

### Optimization of One-Tube Reactions with Supercoiled attB Expression Clones

An Entry clone containing the lacZ open-reading-frame (ORF) but lacking the first ATG codon (pENTR201-no ATG-LacZ, derived from pENTR201 was constructed. The lacZ ORF was then transferred via LR reactions into different Destination Vectors. It was observed by plating on X-Gal plates that blue colonies were generated when this lacZ ORF was cloned into pDEST2 (pEXP2-no ATG-LacZ, see FIG. 22 of U.S. application Ser. No. 09/517,466, filed Mar. 2, 2000 and pDEST8 (EXP8-no ATG-LacZ, Invitrogen Corp., Carlsbad, Calif., Cat. No. 11804-010) while white colonies were generated when cloned into pDEST6 (pEXP6-no ATG-LacZ, see FIG. 26 of U.S. application Ser. No. 09/517,466, filed Mar. 2, 2000 and pDEST14 (pEXP14-no ATG-LacZ, Invitrogen Corp., Carlsbad, Calif., Cat. No. 11801-016). Thus these lacZ Expression clones can be used to assess the efficiency of one-tube transfers from one Destination Vector to another simply by plating on X-Gal.

As shown above in Example 7, supercoiled Expression Clone DNAs react most efficiently in BP reactions with linear attP DONOR Vector and Fis protein. Furthermore, the optimal transfer of inserts into a new Destination Vector would require limiting amounts of the starting Expression Clone DNA in order to minimize the amount of starting Expression Clone DNA contaminating the product of a one-tube reaction. The following experiment was used in part to determine the optimal amounts of linear pDONR vector and BP Clonase required for maximum efficiency of transfer in one-tube reactions.

TABLE 10
BP reactions with 40 ng pEXP8-no ATG-LacZ in a 20 μl final
volume.

| | | | BP | Kan | | |
|---|---|---|---|---|---|---|
| | Lin attP | | Clonase | Colonies | Amp Colonies | Ratio |
| | (ng) | Fis (ng) | (μl) | (cfu/ml) | (cfu/ml) | Kan/Amp |
| 1 | 300 | 50 | 0 | 198 | 298,000 | 0 |
| 2 | 300 | 50 | 4 | 24,700 | 66,500 | 0.4 |
| 3 | 300 | 50 | 8 | 115,300 | 18,950 | 6.1 |
| 4 | 450 | 75 | 8 | 97,000 | 15,800 | 6.1 |
| 5 | 600 | 100 | 8 | 81,500 | 5,560 | 14.7 |
| 6 | 600 | 100 | 10 | 110,000 | 3,600 | 30.6 |

The experiment shows that although the maximum number of Entry Clones produced reaches a plateau with 300 ng of pDONR plasmid, more Expression Clones are reacted by adding more pDONR plasmid and more BP Clonase.

TABLE 11
e-tube reactions with pEXP8-no ATG-LacZ (blue) to pEXP14-
no ATG-LacZ (white)

| | Lin attP | Fis | BP Clonase | White | Blue | Ratio |
|---|---|---|---|---|---|---|
| | (ng) | (ng) | (μl) | Colonies | Colonies | White/Blue |
| 1 | 300 | 50 | 0 | 0 | 160,000 | 0 |
| 2 | 300 | 50 | 4 | 18,500 | 65,000 | 0.3 |
| 3 | 300 | 50 | 8 | 42,650 | 10,600 | 4.0 |
| 4 | 450 | 75 | 8 | 45,300 | 11,800 | 3.8 |
| 5 | 600 | 100 | 8 | 29,200 | 4,175 | 7.0 |
| 6 | 600 | 100 | 10 | 10,825 | 6,025 | 1.8 |

Based on the results shown above, we have chosen to use 600 ng of linear attP DONOR plasmid and 8 μl of BP Clonase in library transfer protocols.

### Example 9

### Escherichia coli Fis Protein Stimulates Integrative Recombination by Bacteriophage Lambda Int

Background

Fis is a 98 amino acid homodimeric protein found in Escherichia coli and Salmonella typhimurium, as well as many other prokaryotes. It was first identified due to its role in regulating DNA recombination reactions carried out by the DNA invertase family (Johnson, R. C. et al. (1986) Cell 46:531-9 and Koch, C. and Kahmann, R. (1986) J. Biol. Chem. 261:15673-8). Fis is a member of a group of proteins known as the NAPS, or nucleoid-associated proteins, which perform numerous regulatory functions in the cell, and are often isolated as part of the mass of protein-DNA which forms the E. coli nucleoid (Pan, C. Q. et al. (1996)₃ J. Mol. Biol. 264:675-95). Most members of this family appear to be involved in specific or non-specific DNA interactions involving bending, looping, or condensation of the DNA substrate. Other roles for Fis were later identified, including its function as a transcriptional activator of a wide number of promoters (Nilsson, L. et al. (1990) EMBO J. 9:727-34; Ross, W. et al. (1990) EMBO J. 9:3733-42; Xu, J. and Johnson, R. C. (1995) J. Bacteriol. 177:5222-31), a repressor of another set of promoters (Ball, C. A. et al. (1992) J. Bacteriol. 174:8043-56; Koch, C. et al. (1991) Nucl. Acids Res. 19:5915-22; Xu, J. and Johnson, R. C. (1995a) J. Bacteriol. 177:938-47), a cofactor for DNA replication (Filutowicz, M. et al. (1992) J. Bacteriol. 174:398-407) and cell division/chromosome separation (Paull, T. T. and Johnson, R. C. (1995) J. Biol. Chem. 270:8744-54), and a participant in site-specific recombination of bacteriophage lambda (Thompson, J. F. et al. (1987) Cell 50:901-8; Ball, C. A. and Johnson, R. C. (1991) J. Bacteriol. 173:4027-31; Ball, C. A. and Johnson, R. C. (1991) J. Bacteriol. 173: 4032-8). Cellular levels of Fis vary dramatically during the E. coli cell cycle depending on the growth stage and the availability of nutrients (Ball, C. A. et al. (1992) J. Bacteriol. 174:8043-56; Thompson, J. F. et al. (1987) Cell 50:901-8). Calculations predict that during log phase growth, enough Fis is present in cells to bind every 500 base pairs along the chromosome. However, as cells enter stationary phase or are deprived of nutrients, levels of Fis drop to almost undetectable amounts (Ball, C. A. et al. (1992) J. Bacteriol. 174:8043-56).

Fis is capable of non-specific binding to DNA in vitro, but it has a considerably higher affinity for a series of sites with a degenerate 15 base pair consensus sequence which loosely resembles an inverted repeat (Pan, C. Q. et al. (1996) J. Mol. Biol. 264:675-95; Bruist, M. F. et al. (1987) Genes Dev. 1:762-72; Bokal, A. J. et al. (1995) J. Mol. Biol. 245:197-207).

DNA footprinting shows clear contacts between the protein and the DNA in these 15 base pair Fis binding sites; however, the DNA sequence alone appears to be a poor predictor of Fis binding affinity, and local DNA structure may influence the activity of a given Fis binding site. Fis bends DNA upon specific binding, and the degree of bending appears to depend upon the particular Fis binding site (Thompson, J. F. and Landy, A. (1988) Nucl. Acids Res. 16: 9687-9705.; Pan, C. Q. et al. (1996) Biochemistry 35: 4326-33). Bend angles between 45 and 90 degrees have been observed in different experiments using different DNA substrates (Thompson, J. F. and Landy, A. (1988) Nucl. Acids Res. 16:9687-9705).

The role of Fis in lambda site-specific recombination was first identified by Thompson et al., who observed a 20-fold stimulation of lambda excision in vitro with Fis in the presence of suboptimal levels of the lambda Xis protein (Thompson, J. F. et al. (1987) Cell 50:901-8). At saturating Xis levels, Fis appeared to have no effect on excision in vitro. Part of the explanation for this effect appears to lie in the overlapping binding sites for the two proteins. The two Xis binding sites, X1 and X2 are on the attR arm of the recombination substrates, and the X2 site overlaps the Fis consensus sequence significantly. Cooperativity in binding is observed with Fis and Xis, just as it is with Xis alone; in fact, Fis appears to simply substitute for Xis in cases where Xis concentration is limiting (Thompson, J. F. et al. (1987) Cell 50:901-8).

Genetic evidence from Ball and Johnson (Ball, C. A. and Johnson, R. C. (1991) J. Bacteriol. 173:4027-31; Ball, C. A. and Johnson, R. C. (1991) J. Bacteriol. 173:4032-8) demonstrated that not only could Fis stimulate excision of phage lambda, but that lysogeny was also enhanced by the presence of Fis. These experiments, carried out in vivo using phage mutated in the F site and/or E. coli lacking Fis, demonstrated a 15-fold drop in lysogenization frequency when Fis was deleted (Ball, C. A. and Johnson, R. C. (1991) J. Bacteriol. 173:4032-8). A part of this decrease is clearly due to the loss of Fis as a regulator in non-recombination related events. However, a mutation of the F site which eliminates Fis binding without affecting Xis binding, still leads to a loss of 2-3 fold in lysogenization frequency, suggesting that Fis plays a role in integration as well as excision. Previous experiments carried out in vitro with Fis to look at integration did not identify any effect of Fis on the reaction (Thompson, J. F. et al. (1987) Cell 50:901-8).

Examples of the Use of Fis to Stimulate Recombination

Addition of between 200 and 500 nM Fis to a standard BP CLONASE™GATEWAY™ reaction will produce optimal stimulation of recombination product formation and number of output colonies. Similar levels of Fis will also stimulate reactions in which the topology of BP substrates are reversed; that is, using a linear P and supercoiled B substrate (library transfer). In both cases, the standard reaction conditions for the BP CLONASE™ reaction can be used. The same optimal range of Fis will also stimulate recombination reactions containing single P and B recombination sites under the same reaction conditions as reactions in the absence of Fis.

Summary of the levels of Fis Stimulation of Recombination

A. Single Recombination Site Reactions

Optimal Fis stimulation is observed over a range of 200-500 nM Fis and 5 nM DNA. Fis stimulates all single-site integration reactions regardless of topology of substrates. The standard reaction using supercoiled attP and linear attB sites is stimulated up to 10-fold in the presence of lower levels of Int. The reverse topology reaction, using supercoiled attB and linear attP sites is stimulated up to 5-fold at various salt concentrations. The reaction between linear attP and linear attB sites is stimulated up to 3-fold by Fis.

B. Dual Recombination Site reactions (GATEWAY™)

Optimal Fis stimulation is observed over a range of 200-500 nM Fis and 5 nM DNA. Fis stimulates the production of BP reaction product up to 3-fold depending on conditions. This stimulation appears to be due entirely to the stimulation of the resolution of the cointegrate, as cointegrate formation is unaffected. Standard GATEWAY™ reactions can be stimulated simply by adding Fis to the reaction under the same conditions as those normally used. In the reverse topology GATEWAY™ reaction (linear P, supercoiled B), Fis stimulates the production of product slightly, but significantly increases the amount of starting B substrate which is converted into cointegrate.

Results

Production of Fis—The E. coli fis gene was cloned into pLDE15 downstream of the lambda P_{L} promoter under control of the heat-inducible lambda cI⁸⁵⁷ repressor. This construct expressed Fis at high levels upon induction at 42° C. and a series of extracts were made to test purification protocols.

A final protocol was developed in which a liter of culture would produce 2-3 milligrams of purified (>90%) Fis. The procedure involved sonication to form a crude extract, followed by chromatography on Heparin sulfate, followed by ion-exchange chromatography on MonoS. The purified protein contains a few minor contaminants which could be further removed, possibly by either heating the extract before purification (as Fis is completely heat stable to boiling for up to 10 minutes), or by crystallization of Fis by complete dilution of salt. Both of these methods have been used in the literature. The final Fis sample was dialyzed into buffer containing 50% glycerol and 0.5M NaCl and was aliquoted into several tubes stored at either −20° C. or −80° C. The purified Fis was assayed for activity using a gel retardation assay similar to those published in the literature and found to have apparent K_{d} values between 10-30 nM.

Effect of Fis on Excisive Recombination—The effect of Fis on excision in vitro was measured using the double-site LR assay using supercoiled pEZ11104 (attL) and linearized pRCAT1 (attR). As shown in FIG. 22, increasing amounts of Fis protein showed a slight stimulation of the amount of recombinant product at high levels of Xis. However, as Xis levels were decreased, the stimulation by Fis was increased, such that at very limiting levels of Xis, maximal Fis stimulation reached 10-15 fold. Maximal stimulation by Fis seemed to occur between 30-125 ng Fis per 20 μl reaction. Because of the rapid conversion of cointegrate into product, it is difficult to analyze whether Fis affects both cointegrate formation and resolution; however, it is likely that stimulation is observed at both steps, and the level of stimulation appears to be similar.

Effect of Fis on Integrative Cointegrate Resolution—FIG. 23 shows the effect of Fis addition to a double-site BP assay using supercoiled pDONR201 (attp) and linearized pBGFP1 (attB). The percentage of recombination products is increased 2-4 fold in the presence of optimal levels of Fis (again, 30-120 ng/reaction). Also, stimulation by Fis is greater at higher salt, which is a condition that normally disfavors cointegrate resolution. There is no observable effect on cointegrate formation in the presence of Fis at any salt concentration (data not shown).

FIG. 24 analyzes the effect of salt concentration in more detail. Once again, the stimulation by Fis is seen at all salt concentrations, but because the control in the absence of Fis is so dramatically affected by salt concentration, the stimulation by Fis at higher salt is much stronger. At 25 mM NaCl, Fis stimulates nearly 2-fold, while at 75 and 100 mM NaCl, Fis stimulation is greater than 7-fold. In no case, however, is the amount of recombinant product at higher salt higher than the optimal Fis-stimulated recombination at 25 mM NaCl.

Effect of Fis on Integrative Recombination—Experiments indicated that Fis has no effect on single-site PxB recombination under standard conditions where attP (pATTP2) is supercoiled, and attB (pATTB2) is linear, at either low or high salt. However, if the levels of Int are reduced to suboptimal concentrations (FIG. 25), Fis is now capable of stimulating this reaction up to 10-fold. In addition, when both substrates are linearized, Fis has a dramatic effect on recombination levels. With linearized pATTP2 and linearized pATTB2, Fis stimulates recombination 2-3 fold at varying salt concentrations, much like the results seen for cointegrate resolution reactions. The most significant effect of Fis seems to be on the reaction between supercoiled pATTB2 and linear pATTP2. This reaction is extremely poor under normal conditions, with barely detectable amounts of product observed even at low salt conditions. However, in the presence of Fis recombination is strongly stimulated.

Discussion

Fis is known to play a role in lambda site-specific recombination. While in vitro roles have been observed only in situations where proteins are limiting, such conditions are highly artificial for a system whose main function is to carry out a single recombination event to introduce or excise one molecule of phage DNA, not to catalyze recombination of vast amounts of plasmid substrates. The in vivo data suggest an essential role for Fis in both integrative and excisive recombination of phage lambda. The dramatic 50-fold drop in phage lysis in the absence of Fis, and the 15-fold drop in lysogenization frequency clearly point to the likely in vivo requirement for Fis. While the role of Fis in lysis is, in some respects, similar to results found using in vitro experiments, explanations for the role of Fis in lysogeny have been considerably more elusive. While some of the 15-fold stimulation obtained by Ball and Johnson can be attributed to other roles of Fis in the cell, a nearly 3-fold effect is still observed from mutation of the F site, which must be directly related to recombinational stimulation.

The results of this study identified the likely source of the stimulation observed in vivo during integration. A 2-3 fold effect is clearly observed in vitro when attP substrates are not supercoiled. It has long been known that supercoiling energy appears to be essential for proper establishment of the protein-DNA structure known as the intasome, which is required to form prior to the onset of recombination. This argument has been used to explain the much lower recombination efficiency observed with non-supercoiled attP substrates in vitro. However, it has been widely shown that DNA in the cell is not supercoiled to the high levels of superhelicity seen in isolated plasmid DNA.

Johnson first proposed the notion that Fis may be used in the cell to enhance integration under conditions where such high superhelicity is not present (Ball, C. A. and Johnson, R. C. (1991b) J. Bacteriol. 173:4032-8). Given the fact that many nucleoid associated proteins appear to be involved in DNA compaction of the nucleoid, it is possible that the ability of Fis to bind and bend DNA may well mimic the compaction of DNA by supercoiling, and such an event may allow proper intasome formation even in the absence of high superhelicity. This may also be the explanation for the stimulation by Fis observed at suboptimal Int concentrations. In the cell, where Int levels are likely to be much lower than the artificially high concentrations used in laboratory in vitro recombination reactions, Fis may be necessary even for a “standard” recombination reaction to proceed.

The ability of F site mutants to promote stronger Fis stimulation of integration is further evidence for the role proposed above. Tighter Fis binding would likely lead to more efficient compaction of the DNA, and an increase in integration stimulation. It remains to be seen whether these effects are manifested at the kinetic level—that is, does the addition of Fis directly speed up intasome formation? Initial studies point towards an increase in the initial rate of the linear attP/supercoiled attB reaction in the presence of Fis, suggesting that indeed Fis may be kinetically acting at the level of intasome formation.

It is not entirely clear why Fis seems to have a greater stimulation of linear P/supercoiled B reactions as compared to reactions in which both substrates are linear. It is believed that integrative intasome formation occurs solely on attP, with capture of attB being a final step in the synapsis process. In this case, it is unclear how the supercoiling state of attB could affect the outcome of intasome formation. Instead, it is possible that Fis interaction with attB somehow makes the attB sites more accessible to the intasome, or aids a downstream post-synapsis step such as isomerization after the first strand cleavage.

Experimental Methods

Oligonucleotides—oligonucleotides were obtained from Life Technologies.

| | | |
|---|---|---|
| DE09: | 5′-GGGGGCTGCAGGCAAGAAGACAAAAATCACCTTGCGC | (SEQ ID NO:55) |
| DE10: | 5′-GGGGGCCCGGGCAGAGGCAGGGAGTGGGACAAAATTG | (SEQ ID NO:56) |
| DE46 (Fis start): | 5′-GGAGGGAATTCAGGAGGTATAAATTAATGTTCGAACAACGCGTAAATTCTG | (SEQ ID NO:57) |
| DE49 (Fis stop): | 5′-GGAGGGGATCCTTATTAGTTCATGCCGTA | (SEQ ID NO:58) |
| DE162: | 5′-GGAAGGAGATCTTGCTCAAAATTTGAGCTACATAATACTGTAAAACAC | (SEQ ID NO:59) |

Recombination Assay Plasmids—pATTP2 was constructed by cloning the lambda attP site into pUC19. pATTB2 was constructed by cloning the E. coli attB site into pUC19. pDONR201 (Life Technologies) contains attP1 and attP2 sites flanking a ccdB gene. pEZ11104 contains attL1 and attL2 sites flanking a CAT gene. pBGFP2 is pUC19 into which a PCR fragment containing the attB1 and attB2 sites flanking the GFP gene has been inserted. pRCAT1 is pUC19 into which a fragment of pEZC8402 containing the attR1 and attR2 sites and the CAT/ccdB cassette has been inserted.

Cloning of E. coli fis—The fis gene was PCR amplified from E. coli DH10B chromosomal DNA using Platinum Taq Hi Fidelity, and primers (DE46 and DE49) corresponding to the 5′ and 3′ ends of the gene. The 5′ primer was constructed to provide a strong Shine-Delgarno initiation sequence prior to the start of the fis gene. The PCR product was digested and cloned into pRAD19, a high copy-number expression vector carrying the lambda P_{L} promoter under the control of the heat-inducible lambda CI⁸⁵⁷ gene. A positive clone (pLDE15) was sequence verified to ensure that no mutations were present, and was introduced into E. coli BL21 for expression.

Induction of E. coli Fis protein—Cells containing pLDE15 were grown overnight at 30° C. in 2 milliliters of LB with 100 μg/ml ampicillin, diluted into 2 milliliters of fresh media, and grown to an OD₆₀₀ of 0.7. The culture was split into 2 tubes, with one remaining at 30°, with the other induced at 42° for 2 hours. After 2 hours, the cultures were spun down, resuspended in loading buffer, and analyzed by SDS-PAGE. The induced cells already had a partially lysed appearance, suggesting that dramatic overexpression of Fis may be lethal to E. coli under these conditions. Induced samples showed a very clearly overexpressed protein band at a molecular weight of around 12 kDa.

Purification of E. coli Fis protein—A 5 ml overnight culture of pLDE15 was diluted into 1 liter LB+Amp in a Fembach flask, and was grown at 30° C. to an OD₆₀₀ of 0.7, induced at 42° C. for 2 hours, and spun down. 7.5 g of wet cells were obtained, and were frozen at −80° C. Cells were thawed and resuspended in 15 milliliters of buffer containing 50 mM Tris-HCl, pH 8.0, 5 mM EDTA, 10% glycerol, 1 M NaCl, and 1 mM DTT. The cell solution was sonicated 4 times for 45 seconds with a ½ inch tip, and debris was removed by centrifugation at 30,000×g for 40 minutes. Extracts were stored at −80° C. 15 milliliters of extract was diluted with 35 milliliters buffer A (20 mM Tris-HCl, pH 8.0, 1 mM EDTA, 10% glycerol, 1 mM DTT) and applied to a Pharmacia Hitrap Heparin column (2×1 ml columns in series) at a flow rate of 0.25 ml/min. The column was washed with 400 mM NaCl in buffer A for 10 CV, and eluted with a 15 CV gradient from 400 mM to 800 mM NaCl in buffer A. A broad peak of Fis was detected by SDS-PAGE and fractions containing Fis were pooled, and dialyzed against buffer A with 200 mM NaCl. This sample was applied to a 1 ml Pharmacia Hitrap MonoS column equilibrated in the same buffer. The column was washed with 15 CV of 200 mM NaCl in buffer A, and eluted with a 20 CV gradient of 200 mM to 1M NaCl in buffer A. Two peaks were observed from the column, with the second sharp peak representing most of the Fis protein. The cleanest fractions were pooled to give a sample containing >90% Fis by Coommassie staining. Purified Fis was obtained at 1 mg/ml concentration after dialysis into Fis storage buffer containing 20 mM Tris-HCl, pH 8.0, 1 mM EDTA, 50% glycerol, 1 mM DTT, 0.5 M NaCl. Fis was stored at −80° C. or −20° C.

Fis activity assay—A gel retardation assay was developed to test for Fis activity. A PCR product consisting of the lambda attP sequence was amplified using primers DE9 and DE10. The 400 base pair product was cut with AvaI and labeled at the ends with ³²P-dCTP using the Klenow fragment of E. coli DNA polymerase I. Reactions were carried out with final conditions of 20 mM Tris-HCl, pH 8.0, 5% glycerol, 25 mM NaCl, 200 μg/ml salmon testis DNA, 1.17 ng (10,000 cpm/fmol) PCR product in a 20 μl reaction. Protein was added, and binding was carried out for 10 minutes at room temperature, and samples were loaded on a Novex 6% gel retardation gel running in 0.5×TBE buffer for 60 minutes at 100 V. Gels were dried and visualized on the Phosphorimager after 2-3 hour exposure. Multiple shifts were observed in assays without competitor DNA. In the presence of competitor, however, a single discrete shift was observed, and allowed the calculation of an apparent Kd value. These PCR products were somewhat impure, containing breakdown products, and the values obtained were therefore slightly error prone; however, the apparent Kd appeared to be between 10-30 nM, which agrees well with published values using the lamdba F site. This suggests that this kind of gel retardation assay would serve as an effective check of the activity of purified Fis protein.

Radioactive assay substrates—Linear substrates for recombination assays were labeled by Klenow fill-in reactions. Linearized substrates (1 μg) were incubated with 0.5 units of Klenow polymerase, 1 mM dATP, 1 mM dGTP, 1 mM dTTP, and 30 μCi of ³²P-dCTP for 14 minutes, 1 mM dCTP was added, incubated for 1 minute, and the labeled DNA was purified using Concert PCR purification columns, and eluted in 50 μl TE.

Recombination assays—Single-site recombination reactions (20 μl) consisted of 25 mM Tris-HCl, pH 8.0, 1 mM EDTA, 6 mM spermidine, 15% glycerol, and 75 mM NaCl (unless indicated otherwise), 100 fmoles of each substrate, and approximately 30,000 cpm of ³²P-labelled linear substrate. Standard integration reactions contained 80 ng IHF and 150 ng Int. Excision reactions contained 35 ng IHF, 50 ng Xis, and 150 ng Int. Reactions were incubated for 45 minutes at 25° C., and stopped by the addition of 50 μg/ml Proteinase K, heated for 15 minutes at 65° C., and electrophoresed on a 0.7% agarose gel. Gels were dried down and visualized on a Molecular Dynamics phosphorimager. Recombination levels were determined by quantitation of substrate and product bands using ImageQuant. GATEWAY™ (2-site) reactions were performed similarly, except that standard BP reactions contained 4 mM spermidine and 25 mM NaCl, and standard LR reactions contained 7.5 mM spermidine and 75 mM NaCl.

### Example 10

### Use of Fis in BP CLONASE™ Reactions

BP recombination reactions were performed for 60-120 minutes at room temp in 20 μl reaction mixtures containing 50 fmol supercoiled pDONR201, 75 mM NaCl, 7.5 mM spermidine, 2 μl BP storage buffer (5 mM EDTA, 1 mg/ml BSA, 22 mM NaCl, 5 mM spermidine, 25 mM Tris-HCl, pH 7.5) and 2 μl BP CLONASE™ (40 ng/μl Int, 20 ng/μl IHF, pH 7.5). The optimal Fis concentration for enhancing the efficiency of BP CLONASE™ catalyzed recombination reaction was found to be about 150 nM.

Further, the above reaction conditions generate a colony output that is similar to the standard reaction (i.e., 300 ng pDONR DNA, 100 ng attB DNA, 4 μl BP CLONASE™, 4 μl BP buffer for a 20 μl reaction), but requires half the amount of enzyme and vector DNA.

In a standard BP recombination reaction, addition of Fis results in a 3-fold increase in colony output as compared to from a standard BP reaction.

Fis is known to exert its effect by stimulating the rate of the second recombination reaction (cointegrate resolution) which is a linear by linear recombination reaction.

While not wishing to be bound by theory, the overall efficiency of BP recombination reactions involving linear and supercoiled nucleic acid molecules is as follows:
Supercoiled P×Linear B>Linear P×Supercoiled B>Linear P×Linear B>Supercoiled P×Supercoiled B

### Example 11

### Optimization of Library Transfer Conditions

A. Construction of attB cDNA libraries

One problem associated with Gateway library construction and transfer is that attB cDNA is generally limiting in BP reactions and standard BP reaction conditions need to be optimized to maximize colony output.

One solution to this problem is to use less supercoiled attP Donor Vector, less BP CLONASE™ and include Fis protein in the reactions with limiting amounts of attB cDNA. For example, to clone 20 ng of attB cDNA, optimal BP reactions contained 75 ng of attP Donor Vector, 0.75 μl of BP CLONASE™ and 84 nM Fis protein in a 20 μl reaction volume. The use of attB1.6 and attB2.10 sites improved colony output and resulted in an increase in the average size of the inserts.

B. Transfer of Expression Clone Libraries

The transfer of Gateway libraries is that BP reactions are most efficient using linear attB and supercoiled attP molecules and the use of restriction enzymes to linearize the library DNA results in some inserts being cut. However, BP reaction efficiency can be increased when linear P molecules are used by using limiting amounts of supercoiled Expression Clone DNA (50 ng/20 μl reaction), an excess of linear attP DNA (450 ng to 600 ng/20 μl reaction), and allowing the reaction to proceed overnight. Use of more BP CLONASE™ (up to 8 μl/20 μl reaction) and Fis protein helps to react more of the starting library away so as to reduce co-transformation and contamination of transferred libraries with starting clones.

C. Colony Output after Electroporation of BP Reactions

Kan colony output after electroporation of pENTR201 Clones (Entry Clones prepared using pDONR201; see FIGS. 26A-26C) is 10% of the expected number. These data are based on a comparison of amp and kan colony output of electroporation with a pENTR201-amp Entry Clone DNA. This phenomenon is specific for electroporation since the amp and kan colony output is identical after chemical transformation.

Two methods can be used to increase colony output. The first is to increase the S.O.C. medium recovery volume. When this was done, the following data was obtained:

Colony output vs Recovery volume with electroporation
of pENTR201-amp
1 ml S.O.C. = 10% kan to amp
2 ml S.O.C. = 30% kan to amp
4 ml S.O.C. = 60% kan to amp

The second method is to replace pDONR201 with pDONR212 (FIGS. 27A-27C). pENTR12-amp clones produced 80% kan to amp colonies using 1 ml S.O.C. medium recovery and 100% kan to amp colonies using 2 ml S.O.C. medium recovery.

D. Heterogeneous Colony Size of pENTR212 Clones

pENTR201 library clones have been found to produce homogeneous sized colonies whereas pENTR212 library clones produce heterogeneous sized colonies. Replacement of the origin of pDONR212 with a full pUC origin (FIGS. 28A-28C) solved this problem. The pENTR212 library clones demonstrate a cold-sensitive phenotype. In particular, clones of such libraries do not form colonies at 30° C. but do form colonies at 37° C. Replacement of the origin of replication did not change the phenotype when the new origin was placed in the same orientation as the original one. However, temperature sensitivity was largely alleviated when the origin was inserted in the opposite orientation (see FIGS. 29A-29C for a description of this construct).

E. Amplification of Primary Entry Clone Libraries

It has also been found that pENTR212 Entry Clone libraries can not be amplified without significantly decreasing the average size of the inserts. This effect was largely alleviated by replacing the origin with a full pUC origin. 300 μg/ml kanamycin was then required for selection of cells which contain the resulting vector in semi-solid medium.

F. One-Tube Reactions

An alternative to amplification of the Entry Clone intermediate, the product of BP reactions can be transferred directly into Destination Vectors in a “one-tube” reaction. The efficiency, however, of one-tube reactions can be low and may produce variable results.

Exonuclease treatment of the BP reaction mixture, ethanol precipitate and set up LR reactions using LR4 buffer conditions (i.e., 51 mM Tris-HCl (pH7.5), 1 mM EDTA, 1 mg/ml Bovine serum albumin, 76 mM NaCl, 7.5 mM spermidine) was shown to both increase transfer efficiency and reproducibility of the results. In some cases, the exonuclease treatment step may be omitted.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

In addition, the following documents are incorporated herein by reference in their entireties: U.S. application Ser. No. 08/486,139, filed Jun. 7, 1995; U.S. application Ser. No. 08/663,002, filed Jun. 7, 1996 (now U.S. Pat. No. 5,888,732); U.S. application Ser. No. 09/005,476, filed Jan. 12, 1998 (now U.S. Pat. No. 6,171,861); U.S. Appl. No. 60/065,930, filed Oct. 24, 1997; U.S. application Ser. No. 09/177,387, filed Oct. 23, 1998; U.S. Appl. No. 60/122,389, filed Mar. 2, 1999; U.S. Appl. No. 60/122,392, filed Mar. 22, 1999; U.S. Appl. No. 60/126,049, filed Mar. 23, 1999; U.S. application Ser. No. 09/233,493 (now U.S. Pat. No. 6,143,557); U.S. application Ser. No. 09/438,358, filed Nov. 12, 1999; U.S. Appl. No. 60/284,528, filed Apr. 19, 2001; U.S. Appl. No. 60/136,744, filed May 28, 1999; U.S. application Ser. No. 09/432,085, filed Nov. 2, 1999; U.S. application Ser. No. 09/498,074, filed Feb. 4, 2000; U.S. Appl. No. 60/108,324, filed Nov. 13, 1998; U.S. application Ser. No. 09/438,358, filed Nov. 12, 1999; U.S. application Ser. No. 09/517,466, filed Mar. 2, 2000; U.S. application Ser. No. 09/732,914, filed Dec. 11, 2000; and PCT Publication No. WO 00/52027.

What is claimed is:

1. A method for inserting a population of nucleic acid molecules into a second target molecule, the method comprising:
(a) mixing at least a first population of nucleic acid molecules comprising one or more recombination sites with at least one first target nucleic acid molecule comprising one or more recombination sites;
(b) causing some or all of the nucleic acid molecules of the at least first population to recombine with some or all of the first target nucleic acid molecules, thereby forming a second population of nucleic acid molecules;
(c) mixing at least the second population of nucleic acid molecules with at least one second target nucleic acid molecule comprising one or more recombination sites; and
(d) causing some or all of the nucleic acid molecules of the at least second population to recombine with some or all of the second target nucleic acid molecules, thereby forming a third population of nucleic acid molecules.

2. The method of claim 1, wherein the first population of nucleic acid molecules comprises a cDNA library.

3. The method of claim 1, wherein the first population of nucleic acid molecules comprises a genomic library.

4. The method of claim 1, wherein the first target nucleic acid molecule is a linear nucleic acid molecule.

5. The method of claim 1, wherein the individual members of the first population of nucleic acid molecules are linear nucleic acid molecules.

6. The method of claim 4, wherein the first target nucleic acid molecule is flanked by two recombination sites.

7. The method of claim 4, wherein the first target nucleic acid molecule is flanked by one recombination site and one restriction endonuclease site.

8. The method of claim 5, wherein the individual members of the population of nucleic acid molecules are flanked by two recombination sites.

9. The method of claim 5, wherein the individual members of the first population of nucleic acid molecules are flanked by one recombination site and one restriction endonuclease site.

10. The method of claim 1, wherein the recombination sites comprise one or more recombination sites selected from the group consisting of:
(a) lox sites;
(b) psi sites;
(c) dif sites;
(d) cer sites;
(e) frt sites;
(f) att sites; and
(g) mutants, variants, and derivatives of the recombination sites of (a), (b), (c), (d), (e), or (f) which retain the ability to undergo recombination.

11. The method of claim 10, wherein the recombination sites which recombine with each other comprise att sites having identical seven base pair overlap regions.

12. The method of claim 11, wherein the first three nucleotides of the seven base pair overlap regions of the recombination sites which recombine with each other comprise nucleotide sequences selected from the group consisting of:
(a) AAA;
(b) AAC;
(c) AAG;
(d) AAT;
(e) ACA;
(f) ACC;
(g) ACG;
(h) ACT;
(i) AGA;
(j) AGC;
(k) AGG;
(l) AGT;
(m) ATA;
(n) ATC;
(o) ATG; and
(p) ATT.

13. The method of claim 11, wherein the first three nucleotides of the seven base pair overlap regions of the recombination sites which recombine with each other comprise nucleotide sequences selected from the group consisting of:
(a) CAA;
(b) CAC;
(c) CAG;
(d) CAT;
(e) CCA;
(f) CCC;
(g) CCG;
(h) CCT;
(i) CGA;
(j) CGC;
(k) CGG;
(l) CGT;
(m) CTA;
(n) CTC;
(o) CTG; and
(p) CTT.

14. The method of claim 11, wherein the first three nucleotides of the seven base pair overlap regions of the recombination sites which recombine with each other comprise nucleotide sequences selected from the group consisting of:
(a) GAA;
(b) GAC;
(c) GAG;
(d) GAT;
(e) GCA;
(f) GCC;
(g) GCG;
(h) GCT;
(i) GGA;
(j) GGC;
(k) GGG;
(l) GGT;
(m) GTA;
(n) GTC;
(O) GTG; and
(p) GTT.

15. The method of claim 11, wherein the first three nucleotides of the seven base pair overlap regions of the recombination sites which recombine with each other comprise nucleotide sequences selected from the group consisting of:
(a) TAA;
(b) TAC;
(c) TAG;
(d) TAT;
(e) TCA;
(f) TCC;
(g) TCG;
(h) TCT;
(i) TGA;
(j) TGC;
(k) TGG;
(l) TGT;
(m) TTA;
(n) TTC;
(o) TTG; and
(p) TTT.

16. The method of claim 1, wherein the recombination in step (b) is caused by mixing the first population of nucleic acid molecules and the first target nucleic acid molecule with one or more recombination proteins under conditions which favor the recombination.

17. The method of claim 16, wherein the one or more recombination proteins comprise one or more proteins selected from the group consisting of:
(a) Cre;
(b) Int;
(c) IHF;
(d) Xis;
(e) Hin;
(f) Gin;
(g) Cin;
(h) Tn3 resolvase;
(i) TndX;
(O) XerC; and
(k) XerD.

18. The method of claim 16, wherein the one or more recombination proteins are in admixture with at least one second protein which (1) has a molecular weight below about 14,000 daltons, (2) contains at least 15% basic amino acid residues, and (3) enhances recombination.

19. The method of claim 18, wherein the one or more second proteins comprises Fis, a ribosomomal protein, or a fragment of either Fis or a ribosomomal protein.

20. The method of claim 19, wherein the ribosomal protein is a prokaryotic ribosomal protein.

21. The method of claim 20, wherein the ribosomal protein is an Escherichia coli ribosomal protein.

22. The method of claim 21, wherein the E. coli ribosomal protein is selected from the group of E. coli ribosomal proteins consisting of S10, S14, S15, S16, S17, S18, S19, S20, S21, L14, L21, L23, L24, L25, L27, L28, L29, L30, L31, L32, L33 and L34.

23. The method of claim 1, wherein the recombination in step (d) is caused by mixing the second population of nucleic acid molecules and the second target nucleic acid molecule with one or more recombination proteins under conditions which favor the recombination.

24. The method of claim 23, wherein the one or more recombination proteins comprise one or more proteins selected from the group consisting of:
(a) Cre;
(b) Int;
(c) IHF;
(d) Xis;
(e) Hin;
(f) Gin;
(g) Cin;
(h) Tn3 resolvase;
(i) TndX;
(j) XerC; and
(k) XerD.

25. The method of claim 16, wherein the one or more recombination proteins are in admixture with at least one second protein which (1) has a molecular weight below about 14,000 daltons, (2) contains at least 15% basic amino acid residues, and (3) enhances recombination.

26. The method of claim 18, wherein the one or more second proteins comprises Fis, a ribosomomal protein, or a fragment of either Fis or a ribosomomal protein.

27. The method of claim 26, wherein the ribosomal protein is a prokaryotic ribosomal protein.

28. The method of claim 27, wherein the ribosomal protein is an Escherichia coli ribosomal protein.

29. The method of claim 28, wherein the E. coli ribosomal protein is selected from the group of E. coli ribosomal proteins consisting of S10, S14, S15, S16, S17, S18, S19, S20, S21, L14, L21, L23, L24, L25, L27, L28, L29, L30, L31, L32, L33 and L34.

30. The method of claim 1, wherein the first target nucleic acid molecule is a vector.

31. The method of claim 30, wherein the vector is selected from the group consisting of:
(a) pDONR201;
(b) pDONR207;
(c) pDONR212;
(d) pDONR212(F); and
(e) pDONR212(R).

32. A composition comprising the third population of nucleic acid molecules prepared by the method of claim 1.

33. The third population of nucleic acid molecules prepared by the method of claim 1.

34. An individual member of the third population of nucleic acid molecules of claim 33.

35. A population of host cells which comprise the third population of nucleic acid molecules of claim 1.

36. An individual host cell of the population of host cells of claim 35.

37. The host cell of claim 36, wherein said host cell is a bacterial cell.

38. The host cell of claim 37, wherein said bacterial cell is E. coli.

39. The host cell of claim 36, wherein said host cell is a eukaryotic cell.

40. The host cell of claim 39, wherein said eukaryotic cell is a yeast cell.

41. The host cell of claim 39, wherein said eukaryotic cell is a plant cell.

42. The host cell of claim 39, wherein said eukaryotic cell is an animal cell.

43. The host cell of claim 42, wherein said animal cell is a mammalian cell.

44. A kit for inserting a population of nucleic acid molecules into a second target molecule according to the method of claim 1, the kit comprising one or more components selected from the group consisting of:
(a) one or more first population of nucleic acid molecules;
(b) one or more first target nucleic acid molecule;
(c) one or more second target nucleic acid molecule;
(d) one or more recombination proteins or compositions comprising one or more recombination proteins;
(e) one or more enzymes having ligase activity;
(f) one or more enzymes having polymerase activity;
(g) one or more enzymes having reverse transcriptase activity;
(h) one or more enzymes having restriction endonuclease activity;
(i) one or more primers;
(j) one or more buffers;
(k) one or more transfection reagents;
(l) one or more host cells;
(m) one or more enzymes having UDG glycosylase activity;
(n) one or more enzymes having topoisomerase activity;
(o) one or more proteins which facilitate homologous recombination; and
(p) instructions for using the kit components.

45. The kit of claim 44, wherein the one or more recombination proteins or composition comprising one or more recombination proteins is capable of catalyzing recombination between att sites.

46. The kit of claim 45, wherein the composition comprising one or more recombination proteins capable of catalyzing a BP reaction, an LR reaction, or both BP and LR reactions.

47. The kit of claim 44, wherein the first population of nucleic acid molecules comprises a library which encodes either variable heavy or variable light domains of antibody molecules.
